(19) United States
(12) Patent Application Publication
(10) Pub. No.: US 2009/0036315 A1
(43) Pub. Date: Feb. 5, 2009

(54) Device and methods for detecting and quantifying one or more target agents

(75) Inventors: Marc R. Labgold, Reston, VA (US); George G. Jokhadze, Mountain View, CA (US); I-Min M. Jen, Sunnyvale, CA (US); Naiping Shen, Saratoga, CA (US); Mark T. Kozlowski, Mountain View, CA (US); Chandramohan V. Ammini, Mountain View, CA (US); David A. Suhy, San Ramon, CA (US); Michael C. Norris, Santa Clara, CA (US); Peter E. Lobban, Los Altos Hills, CA (US)

Correspondence Address: PATTON BOGGS LLP, 8484 WESTPARK DRIVE, SUITE 900, MCLEAN, VA, 22102, US

(73) Assignee: Antara BioSciences Inc., Reston, VA (US)

(21) Appl. No.: 11/703,103

(22) Filed: Feb. 7, 2007

Related U.S. Application Data
(60) Provisional application No. 60/765,740, filed on Feb. 7, 2006.
(60) Provisional application No. 60/801,703, filed on May 19, 2006.
(60) Provisional application No. 60/801,950, filed on May 19, 2006.
(60) Provisional application No. 60/802,002, filed on May 19, 2006.
(60) Provisional application No. 60/802,039, filed on May 19, 2006.
(60) Provisional application No. 60/802,049, filed on May 19, 2006.
(60) Provisional application No. 60/808,862, filed on May 26, 2006.
(60) Provisional application No. 60/812,826, filed on Jun. 12, 2006.
(60) Provisional application No. 60/812,859, filed on Jun. 12, 2006.
(60) Provisional application No. 60/814,566, filed on Jun. 16, 2006.
(60) Provisional application No. 60/815,105, filed on Jun. 20, 2006.
(60) Provisional application No. 60/830,131, filed on Jul. 11, 2006.
(60) Provisional application No. 60/846,318, filed on Sep. 21, 2006.
(60) Provisional application No. 60/848,657, filed on Oct. 2, 2006.
(60) Provisional application No. 60/850,016, filed on Oct. 6, 2006.
(60) Provisional application No. 60/858,831, filed on Nov. 14, 2006.

(51) Int. Cl. C40B 10/00; G01N 33/566; C07H 21/04; G01N 27/26; C40B 30/04; C12Q 1/68

(52) U.S. Cl. 506/1; 436/501; 435/6; 536/24.3; 506/9; 205/787; 205/777.5

(57) ABSTRACT

The present invention provides a device and methods for the detection and quantification of one or more target agents in a sample by rapid and specific electrochemical detection. The present invention includes kits, devices and compositions capable of performing rapid, specific and accurate detection of one or more target agents in a sample.

### I. CROSS REFERENCE TO RELATED APPLICATIONS

This application claims the benefit of U.S. Provisional Patent Application Ser. No. 60/765,740, filed Feb. 7, 2006, currently pending; U.S. Provisional Patent Application Ser. No. 60/801,703, filed May 19, 2006, currently pending; U.S. Provisional Patent Application Ser. No. 60/801,950, filed May 19, 2006, currently pending; U.S. Provisional Patent Application Ser. No. 60/802,002, filed May 19, 2006, currently pending; U.S. Provisional Patent Application Ser. No. 60/802,039, filed May 19, 2006, currently pending; U.S. Provisional Patent Application Ser. No. 60/802,049, filed May 19, 2006, currently pending; U.S. Provisional Patent Application Ser. No. 60/808,862, filed May 26, 2006, currently pending; U.S. Provisional Patent Application Ser. No. 60/812,826, filed Jun. 12, 2006, currently pending; U.S. Provisional Patent Application Ser. No. 60/814,566, filed Jun. 16, 2006, currently pending; U.S. Provisional Patent Application Ser. No. 60/815,105, filed Jun. 20, 2006, currently pending; U.S. Provisional Patent Application Ser. No. 60/830,131, filed Jul. 11, 2006, currently pending; U.S. Provisional Patent Application Ser. No. 60/846,318, filed Sep. 21, 2006, currently pending; U.S. Provisional Patent Application Ser. No. 60/848,657, filed Oct. 2, 2006, currently pending; U.S. Provisional Patent Application Ser. No. 60/850,016, filed Oct. 6, 2006, currently pending; and U.S. Provisional Patent Application Ser. No. 60/858,831, filed Nov. 14, 2006, currently pending, all of which are herein incorporated by reference in their entireties for all purposes.

### II. FIELD

This invention relates to methods and compositions capable of detection of one or more target agents in a sample as well as kits for performing such detection, components thereof, information generated therefrom, and signals carrying the information. The invention further relates to business methods comprising use of the foregoing methods, compositions, kits, components, information, and/or signals.

### III. BACKGROUND

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Enzyme-linked immunosorbent assay (ELISA) is a widely used method for measuring the concentration of a particular molecule (e.g., a hormone or drug) in a fluid such as serum or urine. (See, e.g., Engvall E, Perlman P., “Enzyme-linked immunosorbent assay (ELISA), Quantitative assay of immunoglobulin G,” Immunochemistry, 1971 Sep. 8(9):871-4; and Goldsby, R. A., Kindt, T. J., Osborne, B. A. & Kuby, J., “Enzyme-Linked Immunosorbent Assay,” In: Immunology, 5th ed. (2003), pp. 148-150. W. H. Freeman, New York.) It is also known as enzyme immunoassay or EIA. Typically the molecule is detected by antibodies that have been made against it; that is, colloquially for which it is the antigen. Monoclonal antibodies are often used. Due to the diversity found in the immune system and the production of monoclonal antibodies from immortalized cells of the immune system, first described by Kohler and Milstein in 1975 (Kohler G, Milstein C. “Continuous cultures of fused cells secreting antibody of predefined specificity” Nature 1975 256:495-7). Reproduced in J Immunol 2005; 174:2453-5.), antibodies can be raised against a huge number of different antigens by standard immunological techniques. Potentially, any agent can be recognized by a specific antibody that will not react with any other agent.

An ELISA typically involves coating a vessel, such as the well of a microtiter plate with an antibody specific to a particular antigen to be detected, e.g., a virus or bacteria, adding the sample suspected of containing the particular antigen or agent, allowing the antigen to bind the immobilized antibody and then adding at least one other antibody, specific to another epitope of the same agent to be detected. This use of two antibodies can be referred to as a “sandwich” ELISA. Sometimes, the second antibody or even a third antibody is used that is labeled with a chromogenic or fluorogenic reporter molecule to aid in detection. The procedure may also involve a chemical substrate tethered to one of the antibodies to produce a signal. The need for multiple antibodies, which do not non-specifically cross-react with other antigens, and the incubation steps involved mean that it is difficult to detect more than a single agent in a sample in a short time period.

Another method of detecting the presence of particular agents in a sample involves detecting the presence of nucleic acids. Several methods of detecting nucleic acids are available including PCR and hybridization techniques. PCR is well known in the art and is described in, e.g., U.S. Pat. Nos. 4,683,195 and 4,683,202 to Mullis and Mullis et al., respectively. PCR is used for the amplification of low levels of specific nucleic acid sequences. PCR can be used to directly increase the concentration of the target nucleic acid sequence to a more readily detectable level. A variant of PCR is the ligase chain reaction, or LCR, which uses polynucleotides that are ligated together during each cycle. PCR can suffer from non-specific amplification of non-targeted nucleic acid sequences. Other variants of methods for the amplification of target nucleic acids exist, but none have been as widely accepted as PCR.

Alternatively, nucleic acid sequences can be detected and/or quantified by techniques which utilize hybridization techniques with one or more nucleic acid molecules that have complementary sequences to the target sequence. Detection of hybridization events can be achieved in a variety of ways, including labeling the complementary nucleic acid molecules and observing the signal generated from such a label. Traditional methods of hybridization, including northern and Southern blotting, were developed with the use of radioactive labels which are not amenable to automation. Radioactive labeling has been largely replaced by methods that utilize fluorescent moieties in most hybridization techniques. Representative forms of other hybridization techniques include the cycling probe reaction, branched DNA, Invader™ Assay, and Hybrid Capture.

The cycling probe reaction (CPR) (Duck, P., et al., “Probe amplifier system based on chimeric cycling oligonucleotides,” Biotechniques 1990 Aug., 9(2):142-8) uses a long chimeric oligonucleotide in which a central portion is made of RNA while the two termini are made of DNA. CPR is generally described in, e.g., U.S. Pat. Nos. 5,011,769, 5,403,711, 5,660,988, and 4,876,187, and PCT published applications WO 95/05480, WO 95/1416, and WO 95/00667, which are hereby incorporated by reference. Branched DNA (bDNA), described by Urdea et al. (“A novel method for the rapid detection of specific nucleotide sequences in crude biological samples without blotting or radioactivity; application to the analysis of hepatitis B virus in human serum,” Gene 1987 61:253-264) involves oligonucleotides with branched structures that allow each individual oligonucleotide to carry 35 to 40 labels (e.g., alkaline phosphatase enzymes). While this enhances the signal from a hybridization event, signal from non-specific binding is similarly increased. The Invader™ Assay is based on structure-specific polymerases that cleave nucleic acids in a site-specific manner. Two probes are used: an “invader” probe and a “signaling” probe that adjacently hybridize to a target sequence with a non-complementary overlap. The enzyme cleaves at the overlap due to its recognition of the “flap”, and releases the “flap” with a label. This can then be detected. The Invader™ Assay technology is described, e.g., in U.S. Pat. Nos. 5,846,717; 5,614,402; 5,719,028; 5,541,311; 5,843,669; 5,985,557; 6,001,567; 6,090,543; and 6,348,314, which are hereby incorporated by reference. However, the Invader Assay suffers from serious deficiencies including a lack of sensitivity making it unsuitable for various diagnostic applications including infectious disease applications.

The Hybrid Capture Assay involves hybridizing a sample containing unknown nucleic acid sequences with nucleic acid probes that are specific for a target nucleic acid sequence, such as oncogenic and non-oncogenic HPV DNA sequences. The hybridization complexes are then bound to anti-hybrid antibodies immobilized on a solid phase. Non-hybridized probe is removed by incubating the captured hybrids with an enzyme, such as RNase, that degrades non-hybridized probe. Hybridization is detected by either labeling the probe or using a labeled antibody, specific for the hybridization complex, in a similar manner to a “sandwich” ELISA. The Hybrid Capture Assay is described in U.S. Pat. No. 6,228,578, which technology is hereby incorporated by reference.

Many of these hybridization techniques, while overcoming the problem of non-specific nucleic acid amplification associated with PCR, lack the sensitivity required for many applications, including infectious disease diagnostics. In particular, hybridization detection techniques such as the cycling probe reaction and the Invader™ Assay that produce a linear amplification of the signaling molecule, rather than the exponential target amplification of PCR, lack the ability to be used for the detection of some infectious disease agents that are typically present in low concentrations. Additionally, linear amplification techniques may require comparatively substantially longer periods of time to accumulate a detectable signal.

PCR and hybridization techniques rely on the specificity of nucleic acid sequence complementarity to distinguish between target and non-target nucleic acid. Two single-stranded nucleic acids will only hybridize to each other if they are sufficiently complementary to each other under the specific reaction conditions. It is possible to manipulate the reaction conditions to ensure that only nucleic acid molecules with complete complementarity will hybridize to each other. This manipulation makes it possible to conduct tests simultaneously for many different sequences of nucleic acid that may be present in a sample without any substantial cross reactivity (also known as multiplex analysis); however, the possibility of a particular nucleic acid molecule hybridizing to a non-target nucleic acid that may be present cannot be precluded. Additionally, ascertaining the presence of organisms by detecting specific nucleic acid sequences can involve the extraction and isolation of nucleic acids, which can lead to cross-contamination between samples. Accordingly, even under the most stringent conditions there may be non-specific hybridization and cross-contamination that can give a false positive result when several nucleic acids of unknown sequence are present in a sample. Such false indications frequently arise due to factors including faulty isolation techniques.

Hybridization techniques can also be used to identify a specific sequence of nucleic acid present in a sample by using arrays of known nucleic acid sequences to probe a sample. Such techniques are described, e.g., in U.S. Pat. No. 6,054,270. These techniques generally involve attaching short lengths of single-stranded nucleic acid to a surface, each unique short chain attached in a specific known location and then adding the sample nucleic acid and allowing sequences present in the sample to hybridize to the immobilized strands. Detection of this hybridization is then carried out by the labeling, typically end labeling, of the fragments of the nucleic acid sample to be detected prior to the hybridization. When a sample fragment hybridizes to a complementary specific strand on the array, a signal can be detected from the label, because the position of the hybridization reaction can be detected, and the sequence of the attached strand at that location is known, the sequence of the complementary strand from the sample that has hybridized can be deduced.

The aforementioned hybridization techniques can be coupled with PCR to include amplification of the nucleic acid to be detected. Usually the detection of hybridization is by measuring a fluorescent signal; however, methods of detection using an electrochemical detection method have been disclosed. Electrochemical detection methods, and devices used in electrochemical detection methods, are discussed in, e.g., U.S. Pat. Nos. 5,776,672, 5,972,692, 6,489,160, 6,667,155, 6,670,131, 6,783,935, and 6,818,109. These electrochemical detection techniques can result in a reduced time period compared to fluorescent methods of hybridization detection and hold the potential for greater sensitivity. As discussed above however, whether based on fluorescence or electrochemistry, these hybridization detection methods can be subject to false positive signals due to non-specific hybridization. Additionally, nucleic acid detection techniques requiring steps of nucleic acid extraction, isolation and purification may lengthen the time taken to achieve a result and also decrease the detection level of the test through the loss of nucleic acid molecules in the many washing steps involved in these isolation steps.

The nucleic acid detection techniques, while overcoming the potential problem of multiplexing associated with ELISA (e.g., the limited number of discriminatory signals), are restricted in use to only detecting nucleic acid molecules. Therefore, agents such as proteins, chemical species, drugs, hormones, toxins, and prions, which do not contain nucleic acids, cannot be detected by nucleic acid hybridization techniques.

### IV. BRIEF SUMMARY

Methods for detecting one or more target agents in a sample are taught. In preferred embodiments, target agents in the sample are “captured” by a capture moiety conjugated to an oligonucleotide, wherein the oligonucleotide serves as a proxy for presence of the target agent in the sample, for example, by detectably hybridizing to a complementary oligonucleotide. The oligonucleotides employed in the methods herein can be of many lengths and sequences, but preferably have lengths and sequences that inhibit non-specific hybridization. Such methods typically allow for rapid and accurate detection without the need for nucleic acid purification and/or amplification. In certain preferred embodiments, the target agents are detected using electrochemical, fluorescent, magnetic, or other detection methods known in the art. In certain other embodiments, target nucleic acid sequences can be directly detected electrochemically utilizing structural changes and binding changes that arise when the target and its complement bind. Further, embodiments are not limited to the description listed within the Brief Summary and may include other embodiments and limitations from other parts of the specification.

Certain methods of the present invention solve the problem of multiplex detection for a wide range of target agents by combining the versatility of antibody recognition with the multiplexing capability, speed, and sensitivity of controlled electrochemical detection of nucleic acid hybridization, yet generally minimizing or eliminating the need for nucleic acid isolation/amplification procedures and the problems associated with non-specific nucleic acid hybridization in many embodiments. The non-specific hybridization observed in other detection methods currently known in the art is overcome in these methods by nucleic acid sequences that are rationally designed to minimize the risk of non-specific hybridization, ensuring that sequence-specific hybridization is optimized.

In one aspect of the invention, a method for selecting a set of universal oligos is provided. In another aspect of the invention, a universal oligo chip is provided. One embodiment of the present invention provides a method for selecting universal oligos comprising: generating a candidate oligo of length X; screening the candidate oligo against one or more reference sequences to determine sequence similarity; discarding the candidate oligo if the sequence similarity is equal to or above a first threshold; extending the length X of the candidate oligo if the sequence similarity is below the first threshold; screening the extended candidate oligo against one or more reference sequences to determine sequence similarity; discarding the extended candidate oligo if the sequence similarity is equal to or above a second threshold; extending the length of the extended candidate oligo if the sequence similarity is below the second threshold; repeating the screening, discarding and extending steps until candidate oligo has a length Y; building a first group of candidate oligos of length Y; generating complementary oligos to the candidate oligos; adding the complementary oligos to the first group; screening each candidate and complementary oligo sequence for sequence similarity against all other candidate and complementary sequences in the first group; discarding the candidate and complementary oligos if the sequence similarity is equal to or above a third threshold; and adding the candidate and complementary oligos to a second group if the sequence similarity is below the third threshold, wherein each candidate and complementary oligos in the second group are universal oligos. A universal oligo chip is provided when universal oligos are immobilized at known locations on a substrate.

Yet another aspect of the invention provides methods for using a universal oligo chip in electrochemical detection of target agents. In one embodiment of this aspect, a universal oligo chip is used in a method of electrochemically detecting the presence of a target agent in a sample. This embodiment includes, in varied orders or combinations, the use of (1) an electrode-associated universal oligo, (2) a capture-associated universal oligo that is complementary to the electrode-associated universal oligo, where the capture-associated universal oligo is conjugated to a capture moiety specific for the target agent to be detected, (3) immobilized binding partners specific for the capture moiety, and (4) a sample suspected of containing the target agent. The method includes mixing the sample suspected of containing the target agent with the capture-associated universal oligo conjugated to the capture moiety to allow the capture moiety to bind the target agent to form a mixture. The mixture is then contacted with immobilized binding partners specific for a capture moiety that has not bound a target agent (i.e., an “unreacted capture moiety”). The unreacted capture moiety can react with (e.g., bind to or otherwise associate with) the immobilized binding partners, thereby immobilizing capture-associated universal oligos that are conjugated to unreacted capture moieties (“unreacted capture-associated universal oligos”) from solution. The resultant solution is then contacted with the electrode-associated universal oligo, where a hybridization event between the electrode-associated universal oligo and the capture-associated universal oligo indicates that a target agent was present in the sample. The hybridization event is detected by electrochemical detection. The electrochemical detection can be direct or indirect. In some embodiments of the invention, the electrochemical detection comprises employing an intercalator and an electrochemical enhancing conjugate(s) in a formula such as I—(X)ₘ—(Y)ₙ, where I is an intercalator, X is a linking moiety, and Y is an electrochemical enhancing entity (such as an electron acceptor).

In certain embodiments, it may be beneficial to use isothermal amplification to increase the number of oligos available for binding to the electrode-associated oligos, thus enhancing the signal created through complementary binding. In some embodiments, the capture-associated oligo is used as a template for linear amplification, and the capture-associated oligo is therefore designed to encode a) a sequence identical to a sequence of the corresponding electrode-associated oligo (as opposed to a sequence complementary to a sequence of the electrode-associated oligo, as would be the case if the capture-associated oligo were to be hybridized directly to the electrode-associated oligo), and b) a sequence corresponding to a polymerase recognition sequence at its 3′ end adjacent to or overlapping with the region identical to a sequence of the electrode-associated oligo. Following binding of the target agent to the capture moiety and isolation of the resulting “reacted capture-associated oligo complex” from the sample (using, for example, immobilized binding partners as discussed herein), an oligonucleotide encoding the complement to the polymerase recognition sequence encoded by the capture-associated oligo is introduced to the reacted capture-associated oligo complex, and its binding to the complex creates a double-stranded polymerase recognition site. (Alternatively, as noted above, the capture-associated oligo could be engineered to contain a double-stranded portion comprising the polymerase recognition site, thereby eliminating the step of hybridization of an oligonucleotide to create such a double-stranded site.) The reacted capture-associated oligo comprising a double-stranded polymerase recognition site (whether by design or hybridization) is exposed to an aqueous solution comprising a polymerase and an excess of NTP or dNTP under conditions that allow the polymerase and reactants to create an intermediate duplex comprising a double-stranded DNA (or RNA-DNA hybrid, depending on, e.g., the polymerase and nucleotides used) having a first end that bears a polymerase recognition site (e.g., a phage-encoded RNA recognition site). As this reaction continues, the polymerase displaces the nascent strand of the double-stranded nucleic acid, resulting in multiple oligos that are complementary to the capture-associated oligo and the electrode-associated oligo on the oligo chip. As noted above, in such an embodiment, the electrode-associated oligo will have the same sequence as the capture-associated oligo, and both will be complementary to the linear amplification products. In a preferred embodiment, the polymerase recognition site created by this double-stranded region is a phage-encoded RNA polymerase recognition sequence.

In a preferred embodiment, the present invention allows for the quantification of one or more target agents. In an embodiment in which a single target agent is to be quantified, the method of electrochemically detecting and quantifying the presence of the target agent in a sample is accomplished by providing (1) an electrochemical detection device comprising a plurality of electrodes, where each electrode has an immobilized electrode-associated oligo, where each electrode-associated oligo has a known, predetermined sequence, (2) a set of capture-associated oligos, where each of the capture-associated oligos is complementary in sequence to one of the electrode-associated oligos, and where each of the capture-associated oligos is conjugated to a capture moiety specific for the target agent to be detected (or, alternatively, conjugated to a moiety capable of being selectively captured, i.e., a “capturable moiety”), (3) a set of quantifying oligos, where the quantifying oligos are complementary in sequence to electrode-associated oligos (except the electrode-associated oligos that are complementary to the capture-associated oligos), and where each quantifying oligo is present in a known (e.g., titrated, calibrated, verified, validated, etc.) concentration, (4) a sample suspected of containing one or more target agents, and (5) immobilized binding partners to the capture moiety (or capturable moiety).

The method comprises mixing/contacting the sample with the capture-associated oligos under reaction conditions that allow binding of the capture moiety or capturable moiety to the target agent present in the sample to create a first mixture. The first mixture is mixed/contacted with the immobilized binding partners, thereby immobilizing any unreacted capture-associated oligos (i.e., conjugated to a capture moiety that has not bound a target agent). This results in the formation of an immobilized phase and a solution phase. The immobilized phase comprises the immobilized binding partners and unreacted capture-associated oligos, and the solution phase comprises reacted capture-associated oligos (i.e., capture-associated oligos conjugated to a capture moiety that has bound a target agent). The method further includes contacting/mixing the solution phase with the quantifying oligos thereby resulting in a second mixture containing the reacted capture-associated oligo complex as well as the quantifying oligos, each of which has a known concentration. The second mixture is contacted with the electrochemical detection device under reaction conditions such that the capture-associated oligos hybridize to the electrode-associated oligos on the electrodes where an electrochemical signal is generated by the hybridization event.

Hybridization of the quantifying oligos, each being of known concentration (and in one embodiment, each is of a different known concentration and in a preferred embodiment, each is present in a known graduated concentration with respect to each other), will generate a signal, the magnitude of which corresponds to its respective known concentration. If the target agent is present in the sample tested, the capture-associated oligos from the reacted capture-associated oligo complexes will hybridize with an electrode-associated oligo, thereby resulting in a signal. The magnitude of that electrochemical signal can be used to calculate the concentration of the target agent in the sample by correlation with the magnitude of the electrochemical signal measured for the hybridization of each of the quantifying oligos. This method can easily be adapted to detect multiple target agents in a sample (“multiplexed”), e.g., by using two or more capture-associated oligos, each of which is conjugated to a different capture moiety specific for a different target agent. The electrochemical detection device would comprise a complementary electrode-associated oligo for each capture-associated oligo for detection and quantification of each target agent in the sample, as described above.

In certain embodiments, the electrode-associated oligos are labeled with the detection moiety, and the detection of a target agent is facilitated through the binding of the capture-associated oligo to its corresponding electrode-associated oligo and the creation of a circular structure created by the molecular interactions of the capture-associated oligo with the electrode-associated oligo. Many configurations for creating such structures are well known in the art. For example, the circular structure may be designed such that about five bases at a relative 5′-end and relative 3′-end of the electrode-associated oligo are fully complementary to their corresponding nucleic acids in the relative ends of the capture-associated oligo. The base sequence in the loop region of the electrode-associated oligo may be selected so as to be complementary to the specific base sequence complementary to the capture-associated oligonucleotide. In addition, the use of complementary G-C rich sequences may be desirable to enhance the stability of the bound regions in the circular structures.

In one specific embodiment, the capture moiety comprises a capture-associated oligo that acts as a template for linear amplification, and the electrode-associated oligo comprises a detection moiety at the end opposite the end associated with the electrode. The amplification product from the capture-associated oligo is complementary to both the capture-associated oligo and the electrode-associated oligonucleotide. Binding of the amplification product of the capture-associated oligo to the electrode-associated oligo will bring the detection moiety in closer proximity to the electrode, making a redox reaction with the electrode possible. This can be accomplished, for example, by the creation of a circular double-stranded loop structure. The closer proximity of the detection moiety enables detection of a specific target agent in a sample.

Accordingly, another assay embodiment of the invention comprises in varied orders or combinations: (1) exposing a plurality of capture moieties to a sample, the capture moieties each comprising a target agent binding domain and two or more capture-associated oligos associated with the same detection moiety, where the detection moiety is associated via a linker, e.g., a peptidic spacer (the use of a linker may allow for greater distance between the oligos, which may aid in binding for certain conformations of electrode-associated oligos); (2) allowing any target agents in the sample to bind to the capture moieties; (3) isolating the capture moiety:target agent complexes; (4) isolating the capture-associated oligo:detection moiety complexes from the target agent binding domains of the capture moiety:target agent complexes; and (5) introducing the isolated capture-associated oligo:detection moiety complexes to an electrode having electrode-associated oligos complementary to the capture-associated oligos and in the appropriate conformation to allow binding of the capture-associated oligo:detection moiety complexes, wherein binding of the capture-associated oligo:detection moiety complexes to multiple electrode-associated oligos will bring the detection moiety into proximity with the electrode.

In certain embodiments, the electrode-associated oligos are labeled with the detection moiety, with the detection moiety attached at the end of a hairpin loop created through the specific sequence of the electrode-associated oligo. Detection of a target agent is facilitated by binding of the universal oligo pair, which both disrupts the hairpin loop of the electrode-associated oligo and creates a circular structure to bring the detection moiety in close proximity to the electrode. Many configurations for creating such structures, both the hairpin loop and the circular structure, are well known in the art. The electrode-associated oligo is designed such that about five bases at a relative 5′-end and specific bases within the relative 3′-end of the electrode-associated oligo are fully complementary to one another, and that a portion of this region is also complementary to a portion of the corresponding capture-associated oligo. The base sequence in the loop region of the universal electrode-associated oligo may be selected so as to be complementary to the specific base sequence of the corresponding universal capture-associated oligo. In addition, the use of complementary G-C rich sequences may be desirable to enhance the stability of the bound regions in the circular structures.

Accordingly, another assay embodiment of the invention comprises in varied orders or combinations: (1) exposing a capture moiety to a sample, the capture moiety comprising (i) a target agent binding domain, and (ii) a capture-associated oligo; (2) allowing any target agent in the sample to bind to the capture moiety; (3) isolating the capture moiety:target agent complex; (4) introducing the isolated capture-associated oligo to an electrode having an electrode-associated oligo, each electrode-associated nucleic comprising a detection moiety conjugated to a hairpin loop structure at the unattached end of the electrode-associated oligo, where the electrode-associated oligo is complementary to a specific capture-associated oligo. Binding of the capture-associated oligo to its corresponding electrode-associated oligo will disrupt the hairpin loop structure and position the detection moiety in proximity to the electrode, rendering a redox reaction possible.

Another assay embodiment of the invention comprises in varied orders or combinations: (1) exposing a plurality of capture moieties to a sample, each capture moiety comprising a target agent binding domain and a capture-associated oligo having a polymerase recognition sequence; (2) allowing any target agent in the sample to bind to the capture moieties; (3) isolating the capture moiety:target agent complexes; (4) binding an oligonucleotide complementary to the polymerase recognition sequence on each capture-associated oligo to the capture moiety:target agent complexes; (5) reacting the capture-associated oligo with nucleotides and polymerase under conditions to allow linear amplification; and (6) introducing the isolated capture-associated oligo to an electrode having an electrode-associated oligo, each electrode-associated nucleic comprising a detection moiety conjugated to a hairpin loop structure at the unattached end of the electrode-associated oligo, where the electrode-associated oligo is complementary to a specific capture-associated oligo. Binding of the capture-associated oligo to its corresponding electrode-associated oligo will disrupt the hairpin loop structure and position the detection moiety in proximity to the electrode, rendering a redox reaction possible. When the target agent is a nucleic acid duplex in certain embodiment, a single stranded nucleic acid molecule can be conjugated to one strand of the target agent sequence and a different sequence single stranded nucleic acid molecule can be conjugated to the other strand of the target sequence. Because both strands of the target nucleic acid duplex should be present in equal amounts in a sample embodiments' testing for the presence of each strand sequentially or in different aliquots of the same sample can be used as an internal control of the accuracy of the testing.

### A. BRIEF DESCRIPTION OF THE FIGURES

So that the manner in which the recited features, advantages and objects of the present invention are attained and can be understood in detail, a more particular description of the invention, briefly summarized above, may be had by reference to the embodiments that are illustrated in the appended drawings. It is to be noted, however, that the appended drawings illustrate only certain embodiments of this invention and are therefore not to be considered limiting of its scope, for the present invention may admit to other equally effective embodiments.

FIG. 1 provides a flow diagram showing a method for selecting universal oligos and universal oligo sets.

FIG. 2 provides a flow diagram showing an alternative method for selecting universal oligos and universal oligo sets.

FIG. 3 provides an overview of three embodiments of methods to make loaded scaffolds useful in the present invention.

FIG. 4 is a schematic diagram demonstrating the detection of a target agent using an immobilized binding agent for isolation of a reacted capture-associated oligo complex.

FIG. 5 is a schematic diagram demonstrating the detection of a target agent using an immobilized binding partner for isolation of a reacted capture-associated oligo complex.

FIG. 6 provides an overview of one embodiment of a method of detection that may be performed with a universal oligo chip.

FIG. 7 provides an overview of another embodiment of a method of detection that may be performed with a universal oligo chip.

FIG. 8 provides another embodiment of a method of detecting target agents that may be performed using loaded scaffolds and an oligo chip.

FIG. 9 provides a multiplexed aspect of the method shown in FIG. 3 where two target agents are detected using loaded scaffolds and an oligo chip.

FIG. 10 provides an overview of one embodiment of a method of target agent detection that may be performed using loaded scaffolds and an oligo chip.

FIG. 11 is a simple flow diagram showing the method of one embodiment of the present invention.

FIG. 12 provides another embodiment of a method of detecting target agents that may be performed using loaded scaffolds and an oligo chip.

FIG. 13 is a schematic diagram demonstrating the use of an engineered polymerase recognition site to create multiple copies of a nucleic acid for more sensitive detection of a target agent.

FIG. 14 is a schematic diagram illustrating the combination of isolation using an immobilized binding partner that binds to the target agent and polymerase amplification techniques.

FIG. 15 provides an overview of amplification of capture-associated universal oligos using T7 RNA polymerase.

FIG. 16 is a schematic diagram demonstrating the use of a capture-associated oligo comprising a restriction endonuclease recognition sequence and a polymerase recognition sequence.

FIG. 17 is a schematic diagram demonstrating the use of a capture-associated oligo comprising a restriction endonuclease recognition sequence and a polymerase recognition sequence.

FIG. 18 is a schematic diagram illustrating the combination of isolation using a an immobilized binding partner that binds to a capture moiety/target agent complex, restriction endonuclease cleavage of the reacted capture-associated oligo complex, and polymerase amplification techniques.

FIG. 19 is a schematic diagram illustrating the combination of isolation using an immobilized binding partner that binds to a capture moiety/target agent complex, restriction endonuclease cleavage of the reacted capture-associated oligo, and polymerase amplification techniques.

FIG. 20 is a schematic diagram illustrating the use of an intermediary oligo.

FIG. 21 is a schematic diagram illustrating an assay embodiment capable of detecting a target agent on a universal oligo array, said embodiment comprising a capture moiety, a detection moiety, and an electrode, where the target binding domain of the capture moiety is removed prior to binding of the oligos.

FIG. 22 is a schematic diagram illustrating an assay embodiment capable of detecting a target agent, said embodiment comprising a capture moiety, linear amplification, a detection moiety, and an electrode.

FIG. 23 is a schematic diagram illustrating an assay embodiment capable of detecting a target agent, said embodiment comprising a capture moiety, linear amplification, a detection moiety, and an electrode, where the target binding domain of the capture moiety is removed prior to linear amplification.

FIG. 24 is a schematic diagram illustrating an assay embodiment capable of detecting a target agent, said embodiment comprising a capture moiety, linear amplification, a detection moiety, two detection oligonucleotides and an electrode.

FIG. 25 is a schematic diagram illustrating an assay embodiment capable of detecting a target agent on a universal oligonucleotide array.

FIG. 26 provides an embodiment of a method of detecting target nucleic acids wherein the target nucleic acids are not contacted with a detection device.

While certain of these figures exemplify an antibody/antigen motif, the principles of the invention are not so limited.

### B. DEFINITIONS

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated. To the extent that the definitions presented in this specification differ from any definitions set forth implicitly or explicitly in any reference or priority document cited herein, it is to be understood that those presented herein are to be used in understanding the embodiments of the invention as set forth herein.

The term “oligonucleotides,” or “oligos” as used herein refers to oligomers of natural or modified nucleic acid monomers or linkages, including deoxyribonucleotides, ribonucleotides, anomeric forms thereof, peptide nucleic acid monomers (PNAs), locked nucleotide acids monomers (LNA), and the like and/or combinations thereof, capable of specifically binding to a single-stranded polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g., 8-12, to several tens of monomeric units, e.g., 100-200. Suitable oligonucleotides may be prepared by the phosphoramidite method described by Beaucage and Carruthers (Tetrahedron Lett., 22, 1859-1862, 1981), or by the triester method according to Matteucci, et al. (J. Am. Chem. Soc., 103, 3185, 1981), both incorporated herein by reference, or by other chemical methods such as using a commercial automated oligonucleotide synthesizer. Typically, oligonucleotides are single-stranded, but double-stranded or partially double-stranded oligos may also be used in certain embodiments of the invention. An “oligo pair” is a pair of oligos that specifically bind to one another (i.e., are complementary (e.g., perfectly complementary) to one another). An “oligo chip” is an array of two or more oligos—each from a different oligo pair—that are immobilized at a known location on a surface such as glass, plastic, nylon, silicon, etc. The term “capture-associated oligo” refers to an oligo that is associated with a capture moiety (whether, e.g., conjugated to the capture moiety directly or via a loaded scaffold, for example). Conjugation to the capture moiety (or scaffold) may be at the 3′ or 5′ end of the capture-associated oligo. The term “electrode-associated oligo” refers to an oligo that is associated with an electrode. Association to the electrode may occur at the 3′ or 5′ end, but typically occurs at the 5′ end. The term “chip-associated oligo” refers to an oligo that is associated with a chip coupled to a detection device, and includes electrode-associated oligos. In most embodiments of the present invention, an oligo pair comprises a capture-associated oligo and an electrode- or chip-associated oligo that are complementary or perfectly complementary to each other. In other embodiments, such as those comprising an intermediary oligo as described below, the capture-associated oligo and the electrode- or chip-associated oligo may be partially or completely noncomplementary.

The terms “complementary” and “complementarity” refer to oligonucleotides related by base-pairing rules. Complementary nucleotides are, generally, A and T (or A and U), or C and G. For example, for the sequence “5′-AGT-3′,” the perfectly complementary sequence is “3′-TCA-5′.” Methods for calculating the level of complementarity between two nucleic acids are widely known to those of ordinary skill in the art. For example, complementarity may be computed using online resources, such as, e.g., the NCBI BLAST website (ncbi.nlm.nih.gov/blast/producttable.shtml) and the Oligonucleotides Properties Calculator on the Northwestern University website (basic.northwestem.edu/biotools/oligocalc.html). Two single-stranded RNA or DNA molecules may be considered substantially complementary when the nucleotides of one strand, optimally aligned and with appropriate nucleotide insertions or deletions, pair with at least about 80% of the nucleotides of the other strand, usually at least about 90% to 95%, and more preferably from about 98 to 100%. Two single-stranded oligonucleotides are considered perfectly complementary when the nucleotides of one strand, optimally aligned and with appropriate nucleotide insertions or deletions, pair with 100% of the nucleotides of the other strand. Alternatively, substantial complementarity exists when a first oligonucleotide will hybridize under selective hybridization conditions to a second oligonucleotide. Selective hybridization conditions include, but are not limited to, stringent hybridization conditions. Selective hybridization occurs in one embodiment when at least about 65% of the nucleic acid monomers within a first oligonucleotide over a stretch of at least 14 to 25 monomers pair with a perfectly complementary monomer within a second oligonucleotide, preferably at least about 75%, more preferably at least about 90%. See, M. Kanehisa, Nucleic Acids Res. 12, 203 (1984), incorporated herein by reference. For shorter nucleotide sequences selective hybridization occurs when at least about 65% of the nucleic acid monomers within a first oligonucleotide over a stretch of at least 8 to 12 nucleotides pair with a perfectly complementary monomer within a second oligonucleotide, preferably at least about 75%, more preferably at least about 90%. Stringent hybridization conditions will typically include salt concentrations of less than about 1 M, more usually less than about 500 mM and preferably less than about 200 mM. Hybridization temperatures can be as low as 5° C., and are preferably lower than about 30° C. However, longer fragments may require higher hybridization temperatures for specific hybridization. Hybridization temperatures are generally at least about 2° C. to 6° C. lower than melting temperatures (Tₘ), which are defined below.

The term “universal oligo” generally refers to one oligo of an oligo pair, where each oligo in the pair has been rationally designed to have low complementarity to sequences that may be present in a sample. In certain preferred embodiments, the universal oligos in a universal oligo pair are perfectly complementary to one another. For example, a universal oligo for diagnosis of hepatitis using a human blood sample would be one with low complementarity to human genomic sequences, genomic sequences from hepatitis viruses, as well as genomic sequences of organisms that associate with humans (e.g., human gut flora (Enterococcus faecalis, Enterobacteriaceae, etc.), Candida albicans, Staphylococcus epidermidis, Streptococcus salivarius, Lactobacillus sp., Spirochetes, etc.) For a soil sample, a universal oligo would be one with minimal complementarity to genomic sequences from, e.g., soil flora and fauna. A “universal oligo set” is a set of two or more universal oligo pairs where each oligo in the set has low complementarity to every other universal oligo in the set, with the exception of its complement. A “universal oligo chip” is an array of two or more universal oligos—each from a different universal oligo pair—that are immobilized at a known location on a surface such as glass, plastic, nylon, silicon, etc. The term “capture-associated universal oligo” refers to a universal oligo that is associated with a capture moiety (whether, e.g., conjugated to the capture moiety directly or via a loaded scaffold, for example). The term “electrode-associated universal oligo” refers to a universal oligo that is associated with an electrode. The term “chip-associated universal oligo” refers to a universal oligo that is associated with a chip coupled to a detection device, and includes electrode-associated universal oligos. In most embodiments of the present invention, a universal oligo pair comprises a capture-associated universal oligo and an electrode- or chip-associated universal oligo that are complementary (e.g., perfectly complementary) to each other. In other embodiments, such as those comprising an intermediary universal oligo as described below, the capture-associated universal oligo and the electrode- or chip-associated universal oligo may be partially or completely noncomplementary.

A “capture moiety” refers to a molecule or a portion of a molecule that can be used to preferentially bind and separate a molecule of interest (a “target agent”) from a sample. The term “capture moiety” as used herein refers to any molecule, natural, synthetic, or recombinantly-produced, or portion thereof, with the ability to bind to or otherwise associate with a target agent in a manner that facilitates detection of the target agent in the methods of the present invention. For example, in certain embodiments the binding affinity of the capture moiety is sufficient to allow collection, concentration, or separation of the target agent from a sample. Suitable capture moieties include, but are not limited to nucleic acids, antibodies, antigen-binding regions of antibodies, antigens, epitopes, cell receptors (e.g., cell surface receptors) and ligands thereof, such as peptide growth factors (see, e.g., Pigott and Power (1993), The Adhesion Molecule Facts Book (Academic Press New York); and Receptor Ligand Interactions: A Practical Approach, Rickwood and Hames (series editors) Hulme (ed.) (IRL Press at Oxford Press NY)). Similarly capture moieties may also include but are not limited to toxins, venoms, intracellular receptors (e.g., receptors which mediate the effects of various small ligands, including steroids, hormones, retinoids and vitamin D, peptides) and ligands thereof, drugs (e.g., opiates, steroids, etc.), lectins, sugars, oligosaccharides, other proteins, phospholipids, and structured nucleic acids such as aptamers and the like. Those of skill in the art readily will appreciate that molecular interactions other than those listed above are well described in the literature and may also serve as capture moiety/target agent interactions. In certain embodiments, capture moieties are associated with scaffolds, and in other embodiments capture moieties are conjugated to capture-associated oligos.

The term “binding partner” as used herein refers to any molecule, natural, synthetic, or recombinantly-produced, with the ability to bind to a target agent and/or capture moiety in the methods of the present invention. For example, in some embodiments a “binding partner” is a molecule or portion thereof that preferentially binds to a moiety of the target agent different from a moiety of the target agent that is bound by a capture moiety, such that both the capture moiety and the binding partner may be simultaneously bound to the target agent. In other embodiments, a “binding partner” may preferentially bind to a capture moiety/target agent complex. Alternatively, in certain embodiments immobilized binding partners will bind unreacted capture moieties (i.e., those that have not bound to target agent). The binding affinity of the binding partner must be sufficient to allow collection of the target agent and/or capture moiety from a sample and/or sample mixture. Suitable binding moieties include, but are not limited to, antibodies, antigen-binding regions of antibodies, antigens, epitopes, cell receptor ligands, such as peptide growth factors (see, e.g., Pigott and Power (1993), The Adhesion Molecule Facts Book (Academic Press New York); and Receptor Ligand Interactions: A Practical Approach, Rickwood and Hames (series editors) Hulme (ed.) (IRL Press at Oxford Press NY)). Similarly, binding partners may also include but are not limited to toxins, venoms, intracellular receptors (e.g., receptors which mediate the effects of various small ligands, including steroids, hormones, retinoids and vitamin D, peptides), drugs (e.g., opiates, steroids, etc.), lectins, sugars, oligosaccharides, other proteins, and phospholipids. Those of skill in the art readily will appreciate that a number of binding partners based upon molecular interactions other than those listed above are well described in the literature and may also serve as binding partners. The binding partners can be affixed/immobilized directly or indirectly to a matrix such as a vessel wall, to particles or beads (as described in more detail infra), or to other suitable surfaces to form “immobilized binding partners.” Those of skill in the art will readily understand the versatility of the nature of this immobilized binding partner. Essentially, any ligand and receptor can be utilized to serve as capture moieties, target agents and binding partners, as long as the target agent is appropriate for detection for the pathology or condition interrogated. Suitable ligands and receptors include an antibody or fragment thereof to be recognized by a corresponding antigen or epitope, a hormone to be recognized by its receptor, an inhibitor to be recognized by its enzyme, a co-factor portion to be recognized by a co-factor enzyme binding site, a binding ligand to be recognized by its substrate, and the like.

By “preferentially binds” it is meant that a specific binding event between a first and second molecule occurs at least 20 times or more, preferably 50 times or more, more preferably 100 times or more, and even 1000 times or more often than a nonspecific binding event between the first molecule and a molecule that is not the second molecule. For example, a capture moiety can be designed to preferentially bind to a given target agent at least 20 times or more, preferably 50 times or more, more preferably 100 times or more, and even 1000 times or more often than to other molecules in a biological solution. Also, an immobilized binding partner, in certain embodiments, will preferentially bind to a target agent, capture moiety, or capture moiety/target agent complex. While not wishing to be limited by applicants present understanding of the invention, it is believed binding will be recognized as existing when the Kₐ is at 10⁷ l/mole or greater, preferably 10⁸ l/mole or greater. In the embodiment where the capture moiety is comprised of antibody, the binding affinity of 10⁷ l/mole or more may be due to (1) a single monoclonal antibody (e.g., large numbers of one kind of antibody) or (2) a plurality of different monoclonal antibodies (e.g., large numbers of each of several different monoclonal antibodies) or (3) large numbers of polyclonal antibodies. It is also possible to use combinations of (1)-(3). The differential in binding affinity may be accomplished by using several different antibodies as per (1)-(3) above and as such some of the antibodies in a mixture could have less than a four-fold difference. For purposes of most embodiments of the invention an indication that no binding occurs means that the equilibrium or affinity constant Kₐ is 10⁶ l/mole or less. Antibodies may be designed to maximize binding to the intended antigen by designing peptides to specific epitopes that are more accessible to binding, as can be predicted by one skilled in the art.

A “target agent” is a molecule of interest in a sample that is to be detected by the methods of the instant invention. For example, in certain embodiments the target agent is captured through preferential binding with a capture moiety. In one such embodiment, the capture moiety is an antibody and the target agent is any molecule which contains an epitope against which the antibody is generated, or an epitope specifically bound by the antibody. In another embodiment, the capture moiety is a protein specifically bound by an antibody, and the antibody itself is the target agent. Target agents also may include but are not limited to receptors (e.g., cell surface receptors) and ligands thereof, nucleic acids, intracellular receptors (e.g., receptors which mediate the effects of various small ligands, including steroids, hormones, retinoids and vitamin D, peptides) and ligands thereof, metabolites, steroids, hormones, lectins, sugars, oligosaccharides, proteins, phospholipids, toxins, venoms, drugs (e.g., opiates, steroids, etc.), and the like. Those of skill in the art readily will appreciate that molecular interactions other than those listed above are well described in the literature and may also serve as capture moiety/target agent interactions.

The term “sample” in the present specification and claims is used in its broadest sense and can be, by non-limiting example, any sample that is suspected of containing a target agent(s) to be detected. It is meant to include specimens or cultures (e.g., microbiological cultures), and biological and environmental specimens as well as non-biological specimens. Biological samples may comprise animal-derived materials, including fluid (e.g., blood, saliva, urine, lymph, etc.), solid (e.g., stool) or tissue (e.g., buccal, organ-specific, skin, etc.), as well as liquid and solid food and feed products and ingredients such as dairy items, vegetables, meat and meat by-products, and waste. Biological samples may be obtained from, e.g., humans, any domestic or wild animals, plants, bacteria or other microorganisms, etc. Environmental samples can include environmental material such as surface matter, soil, water (e.g., contaminated water), air and industrial samples, as well as samples obtained from food and dairy processing instruments, apparatus, equipment, utensils, disposable and non-disposable items. These examples are not to be construed as limiting the sample types applicable to the present invention. Those of skill in the art would appreciate and understand the particular type of sample required for the detection of particular target agents (Pawliszyn, J., Sampling and Sample Preparation for Field and Laboratory, (2002); Venkatesh Iyengar, G., et al., Element Analysis of Biological Samples: Principles and Practices (1998); Drielak, S., Hot Zone Forensics: Chemical, Biological, and Radiological Evidence Collection (2004); and Nielsen, D. M., Practical Handbook of Environmental Site Characterization and Ground-Water Monitoring (2005)).

The term “antibody” as used herein refers to an entire immunoglobulin or antibody or any fragment of an immunoglobulin molecule which is capable of specific binding to a target agent of interest (an antigen). Examples of such antibodies include complete antibody molecules, antibody fragments, such as Fab, F(ab′)₂, CDRS, V_{L}, V_{H}, and any other portion of an antibody which is capable of specifically binding to an antigen. An IgG antibody molecule is composed of two light chains linked by disulfide bonds to two heavy chains. The two heavy chains are, in turn, linked to one another by disulfide bonds in an area known as the hinge region of the antibody. A single IgG molecule typically has a molecular weight of approximately 150-160 kD and contains two antigen binding sites. An F(ab′)₂ fragment lacks the C-terminal portion of the heavy chain constant region, and has a molecular weight of approximately 110 kD. It retains the two antigen binding sites and the interchain disulfide bonds in the hinge region, but it does not have the effector functions of an intact IgG molecule. An F(ab′)₂ fragment may be obtained from an IgG molecule by proteolytic digestion with pepsin at pH 3.0-3.5 using standard methods such as those described in Harlow and Lane, supra. Preferred antibodies for assays of the invention are immunoreactive or immunospecific for, and therefore specifically and selectively bind to, a protein (antigen) of interest and are not limited to the G class of immunoglobulin used in the above cited example. A “purified antibody” refers to that which is sufficiently free of other proteins, carbohydrates, and lipids with which it is naturally associated to measure any difference.

A substance is commonly said to be present in “excess” or “molar excess” relative to another component if that component is present at a higher molar concentration than the other component. Often, when present in excess, the component will be present in at least a 10-fold molar excess and commonly at 100-1,000,000 fold molar excess. Those of skill in the art would appreciate and understand the particular degree or amount of excess preferred for any particular reaction or reaction conditions. Such excess is often empirically determined and/or optimized for a particular reaction or reaction conditions.

The term “reacted capture-associated oligo” or “reacted capture-associated universal oligo” is commonly used in reference to capture-associated oligos or capture-associated universal oligos, respectively, associated with a capture moiety for a particular target agent, where the capture moiety has bound to the target agent, e.g., due to the presence of the target agent in a sample contacted with the capture moiety. The term “unreacted capture-associated oligo” or “unreacted capture-associated universal oligo” is used in reference to capture-associated oligos or capture-associated universal oligos, respectively associated with a capture moiety for a particular target agent, where the capture moiety has not bound to the target agent, e.g., due to a deficiency of the target agent in a sample contacted with the capture moiety. The term “reacted loaded scaffolds” is used in reference to loaded scaffolds comprising a capture moiety bound to a target agent from a sample. The term “unreacted loaded scaffolds” is used in reference to loaded scaffolds comprising a capture moiety not bound to a target agent.

The term “capture reaction” is commonly used in reference to the mixing/contacting of capture-associated oligos associated with a capture moiety and a sample under conditions that allow the capture moiety to attach to, bind or otherwise associate with a target agent in the sample. For example, a “capture reaction” can involve mixing/contacting of one or more loaded scaffolds (or immobilized binding partner in the reverse bead/scaffold capture method) and a sample under conditions that allow a capture moiety of the loaded scaffold (or immobilized binding agent on the, e.g., bead in the reverse bead/scaffold capture method) to attach to, bind or otherwise associate with a target agent in the sample.

The term “melting temperature” or Tₘ is commonly defined as the temperature at which half of the population of double-stranded nucleic acid molecules becomes dissociated into single strands. The equation for calculating the Tₘ of nucleic acids is well known in the art. As indicated by standard references, a simple estimate of the Tₘ value may be calculated by the equation: Tₘ=81.5+16.6(log₁₀[Na⁺])0.41(%[G+C])−675/n−1.0 m, when a nucleic acid is in aqueous solution having cation concentrations of 0.5 M, or less, the (G+C) content is between 30% and 70%, n is the number of bases, and m is the percentage of base pair mismatches (see e.g., Sambrook J et al., “Molecular Cloning, A Laboratory Manual,” 3^{rd} Edition, Cold Spring Harbor Laboratory Press (2001)). Other references include more sophisticated computations, which take structural as well as sequence characteristics into account for the calculation of Tₘ.

The term “matrix” means any surface.

A “restriction endonuclease” is any enzyme capable of recognizing a specific sequence on a double- or single-stranded polynucleotide and cleaving the polynucleotide at or near the site. Examples of site-specific restriction endonucleases, the nucleotide sequences recognized by them, and their products of cleavage are well known to those of ordinary skill in the art and are available, e.g., in the 2006 New England Biolabs, Inc. catalog, including the 2006 New Products Catalog Supplement, which is incorporated herein by reference.

As used herein “nucleotide” refers to a base-sugar-phosphate combination. Nucleotides are monomeric units of a nucleic acid sequence (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP, and nucleotide derivatives that confer nuclease resistance on the nucleic acid molecule containing them. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels. Fluorescent labels of nucleotides may include, but are not limited to, fluorescein, 5-carboxyfluorescein (FAM), 2′,7′-dimethoxy-4′,5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP, available from Perkin Elmer, Foster City, Calif. FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham Arlington Heights, Ill.; Fluorescein-15-dATP, Fluorescein-12-dUTP, Tetramethyl-rodamine-6-dUTP, IR₇₇₀₋₉-dATP, Fluorescein-12-ddUTP, Fluorescein-12-UTP, and Fluorescein-15-2′-dATP available from Boehringer Mannheim Indianapolis, Ind.; and ChromaTide Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg.

The terms “SAM” and “self-assembled monolayer”, as used interchangeably throughout the specification, refers to crystalline chemisorbed organic single layers formed on a solid substrate by spontaneous organization of the molecules.

An “epitope” as used herein refers to any portion of a molecule that is capable of preferentially binding to a capture moiety, a binding partner, or a target agent. For example, an epitope can be a site on an antigen that is recognized by an antibody or a region of a protein that is recognized by a receptor.

A “biosensor” is defined as being a unique combination of (1) one or more moieties for molecular recognition, e.g., a chip-associated oligo that preferentially binds to a capture-associated oligo; (2) a surface on to which the moieties for molecular recognition are associated; and (3) a transducer for transmitting the interaction information to processable signals; e.g., an electrode. In certain embodiments, a biosensor for use in the methods of the invention is an electrochemical detection device, which comprises an electrode and an electrode-associated oligo.

An “anchoring group” as defined herein refers to a component of a SAM that is associated with a moiety for molecular recognition. The anchoring group serves to attach a moiety for molecular recognition (e.g., an oligo) to a signal transducer (e.g., an electrode).

A “diluent group” as defined herein refers to any component of a SAM that is not associated with a moiety for molecular recognition.

The term “scaffold” as used herein describes a solid support upon which capture-associated oligos and/or capture moieties may be bound. Such support can include, but is not limited to, such structures as gold, aluminum, copper, platinum, silica, titanium dioxide, carbon nanotubes, polystyrene particles, polyvinyl particles, acrylate and methacrylate particles, glass particles, latex particles, Sepharose beads and other like particles, polymer coated magnetic beads, semiconducting materials, and radio frequency identification substrates. The term “loaded scaffold” refers to a scaffold that comprises both capture-associated oligos and capture moieties affixed or otherwise associated with the scaffold.

The term “chip” as used herein refers to an object for detection of the hybridization between two oligos of an oligo pair, where a chip comprises a surface and one or more oligos associated with the surface.

A “detection moiety” is any one or a plurality of chemical moieties capable of enabling the molecular recognition on a biosensor (e.g., an electrochemical hybridization detector). In certain embodiments, the detection moiety can be any chemical moiety that is stable under assay conditions and can undergo reduction and/or oxidation. Examples of such detection moieties include, but are not limited to, purely organic labels, such as viologen, anthraquinone, ethidium bromide, daunomycin, methylene blue, and their derivatives, organo-metallic labels, such as ferrocene, ruthenium, bis-pyridine, tris-pyridine, bis-imidizole, and their derivatives, and biological labels, such as cytochrome c, plastocyanin, and cytochrome c′. Specific electroactive agents for use in the invention include a large number of ferrocene (Brazill, S. A., Kim, P. H. & Kuhr, W. G., Anal. Chem. 73, 4882-4890 (2001)) and viologen derivatives (Fan, C., Hirasa, T., Plaxco, K. W. and Heeger, A. J. (2003)) and any other stable agent capable of oxidation-reduction reactions. In specific embodiments, the detection moiety is comprised of a plurality of electrochemical hybridization detectors (e.g., ferrocene), optionally linked to a hydrocarbon molecule. Such molecules include but are not limited to ferrocene-hydrocarbon mixtures; such as ferrocene-methane, ferrocene-acetylene, and ferrocene-butane. In one particular embodiment, the detection moiety is Fe(CN)63-/4-. Further examples of methods using electrochemical hybridization detectors are provided herein. In yet other embodiments, the detection moiety is a fluorescent label moiety. The fluorescent label may be selected from any of a number of different moieties. The preferred moiety is a fluorescent group for which detection is quite sensitive. Various different fluorescence labels techniques are described, for example, in Cambara et al. (1988) “Optimization of Parameters in a DNA Sequenator Using Fluorescence Detection,” Bio/Technol. 6:816 821; Smith et al. (1985) Nucl. Acids Res. 13:2399 2412; and Smith et al. (1986) Nature 321:674 679, each of which is hereby incorporated herein by reference. Fluorescent labels exhibiting particularly high coefficients of destruction may also be useful in destroying nonspecific background signals. In yet other embodiments, the detection moiety is a detection antibody reagent, where the antibody is labeled with a molecular entity which allows detection of nucleic acid binding. Examples of such reagents include, but are not limited to, antibody reagents that preferentially bind to RNA:DNA complexes.

It should be understood by those skilled in the art that terms such as “target”, “agent”, “moiety”, “antigen”, “antibody”, “molecule” and the like should be interpreted in the context in which they appear, and should be given the broadest interpretation possible unless specifically indicated.

### V. DETAILED DESCRIPTION

The present invention relates to oligos, oligo chips, biosensors, scaffolds, and methods of use thereof for detecting the presence of target agents in a sample. The target agents that can be detected include, but are not limited to, nucleic acids, potentially infectious or disease-causing agents, chemical or biological toxins, pathogenic agents, drugs (e.g., opiates, steroids, etc.), drug metabolites, other metabolites, receptors (e.g., cell surface receptors) and ligands thereof, intracellular receptors (e.g., receptors which mediate the effects of various small ligands, including steroids, hormones, retinoids and vitamin D, peptides) and ligands thereof, steroids, hormones, lectins, sugars, nucleic acids, oligosaccharides, proteins, phospholipids, toxins, venoms, environmental contaminants, combinations thereof, and the like. In certain embodiments, the methods contemplate the use of an oligo conjugated to a capture moiety that specifically binds or otherwise associates with a target agent in a sample. Such a capture moiety can be or include, for example, an antibody, antigen, or other ligand specific for a particular target agent. The capture moiety can even be a nucleic acid sequence, especially if its complement is contained in the target agent. A “capture-associated oligo” (i.e., conjugated to a capture moiety) is contacted/mixed with a sample that is suspected of containing a target agent, under conditions that if a target agent is present, the capture moiety can react with, i.e., bind with/to, the target agent. The capture-associated oligo may be added in excess relative to the amount of target agent suspected to be present in the sample.

In certain preferred embodiments, after the capture reaction in which the capture moiety reacts with the target agent, the reacted capture-associated oligos (i.e., conjugated to a capture moiety associated with a target agent) are separated from the unreacted capture-associated oligos (i.e., conjugated to a capture moiety not associated with a target agent) and are contacted with a detection device comprising oligos complementary to the capture-associated oligos. Hybridization between the capture-associated oligos and the complementary oligos on the detection device is detected, indicating the presence of the target agent in the sample. Although the examples and embodiments typically recite an electrochemical detection device, the invention should by no means be limited to such a detection device. Other detection devices can also be used with the methods disclosed herein. For example, fluorescence-detection devices may be used where the capture-associated oligos have been labeled with a fluorescent tag such that hybridization to an array of complementary oligos produces a detectable signal that serves as a proxy for presence of a target agent in a sample. In other embodiments, Such methods include the use of oligonucleotide microarrays (e.g., from Affymetrix, Inc. (Santa Clara, Calif.) and Illumina (San Diego, Calif.)) and ELISA techniques, which are widely known in the art.

In other embodiments, an array comprising embedded magnetic sensors (e.g., MagArray™) may be used to detect target agent in a sample. The MagArray comprises an array of biomolecules that specifically interact (e.g., bind) with a target agent of interest. These biomolecules are attached to ferromagnetic sensors arrayed on the chip, and these sensors are specially designed so that their electrical resistance will change in the presence of a particular magnetic field. Sample is added to the chip under conditions that allow components of interest in the sample (e.g., proteins, nucleic acids, etc.) to bind to the biomolecules. Magnetically sensitive nanoparticles comprising agents that will bind to the components of interest are added to the chip, and in the presence of an applied magnetic field the nanoparticles emit their own field, which changes the resistance of the sensor thereby allowing detection of the components of interest on the array (Li, G. et al. (2006) Sensors and Actuators A: Physical 126(1):98-106). In certain embodiments of the present invention, such ferromagnetic sensor arrays comprise chip-associated oligos and the components of interest are capture-associated oligos, and the magnetically sensitive nanoparticles bind specifically to the capture-associated oligo/chip-associated oligo hybridization complex, thereby changing the resistance of the sensor and allowing detection of the capture-associated oligos on the array, and, therefore, target agent in the sample.

Therefore, although the disclosure contains various examples of the methods of the invention using electrode-associated oligos, the invention is by no means to be limited to the use of electrode-associated oligos and other types of oligos complementary to the capture-associated oligos (e.g., other types of chip-associated oligos) may optionally be used in the methods presented herein.

### Universal Oligos, Universal Oligo Sets, and Universal Oligo Chips

In certain embodiments, the oligos used are universal oligos. Universal oligos of the present invention are oligonucleotides from a complementary oligonucleotide pair (i.e., each is the complement of the other), where each oligo in the pair has been rationally designed to have low complementarity to nucleotide sequences that may be present in a given sample, e.g., as described in detail below. A “universal oligo set” is a set of two or more universal oligo pairs where each oligo in the set has low complementarity to every other universal oligo in the set, with the exception of its complement. Use of universal oligo chips for detecting target agents has many advantages. For example, the universal oligo chips can be used with virtually any upstream application (e.g., the front end assay can capture (e.g., bind to or otherwise associate with, isolate from a sample, concentrate or purify, etc.) target agents such as, e.g., antibodies, antigens, chemical or biological toxins, pathogenic agents, drugs, drug metabolites, other metabolites, environmental contaminants, etc.), yet the chips have standardized hybridization conditions independent of the target agent. However, the universal oligo chip system can be flexible as well, as it is envisioned that it may be advantageous to have universal oligo chips that comprise different universal oligo sets and act as the detector component for different assays, with the identity of the universal oligo set members as unique identifiers for specific moieties within each assay set. For example, a particular universal oligo chip may have electrode-associated oligos with melting temperatures and/or lengths of X (e.g., “reaction profile A”) and another universal oligo chip may have electrode-associated oligos with melting temperatures and/or lengths of Y (e.g., “reaction profile B”). In certain embodiments, a single universal oligo chip may contain different sets of electrode-associated oligos with distinct reaction profiles so that the hybridization conditions employed would determine which set of electrode-associated universal oligos would react with a given mixture (e.g., a solution containing capture-associated universal oligos). In addition, the universal oligos of the present invention can be engineered to contain sequences for enzyme cleavage and/or polymerase binding for use in some embodiments.

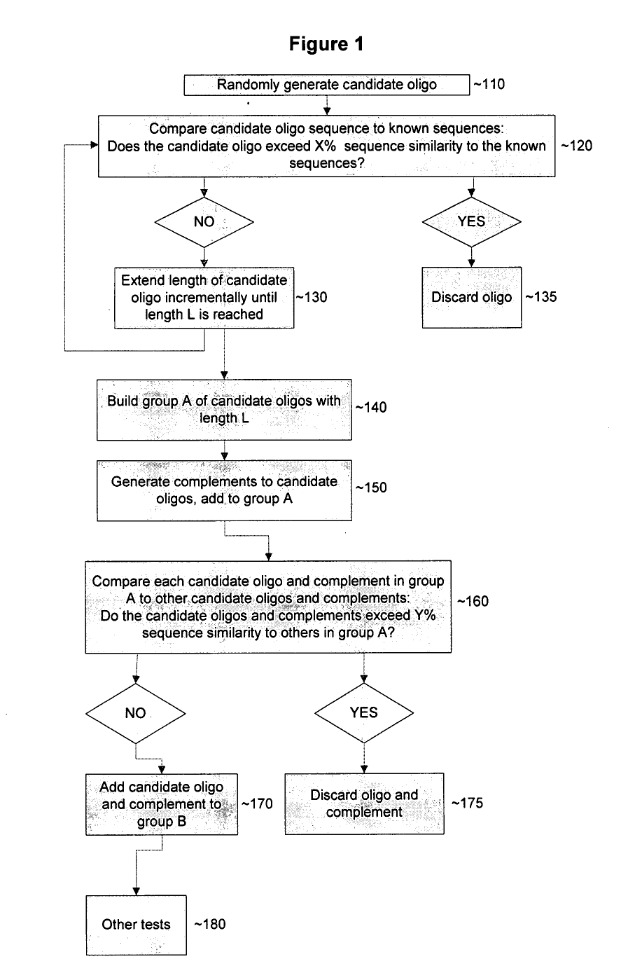

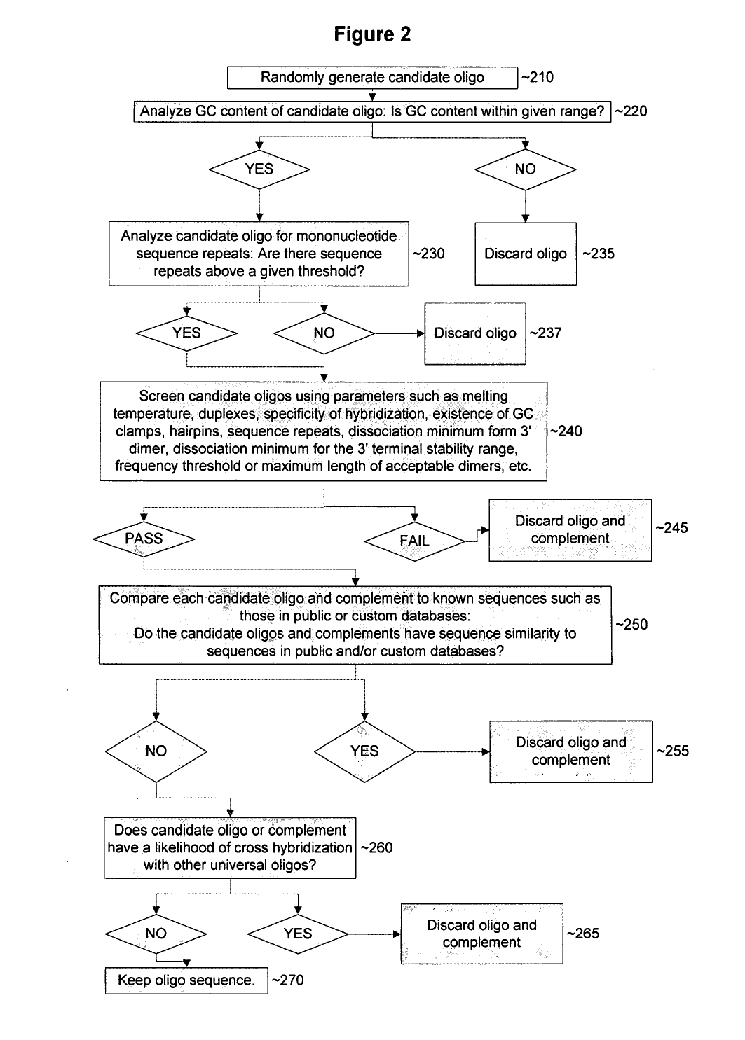

FIG. 1 is a flow chart showing the steps of creating universal oligos and a universal oligo set. In step 110, candidate oligo sequences are randomly generated. Typically, such randomly generated sequences will be short, for example, 8-25 nucleotides in length. In one embodiment of the invention, all possible variations of 15-mers (consisting only of nucleotides A, T, G and C) are generated and stored in a database. At step 120, each candidate sequence is compared to known sequences, typically, by comparing the candidate sequence to sequences stored in publicly-available and/or custom databases. Custom databases may be databases populated with information from publicly-available databases, databases licensed from a third party, databases generated by the practitioner of the methods presented herein, or a combination thereof. Major publicly-available sequence repositories include DDBJ: DNA databank of Japan, EMBL: maintained by EMBL, and GenBank: maintained by NCBI; organelle databases include OGMP: the organelle genome megasequencing program, GOBASE: an organelle genome database, and MitoMap: a human mitochondrial genome database; RNA databases include Rfam: an RNA family database, RNA base: a database of RNA structures, tRNA database: a database of tRNAs, tRNA: tRNA sequences and genes, and sRNA: a small RNA database; comparative and phylogenetic databases include COG: phylogenetic classification of proteins, DHMHD: a human-mouse homology database, HomoloGene: a database of gene homologies across species, Homophila: a human disease to Drosophila gene database, HOVERGEN: a database of homologous vertebrate genes, TreeBase: a database of phylogenetic knowledge, XREF: a database that cross-references human sequences with model organisms; SNP, mutation and variation databases include ALPSbase: a database of mutations causing human ALPS, dbSNP: the single nucleotide polymorphism database at NCBI, and HGVbase: a human genome variation database; alternative splicing databases include ASDB: a database of alternatively spliced genes, ASAP: an alternate splicing analysis tool, ASG: an alternate splicing gallery, HASDB: a human alternative splicing database, AsMamDB: a database of alternatively spliced genes in human, mouse and rat, and ASD: an alternative splicing database at CSHL; and scores of specialized databases include ACUTS: a database of ancient conserved untranslated sequences, AGSD: an animal gehome database, AmiGO: a gene ontology database, ARGH: an acronym database, BACPAC: BAC and PAC a database of genomic DNA library info, CHLC: a database of genetic markers on chromosomes, COGENT: a complete genome tracking database, COMPEL: a database of composite regulatory elements in eukaryotes, CUTG: a codon usage database, dbEST: a database of expressed sequences or mRNA, dbGSS: genome survey sequence database, dbSTS: a database of sequence tagged sites (STS), DBTSS: a database of transcriptional start sites, DOGS: a database of genome sizes, EID: the exon-intron database, Exon-Intron: an exon-intron database, EPD: a eukaryotic promotor database, FlyTrap: a HTML-based gene expression database, GDB: the genome database, GeneKnockouts: a database of gene knockout information, GENOTK: a human cDNA database, GEO: a gene expression omnibus NCBI, GOLD: a database of information on genome projects around the world, GSDB: the Genome Sequence DataBase, HGI: TIGR human gene index, HTGS: a database of genomic sequences at NCBI, IMAGE: a database of the largest collection of DNA sequences clones, IMGT: a database of the international ImMunoGeneTics information system, LocusLink: single query interface to sequence and genetic loci, TelDB: a telomere database, MitoDat: a database of mitochondrial nuclear genes, Mouse EST: a database with information from the NIA mouse cDNA project, MPSS: searchable databases of several species, NDB: a nucleic acid database, NEDO: a human cDNA sequence database, NPD: a nuclear protein database, PLACE: a database of plant cis-acting regulatory DNA elements, RDP: a ribosomal database, RDB: a receptor database at NIHS, Japan, Refseg: the NCBI reference sequence project, RHdb: a database of radiation hybrid physical map of chromosomes, SpliceDB: a database of canonical and non-canonical splice site sequences, STACK: a database of consensus human EST database, TAED: the adaptive evolution database, TIGR: curated databases of microbes, plants and humans, TRANSFAC: the transcription factor database, TRRD: a transcription regulatory region database, UniGene: a database of cluster of sequences for unique genes at NCBI, and UniSTS: a database of nonredundent STS.

For sequence comparison, known sequences act as reference sequences to which the candidate sequences are compared to determine “sequence similarity” between the reference sequences and the candidate sequences. The level of sequence similarity between two sequences may be defined in different ways well known to those of ordinary skill in the art, depending on the purpose of the sequence comparison. For example, sequence similarity may be defined as sequence identity, which is a measure of how identical are the two sequences to one another. In another example, sequence similarity may be defined as sequence complementarity, which is a measure of how complementary are the two sequences to one another. Of course, the determination of any measure of sequence similarity must take into consideration the value of a matching (e.g., identical or complementary) position as well as the value of a nonmatching (e.g., nonidentical or noncomplementary) position, and different types of nonmatching bases may be afforded different values. For example, a purine substituted for another purine may be valued differently than a purine substituted for a pyrimidine. In a similar manner, methods will often also address base stacking energies for the proposed duplex. Numerous methods of computing sequence similarity are widely known and used by those of ordinary skill in the art, as described below.

When using a sequence comparison algorithm, known and candidate sequences are input into a computer, subsequence coordinates are designated if appropriate, and sequence algorithm program parameters are designated. The sequence comparison algorithm calculates a sequence similarity between a candidate sequence relative to a known reference sequence or set thereof, based on the designated program parameters. In certain embodiments, the sequence comparison algorithm calculates the percent sequence identity or regions of sequence identity for the candidate sequence relative to the known reference sequence, based on the designated program parameters. In other embodiments, the sequence comparison algorithm calculates the percent sequence complementarity or regions of sequence complementarity for the candidate sequence relative to the known reference sequence, based on the designated program parameters.

In certain embodiments of the present invention, universal oligos are designed for use in human diagnostics, prognostics, or theranostics. As such, candidate sequences are screened against sequences that may be found in a human sample, e.g., sequences from mammals, and viruses and bacteria commonly associated with or infecting humans, all of which may be contained in a custom database (e.g., containing information from multiple databases), one or more publicly-available databases, or a combination thereof.

The determination of sequence similarity between two or more sequences can be accomplished using a mathematical algorithm. Non-limiting examples of such mathematical algorithms are the algorithm of Myers and Miller (“Optimal alignments in linear space,” Comput Appl Biosci 4(1):11-17, 1988); the search-for-similarity-method of Pearson and Lipman (“Improved tools for biological sequence comparison,” Proc Natl Acad Sci USA 85(8):2444-8, 1988); and that of Karlin and Altschul (“Applications and statistics for multiple high-scoring segments in molecular sequences,” Proc Natl Acad Sci USA 90(12):5873-7, 1993). Preferably, computer implementations of these mathematical algorithms are utilized. Such implementations include, but are not limited to: CLUSTAL in the PC/Gene program (available from Intelligenetics, Mountain View, Calif.); the ALIGN program (Version 2.0), GAP, BESTFIT, BLAST, FASTA, Megalign (using Jotun Hein, Martinez, Needleman-Wunsch algorithms), DNAStar Lasergene (see dnastar.com) and TFASTA in the Wisconsin Genetics Software Package, Version 8 (available from Genetics Computer Group (GCG), 575 Science Drive, Madison, Wis., USA). Alignments using these programs can be performed using the default parameters or parameters selected by the operator. The CLUSTAL program is well described by Higgins. The ALIGN program is based on the algorithm of Myers and Miller; and the BLAST programs are based on the algorithm of Karlin and Altschul. Software for performing BLAST analyses is publicly available through the National Center for Biotechnology Information (ncbi.nlm.nih.gov). These resources utilize parameters to gauge the stability of the resulting duplex based on the identity of two strands and any loss of stability due to mismatches, stacking, and insertions. To the extend these precise parameters are required to determine the scope and control of the claimed subject matter, the parameters and algorithms in general use by these resources on Feb. 7, 2006 should be adopted and are incorporated by reference.

If a candidate sequence is found to have sequence similarity equal to or above a given threshold (however this threshold is defined, e.g., X % identity over an entire sequence or over a stretch thereof) during the screening against known sequences, the candidate sequence will be discarded (step 135). If a candidate sequence is found to have sequence similarity below a given threshold during the screening against known sequences, the candidate sequence will be extended by one or more nucleotides (step 130) and will go through the screening process again. (The use of “equal to or above” vs. “below” for a given threshold is by no means intended to be limiting and an practitioner of the instant invention may optionally compare “above” vs. “equal to or below,” depending on how a given threshold is determined and/or defined.)

In a preferred embodiment, the candidate sequence will be extended by one nucleotide at a time, but will be extended by each of A, T, G and C. For example, if candidate sequence XXXXXXXXXXXXXXX is determined to have sequence homology below the given threshold, candidate sequence XXXXXXXXXXXXXXX will then be extended by one nucleotide four times, that is, candidate sequence XXXXXXXXXXXXXXX will be extended to candidate sequence XXXXXXXXXXXXXXXA, candidate sequence XXXXXXXXXXXXXXXT, candidate sequence XXXXXXXXXXXXXXXG and candidate sequence XXXXXXXXXXXXXXXC and each of these candidate sequences will be screened as described previously (step 120). The process continues until a length L is achieved. Once a candidate sequence of length L is found, where in preferred embodiments, L is greater than 25 nucleotides, 30 nucleotides, 35 nucleotides, 40 nucleotides, or 45 nucleotides, or, in even more preferred embodiments L is greater than 50 nucleotides, 55 nucleotides, 60 nucleotides or more, it is placed in a first group of candidate sequences (step 140), and these candidate sequences in the first group are used to build a universal oligo set.

In building a universal oligo set, sequences complementary to the candidate sequences in the first group are generated and added to the candidate sequences in the first group (step 150). At step 160, each candidate sequence and complement thereof in the first group is compared to each other candidate sequence and each other complement thereof in the first group to determine the extent of sequence similarity (however “sequence similarity” is defined). If a candidate or complement sequence is found to have sequence similarity equal to or above a given threshold (again, however “sequence similarity” is defined) during the screening at step 160, the candidate sequence and its complement will be discarded (step 175). If it is determined that a candidate sequence and its complement are found to have sequence similarity below a given threshold during the screening at step 160, the candidate sequence and complement will be added to a second group (step 170). The threshold at step 160 may be the same as that used in step 120, or may be different. The candidate and complementary sequences in the second group may then be subjected to further screening (step 180), using various parameters such as, e.g., melting temperature (Tₘ), existence of duplexes, specificity of hybridization, existence of a GC clamp, existence of hairpins, existence of sequence repeats, dissociation minimum for a 3′ dimer, dissociation minimum for the 3′ terminal stability range, frequency threshold, and/or maximum length of acceptable dimers, and the like.

Alternatively, universal oligos may be generated using a modified algorithm as shown in FIG. 2. In step 210, candidate oligo sequences are randomly generated. Typically, such randomly generated sequences will be short, for example, between about 40 and 100 nucleotides in length, or between about 50 and 80 nucleotides in length, or about 60 nucleotides in length. In step 220, the GC content of the sequence and its complement are analyzed, and at step 235 oligos are removed that have a GC content above or below a given threshold. The GC content threshold(s) will depend on the needs of the user and may be, for example, GC content of less than about 40% or greater than or equal to about 60% (or, similarly, less than or equal to about 40% or greater than about 60%). In step 230, the sequence and its complement are analyzed for mononucleotide sequence repeats, and at step 237 oligos are removed that have a mononucleotide sequence repeat of greater than a given threshold, such as, for example about 5 bases. The remaining candidate and complementary sequences may then be subjected to further screening using various parameters such as melting temperature (Tₘ), existence of duplexes, specificity of hybridization, existence of a GC clamp, existence of hairpins, existence of sequence repeats, dissociation minimum for a 3′ dimer, dissociation minimum for the 3′ terminal stability range, frequency threshold, or maximum length of acceptable dimers and the like (step 240). Oligos that fail the further screening are discarded at step 245. At step 250, each candidate sequence and its complement are compared to known sequences, typically, by comparing the candidate sequence to sequences stored in publicly-available and/or custom databases. If a candidate sequence and/or its complement are found to have sequence similarity equal to or above a given threshold (however this threshold is defined, e.g., X % identity over an entire sequence or over a stretch thereof) during the screening against known sequences, the candidate sequence and its complement will be discarded (step 255). At step 260, the sequences are analyzed to determine the likelihood of cross-hybridization with other candidate oligos, and if a sequence is found not to have a likelihood of cross-hybridization (as defined by the thermodynamics in conditions similar to hybridization conditions of the assay) with the other remaining candidate sequences and their complements then the sequence is added to the Universal Oligo Set at step 270. Sequences found to have a likelihood of cross-hybridization are discarded at step 265. These steps are repeated until the Universal Oligo Set contains a desired number of oligos.

It is to be noted that the steps of either algorithm can be practiced in varied orders or combinations, steps may be added or removed, and thresholds or stringency conditions of the steps may be increased or decreased depending on the needs of the user.

The oligos for use in the methods disclosed herein (e.g., universal oligos) can be 1 to 10000 bases in length, preferably 10 to 1000 bases in length, more preferably 10-500 bases in length and more preferably about 25 to about 100 bases in length. Additionally, the oligos may be DNA, RNA or PNA (peptide nucleic acid), or any chemically-modified variant thereof, and can include non-naturally occurring subunits, sequences and/or moieties. PNA includes peptide nucleic acid analogs. The backbones of PNA are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tm) for mismatched versus perfectly matched base pairs. DNA and RNA typically exhibit a 2-4° C. drop in Tₘ for an internal mismatch. With the non-ionic PNA backbone, the drop is closer to 7-9° C. This allows for better detection of mismatches. Similarly, due to their non-ionic nature, hybridization of the bases attached to these backbones is relatively insensitive to salt concentration. This is advantageous, as a reduced salt hybridization solution has a lower Faradaic current than a physiological salt solution (in the range of 150 mM). Table 1 provides a listing of 200 exemplary universal oligos, each of which is 60 bases in length. Oligos perfectly complementary (i.e., no mismatches) to those provided in Table 1 are also exemplary universal oligos.

### Hybridization of Oligos

The hybridization of capture-associated oligos to electrode-associated oligos is employed as a means of indicating the presence of the particular target agent. When multiple capture-associated oligos are used, they must be sufficiently different from one another to preclude the possibility of hybridizing to one another. Likewise, the sequences of the electrode-associated oligos must be sufficiently different from one another and from the capture-associated oligos (with the exception of the complementary capture-associated oligos) to preclude the possibility of hybridizing to other electrode-associated oligos, or to more than one of the capture-associated oligos. Those of skill in the art would appreciate and understand that this specific hybridization can be achieved in a number of ways, including, but not limited to, the use of specifically designed/predetermined sequences, varying the temperature at which the hybridization takes place, varying the concentration of certain constituents of the hybridization buffer, such as divalent and monovalent metal ions, and by varying the length of the nucleic acid molecules. Further, in most embodiments, it is preferred to avoid unintended hybridization with sequences that may be found in the sample (e.g., human genomic sequences and genomic sequences of pathogens) in designing oligo pairs, as described above.

The hybridization reaction between the capture-associated oligos and the complementary oligos on the detection device (e.g., electrode-associated oligos) is typically performed in a solution where the metal ion concentration of the buffer is between 0.01 mM to 5 M and a pH range of pH 5 to pH 10. Other components can be added to the buffer to promote hybridization such as dextran sulfate, EDTA, surfactants, etc. The hybridization reaction can be performed at a temperature within the range of 10° C. to 90° C., preferably at a temperature within the range of 25° C. to 60° C., and most preferably at a temperature within the range of 30° C. to 50° C. Alternatively, the temperature is chosen relative to the melting temperatures (Tₘs) of the nucleic acid molecules employed. The reaction is typically performed at an incubation time from 10 seconds to about 12 hours, and preferably an incubation time from 30 seconds to 5 minutes. A variety of hybridization conditions may be used in the present invention, including high, moderate and low stringency conditions; see for example Maniatis et al, Molecular Cloning: A Laboratory Manual, 3rd Edition (2001), hereby incorporated by reference. Persons of ordinary skill in the art will recognize that stringent conditions are sequence-dependent and are dependent upon the totality of the conditions employed. Longer sequences typically hybridize specifically at higher temperatures. Generally, stringent conditions are selected to be about 5-10° C. lower than the thermal melting point (Tₘ) for the specific sequence at a defined ionic strength pH. Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide. The hybridization conditions may also vary when a non-ionic backbone, e.g., PNA is used, the advantages of using PNA are discussed above. The hybridization reaction can also be controlled electrochemically by applying a potential to the electrodes to speed up the hybridization. Alternatively, the potential can be adjusted to ensure specific hybridization by increasing the stringency of the conditions.

### Attachment of Oligos to Other Molecules and Surfaces

Conjugation of an oligo (e.g., a universal oligo) to a capture moiety may be performed in numerous ways, providing it results in a capture moiety possessing both specific binding to capture the target agent as well as providing it does not restrict nucleic acid hybridization functionalities (e.g., hybridization of the capture-associated oligo to a chip- or electrode-associated oligo) in embodiments where a cleavage is not performed (e.g., where the capture moiety is not cleaved from the capture-associated oligo), to allow detection of the bound target agent. For example, nucleic acid-antibody conjugates can be synthesized by using heterobifunctional cross-linker chemistries to covalently attach single-stranded DNA labels through amine or sulfhydryl groups on an antibody to create a capture agent of the invention. (See, e.g., Hendricksen E R, Nucleic Acids Res. (1995) Feb. 11; 23(3):522-9.) In another example, covalent single-stranded DNA-streptavidin conjugates, capable of hybridizing to complementary surface-bound oligonucleotides, are utilized for the effective immobilization of biotinylated capture moieties. Niemeyer C M, et al., Nucleic Acids Res. 2003 Aug. 15; 31(16):90. Many other nucleic acid molecular conjugates are described in, e.g., Heidel J et al., Adv Biochem Eng Biotechnol. (2005); 99:7-39. Additional methods of creating capture moiety-oligo conjugates, both those existing and under development, will be apparent to one skilled in the art upon reading the present disclosure, and such methods are intended to be captured within the methods of the invention.

In certain embodiments, a capture-associated oligo may be conjugated to a capture moiety via a scaffold. Scaffolds can be comprised of any substrate capable of supporting oligonucleotides and capture moieties. Conjugation of a capture-associated oligo and a capture moiety to a scaffold may be performed in numerous ways, providing it results in a loaded scaffold possessing both affinity to capture a target agent as well as a capture-associated oligo available for nucleic acid hybridization with an electrode-associated oligo in embodiments where a cleavage reaction or nucleic acid amplification is not performed, to allow determination of the presence of the target agent in a sample. Methods of creating loaded scaffolds, both those existing and under development, described herein infra, will be apparent to one skilled in the art upon reading the present disclosure, and are intended to be captured within the methods of the invention.

In one embodiment, the scaffold is comprised of a nanoparticle. Nanoparticles useful in the practice of the invention include metal (e.g., gold, silver, copper and platinum), semiconductor (e.g., CdSe, CdS, and CdS or CdSe coated with ZnS) and magnetic (e.g., ferromagnetite) colloidal materials. Other nanoparticles useful in the practice of the invention include ZnS, ZnO, TiO₂ AgI, AgBr, HgI₂, PbS, PbSe, ZnTe, CdTe, In₂S₃, In₂Se₃, Cd₃P₂, Cd₃As₂, InAs, and GaAs. The size of the nanoparticles is preferably from about 5 nm to about 150 nm (mean diameter), more preferably from about 5 to about 50 nm, most preferably from about 10 to about 30 nm. The nanoparticles may also be rods.

Methods of making metal, semiconductor and magnetic nanoparticles are well-known in the art. See, e.g., Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Methods of making ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshavsky et al., J. Am. Chem. Soc., 112, 9438 (1990); Ushida et al., J. Phys. Chem., 95, 5382 (1992). Suitable nanoparticles are also commercially available from, e.g., Ted Pella, Inc. (gold), Amersham Corporation (gold) and Nanoprobes, Inc. (gold).

Loaded scaffolds are made by affixing or otherwise associating oligonucleotides and capture moieties onto a suitable substrate. Methods of attaching or associating oligonucleotides and capture moieties such as antibodies to substrates such as gold particles are well known in the art. A brief example of such methods using gold nanoparticles for the scaffold is as follows: Gold colloid of a particle size suited to the needs of the user is prepared using well known methods (Beesley J., (1989), “Colloidal Gold. A new perspective for cytochemical marking”. Royal Microscopical Society Handbook No 17. Oxford Science Publications. Oxford University Press). In such a method, 100 mL of 0.01% gold chloride solution is adjusted to pH 9.0. Antibody solution is prepared by making a 0.1 ug/ul solution of antibody in 2 mM borax and dialyzing for at least 4 hours against 1 liter of borax at pH 9.0. The antibody solution is centrifuged at 100,000 g for 1 hour at 4° C. immediately prior to use. The dialyzed and centrifuged antibody solution (0.1 ug/ul) is adjusted to pH 9.2, and appropriate amount of antibody solution is then added dropwise to 100 mL of the gold solution while stirring rapidly. After 5 minutes, 5 mL of filtered 10% BSA at pH 9.0 is added to the antibody-gold particle solution and stirred gently for 10 minutes. The solution is then purified by centrifugation to form an antibody-gold particle scaffold conjugate.

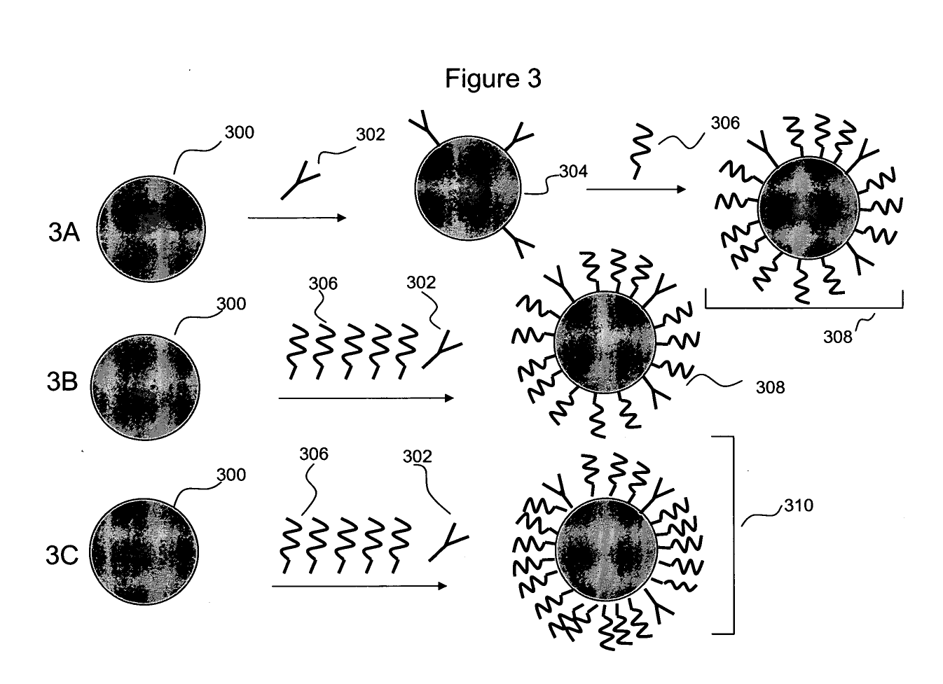

For example, FIG. 3 illustrates one embodiment of the generation of a loaded scaffold. In FIG. 3A, a scaffold (300) is mixed or otherwise contacted with a capture moiety (302) to form a scaffold with an associated capture moiety (304). This scaffold with capture moiety (304) is then mixed or otherwise contacted with capture-associated oligos (306) to form a loaded scaffold (308). Loaded scaffold (308) now comprises scaffold (300) with capture moiety (302) and with capture-associated oligos (306). In an alternative aspect of this embodiment, capture-associated oligos (306) may be added to scaffold (300) first, with capture moieties (304) added subsequently.

In FIG. 3B, an alternative embodiment to the method for generating a loaded scaffold (308) is illustrated. Scaffold (300) is mixed or otherwise simultaneously contacted with capture-associated oligos (306) and capture moiety (302) to form loaded scaffold (308). The embodiment shown in FIG. 3B differs from that of FIG. 3A in that the capture-associated oligo (306) and the capture moiety (302) are simultaneously mixed with scaffold (300) in FIG. 3B versus stepwise in FIG. 3A.

In FIG. 3C, an alternative embodiment to the method for generating a loaded scaffold (308) is illustrated. Scaffold (300) is mixed or otherwise contacted with capture-associated oligos (306) and capture moiety (302) to form a loaded scaffold (310). The embodiment shown in FIG. 3C differs from that of FIG. 3B in that the loaded scaffold (310) of FIG. 3C is comprised of an increased ratio of capture-associated oligo (306) to capture moiety (302) as compared to the loaded scaffold (308) of FIG. 3B. Ratios of capture-associated oligos to capture moieties may be varied as needed to optimize detection of various target agents.

Oligonucleotides can be attached to the antibody-gold particle scaffold through the use of functionalized chemical groups such as alkanethiol, alkylthiol, or other functionalized thiols attached to either terminal end of the oligonucleotide. Methods for attaching oligonucleotides to antibody-modified gold particles are well known in the art. An example of such preparation is as follows: alkylthiol functionalized oligonucleotides are reacted with an appropriate amount of antibody-gold particle scaffold solution for 16 hours and then stabilized with salt to 0.1M NaCl. 10% BSA is then added to the solution for 30 minutes to stabilize the gold particle scaffolds. This solution is then purified via centrifugation at 20,000 g for one hour at 4° C., the supernatant is removed, and the centrifugation is repeated. 0.1M NaCl/0.01M phosphate buffer solution at pH 7.4 is used to resuspend the pellet. The loaded scaffold in the solution comprises antibodies and oligonucleotides associated with a gold particle scaffold.

Other nanoparticles may be used as substrates for oligonucleotide binding, and methods for binding oligonucleotides to such substrates is well known in the art. Briefly, the following references describe other substrates and linking agents that can be used to bind oligonucleotides to nanoparticles: Nuzzo et al., J. Am. Chem. Soc., 109, 2358 (1987) (disulfides for oligo attachment on gold); Allara and Nuzzo, Langmuir, 1, 45 (1985) (carboxylic acids for oligo attachment on aluminum); Allara and Tompkins, J. Colloid Interface Sci., 49, 410-421 (1974) (carboxylic acids for oligo attachment on copper); Iler, The Chemistry Of Silica, Chapter 6, (Wiley 1979) (carboxylic acids for oligo attachment in silica); Timmons and Zisman, J. Phys. Chem., 69, 984-990 (1965) (carboxylic acids for oligo attachment on platinum); Soriaga and Hubbard, J. Am. Chem. Soc., 104, 3937 (1982) (aromatic ring compounds for oligo attachment on platinum); Hubbard, Acc. Chem. Res., 13, 177 (1980) (sulfolanes, sulfoxides and other functionalized solvents for oligo attachment on platinum); Hickman et al., J. Am. Chem. Soc., 111, 7271 (1989) (isonitriles for oligo attachment on platinum); Proupin-Perez et al., Nucleosides Nucleotides and Nucleic Acids, 24, 1075 (2005) (maleimides for oligo attachment on silica); Eltekova and Eltekov, Langmuir, 3, 951 (1987) (aromatic carboxylic acids, aldehydes, alcohols and methoxy groups for oligo attachment on titanium dioxide and silica); Lec et al., J. Phys. Chem., 92, 2597 (1988) (rigid phosphates for oligo attachment on metals); Jung et al., Langmuir 20, 8886 (2004) (carboxylic acids for oligo attachment on carbon nanotubes).

Other particles capable of binding oligonucleotides include polymeric particles (such as polystyrene particles, polyvinyl particles, acrylate and methacrylate particles), glass particles, latex particles, Sepharose beads and other like particles. The conjugation of these particles with oligonucleotides is well known in the art. Functional groups used to mediate the transfer of oligonucleotides onto the particle include carboxylic acids, aldehydes, amino groups, cyano groups, ethylene groups, hydroxyl groups, mercapto groups, and other similar functional groups. The following references describe the transfer of oligonucleotides onto these particles: Chrisey et al., Nucleic Acids Research, 24, 3031-3039 (1996) (glass) and Charreyre et al., Langmuir, 13, 3103-3110 (1997), Fahy et al., Nucleic Acids Research, 21, 1819-1826 (1993), Elaissari et al., J. Colloid Interface Sci., 202, 251-260 (1998), Kolarova et al., Biotechniques, 20, 196-198 (1996) and Wolf et al., Nucleic Acids Research, 15, 2911-2926 (1987).

Magnetic, polymer-coated magnetic, and semiconducting particles can also be used as substrates for attachment of oligonucleotides. The conjugation of these particles with oligonucleotides is well known in the art. For reference, see Chan et al., Science, 281, 2016 (1998); Bruchez et al., Science, 281, 2013 (1998); Kolarova et al., Biotechniques, 20, 196-198 (1996). Use of functionalized polymer-coated magnetic particles (Fe₃O₄) are well known in the art and available from Dynal (Dynabeads™) and silica-coated magnetic Fe₃O₄ nanoparticles may be modified (Liu et al., Chem. Mater., 10, 3936-3940 (1998)) using well-developed silica surface chemistry (Chrisey et al., Nucleic Acids Research, 24, 3031-3039 (1996)) and employed as magnetic probes as well.

Radio Frequency Identification (RFID) tags may also be incorporated into the scaffold substrate or, derivatized, may serve as the scaffold substrate itself. RFID is an automatic identification method, relying on storing and remotely retrieving data using devices called RFID tags or transponders. Use of such RFID tags has been discussed in detail in the co-pending applications: U.S. Ser. No. 60/834,951, filed Aug. 2, 2006, entitled “Diagnostic Devices and Methods of Use;” U.S. Ser. No. 60/851,697, filed Oct. 13, 2006, entitled “Methods and Compositions for Detecting One or More Target Agents Using Radio Frequency Identification Devices;” and U.S. Ser. No. 60/853,697, filed Oct. 23, 2006, entitled “Methods and Compositions for Detecting One or More Target Agents Using Radio Frequency Identification Devices,” all of which are hereby incorporated by reference in their entirety.

A basic RFID system includes two components: an interrogator or reader and a transponder (commonly called an RF tag). The interrogator and RF tag include respective antennas. In operation, the interrogator transmits through its antenna a radio frequency interrogation signal to the antenna of the RF tag. In response to receiving the interrogation signal, the RF tag produces an amplitude-modulated response signal that is transmitted back to the interrogator through the tag antenna by a process known as backscatter.

The RFID tags used in the devices of the present invention are preferably small, so as to reduce the amount of scaffolding material, capture-associated universal oligos, and capture moieties needed per device, as well as reduce reaction volumes allowing for decreased cost. For example, Hitachi, Ltd. offers both a 0.15×0.15 millimeter (mm), 7.5 micrometer (μm) thick device and a 0.4×0.4 mm (“μ-Chip™”) device.

In one embodiment, the device comprises: a) an RFID tracking device; b) a scaffold matrix to which the tracking device is affixed, embedded, and/or associated with or in a preferred embodiment the tracking device itself acts as the matrix for association or affixment of the capture moiety and capture-associated universal oligos; c) a polymer that is uniformly distributed on at least one surface of the matrix; d) and a plurality of capture moieties and capture-associated universal oligos on the scaffold. The polymers permit the attachment, conjugation or association of the capture moieties and capture-associated universal oligos to the matrix. In a specific embodiment, the polymer used in the device is a biocompatible polymer. Examples of such polymers include, but are not limited to, polytetrafluoroethylene (PTFE), Sephadex, polystyrene, polyethylene, and polypropylene. In specific embodiments, the RFID scaffold also comprises an adapter molecule associated with the polymer, e.g., a coupling agent such as avidin or strepavidin. The adaptor molecules may be conjugated directly to the polymer, or via a linker, e.g. a peptidic spacer.

Once the RFID loaded scaffold of the present invention is contacted with a sample suspected of containing target agent, any target agent within the sample will preferentially bind to its corresponding capture moiety on the RFID loaded scaffold. The target agent and the capture moiety will thus comprise a binding pair, and the reacted RFID loaded scaffold can be isolated based on this binding. For example, the reaction mixture comprising the reacted RFID loaded scaffolds can be further contacted with an immobilized binding partner that preferentially binds the reacted RFID loaded scaffold/target agent complex. The RF tag of a reacted RFID loaded scaffold can be read and identified using an interrogator device with the ability to identify the particular RF tag.

In certain preferred embodiments, the reaction is multiplexed by the use of multiple different RFID loaded scaffolds where each different capture moiety and capture-associated universal oligo loaded on a scaffold is associated with a different RF tag. In this manner, multiple target agents can be screened and detected in a single reaction. In such embodiments, different RFID frequencies are employed for each particular RF tag, allowing the reporting of multiple different signals when interrogated.

Those having skill in the art can readily contemplate different sizes of the scaffold depending on the needs of the user. Where a nanoparticle is used as the scaffold substrate, the size of the substrate can be 1 nm to 1000 nm, preferably 5 nm to 80 nm, and even more preferably 10 nm to 30 nm. Nanoparticles made from other materials may have different sizes, as is known to those with skill in the art.

Similarly, the density of the oligonucleotides on the scaffold can vary depending on the needs of the user. Those having skill in the art can readily contemplate different densities of oligos on the scaffold depending on the needs of the user. Similarly, the ratio of capture moieties to capture-associated universal oligos loaded onto a loaded scaffold can vary. For example, in some instances a 1:1 ratio of capture moieties to capture-associated universal oligos may be desired. On the other hand, ratios of 1:10, 1:100, 1:1000, 1:10000, 1:100000 or more may be desired and a larger ratio of capture-associated universal oligos (reporting molecules) to capture moieties is preferred. The larger the ratio of capture-associated universal oligos to capture moieties, the less likely an amplification step will be used.

In accordance with the present invention, one oligo of an oligo pair (e.g., a universal oligo pair), the electrode-associated oligo, is immobilized (directly or indirectly) onto an electrochemical surface. Although a metal electrode (e.g., gold, aluminum, platinum, palladium, rhodium, ruthenium, any metal or other material having a free electron in its outer most orbital) is preferably employed as the surface for immobilizing the electrode-associated oligo, other surfaces such as photodiodes, thermistors, ISFETs, MOSFETs, piezo elements, surface acoustic wave elements, and quartz oscillators may also be employed. By “electrode” herein is meant a composition, which, when connected to an electronic device, is able to conduct, transmit, receive or otherwise sense a current or charge. This current or charge is subsequently converted into a detectable signal. Alternatively an electrode can be defined as a composition, which can apply a potential to and/or pass electrons to or from a chemical moiety. Electrodes are known in the art and include, but are not limited to, certain metals and their oxides, including gold; platinum; palladium; silicon; aluminum; titanium, metal oxide electrodes including platinum oxide, titanium oxide, tin oxide, indium tin oxide, palladium oxide, silicon oxide, aluminum oxide, molybdenum oxide (Mo₂, O₆), tungsten oxide (WO₃) and ruthenium oxides; carbon (including glassy carbon electrodes, graphite, pyrolytic graphite, carbon fiber, and carbon paste); and semiconductor electrodes, such as Si, Ge, ZnO, CdS, TiO₂ and GaAs. The electrode may also be covered with conductive compounds to enhance the stability of the electrodes immobilized with probes or nonconductive (e.g., insulating) materials. Monomolecular films or biocompatible materials may also be employed to coat or partially coat the electrodes.

The electrodes described herein are presumed to be a flat surface, which is only one of the possible conformations of the electrode. The conformation of the electrode depends upon the detection method employed. For example, flat planar electrodes may be preferred for electrochemical detection methods, thus requiring addressable locations for synthesis and/or detection. In certain embodiments, the detection electrodes are formed on a glass or polymer substrate (e.g., a semi-flexible polymer substrate).

The discussion herein is generally directed to the formation of gold electrodes, but as will be appreciated by those in the art, other electrodes can be used as well. The substrate can comprise a wide variety of materials, as will be appreciated by those in the art, with glass, polymers and printed circuit board (PCB) materials being particularly preferred. Thus, in general, the suitable substrates include, but are not limited to, fiberglass, Teflon™, ceramics, glass, silicon, mica, plastic (including acrylics, polystyrene and copolymers of styrene and other materials, polypropylene, polyethylene, polybutylene, polycarbonate, polyurethanes, Teflon™, and derivatives thereof, etc.), GETEK (a blend of polypropylene oxide and fiberglass), and other materials typically employed and readily known to those of ordinary skill in the art.

In a specific embodiment, the electrode designs of the present invention utilize a conductive layer deposited on a stable, semi-flexible, plastic-like material. The term “semi-flexible” refers to a material that must be capable of slight flexure, yet must be relatively stiff or rigid, so as to resist any stretching or permanent deformation during use. Should stretching or deformation occur, this would result in fracture or interruption in the continuity of the conductive layer, and thereby destroy its effectiveness as a conductive element. Suitable materials for use include polyimide and polyester flexible materials, such as those used by the companies All Flex, Inc. (Northfield, Minn.) and Minco (Minneapolis, Minn.).

In one preferred embodiment, the relatively thin, clear, plastic-like film onto which the conductive material is deposited is comprised of polyethylene terephthalate, which is sold under the trade name “MYLAR™”. The MYLAR™ is preferably in the range of ½ mil (0.00127 cm) to 20 mils (0.0508 cm) in thickness and conductive layer deposited onto the MYLAR™ is preferably comprised of a metal as described above, e.g., gold or platinum.

The use of a semi-flexible material has a number of advantages over other substrates, such as glass. It is more cost-effective and less fragile than glass, and its physical properties allow the construction of multiple electrodes on large sheets of flexible material to allow for more cost-efficient manufacturing. The flexibility of the material also allows it to conform to a number of different shapes, providing multiple potential conformations for the electrode.

The conformation of the electrode depends upon the detection method employed. For example, flat planar electrodes may be preferred for electrochemical detection methods, thus requiring addressable locations for synthesis and/or detection. In a particular embodiment, the semi-flexible material is conformed to a tubular shape to allow flow-through detection of a target agent. Such a conformation increases the surface area available for binding compared to a planar conformation of an electrode of approximately the same dimensions, and such a conformation may be preferable for detection of target agents that are predicted to be in low abundance in a sample. Other conformations, such as spirals, u-shapes and the like, will be apparent to one skilled in the art upon reading the present specification and are intended to be included in the scope of the invention.

In another specific embodiment, the electrode comprising the semi-flexible material is a double sided electrode with the conductive layer on one side of the material and an additional functional element adhered to the same material and associated with the electrode, e.g., adjacent to the electrode or on the opposite surface. Exemplary functional elements include heating sensors and microheating elements. A microsensor can improve the quality control of any detection reactions by measuring parameters such as temperature, pH, presence of contaminants, etc., thus ensuring accurate and fast readout of binding conditions without disrupting the binding abilities of the electrode surface. A microheater can directly control the temperature at which the desired detection reaction is occurring. These functional elements are especially useful in an integrated detection system to provide feedback to the control elements and ensure the optimum binding reaction conditions are maintained. Such microsensors and microheaters produced on flexible materials are available, for example, from the company Minco (Minneapolis, Minn.).

As is generally known in the art, one or a plurality of layers may be used, to make either “two-dimensional” (e.g., all electrodes and interconnections in a plane) or “three dimensional” substrates. Three-dimensional systems frequently rely on the use of drilling or etching, followed by electroplating with a metal such as copper, such that the “through board” interconnections are made, or comprise porous structures similar to xeolites in structure.

Accordingly, in a preferred embodiment, the present invention provides oligo chips (e.g., universal oligo chips, biosensors, etc.) that comprise substrates comprising a plurality of electrodes, preferably gold, platinum, palladium or semiconductor electrodes. In addition, each electrode has an interconnection that is attached to the electrode at one end and is ultimately attached to a device that can control the electrode and/or receive the signal transmitted via conductive means in contact with the electrode. That is, each electrode is independently addressable. The substrates can be part of a larger device comprising a detection chamber that exposes a given volume of a solution (e.g., comprising capture-associated oligos) to the detection electrode. Generally, the detection chamber ranges from about 1 μl (picoliter) to 1 mL (milliliter), with about 10 μl (microliter) to 500 μl being preferred. As will be appreciated by those in the art, depending on the experimental conditions and assay, smaller or larger volumes may be used. The volumes and concentrations employed are typically empirically determined using methods readily known to those of ordinary skill in the art.

In certain embodiments, the detection chamber and electrode are part of a cartridge that can be placed into a device comprising electronic components selected from the group comprising potentiometers, AC/DC voltage source, ammeters, processors, displays, temperature controllers, light sources, and the like. In a typical embodiment, the interconnections from each electrode are positioned such that upon insertion of the cartridge into the device, connections between the electrodes and the electronic components are established. The device can also comprise a means for controlling the temperature, such as a peltier block, that facilitates the conditions employed in the hybridization reaction.

In certain embodiments, the electrode is first coated with a biocompatible substance (such as dextran, carboxylmethyldextran, other hydrogels, polypeptides, polynucleotides, biocompatible and/or bio-inert matrices or the like). The electrode-associated oligo is immobilized to the biocompatible substance.

The electrode-associated oligos may be immobilized onto the electrodes directly or indirectly by covalent bonding, ionic bonding and physical adsorption. Examples of immobilization by covalent bonding include a method in which the surface of the electrode is activated and the nucleic acid molecule is then immobilized directly to the electrode or indirectly through a cross linking agent. Yet another method using covalent bonding to immobilize an electrode-associated oligo includes introducing an active functional group into an oligo followed by direct or indirect immobilization. The activation of the surface may be conducted by electrolytic oxidation in the presence of an oxidizing agent, or by air oxidation or reagent oxidation, as well as by covering with a film. Useful cross-linking agents include, but are not limited to, silane couplers such as cyanogen bromide and gamma-aminopropyl triethoxy silane, carbodiimide and thionyl chloride and the like. Useful functional groups to be introduced to the oligo may be, but are not limited to, sulfide, disulfide, amino, amide, amido, carboxyl, hydroxyl, carbonyl, oxide, phosphate, sulfate, aldehyde, keto, ester and mercapto groups. Other highly reactive functional groups may also be employed using methods readily known to those of ordinary skill in the art.

### Electrochemical Detection

To detect multiple target agents in a sample, multiple electrodes, or an electrode with multiple different electrode-associated oligos are employed. For example, nucleic acid detection sensors, which use an electrochemical technique, can comprise an oligo array or other structural arrangement to detect the multiple agents. In certain embodiments, the multiple different electrode-associated oligos may be attached in a predetermined configuration, or each different electrode-associated oligo may bind a complementary oligo (e.g., a capture-associated oligo) under experimental conditions that are different than those for any of the other different electrode-associated oligos. In some embodiments, a plurality of electrodes each having a distinct electrode-associated oligo affixed thereto or otherwise associated therewith are arranged in predetermined configuration. In a preferred embodiment, the voltage applied to each electrode is equal. Additionally, to verify the hybridization of a particular electrode-associated oligo to a complementary oligo (e.g., a capture-associated oligo), the electrochemical detection device preferably includes a switch circuit, a decoder circuit, and/or, a timing circuit to apply the voltage to the individual electrodes and to receive the output signal from each of the electrodes.

Electrochemical detection of a hybridization event can be enhanced by the use of an electrochemical hybridization detector. In certain preferred embodiments, an electrochemical hybridization detector is an agent that binds to double-stranded nucleic acid, but does not bind to single-stranded nucleic acid. In such embodiments, the electrochemical hybridization detector would only bind to the electrode-associated oligo if it has hybridized with a complementary oligo (e.g., a capture-associated oligo) to create a double-stranded nucleic acid. An electrochemical hybridization detector can be, for example, an intercalating agent characterized by a tendency to intercalate specifically into double-stranded nucleic acids such as double-stranded DNA. Intercalating agents have in their molecules a flat (planar) intercalating group such as a phenyl group, which preferentially intercalates between the base pairs of the double-stranded nucleic acid. Most intercalating agents comprise conjugated electron structures and are therefore optically active; some are commonly used in the quantification or visualization of nucleic acids. Certain intercalating agents exhibit an electrode response, thereby generating or enhancing an electrochemical response. As such, determination of physical change, especially electrochemical change, may serve to detect the intercalating agents bound to a double-stranded nucleic acid and so enhance the detection of a hybridization reaction. In other embodiments, an electrochemical hybridization detector is an agent that binds differently to double-stranded nucleic acid than it does to single-stranded nucleic acid in such a way that the electrochemical signal produced from a double-stranded nucleic acid bound to the agent is stronger or otherwise enhanced relative to a single-stranded nucleic acid bound to the agent.

Electrochemically active intercalating agents useful in the present invention are, but are not limited to, ethidium, ethidium bromide, acridine, aminoacridine, acridine orange, proflavin, ellipticine, actinomycin D, daunomycin, mitomycin C, Hoechst 33342, Hoechst 33258, aclarubicin, DAPI, Adriamycin, pirarubicin, actinomycin, tris (phenanthroline) zinc salt, tris(phenanthroline) ruthenium salt, tris(phenanthroline) cobalt salt, di(phenanthroline) zinc salt, di(phenanthroline) ruthenium salt, di(phenanthroline) cobalt salt, bipyridine platinum salt, terpyridine platinum salt, phenanthroline platinum salt, tris(bipyridyl) zinc salt, tris(bipyridyl) ruthenium salt, tris(bipyridyl) cobalt salt, di (bipyridyl) zinc salt, di(bipyridyl) ruthenium salt, di(bipyridyl) cobalt salt, and the like. Other useful intercalating agents are those listed in Published Japanese Patent Application No. 62-282599. Some of these intercalators contain metal ions and can be considered transition metal complexes. Although the transition metal complexes are not limited to those listed above, complexes which comprise transition metals having oxidation-reduction potentials not lower than or covered by that of nucleic acids are less preferable. The concentration of the intercalator depends on the type of intercalator to be used, but it is typically within the range of 1 ng/mL to 1 mg/mL. Some of these intercalators, specifically Hoechst 33258, have been shown to be minor-groove binders and specifically bind to double-stranded DNA. The use of such electrochemically active minor groove binders is useful for detection of hybridization in electrochemical detection methods. Thus, in accordance with the present invention, the term “intercalator” is not intended to be limited to those compounds that “intercalate” into the rungs of the DNA ladder structure, but is also intended to include any moiety capable of binding to or with nucleic acids including major and minor groove-binding moieties.

Additionally, intercalators may be used for electrochemical detection where the intercalator molecule itself may or may not be able to enhance electrochemical detection, but where the intercalator is conjugated to molecules that enhance electrochemical detection (electrochemical enhancing conjugates) in a formula such as I—(X)ₘ—(Y)ₙ, where I is an intercalator, X is a linking moiety, and Y is an electrochemical enhancing entity (such as an electron acceptor). For example, the minor groove binder Hoechst 33258, itself an electrochemical detection enhancer, may be conjugated to additional molecules of Hoechst 33258, another intercalator, an organometallic electrochemical detection enhancer, metallocene, or any other electrochemical enhancing entity. The electrochemical enhancing entities can be attached to the intercalator by covalent or non-covalent linkages. If the electrochemical enhancing entities are attached covalently, the functional groups include haloformyl, hydroxy, oxo, alkyl, alkenyl, alkynyl, amide, amino, ammonio, azo, benzyl, carboxy, cyanato, thiocyanato, alkoxy, halo, imino, isocyano, isothiocyano, keto, cyano, nitro, nitroso, peroxy, phenyl, phosphino, phosphono, phospho, pyridyl, sulfonyl, sulfo, sulfinyl, or mercaptosylfanyl, with preferred functional groups being amino, carboxy, oxo, and thiol groups, and with amino groups being particularly preferred. In addition, homo- or hetero-bifunctional linkers may be used and are well known in the art. As will be appreciated by those in the art, a wide variety of intercalators, electrochemical enhancing entities and functional groups may be used.

Transition metals are those whose atoms have a partial or complete d orbital shell of electrons. Suitable transition metals for use in conjunction with the present invention include, but are not limited to, cadmium (Cd), copper (Cu), cobalt (Co), palladium (Pd), zinc (Zn), iron (Fe), ruthenium (Ru), rhodium (Rh), osmium (Os), rhenium (Re), platinum (Pt), scandium (Sc), titanium (Ti), vanadium (V), chromium (Cr), manganese (Mn), nickel (Ni), molybdenum (Mo), technetium (Tc), tungsten (W), and iridium (Ir). That is, the first series of transition metals, the platinum metals (Ru, Rh, Pd, Os, Ir and Pt), along with Fe, Re, W, Mo and Tc, are preferred. Particularly preferred are ruthenium, rhenium, osmium, platinum, cobalt and iron.

The transition metals are commonly complexed with a variety of ligands, to form suitable transition metal complexes. As will be appreciated by those in the art, the number and nature of the co-ligands will depend on the coordination number of the metal ion. Mono-, di- or polydentate co-ligands may be used at any position. Suitable ligands fall into two categories: ligands, which use nitrogen, oxygen, sulfur, carbon or phosphorus atoms (depending on the metal ion) as the coordination atoms (generally referred to in the literature as sigma (Σ) donors) and organometallic ligands such as metallocene ligands (generally referred to in the literature as pi (π) donors). Suitable nitrogen donating ligands are well known in the art and include, but are not limited to, NH₂; NHR; NRR′; pyridine; pyrazine; isonicotinamide; imidazole; bipyridine and substituted derivatives of bipyridine; terpyridine and substituted derivatives; phenanthrolines, particularly 1,10-phenanthroline (abbreviated phen) and substituted derivatives of phenanthrolines such as 4,7-dimethylphenanthroline and dipyridol[3,2-a:2′,3′-c]phenazine (abbreviated dppz); dipyridophenazine; 1,4,5,8,9,12-hexaazatriphenylene (abbreviated hat); 9,10-phenanthrenequinone diimine (abbreviated phi); 1,4,5,8-tetraazaphenanthrene (abbreviated tap); 1,4,8,11-tetra-azacyclotetradecane (abbreviated cyclam), EDTA, EGTA and isocyanide. Substituted derivatives, including fused derivatives, may also be used. In some embodiments, porphyrins and substituted derivatives of the porphyrin family may be used. See for example, Comprehensive Coordination Chemistry, Ed. Wilkinson et al., Pergammon Press, 1987, Chapters 13.2 (pp. 73-98), 21.1 (pp. 813-898) and 21.3 (pp. 915-957), all of which are hereby expressly incorporated by reference.

Suitable sigma donating ligands using carbon, oxygen, sulfur and phosphorus are known in the art. For example, suitable sigma carbon donors are found in Cotton and Wilkinson, Advanced Organic Chemistry, 5th Edition, John Wiley & Sons (1988), hereby incorporated by reference; see, e.g., page 38. Similarly, suitable oxygen ligands include crown ethers, water and others known in the art. Phosphines and substituted phosphines are also suitable; see, e.g., page 38 of Cotton and Wilkinson. The oxygen, sulfur, phosphorus and nitrogen-donating ligands are attached in such a manner as to allow the heteroatoms to serve as coordination atoms.

Such organometallic ligands include cyclic aromatic compounds such as the cyclopentadienide ion [C₅H₅ (−1)] and various ring substituted and ring fused derivatives, such as the indenylide (−1) ion, that yield a class of bis(cyclopentadieyl) metal compounds, (e.g. the metallocenes); see, e.g., Robins et al., J. Am. Chem. Soc. 104:1882-1893 (1982); and Gassman et al., J. Am. Chem. Soc. 108:4228-4229 (1986), incorporated by reference. Of these, ferrocene [(C₅H₅)₂ Fe] and its derivatives are prototypical examples, which have been used in a wide variety of chemical (Connelly et al., Chem. Rev. 96:877-910 (1996), incorporated by reference) and electrochemical (Geiger et al., Advances in Organometallic Chemistry 23:1-93; and Geiger et al., Advances in Organometallic Chemistry 24:87, incorporated by reference) electron transfer or “redox” reactions. Metallocene derivatives of a variety of the first, second and third row transition metals are potential candidates as redox moieties that are covalently attached to the nucleic acid. Other potentially suitable organometallic ligands include cyclic arenes such as benzene, to yield bis(arene) metal compounds and their ring substituted and ring fused derivatives, of which bis(benzene)chromium is a prototypical example. Other acyclic pi-bonded ligands such as the allyl(−1) ion, or butadiene yield potentially suitable organometallic compounds, and all such ligands, in conjunction with other pi-bonded and delta-bonded ligands constitute the general class of organometallic compounds in which there is a metal to carbon bond. Electrochemical studies of various dimers and oligomers of such compounds with bridging organic ligands, and additional non-bridging ligands, as well as with and without metal-metal bonds are potential candidate redox moieties in nucleic acid analysis.

When one or more of the co-ligands is an organometallic ligand, the ligand is generally attached via one of the carbon atoms of the organometallic ligand, although attachment may be via other atoms for heterocyclic ligands. Preferred organometallic ligands include metallocene ligands, including substituted derivatives and the metalloceneophanes (see page 1174 of Cotton and Wilkenson, supra). For example, derivatives of metallocene ligands such as methylcyclopentadienyl, with multiple methyl groups being preferred, such as pentamethylcyclopentadienyl, can be used to increase the stability of the metallocene. In a preferred embodiment, only one of the two metallocene ligands of a metallocene is derivatized.

Alternatively, in some embodiments, a capture-associated oligo may be labeled with an electroactive marker. These markers may serve to enhance or otherwise facilitate detection of hybridization between an electrode-associated oligo and an capture-associated oligo. For example, these markers may enhance an electrochemical signal generated when hybridization has occurred on an electrode. Such electroactive markers can include, but are not limited to, ferrocene derivatives, anthraquinone, silver and silver derivatives, gold and gold derivatives, osmium and osmium derivatives, ruthinium and ruthinium derivatives, cobalt and cobalt derivatives, and the like. In some embodiments, one or more electroactive markers may be used in combination with one or more electrochemical hybridization detectors to enhance detection of a hybridization event between a capture-associated oligo and a chip-associated oligo. For example, an intercalator may be used in combination with an electroactive marker in a formula (I—(X)ₘ—(Y)ₙ, where I is the intercalator, X is a linking moiety, and Y is the electroactive marker.

Electrochemical detection of a hybridization event can be enhanced by the use of an agent to reduce background signal from, for example, nonspecific binding of a electrochemical hybridization detector to single-stranded electrode-associated oligos. Such binding may result in an increase of signal at an electrode comprising electrode-associated oligos that are not hybridized to any capture-associated, thereby increasing background signal and potentially obscuring signal produced from actual hybridization events, which can hinder quantification of target agent in the sample. An agent to reduce background signal may be, for example, a single-stranded nuclease such as mung bean nuclease, nuclease P1, exonuclease I, exonuclease VII, or S1 nuclease, all of which are specific for digestion of single-stranded DNA (see, e.g., Desai, N. A. et al. (2003) FEMS Microbiol Review 26(5):457-91; and Sambrook, J. et al. (1989) Molecular Cloning: A Laboratory Manual (2^{nd} ed.), New York: Cold Spring Harbor Laboratory Press) The use of a single-strand-specific exonuclease would serve to remove oligos that did not hybridize with complementary oligos from the array prior to detection of signal. In some embodiments, exonuclease treatment precedes addition of an electrochemical hybridization detector. In other embodiments, a single-strand-specific binding protein may be used to block binding of an electrochemical hybridization detector to single-stranded DNA. For example, E. coli single-stranded DNA binding protein (SSB) may be used, preferably prior to addition of an electrochemical hybridization detector (see e.g., Krauss, G. et al. (1981) Biochemistry 20:5346-5352 and Weiner, J. H. et al. (1975) J. Biol. Chem. 250:1972-1980).

### Binding Partners

As described throughout this specification, the removal of excess, unreacted capture-associated oligo complexes can be achieved by providing immobilized binding partner(s) to the capture moiety that is conjugated to the capture-associated oligo (e.g., via loaded scaffolds). The immobilized binding partner may be bound to a matrix such as a vessel wall or floor. Alternatively, the matrix may be a column or filter, such as Sepharose 2B, Sepharose 4B, Sepharose 6B, CNBR-activated Sepharose 4B, AH-Sepharose 4B, CH-Sepharose 4B, Activated CH-Sepharose 4B, epoxy-Activated Sepharose 6B, Activated Thiol-Sepharose 4B, Sephadex, CM-Sephadex, ECH-Sepharose 4B, EAH-Sepharose 4B, NHS-activated Sepharose or Thiopropyl Sepharose 6B, etc., all of which are supplied by Pharmacia; BIO-GEL A, Cellex, Cellex AE, Cellex-CM, Cellex PAB, BIO-GEL P, Hydrazide BIO-GEL P, Aminoethyl BIO-GEL P, BIO-GEL CM, AFFI-GEL 10, AFFI-GEL 15, AFFI-PREP10, AFFI-GEL HZ, AFFI-PREP HZ, AFFI-GEL 102, CM BIO-GEL A, AFFI-GEL herparin, AFFI-GEL 501 OR AFFI-GEL 601, etc., all of which are supplied by Bio-Rad; Chromagel A, Chromagel P, Enzafix P-HZ, Enzafix P-SH or Enzafix P-AB, etc., all of which are supplied by Wako Pure Chemical Industries Ltd.; AE-Cellurose, CM-Cellurose or PAB Cellurose etc., all of which are supplied by Serva, over which the mixture of reacted and unreacted loaded scaffolds can be passed. The matrix may include a suspension of particulate matter in a solution, such as microscopic and/or macroscopic beads/particles, including magnetic particles, where the immobilized binding partner is immobilized on the beads or particle such as polystyrene-, cellulose-, latex-, silica-, polyaminostyrene-, agarose-, polydimethylsiloxane-, or polyvinyl-based beads. The immobilized binding partner may be associated with a Radio Frequency Identification (RFID) tag. See discussion of RFID tags herein. In a method using particles, the unreacted capture-associated oligo complexes will be retained on the semi-solid support created by the particles, whereas the reacted capture-associated oligo complexes will be eluted through the semi-solid support. Thus, only those capture-associated oligo complexes that have bound the particular target agent will be available for hybridization. Alternatively, the particles can include an immobilized binding partner specific for the target agent or for the target agent/capture moiety complex. In this embodiment, only those capture-associated oligo complexes comprising an antibody that has reacted with the target agent in the sample will be retained on the particles or matrix, and the unreacted capture-associated oligo complexes will pass through. The retained, reacted capture-associated oligo complexes then may be selectively released/eluted by known methods including but not limited to a cleavage step, discussed in detail below. Alternatively, the capture-associated oligos may be amplified as described elsewhere herein before hybridization to the electrode-associated oligo. Beads and particles can be separated from solution by using centrifugation, filtration, size exclusion chromatography, magnetism or other techniques known in the art.

In one embodiment of the present invention, magnetic particles (e.g., “beads”) may be used as the substrate on which a binding partner is immobilized and the immobilized binding partners attached to the substrate may be antibodies. The use of magnetic beads is well known in the art and they are commercially available from such sources as Ademtech Inc. (New York, N.Y.), Invitrogen (San Diego, Calif.), Bioclone Inc. (San Diego, Calif.) and Promega U.S. (Madison, Wis.). Magnetic beads typically range in size from 50 nm to 20 μm in diameter. The magnetic core of the beads may be encapsulated by a polymer shell, and further modified by surface chemistry to assist the immobilization of molecules such as antibodies on the bead. Magnetic beads may be physically manipulated via the application of a magnetic field which will draw the magnetic beads toward the field, and immobilize them, for instance, on the wall of a test tube adjacent to the magnetic field. Accordingly, with the magnetic beads immobilized, molecules not attached to the magnetic beads may be separated by such methods as aspiration. In one embodiment, antibodies corresponding to the suspected target agent in the sample are assembled on the magnetic bead. The conjugation of antibodies on the surface of magnetic beads is well known in the art, and is described in the Examples section, infra. Briefly, magnetic beads from Ademtech Inc., are washed according to protocol with the provided buffer solution. The surface of the beads is then prepared for coupling with the antibodies by treating it with EDC (1-ethyl-3-(3-dimethylaminopropyl) carbodiimide hydrochloride). Antibodies are added to a solution containing the treated beads and incubated for 1 hour at 37° C. under shaking. Bovine serum albumin is then added to the solution and incubated for 30 minutes under shaking. The beads are washed twice with the provided storage buffer. The resulting magnetic beads have antibodies coupled with the surface.

As noted above, although the examples and embodiments typically recite an electrochemical detection device, other detection devices can also be used with the methods disclosed herein. Therefore, other markers, hybridization detectors, and/or background signal reducers specific for the other detection devices may also be used with the methods presented herein. Such detection devices, markers, hybridization detectors, and/or background signal reducers are widely known and used by those of ordinary skill in the art.

### Methods of Use

In certain preferred embodiments, universal oligos (e.g., capture-associated universal oligos, electrode-associated universal oligos, and universal oligo chips) are used. Although the disclosure contains various examples of the methods of the invention using universal oligos, the invention is by no means to be limited to the use of universal oligos and oligos that are not universal oligos may optionally be used in the methods presented herein. Likewise, universal oligos may optionally be used in any examples herein that disclose the use of oligos that are not specifically indicated to be universal oligos.

The oligos and oligo chips may be used in a system comprising capture-associated oligos, where the capture moiety is, for example, an antibody, antigen or other ligand specific for a particular target agent. Such a system may also include loaded scaffolds comprising both capture-associated oligos and capture moieties. Briefly, the capture-associated oligos (whether on loaded scaffolds or not) are contacted/mixed with a sample that is suspected of containing the target agents, under conditions that if a target agent is present, the capture moiety can react with, e.g., bind with/to the specific target agent. The capture-associated oligos associated with the capture moiety may be added in excess relative to the amount of target agent suspected to be present in the sample.

In certain preferred embodiments, where an excess of capture-associated oligos is added to the sample, unreacted capture-associated oligos (i.e., those associated with capture moieties that have not bound target agent) are separated from the reacted capture-associated oligos (i.e., those associated with capture moieties that have bound target agent) prior to the hybridization reaction. This can be accomplished a number of ways. For example, the separation of excess, unreacted capture-associated oligos from reacted capture-associated oligos can be achieved by providing one or more immobilized binding partners that bind to a) capture moieties not bound to target agent, or b) capture moieties bound to target agent (or to the target agent itself), thereby immobilizing the a) unreacted capture-associated oligos, or b) reacted capture-associated oligos, respectively, and allowing removal of the oligos that are not immobilized. For example, the immobilized binding partner(s) can bind to a capture moiety that is associated with a capture-associated oligo. In certain embodiments, only those capture moieties that have not bound to target agent can bind to the immobilized binding partner(s). In other embodiments, only those capture moieties that have bound to target agent can bind to the immobilized binding partner(s).

In embodiments in which unreacted capture-associated oligos are immobilized by immobilized binding partners, reacted capture-associated oligos can be separated from the immobilized unreacted capture-associated oligos by any method known in the art (e.g., decanting, washing, aspirating, etc.) Optionally, the immobilized unreacted capture-associated oligos may be washed to remove any remaining reacted capture-associated oligos prior to exposure of the reacted capture-associated oligos to electrode-associated oligos. In embodiments in which reacted capture-associated oligos are immobilized by immobilized binding partners (whether via the capture moiety, target agent, or a complex thereof), unreacted capture-associated oligos can be removed from immobilized reacted capture-associated oligo complexes by any method known in the art (e.g., decanting, washing, aspirating, etc.) Optionally, the immobilized reacted capture-associated oligo complexes may be washed to remove any remaining unreacted capture-associated oligos prior to exposing the reacted capture-associated oligos to electrode-associated oligos. The immobilized binding partners can be affixed/immobilized directly or indirectly to a matrix such as a vessel wall, to particle(s) or bead(s) (including, but not limited to solid beads, semi-solid beads, porous beads, magnetic beads, or the like), or to other suitable surfaces (as described in more detain infra).

In various embodiments, the immobilized binding partner is bound to a matrix that is, e.g., a vessel wall or floor. Alternatively, the matrix may be macroscopic particles which may be used to construct a column or filter over which a mixture of reacted and unreacted capture-associated oligo complexes can be passed. Such macroscopic particles include, but are not limited to, Sephadex®, Sepharose 2B, Sepharose 4B, Sepharose 6B, CNBR-activated Sepharose 4B, AH-Sepharose 4B, CH—Sepharose 4B, Activated CH-Sepharose 4B, epoxy-Activated Sepharose 6B, Activated Thiol-Sepharose 4B, Sephadex, CM-Sephadex, ECH-Sepharose 4B, EAH-Sepharose 4B, NHS-activated Sepharose or Thiopropyl Sepharose 6B, etc., all of which are supplied by Pharmacia; BIO-GEL A, Cellex, Cellex AE, Cellex-CM, Cellex PAB, BIO-GEL P, Hydrazide BIO-GEL P, Aminoethyl BIO-GEL P, BIO-GEL CM, AFFI-GEL 10, AFFI-GEL 15, AFFI-PREP10, AFFI-GEL HZ, AFFI-PREP HZ, AFFI-GEL 102, CM BIO-GEL A, AFFI-GEL herparin, AFFI-GEL 501 OR AFFI-GEL 601, etc., all of which are supplied by Bio-Rad; Chromagel A, Chromagel P, Enzafix P-HZ, Enzafix P-SH or Enzafix P-AB, etc., all of which are supplied by Wako Pure Chemical Industries Ltd.; AE-Cellurose, CM-Cellurose or PAB Cellurose etc., all of which are supplied by Serva, over which the mixture of reacted and unreacted conjugated nucleic acid molecules can be passed. Similarly, the matrix may include a suspension of particulate matter in a solution, such as microscopic and/or macroscopic beads/particles, where the immobilized binding partner is immobilized on the beads or particle such as polystyrene-, cellulose-, latex-, silica-, polyaminostyrene-, agarose-, polydimethylsiloxane-, or polyvinyl-based beads. For example, in some methods using particles, the unreacted capture-associated oligo complexes can be retained on the semi-solid support created by the particles, whereas the reacted capture-associated oligo complexes will be eluted through the semi-solid support. Thus, only those capture-associated oligos that have bound the particular target agent will pass through the support and therefore be available for hybridization. Alternatively, in certain embodiments the particles can include an immobilized binding partner specific for the target antigen or for the capture moiety/target agent complex. In these embodiments, only those capture-associated oligos conjugated to a capture moiety that has reacted with the target antigen in the sample will be retained on the particles or matrix, and the unreacted nucleic acid molecules will pass through. The retained, reacted capture-associated oligos may be selectively released/eluted by known methods including but not limited to a cleavage step, discussed in detail herein. Beads and particles can be separated from solution by using centrifugation, filtration, size exclusion chromatography, magnetism or other techniques known in the art.

When employing suspensions of particulate matter in a solution, unreacted capture-associated oligos can be separated from the reacted capture-associated oligos by techniques such as centrifugation, size exclusion chromatography, filtration and the like. In a method using beads, in particular magnetic beads, the separation step can be achieved by applying a magnetic field to the magnetic beads. In some embodiments, the beads will bind with the unreacted capture moieties, leaving the reacted capture-associated oligo complexes (comprising an oligo, a capture moiety, and a target agent) in solution and available for hybridization. In other embodiments, the beads will bind with the reacted capture-associated oligo complexes (comprising an oligo, a capture moiety, and a target agent), leaving the unreacted capture moieties in solution. In addition, either the suspension or bead techniques can employ a particle or bead having a secondary capture moiety specific for the target agent to be detected. In this instance only those capture-associated oligos conjugated to capture moieties that have reacted with the target agent in the sample will be retained on the beads, and the unreacted capture-associated oligos are separated from the suspension by known techniques including, but not limited to, centrifugation, size exclusion chromatography, filtration, magnetism and the like. As discussed above, in this particular embodiment of the invention, the retained, reacted capture-associated oligos can be selectively released/eluted by known methods including, but not limited to, a cleavage step, discussed in detail herein.

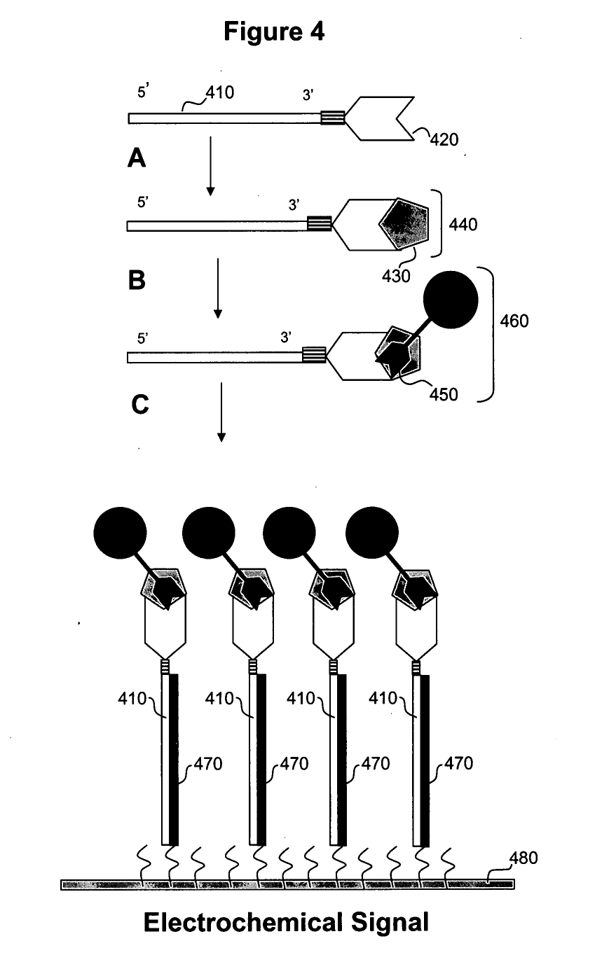

In an exemplary embodiment, an immobilized binding partner recognizes and binds to a capture moiety/target agent complex, but not to unreacted capture moiety or target agent not bound by a capture moiety. For example, FIG. 4 is a schematic diagram demonstrating the detection of a target agent (430) using an immobilized binding agent (450) for isolation of a reacted capture-associated oligo complex (440). In certain embodiments the immobilized binding partner (450) binds to the capture moiety (420)/target agent (230) complex. In step A, a capture-associated oligo (410) conjugated to the capture moiety (420) is exposed to a sample comprising target agent (430). In step B, a reacted capture-associated oligo complex (i.e., bound to target agent) (440) is exposed to an immobilized binding agent (450) to create immobilized reacted capture-associated oligo complex (460). In step C, immobilized reacted capture-associated oligo complex (460) is introduced to the electrode-associated oligos (470) on oligo chip (480). The binding of the immobilized reacted capture-associated oligo complex (460) comprising capture-associated oligo (410) to the complementary electrode-associated oligos (470) generates a signal in an electrochemical detection device.

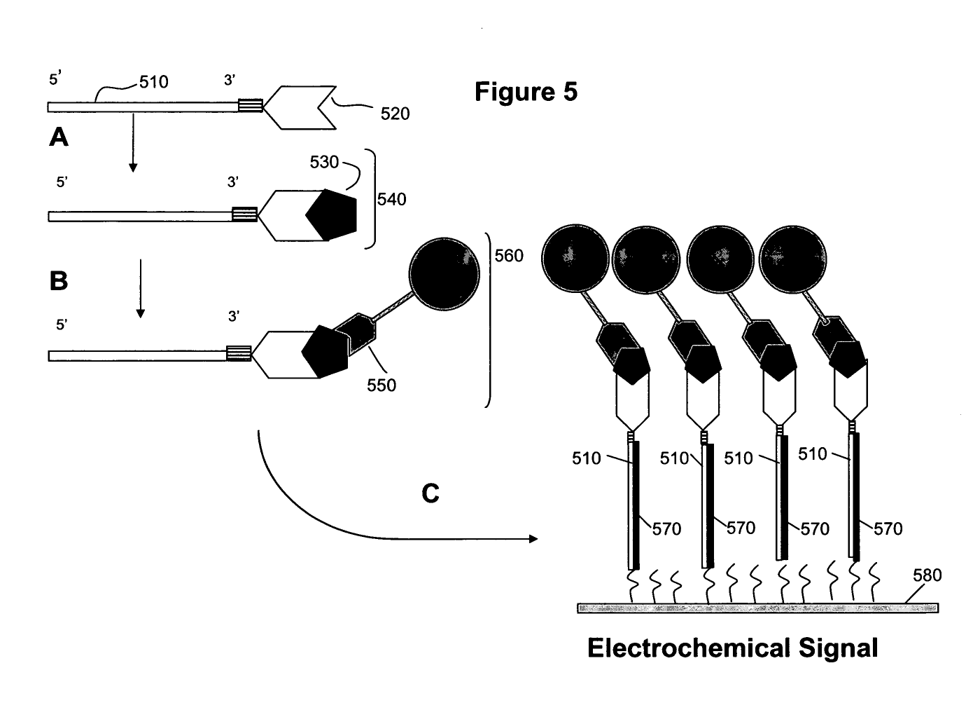

In a further embodiment, an immobilized binding partner binds to the target agent at an epitope not bound by the capture moiety. For example, FIG. 5 is a schematic diagram demonstrating the detection of a target agent (530) using an immobilized binding partner (550) for isolation of a reacted capture-associated oligo complex (540). Step A comprises exposure of a capture-associated oligo (510) conjugated to a capture moiety (520) to a sample comprising target agent (530) to create reacted capture-associated oligo complex (540). Step B comprises exposing reacted capture-associated oligo complex (540) to immobilized binding partner (550), which specifically binds to a different epitope of target agent (530) than does capture moiety (520) to create immobilized reacted capture-associated oligo complex (560). Step C comprises exposing immobilized reacted capture-associated oligo complex (560) to electrode-associated oligos (570) on oligo chip (580). The binding of the immobilized reacted capture-associated oligo complex (560) comprising capture-associated oligo (510) to a complementary electrode-associated oligo (570) generates a signal in an electrochemical detection device.

Those of skill in the art will readily understand the versatility of the nature of capture moieties and immobilized binding partner. Essentially, any ligand and its receptor can be utilized to serve as capture moieties, target agents and immobilized binding partners, as long as the target agent is appropriate for detection of the pathology or condition of interest. Suitable ligands and receptors include an antibody or fragment thereof and a corresponding antigen or epitope; a hormone and its receptor; an inhibitor and its enzyme, a co-factor portion and a co-factor enzyme binding site, a binding ligand and the substrate to which it binds, two halves of a heterodimer, and the like.

For example, if the capture moiety is an antibody specific for a particular infectious target agent (such as a bacterial or viral agent), the immobilized binding partner can be a naturally-occurring or synthetic epitope of the bacterial or viral antigen with which the antibody recognizes and interacts in a specific manner. In another example, if the capture moiety is an antigen specific for a particular antibody (target agent), the immobilized binding partner can be a naturally-occurring or synthetic antibody or functional fragment thereof with which the antigen recognizes and interacts in a specific manner. If multiple capture-associated oligos are used, each associated with a capture moiety specific for a different target agent or different epitope of the same target agent, multiple immobilized binding partners may be used to facilitate the removal/separation of unreacted capture-associated oligos (those associated with capture moieties that did not react with target agent in the sample). In such a detection method, multiple different target agents (e.g., agents specific to different viruses and/or bacteria) may be screened/detected simultaneously.

The target agents to be detected can be any target agent that is indicative of existence of or susceptibility to a phenotype of interest, for example, a pathological or otherwise observable or detectable condition, e.g., in humans or animals. In certain nonlimiting examples, such a condition is a disease or other physical or mental disorder, infection with a microorganism (bacterial, viral, or otherwise), an unhealthy state (e.g., obesity, suboptimal blood lipid levels), or a drug response (e.g., related to efficacy or adverse events). For example, target agents to be detected can be one or more target agents a) suspected of causing or capable of causing the condition, b) that increase or otherwise indicate predisposition or susceptibility to the condition, c) produced in an organism as a result of the condition, or a combination thereof. For example, the target agents can include, but are not limited to, bacteria, viruses, nucleic acids, proteins, proteinaceous agents (such as prions, antibodies, etc.), nucleic acids, metabolites, biological agents, chemical agents, and/or portions and/or combinations thereof. Again, those of skill in the art would appreciate and understand the particular type of target agent to be found in a particular sample and that is suspected of being related to or indicative of a particular phenotype of interest, e.g., a physiological condition or state. Other target agents that can be detected include air-borne, food-borne and water-borne agents, including biological and chemical toxins. A particular target agent need only be detectable by the methods disclosed herein.

The detection methods provided herein may be optionally multiplexed to allow simultaneous screening and detection of multiple target agents in a sample. The multiple target agents detected may be of a similar chemical composition (e.g., proteins, nucleic acids, antibodies, metabolites, etc.) or may be a mixture of target agents of different chemical compositions. For example, proteomic, genetic, metabolic, and/or immunologic markers may be combined for use in a single diagnostic, theranostic, and/or prognostic application. In certain embodiments, a multiplexed assay includes a different capture-associated oligo complex for each target agent to be detected; a detector (e.g., electrochemical detection device) comprising a plurality of oligos (e.g., electrode-associated oligos), each of which is complementary to one of the capture-associated oligos; and a set of immobilized binding partners specific for either the reacted or unreacted capture-associated oligo complexes. The methods for performing the capture reactions, hybridization reactions, etc., are described elsewhere herein. In certain embodiments, all target agents can be captured by their corresponding capture moiety under the same experimental conditions so a single capture reaction may be performed to capture all target agents. In other embodiments, different target agents require different reaction conditions to bind or otherwise associate with their corresponding capture moiety; in such embodiments, serial capture reactions may be performed to capture different target agents in the sample. For example, a first capture reaction may allow capture moiety A to bind target agent A, but capture moiety B is unable to bind target agent B. An immobilized binding partner A specific for reacted capture moiety A/target agent A complex immobilizes all reacted capture-associated oligo A complex leaving unreacted capture-associated oligo B complex and target agent B in the liquid phase. The liquid phase is removed and subjected to conditions that promote binding of capture moiety B to target agent B, resulting in the production of reacted capture-associated oligo B complexes, which are subsequently captured by an immobilized binding partner B specific for reacted capture moiety A/target agent A complex, thereby immobilizing reacted capture-associated oligo B complexes. The liquid phase can then be removed and the two immobilized complexes can be released and combined before contacting with a detection device, thereby allowing simultaneous detection of the two target agents in the sample. One of skill will readily understand that multiplexing is also applicable to other embodiments of the present invention and should not be limited by the exemplary embodiment presented above. For example, binding partners specific for the target agent or the unreacted capture-associated oligo complexes may be used in variations of the above embodiment.

The advantage of a simultaneous accurate detection method includes an increased speed at which multiple suspected target agents can be eliminated. For example, a patient can provide a sample that can quickly be tested for the presence of multiple suspected target agents (e.g., toxins, genetic loci, metabolites, strains of bacteria and/or viruses, combinations thereof, etc.). Such a rapid and accurate test can aid in the treatment of the condition, e.g., where no bacterial infection is found there is no need to treat with antibiotics. Similarly, improper use of antibiotics can be reduced or eliminated by ensuring that the proper antibiotic, specific for the detected infectious agent, is administered. Likewise, the cause of potential food-poisoning outbreaks, or terrorist attacks can be ascertained in a short space of time, and the relevant treatment regimen implemented, e.g., antibiotics for bacterial causes, antivirals for viral causes, and chemical antidotes for toxin causes. Additionally, the construction of complete test panels that can be specific for the particular type of sample, or for the particular suspected underlying diseases or agents is another advantage of this particular method. For example, one could construct a test panel for sexually transmitted diseases, another panel for common blood borne diseases, yet another for airborne pathogens, yet another for terrorist agents (biological and/or chemical), yet another for common childhood disease. These are only representative examples of possible test panels and are not intended to limit the scope of the invention in any way. Those of skill in the art would appreciate and understand the particular pathogens/agents and combinations that could be used in a particular test panel.

In some embodiments, the panel is selected so as to provide an indication of the particular strain of one or more pathogenic agents and, in particular, to provide an accurate indication of the proper antibiotic (or other treatment(s)) that is to be administered. For example, a panel of capture-associated oligos conjugated to antibodies is prepared, wherein the antibodies are monoclonal antibodies capable of distinguishing between various strains of a particular bacterial species (e.g., Staphylococcus aureus) characterized by, inter alia, their resistance to antibiotics (e.g., methicillin-resistant Staphylococcus aureus (MRSA)). Thus, by employing the present invention, a rapid and accurate screen can be performed whereby strains are identified and the proper antibiotic can be administered, resulting in both an effective treatment and a reduction in the overuse and/or improper use of antibiotics. In other embodiments, the panel can be employed to distinguish between, inter alia, bacterial and viral pathogens which present the same way, thereby allowing the physician to ensure that antibiotics are only used when required and, when used, that the proper antibiotic is administered.

With these concepts in mind, in one application of one embodiment of the invention, capture-associated universal oligos are conjugated (e.g., directly or via loaded scaffolds) to antibodies (capture moieties) and the target agent of interest is an antigen. In accordance with this embodiment of the invention the following elements are included: (1) a electrode-associated universal oligo immobilized on a surface, where the surface comprises an electrode, (2) a capture-associated universal oligo that is complementary to the electrode-associated universal oligo, where the capture-associated universal oligo is conjugated to an antibody corresponding to the target agent, (3) immobilized binding partners, and (4) a sample suspected of containing the target agent. In one aspect, the capture-associated universal oligo is contacted with the sample to form a first mixture, and the first mixture is contacted with the immobilized binding partners (antibodies specific for capture moieties that have not bound the target agent). The unbound capture moieties bind to the immobilized binding partners, thereby immobilizing the unreacted capture-associated universal oligos and removing the unreacted capture-associated universal oligos from solution. The solution phase of the mixture is then contacted with the electrode-associated universal oligos, followed by electrochemical detection as otherwise described herein. Alternatively, the reacted capture-associated universal oligos can be immobilized with an immobilized binding partner that binds the capture moieties bound to the target agent, (or a different epitope of the target agent than that bound by the capture moiety) leaving the unreacted capture-associated oligos in solution. Other variations on this preferred embodiment include one or more other aspects of the invention described herein or such other modification known to those of ordinary skill in the art.

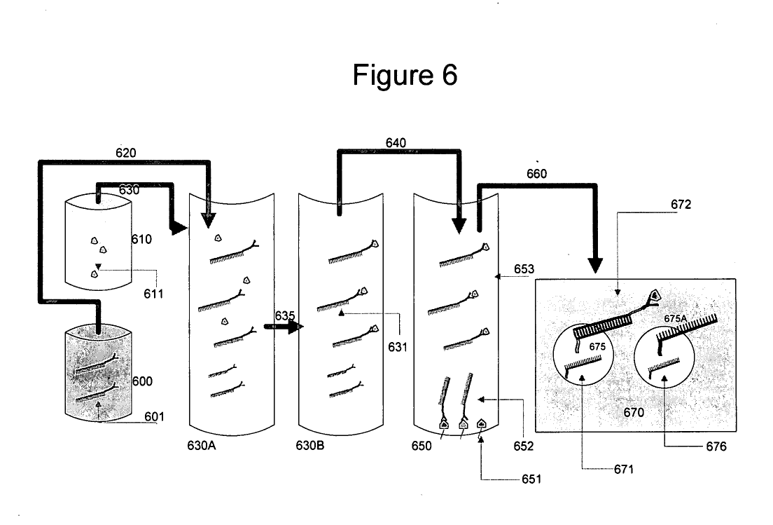

FIG. 6 shows a sample (610) suspected of having a target agent represented as antigen (611). The sample is mixed or otherwise contacted with a reagent (600) comprising one or more capture-associated universal oligos (601). Reagent (600) is added (620) to test tube (630A) and the sample (610) is also added (630) to the test tube (630A). In practice, it is not necessary to use a separate tube (630A), as the sample and reagent can be contacted or mixed in any fashion. After allowing the mixture of reagent (600) and sample (610) to react (time indicated by arrow (635)), the capture moieties conjugated to the capture-associated universal oligos (601) will bind with the antigen (611) to form a reacted capture-associated universal oligo complex (631).

In the embodiment shown, the reaction mixture containing reacted capture-associated universal oligo complex (631) is transferred (640) to a vessel (shown here as test tube (650)), which comprises an immobilized binding partner, represented as antigen (651). Any capture-associated universal oligo (601) that has not formed the reacted capture-associated universal oligo complex (631) will bind to the immobilized antigen (651), thereby resulting in removal of unreacted capture-associated universal oligos (601) from solution through the formation of immobilized unreacted capture-associated universal oligos (652).

The solution phase (653) of the reaction performed in test tube (650) is then transferred (660) to a universal oligo chip (670). The universal oligo chip (670) comprises one or more electrodes (675 and 675A) on which an electrode-associated universal oligo (671 and 676 respectively) has been immobilized. Electrode-associated universal oligo (671) is complementary to reacted capture-associated universal oligo (631) present in solution phase (653). Hybridization of electrode-associated universal oligo (671) with the reacted capture-associated universal oligo results in double-stranded nucleotide species (672) which is subsequently detected. Electrode-associated universal oligo (676) is not complementary to any capture-associated universal oligo present in solution phase (653), so no capture-associated universal oligo hybridizes to electrode-associated universal oligo (676). In most instances electrode-associated universal oligo 676 immobilized on electrode (675A) will have a different sequence than electrode-associated universal oligo (671) immobilized on electrode (675). Both electrodes are utilized if a multiplexed system is employed. For example, in a multiplexed system a second target agent is present in sample (610), and a second capture-associated universal oligo is conjugated to a second capture moiety that will specifically associate with the second target agent is present in reagent (600). When sample (610) and reagent (600) are mixed, the second capture moiety binds to the second target agent to form a second reacted capture-associated universal oligo complex. Any of the second capture moiety that fails to bind the second target agent (i.e., remains unreacted) will be bound by a second immobilized antigen in test tube (650). Thus, the second reacted capture-associated universal oligo complex (along with reacted capture-associated universal oligo complex (631)) remains in the solution phase (653) and is subsequently contacted with universal oligo chip (670). If electrode-associated universal oligo (676) is complementary to the second reacted capture-associated universal oligo present in solution phase (653), hybridization of electrode-associated universal oligo (676) with the second reacted capture-associated universal oligo results in a second double-stranded nucleotide species on universal oligo chip (670) which is subsequently detected simultaneously (or sequentially) with double-stranded species (672).

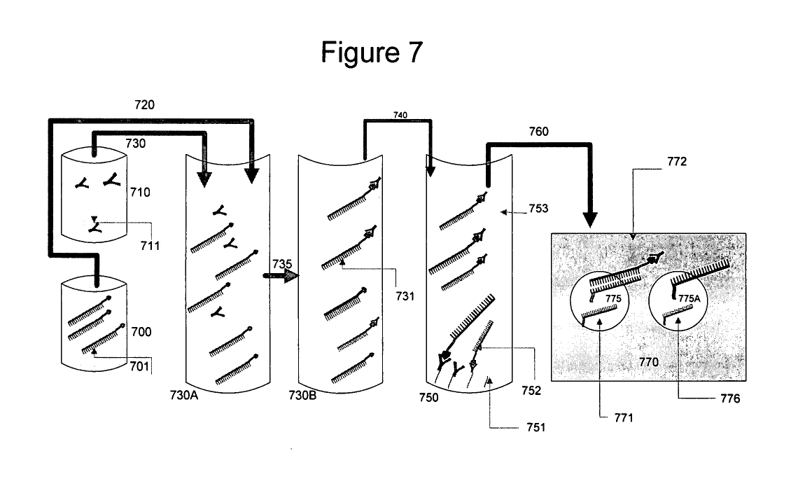

FIG. 7 shows an alternative embodiment of the present invention where a sample (710) suspected of having a target agent is provided. The target agent in this instance is an antibody (711). The sample is mixed or otherwise contacted with a reagent (700) having one or more capture-associated universal oligos (701), where the capture moiety is an antigen. Reagent (700) is added (720) to test tube (730A) and the sample (710) is also added (730) to the test tube (730A). In practice, it is not necessary to use a separate tube (730A) but instead the sample and reagent can be contacted or mixed in any fashion. After allowing the mixture of reagent (700) and sample (710) to react (time indicated by arrow (735)), the capture moieties conjugated to the capture-associated universal oligos (701) will bind with the antibody (711) to form a reacted capture-associated universal oligo complex (731).

In the embodiment shown, the reaction mixture containing the reacted capture-associated universal oligo complex (731) is transferred (740) to a vessel (shown here as test tube (750)), which comprises immobilized antibody (751). Any capture moiety that has not reacted with an antibody (711) will bind to the immobilized antigen (751), thereby resulting in removal of unreacted capture-associated universal oligos (701) from solution through the formation of immobilized unreacted capture-associated universal oligos (752).

The solution phase (753) of the reaction performed in test tube (750) is then transferred (760) to a universal oligo chip (770). The universal oligo chip (770) comprises one or more electrode surfaces (775 and 775A) on which electrode-associated universal oligos (771 and 776) have been immobilized. Electrode-associated universal oligo (771) is complementary to reacted capture-associated universal oligo present in solution phase (753). Hybridization of electrode-associated universal oligo (771) with reacted capture-associated universal oligo results in double-stranded nucleotide species (772) which is subsequently detected. Electrode-associated universal oligo (776) is not complementary to any capture-associated universal oligo present in solution phase (753), so no capture-associated universal oligo hybridizes to electrode-associated universal oligo (776). In most instances electrode-associated universal oligo (776) immobilized on electrode (775A) will have a different sequence than electrode-associated universal oligo (771) immobilized on electrode (775). Both electrodes are utilized if a multiplexed system is employed. For example, in a multiplexed system a second target agent is present in sample (710), and a second capture-associated universal oligo conjugated to a second capture moiety that will specifically associate with the second target agent is present in reagent (700). When sample (710) and reagent (700) are mixed, the second capture moiety binds to the second target agent to form a second reacted capture-associated universal oligo complex. Any of the second capture moiety that fails to bind the second target agent (i.e., remains unreacted) will be bound by a second immobilized antigen in test tube (750). Thus, only the second reacted capture-associated universal oligo complex (along with reacted capture-associated universal oligo complex (731)) remains in the solution phase (753) and is contacted with universal oligo chip (770). Electrode-associated universal oligo (776) is complementary to the second reacted capture-associated universal oligo present in solution phase (753). Hybridization of electrode-associated universal oligo (776) with the second reacted capture-associated universal oligo results in a second double-stranded nucleotide species on universal oligo chip (770) which is subsequently detected simultaneously (or sequentially) with double-stranded species (772).

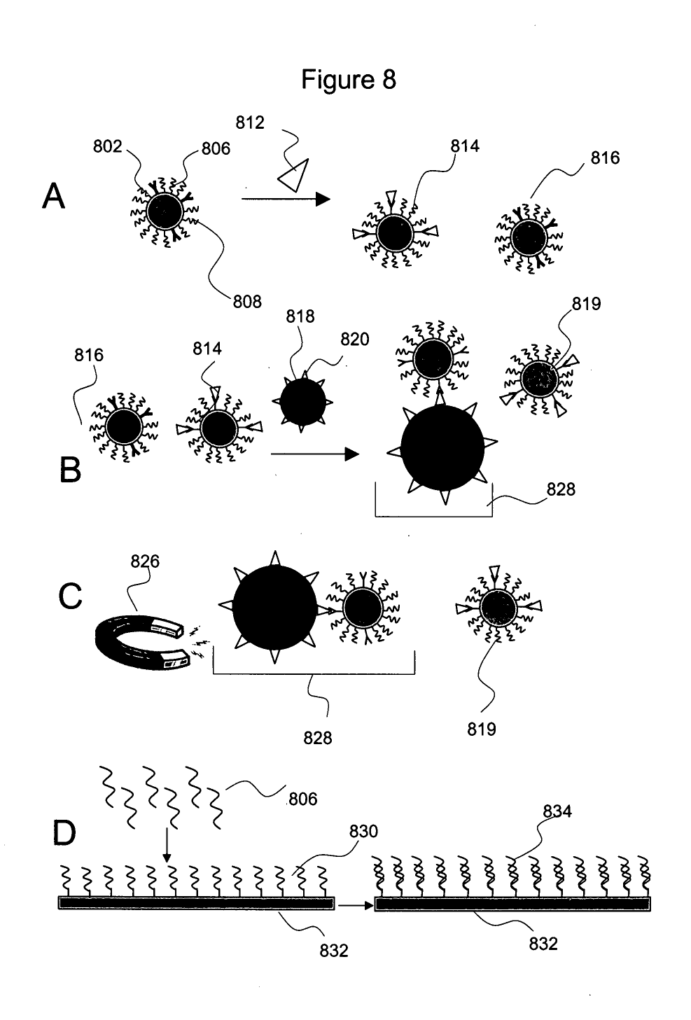

FIG. 8 illustrates an additional embodiment of the present invention for determining the presence of target agent in a sample by electrochemical detection using loaded scaffolds. Capture-associated universal oligos (806) and capture moieties (802) are affixed to the surface of the loaded scaffold (808) (the manufacture of which is described in FIG. 3). In FIG. 8 step A, loaded scaffold (808) is mixed with or otherwise contacted with a sample suspected of containing target agent (812) to form reacted loaded scaffold (814) and unreacted loaded scaffold (816). The reacted loaded scaffold (814) comprises loaded scaffold (808) with at least one target agent (812) bound to a capture moiety (802) on the loaded scaffold (808). The unreacted loaded scaffold (816) comprises loaded scaffold (808) with capture moieties (802) that did not bind to a target agent (812).

In FIG. 8 step B, the products from FIG. 8 step A (reacted loaded scaffold (814) and unreacted loaded scaffold (816)) are mixed or otherwise contacted with immobilized binding partner complex (818) to form immobilized binding partner/unreacted loaded scaffold complexes (828) and free reacted loaded scaffolds (819). The immobilized binding partner complex (818) has binding partners (820) affixed or otherwise attached to the surface of the immobilized binding partner complex (818). In this embodiment, the immobilized binding partner complex (818) further comprises a magnetic core. The binding partners (820) of the immobilized binding partner complex (818) in this embodiment are designed to bind to the capture moiety (802) of the loaded scaffolds (808) to form immobilized binding partner/unreacted loaded scaffold complexes (828). Since the capture moieties (802) on the unreacted loaded scaffolds (816) have not reacted with target agents (812), they are available to bind to the binding partner (820) of the immobilized binding partner complex (818).

In FIG. 8 step C, a magnetic field (826) is applied across the products of FIG. 8 step B (immobilized binding partner/unreacted loaded scaffold complexes (828) and free reacted loaded scaffolds (819)). The magnetic core of the immobilized binding partner complex (818) of the immobilized binding partner/unreacted loaded scaffold complex (828) is drawn to the magnetic field. The free reacted loaded scaffold (819) is not bound to an immobilized binding partner complex (818) and therefore remains in solution. In practice, the reaction represented by FIG. 8 step C may be performed in a reaction container such as a test tube (not shown). Application of the magnetic field (826) on a side of test tube will draw the magnetized immobilized binding partner/unreacted loaded scaffold complexes (828) to the side of the test tube wall most proximate to the magnetic field (826), and leave the unmagnetized free reacted loaded scaffolds (819) in solution where they may be separated by methods such as aspiration.

In FIG. 8 step D, capture-associated universal oligos (806) from the free reacted loaded scaffolds (819) that were magnetically separated from the immobilized binding partner/unreacted loaded scaffold complexes (828) in FIG. 8 step C are released from the free reacted loaded scaffolds (819) and applied to an electrochemical detection device (832). The electrochemical detection device (832) comprises one or more electrodes on which electrode-associated universal oligos (830) have been applied. Electrode-associated universal oligos (830) are complementary to the capture-associated universal oligos (806). Hybridization of electrode-associated universal oligos (830) with capture-associated universal oligos (806) results in a double-stranded nucleotide species (834) which is subsequently detected.

This embodiment can be employed in a multi-target (so-called multiplexed) format, allowing for the screening of multiple target antigens simultaneously. Such embodiments include providing (1) an electrochemical detection device comprising electrode-associated universal oligos, (2) a set of capture-associated universal oligos conjugated to capture moieties, (3) a sample suspected of containing the target agents, and (4) immobilized binding partners of the capture moieties conjugated to the capture-associated universal oligos. The method comprises mixing/contacting the sample with the capture-associated universal oligos under reaction conditions that allow the capture moieties to capture target agent present in the sample to form a first mixture. The first mixture is mixed/contacted with the immobilized binding partners of the capture moieties where the capture moieties that have not reacted with target agents in the sample react with the immobilized binding partners to form an immobilized phase and a solution phase. The solution phase comprises the capture-associated universal oligos conjugated to capture moieties that have reacted with target agents in the sample and the immobilized phase comprises the capture-associated universal oligos conjugated to capture moieties that did not bind target agents and instead bound the immobilized binding partners. The solution is introduced to a universal oligo chip and an electrochemical detection device under conditions such that a capture-associated universal oligo present in the solution phase will hybridize to a complementary electrode-associated universal oligo, generating an electrochemical signal. Alternatively, the reacted capture-associated universal oligos can be immobilized (e.g., by an antibody that recognizes a different epitope of the target antigen than that recognized by the capture moiety, or the capture moiety/target agent complex) leaving the unreacted capture-associated universal oligos in solution. The immobilized phase is separated, and the reacted capture-associated universal oligos are then released into solution and introduced to a universal oligo chip and an electrochemical detection device under reaction conditions such that the capture-associated universal oligos and electrode-associated universal oligos may hybridize to each other. Different electrode-associated universal oligos are present for each different capture-associated universal oligo corresponding to each different target agent to be detected (or not detected) in the sample. An electrochemical signal generated by the hybridization of complementary capture-associated universal oligos and electrode-associated universal oligos.

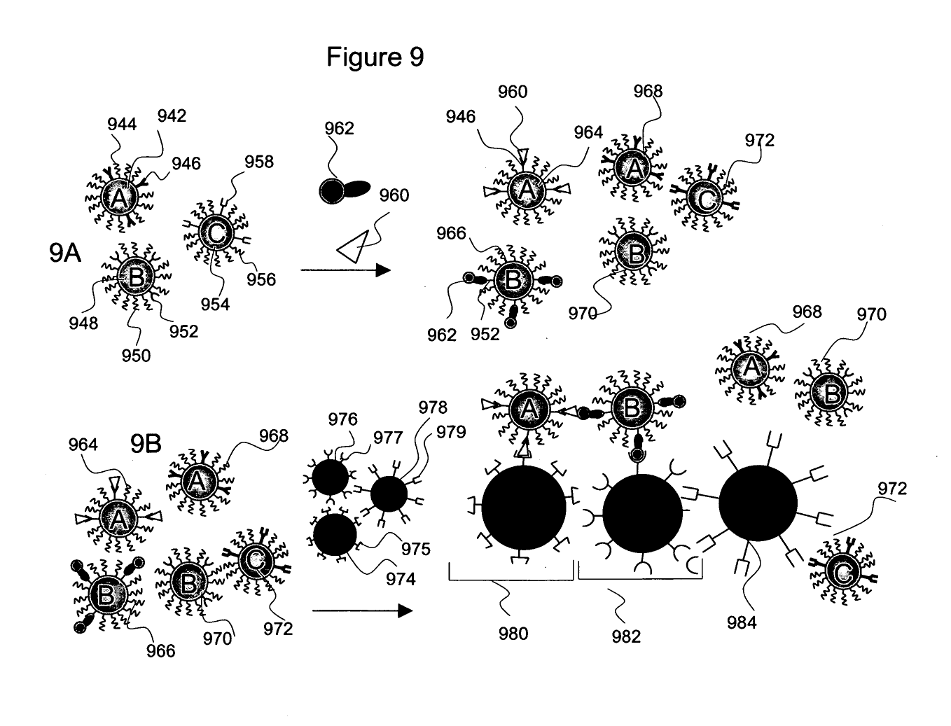

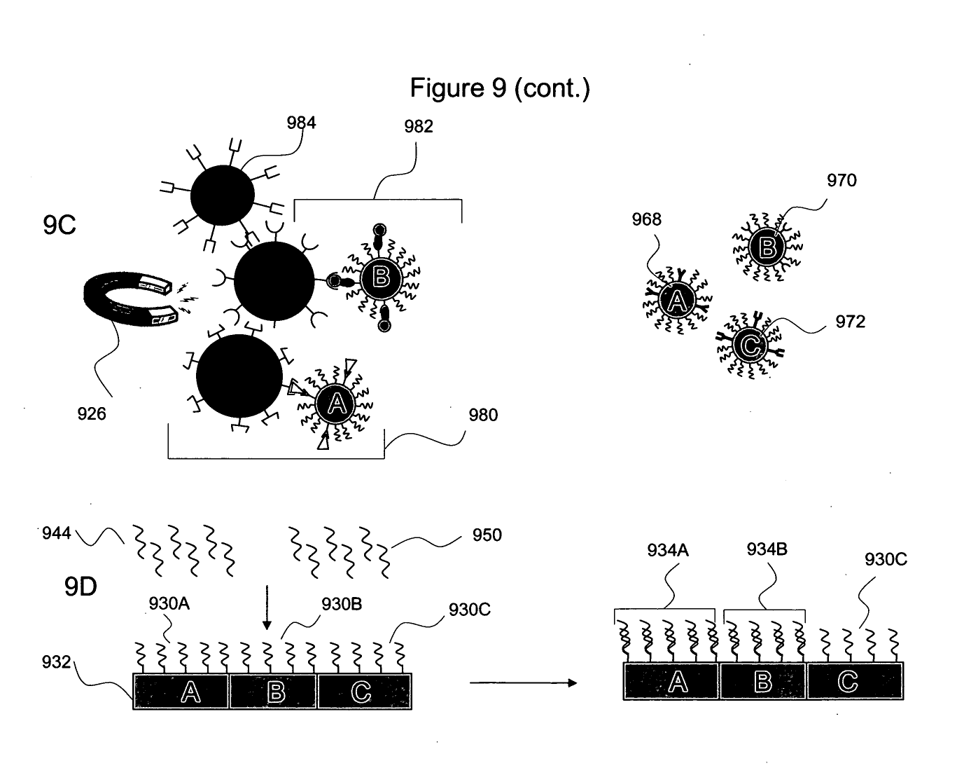

FIG. 9 illustrates a method of detection using multiple scaffold-bound capture moieties in a multiplexed type of assay. In FIG. 9A, loaded scaffold A (942) is comprised of capture-associated universal oligo A (944) and capture moiety A (946) (the manufacture of which is described in FIG. 3). Loaded scaffold B (948) is comprised of capture-associated universal oligo B (950) and capture moiety B (952) (the manufacture of which is described in FIG. 3). Loaded scaffold C (954) is comprised of capture-associated universal oligo C (956) and capture moiety C (958) (the manufacture of which is described in FIG. 3). Capture moiety A (946) is designed to bind to target agent A (960), capture moiety B (952) is designed to bind to target agent B (962), and capture moiety C (958) is designed to bind to another target agent (not shown). Loaded scaffold A (942), loaded scaffold B (948), and loaded scaffold C (954) are mixed or otherwise contacted with a sample suspected of containing target agents, here shown as target agent A (960) and target agent B (962). The reaction forms reacted loaded scaffold A (964), reacted loaded scaffold B (966), unreacted loaded scaffold A (968) (due to excess loaded scaffold A (964) in relation to target agent A (960), unreacted loaded scaffold B (970) (due to excess loaded scaffold B (948) in relation to target agent B (962), and unreacted loaded scaffold C (972), due to lack of target agent C. Reacted loaded scaffold A (964) is comprised of loaded scaffold A (942) and target agent A (960) bound to capture moiety A (946). Reacted loaded scaffold B (966) is comprised of loaded scaffold B (948) and target agent B (962) bound to capture moiety B (952).

In FIG. 9B, the products from the reaction in FIG. 9A are mixed or otherwise contacted with immobilized binding partner complex A (974), immobilized binding partner complex B (976) and immobilized binding partner complex C (978). Immobilized binding partner complex A (974) has binding partners A (975) affixed to its surface. Immobilized binding partner complex B (976) has binding partners B (977) affixed to its surface. Immobilized binding partner complex C (978) has binding partners C (979) affixed to its surface. Once mixed, this reaction produces immobilized binding partner/reacted loaded scaffold complex A (980), immobilized binding partner/reacted loaded scaffold complex B (982), unbound immobilized binding partner complex C (984), free unreacted loaded scaffold A (968), free unreacted loaded scaffold B (970) and free unreacted loaded scaffold C (972). Immobilized binding partner/reacted loaded scaffold complex A (980) represents immobilized binding partner complex A (974) bound to a different portion of target agent A (960) than capture moiety A (946) of reacted loaded scaffold A (964), to form immobilized binding partner/reacted loaded scaffold complex A (980). Immobilized binding partner/reacted loaded scaffold complex B (982) represents immobilized binding partner complex B (976) bound to a different portion of target agent B (962) than capture moiety B (952) of reacted loaded scaffold B (966), to form immobilized binding partner/reacted loaded scaffold complex B (982). Unbound immobilized binding partner complex C (984), free unreacted loaded scaffold A (968), free unreacted loaded scaffold B (970) and free unreacted loaded scaffold C (972) did not react to form complexes.

In FIG. 9C, a magnetic field (926) is applied across the products of the reaction in FIG. 9B. The magnetic cores of immobilized binding partner complex A (974) in immobilized binding partner/reacted loaded scaffold complex A (980), immobilized binding partner complex B (976) in immobilized binding partner/reacted loaded scaffold complex B (982), and unbound immobilized binding partner complex C (984) are drawn to the magnetic field. Free unreacted loaded scaffold A (968), free unreacted loaded scaffold B (970) and free unreacted loaded scaffold C (972) are not bound to an immobilized binding partner complex (974, 976, or 978) and therefore remain in solution. In practice, the reaction represented by FIG. 9C may be performed in a reaction container such as a test tube (not shown). Application of the magnetic field (926) on a side of test tube will draw immobilized binding partner/reacted loaded scaffold complex A (980), immobilized binding partner/reacted loaded scaffold complex B (982), and unbound immobilized binding partner complex C (984) to the side of the test tube wall most proximate to the magnetic field (926), and leave the unreacted loaded scaffolds (968, 970, and 972) in solution where they may be separated by methods such as aspiration.

In FIG. 9D, capture-associated universal oligo A (944) released from immobilized binding partner/reacted loaded scaffold complex A (980) and capture-associated universal oligo B (950), released from immobilized binding partner/reacted loaded scaffold complex B (982) that were magnetically separated from the free unreacted loaded scaffolds (968, 970, and 972) in FIG. 9C are applied to an electrochemical detection device (932). The electrochemical detection device (932) comprises a plurality of electrodes on which electrode-associated universal oligos (930A, 930B and 930C) have been applied. Electrode-associated universal oligos A (930A) are complementary to capture-associated universal oligos A (944). Electrode-associated universal oligos B (930B) are complementary to capture-associated universal oligos B (950). Electrode-associated universal oligos C (930C) are complementary to capture-associated universal oligos C (956). Hybridization of electrode-associated universal oligos (930A, 930B and 930C) with capture-associated universal oligos (944 and 950) results in double stranded nucleotide species (934A and 934B) which are subsequently detected. Double-stranded nucleotide species A (934A) represents the hybridization of capture-associated universal oligo A (944) to electrode-associated universal oligos A (930A). Double-stranded nucleotide species B (934B) represents the hybridization of capture-associated universal oligo B (950) to electrode-associated universal oligos B (930B). In the particular embodiment shown in FIG. 9A through 9D, the target agent corresponding to capture moiety C (958) of loaded scaffold C (954) was not present in the sample. Therefore, in the magnetic separation step shown FIG. 9C, though the immobilized binding partner complex C (984) is captured by magnetic field (926), there was no associated loaded scaffold C (954); therefore no capture-associated universal oligo C (956) is available to bind to electrode-associated universal oligo C (930C) on electrochemical device (932).

The capture reaction (e.g., the binding of the capture moiety to the target agent, such as an antibody binding reaction) is performed in solution, typically in a physiological buffer such as phosphate buffered saline (PBS) supplemented with a non-specific blocking agent, such as fetal or new-born calf serum, and may be used when the target agent to be detected is normally found under physiological conditions. However, the methods of the present invention are not limited to detecting target agents only found in physiological conditions. Those of skill in the art would appreciate and understand that different capture moieties may be used in different conditions without affecting the ability to bind the particular target agent to be detected. The capture reaction can be performed at a temperature within the range of 0° C. to 100° C., preferably at a temperature between 2° C. and 40° C., and more preferably within the range of about 4° C. to about 37° C., and most preferably within the range of about 18° C. to about 25° C. The capture reaction is typically conducted from about 5 minutes to 12 hours, preferably from about 10 minutes to 6 hours, and more preferably from about 15 minutes to 1 hour. The duration of the capture reaction depends on several factors, including the temperature, suspected concentration of the target agent, ionic strength of the sample, and the like. For example, a capture reaction may require an incubation at a temperature of 18° C. for 15 minutes, or an incubation at a temperature of 4° C. for 30 minutes. Often the immobilization reaction between the reacted or unreacted capture-associated universal oligo complexes and the immobilized binding partners is performed under conditions much like the capture reaction. Those of skill in the art would appreciate and understand the particular conditions and time required for the capture and immobilization reactions to be performed.

The capture-associated universal oligos preferably are provided in excess, with the excess capture-associated universal oligos (e.g., those conjugated to capture moieties that have not bound target agent) being removed prior to hybridization. This excess is typically determined relative to the suspected level of target agent present in the sample. This relative excess can be from about 1:1 to 1000000:1, preferably 2:1 to about 10000:1, and more preferably from about 4:1 to 1000:1, and most preferably from 5:1 to 100:1. For example, when the capture moiety is an antibody, typically, an excess of capture moiety can be created by adding 10 g of the capture-associated universal oligo to a sample suspected of containing up to 1 million target agents to be detected. This ratio gives rise to a molar ratio of typically about 4:1, but can vary dependant upon the molecular mass of the antibody and the target agent to be detected.

In some embodiments of the invention, separation (via e.g., cleavage, degradation, etc.) of capture moieties (and/or any target bound thereto) from capture-associated universal oligos is performed, e.g., following separation of reacted and unreacted capture-associated universal oligos, but prior to hybridization of the universal oligos to an oligo chip. For example, such separation can be useful when reacted capture-associated universal oligos are conjugated to a capture moiety that interferes with hybridization or electrochemical detection, e.g., because of the physical size or the presence of local areas of electron density on the surface of the capture moiety and/or target agent. Separation can be achieved, for example, by using a digestive enzyme or an enzyme that causes hydrolysis of a bond in a molecule (e.g., proteolytic enzymes, lipases, phosphatases, phosphodiesterases, esterases, etc.), endonucleases (specific for single-stranded or double-stranded sequences), exonucleases, a restriction endonuclease (e.g., EcoRI, HaeIII), or a flap endonuclease (e.g., FEN-1, RAD2, XPG, etc.). The choice of separation method will depend on the nature of the capture moiety and/or target agent and its conjugation to the universal oligo. Those of skill in the art will readily appreciate and understand the circumstances under which one particular method of separation would be preferred over another method of separation.

In some embodiments, a cleavage reaction is performed on a reacted capture-associated universal oligo complex (comprising a universal oligo, a capture moiety, a target agent, and, in some embodiments, a scaffold) to separate the universal oligo from the reacted capture-associated universal oligo complex. Such a cleavage reaction preferably removes any portion of the reacted capture-associated universal oligo complex that may interfere with hybridization and/or detection of the universal oligo on the oligo chip. In certain embodiments, such a cleavage reaction involves cleaving the reacted capture-associated universal oligo complex in a region between the capture moiety and the portion of the universal oligo that will hybridize to the electrode-associated oligo on the oligo chip. In other embodiments, such a cleavage reaction involves cleaving the capture moiety, for example, to remove a portion that obstructs or otherwise inhibits detection of the universal oligo on an oligo chip. In still further embodiments, such a cleavage reaction involves cleaving the target agent, for example, to remove a portion that obstructs or otherwise inhibits detection of the universal oligo on an oligo chip. Such cleavage may be carried out by any method known to those of ordinary skill in the art. For example, photocleavage may be employed where a photocleavable phosphoramidite exists or is engineered at an appropriate location within the reacted capture-associated universal oligo complex, cleavage by a restriction endonuclease may be employed where a restriction endonuclease recognition site exists or is engineered at an appropriate location within the reacted capture-associated universal oligo complex, or cleavage by a protease may be employed where a protease recognition site exists or is engineered at an appropriate location within the reacted capture-associated universal oligo complex. Such an appropriate location, as described above, may be, e.g., within the capture-associated universal oligo, between the capture-associated universal oligo and the capture moiety, within the capture moiety, or within the target agent. Dithiothreitol (DTT), which is provided in the reaction buffer of T7 RNA polymerase amplification, may also be used to uncouple oligonucleotide linkage on gold particles. Those of skill in the art will readily appreciate and understand the circumstances under which one particular method of cleavage would be preferred over another method of cleavage.

For example, a digestive enzyme (e.g., trypsin, proteinase K, Staphylococcus aureus V8-proteinase, and other proteinases known in the art) can be used when the antibody is conjugated to the capture-associated universal oligo through some peptide linkage (with or without a scaffold); a restriction endonuclease can be used when there is a specific sequence present in the capture-associated universal oligo, susceptible to the particular restriction endonuclease, between the portion of the capture-associated universal oligo that is complementary to the electrode-associated universal oligo molecule and the portion of the capture-associated universal oligo that is conjugated to the capture moiety. In some embodiments, restriction endonuclease recognition sites and restriction endonucleases are chosen that allow cleavage of double-stranded nucleic acids. In other embodiments, restriction endonuclease recognition sites and restriction endonucleases are chosen that allow cleavage of single-stranded nucleic acids. Likewise, a flap endonuclease, such as RAD2, or XPG, could be used when there is a specific structure present in the capture-associated universal oligo, susceptible to the particular flap endonuclease, between the portion of the capture-associated universal oligo that is complementary to the electrode-associated universal oligo molecule and the portion of the capture-associated universal oligo that is conjugated to the capture moiety. Those of skill in the art would appreciate and understand the particular types of structure susceptible to flap endonuclease cleavage.

For example, in certain embodiments where it is intended that a restriction endonuclease will be used to separate the capture moiety from the capture-associated universal oligo, the capture-associated universal oligo will be engineered to contain a specific restriction endonuclease recognition sequence between the portion of the capture-associated universal oligo that is complementary to the electrode-associated universal oligo molecule and the portion of the capture-associated universal oligo that is conjugated to the capture moiety. This restriction endonuclease recognition sequence will be designed, and the appropriate restriction endonuclease selected, to cleave only between the portion of the capture-associated universal oligo that is complementary to the electrode-associated universal oligo molecule and the portion of the capture-associated universal oligo that is conjugated to the capture moiety, and not in the region of the capture-associated universal oligo that is complementary to the electrode-associated universal oligo. For restriction endonucleases that require a double-stranded recognition site, an oligonucleotide that is complementary to the restriction endonuclease recognition sequence must be hybridized to the capture-associated universal oligo to form the double-stranded restriction endonuclease recognition site.

In some embodiments where such cleavage is performed, the cleavage reaction is performed after the capture reaction has been completed and after a selective purification reaction is employed in order to segregate the desired reaction product (e.g., comprising reacted capture-associated universal oligo, capture moiety and target agent). For example, the reaction product can be subjected to a secondary capture using immobilized binding partners (e.g., secondary immobilized antibodies) that are designed to immobilize reacted capture-associated universal oligo complexes, but not unreacted capture-associated oligos. Separation procedures well-known to those of ordinary skill in the art (e.g., washing, eluting, etc.) may be used to separate unreacted capture-associated universal oligos from the immobilized reacted capture-associated universal oligo complexes. An oligo complementary to the restriction endonuclease restriction sequence is hybridized to the capture-associated universal oligo, and a cleavage reaction may then be employed to separate the universal oligos from the immobilized capture-associated universal oligo complexes, and the resulting solution containing the purified universal oligos can be transferred to the electrochemical detection device for signal detection.

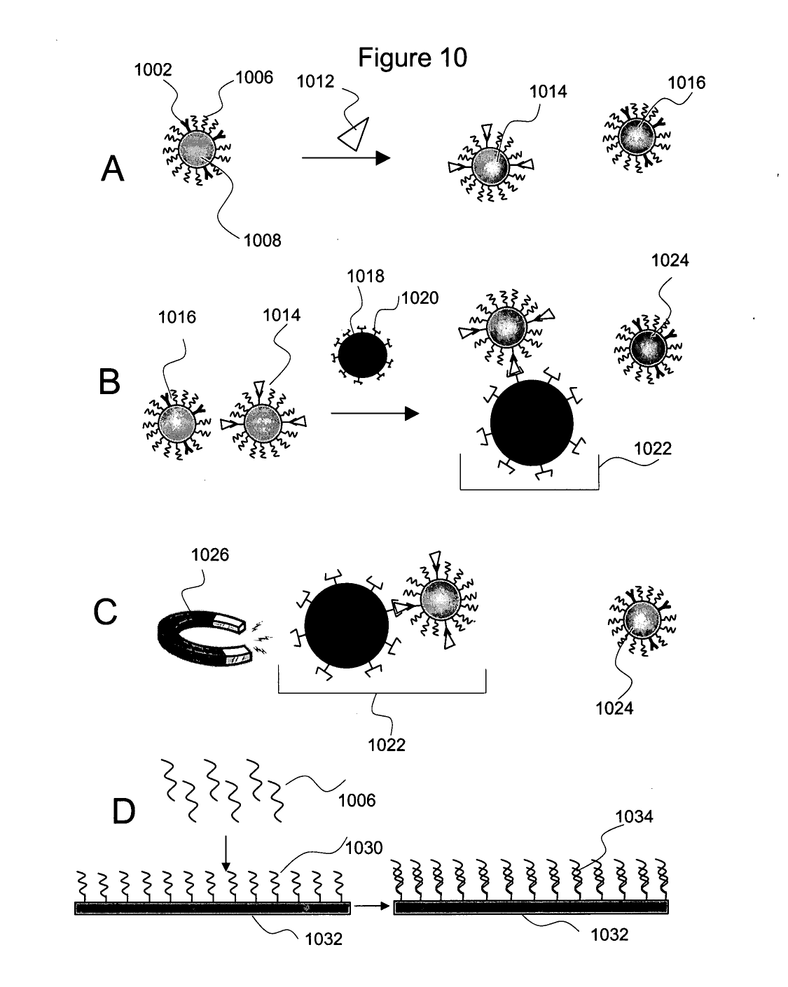

FIG. 10 illustrates one embodiment of the present invention for determining the presence of target agent in a sample by electrochemical detection that uses loaded scaffolds comprising capture-associated universal oligos and capture moieties. Capture-associated universal oligos (1006) and capture moieties (1002) are affixed to the surface of the loaded scaffold (1008) (the manufacture of which is described in FIG. 3). In FIG. 10 step A, loaded scaffold (1008) is mixed with or otherwise contacted with a sample containing target agent (1012) to form reacted loaded scaffold (1014) and unreacted loaded scaffold (1016). The reacted loaded scaffold (1014) comprises loaded scaffold (1008) with at least one target agent (1012) bound to a capture moiety (1002) on the loaded scaffold (1008). The unreacted loaded scaffold (1016) comprises loaded scaffold (1008) with capture moieties (1002) that did not bind to a target agent (1012).

In FIG. 10 step B, the products from FIG. 10 step A (reacted loaded scaffold (1014) and unreacted loaded scaffold (1016)) are mixed or otherwise contacted with immobilized binding partner complex (1018) to form immobilized binding partner/reacted loaded scaffold complexes (1022) and free unreacted loaded scaffolds (1024). The immobilized binding partner complex (1018) has binding partners (1020) affixed or otherwise attached to the surface of the immobilized binding partner complex (1018). In this embodiment, the immobilized binding partner complex (1018) further comprises a magnetic core. The binding partners (1020) of the immobilized binding partner complex (1018) in this embodiment are designed to bind to a different portion of the target agent (1012) than the capture moiety (1002) of the loaded scaffolds (1008) to form immobilized binding partner/reacted loaded scaffold complexes (1022). The free unreacted loaded scaffolds (1024) comprise unreacted loaded scaffolds (1016) that did not form an immobilized binding partner/reacted loaded scaffold complex (1022) due to the fact that unreacted loaded scaffolds (1016) did not bind a target agent (1012) that is recognized by the immobilized binding partner (1020).

In FIG. 10 step C, a magnetic field (1026) is applied across the products of FIG. 10 step B (immobilized binding partner/reacted loaded scaffold complexes (1022) and free unreacted loaded scaffolds (1024)). The magnetic core of the immobilized binding partner complex (1018) of the immobilized binding partner/reacted loaded scaffold complex (1022) is drawn to the magnetic field. The free unreacted loaded scaffold (1024) is not bound to an immobilized binding partner complex (1018) and therefore remains in solution. In practice, the reaction represented by FIG. 10 step C may be performed in a reaction container such as a test tube (not shown). Application of the magnetic field (1026) on a side of test tube will draw the magnetized immobilized binding partner/reacted loaded scaffold complexes (1022) to the side of the test tube wall most proximate to the magnetic field (1026), and leave the unmagnetized free unreacted loaded scaffolds (1024) in solution where they may be separated by methods such as aspiration.

In FIG. 10 step D, capture-associated universal oligos (1006) from the immobilized binding partner/reacted loaded scaffold complexes (1022) that were magnetically separated from the free unreacted loaded scaffolds (1024) in FIG. 10 step C are released from the loaded scaffolds (1008) and applied to an electrochemical detection device (1032). The electrochemical detection device (1032) comprises one or more electrodes on which electrode-associated universal oligos (1030) have been applied. Electrode-associated universal oligos (1030) are complementary to the capture-associated universal oligos (1006). Hybridization of electrode-associated universal oligos (1030) with capture-associated universal oligos (1006) results in a double stranded nucleotide species (1034) which is subsequently detected.

In yet an additional embodiment of the invention (a “reverse antibody capture” scenario), the capture-associated universal oligo is conjugated to an antigen instead of an antibody and the target agent of interest is an antibody. In this manner, it is possible to use the other methodologies described herein to test for target agents (e.g., agents indicative of disease, microorganisms, drug response, etc.) that may be present in very low amounts or which are otherwise undetectable. By way of example, infection with certain viruses such as hepatitis or HIV may not lead to detectable viral titer for extended periods of time. Nonetheless, the presence of the viral infection results in the generation of detectable levels of antibodies, typically over a period of 3-12 months. In this embodiment of the invention, a capture-associated universal oligo having an antigen as a capture moiety is employed to facilitate the detection of the antibodies in question shortly after infection as opposed to months or years as presently experienced.

In accordance with this embodiment, use of the universal oligo chip involves the following elements: (1) electrode-associated universal oligos immobilized on a surface, wherein the surface comprises an electrode, (2) a capture-associated universal oligo conjugated to an antigen corresponding to a target antibody (e.g., in the case of a test for HIV infection, the antigen is an HIV antigen), (3) a sample from an individual suspected of hosting the target agent, and (4) immobilized antibodies to the antigen. In one aspect, the capture-associated universal oligo is contacted with the sample in a first vessel to form a first mixture, and the first mixture is contacted with immobilized antibodies to the antigen (in the HIV example antibodies to the particular HIV antigen) resulting in an immobilized phase comprising the unreacted capture-associated universal oligos and a solution phase comprising reacted capture-associated universal oligos (i.e., conjugated to capture moieties that are bound to target antibodies from the sample). The solution phase of the resultant reaction mixture is then contacted with the universal oligo chip, followed by electrochemical detection as otherwise described herein.

Alternatively, the reacted capture-associated universal oligo complexes (each comprising a universal oligo, a capture moiety, and a target agent) can be immobilized while leaving the unreacted capture-associated universal oligos in solution. In such a case, the immobilized binding agent may be a general antibody binding agent, such as Protein A, Protein G, a thiophilic resin, and the like, which nonspecifically binds antibodies in the mixture. The only capture-associated universal oligos that are immobilized are those conjugated to a capture moiety that has bound to the target antibody, and capture-associated universal oligos not conjugated to reacted capture moieties remain in solution and can be removed from the immobilized capture-associated universal oligos by methods known in the art and/or described herein. In other embodiments, if the class of the target antibody is known, an anti-class-specific antibody can be used. In still further embodiments, an antibody specific for the capture moiety/target agent complex or an antibody specific for the target agent can be used, e.g., specific for an epitope other than that bound by the capture moiety. Other variations on this embodiment include one or more other aspects of the invention described herein or such other modification know to those of ordinary skill in the art. In addition to the detection of antibodies to scarce or low level targets, this embodiment can be employed to detect any moiety capable of generating an antibody response, doing so in a manner which is more facile and rapid than existing or previously known methods. This alternative embodiment may be employed in a multi-target (so-called multiplexed) format, thereby allowing for the screening of multiple target antibodies simultaneously.

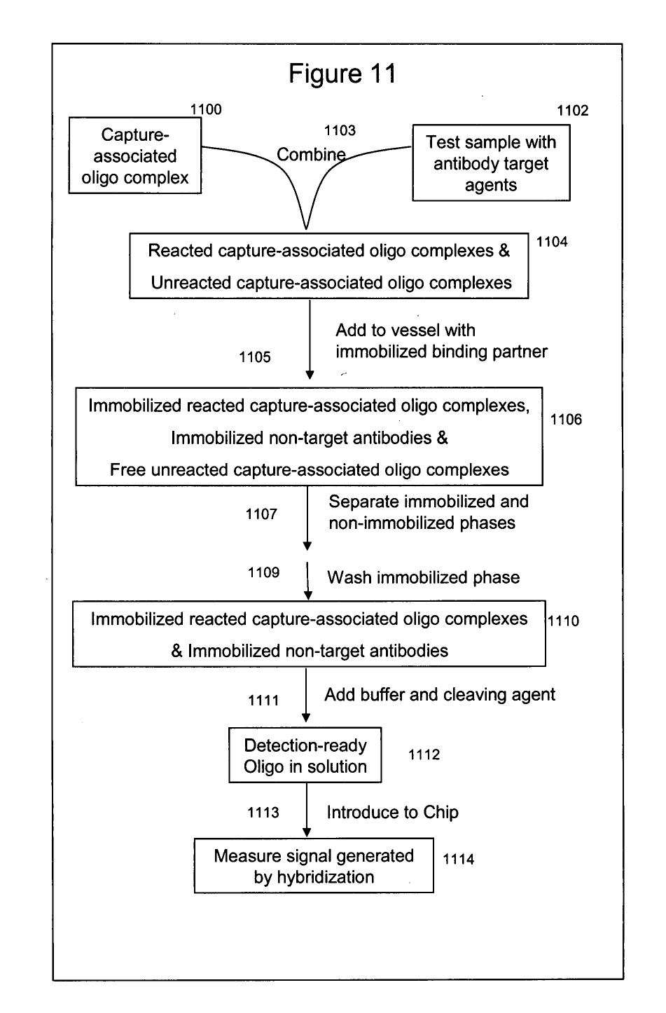

A simple flow chart of an example of a “reverse antibody capture” embodiment of the present invention is shown in FIG. 11. First, a capture-associated oligo complex comprising an antigen (capture moiety) (1100) and a test sample suspected of containing antibody target agents (1102) are combined (1103), resulting in the formation of reacted capture-associated oligo complexes (i.e., bound to target antibody) and unreacted capture-associated oligo complexes (i.e., not bound to target antibody) (1104). The reacted capture-associated oligo complexes and unreacted capture-associated oligo complexes (1104) are added to a vessel or otherwise contacted with immobilized binding partners (1105), which can be a general protein binding agent such as Protein A, or a more specific binding agent that binds the reacted capture-associated oligo complexes and any free antibodies from the sample to create a mixture comprising a) immobilized reacted capture-associated oligo complexes, b) immobilized non-target antibodies, and c) free unreacted capture-associated oligo complexes (1106). At step 1107, unreacted capture-associated oligo complexes (in “non-immobilized phase”) are removed to produce a mixture comprising the immobilized reacted capture-associated oligo complexes and non-target antibodies in an “immobilized phase” (1110) (this separation may include one or more wash steps (1109)). At step 1111 a buffer is added to the immobilized phase along with an agent (a “cleaving agent,” e.g., a restriction endonuclease) to remove the capture-associated oligo from the immobilized reacted capture-associated oligo complex. The released oligo will then be in solution (1112), and may be added to the chip (1113). A signal generated by the electrochemical detection device is measured (1114).

Loaded scaffolds can also be used in alternative embodiments of a reverse antibody capture method. In some such embodiments, the immobilized binding partner is an immobilized antigen, the target agent is an antibody, and the method involves the following elements: (1) electrode-associated oligos immobilized on a surface, wherein the surface comprises an electrode, (2) a loaded scaffold associated with a general antibody binding agent such as Protein A, Protein G, a thiophilic resin, and the like, or if the class of the target antibody is known, a anti-class-specific antibody (3) a sample from an individual suspected of hosting the target antibody, and (4) immobilized antigens corresponding to the target antibody (i.e., in the case of a test for HIV infection, the antigen is an HIV antigen). In one aspect, the immobilized binding partners are contacted with the sample in a first vessel to form a first mixture, and the first mixture is contacted with loaded scaffolds with capture moieties to the target antibody. The resulting solution contains an immobilized phase containing the reacted loaded scaffolds which have bound to the target antibodies bound to the immobilized binding partners, and a solution phase containing unreacted loaded scaffolds, which is subsequently removed. The capture-associated oligos associated with the reacted loaded scaffolds undergo a cleavage and/or linear or logarithmic amplification step to release oligos into solution, as described elsewhere herein. The solution phase of the resultant reaction mixture is then contacted with the electrode-associated oligos, followed by electrochemical detection as otherwise described herein.

In yet another embodiment, a reverse bead/scaffold capture method is used where an immobilized binding partner is contacted with a sample to form a first mixture under conditions that promote binding of the binding partner to a target agent in the sample. The first mixture is contacted with a loaded scaffold, and a capture moiety on the loaded scaffold binds to a different epitope of the target agent or to the target agent/binding partner complex, and is thereby immobilized leaving the unreacted loaded scaffolds in solution with detection proceeding as described elsewhere herein. Other variations on this preferred embodiment include one or more other aspects of the invention described herein or such other modification known to those of ordinary skill in the art.

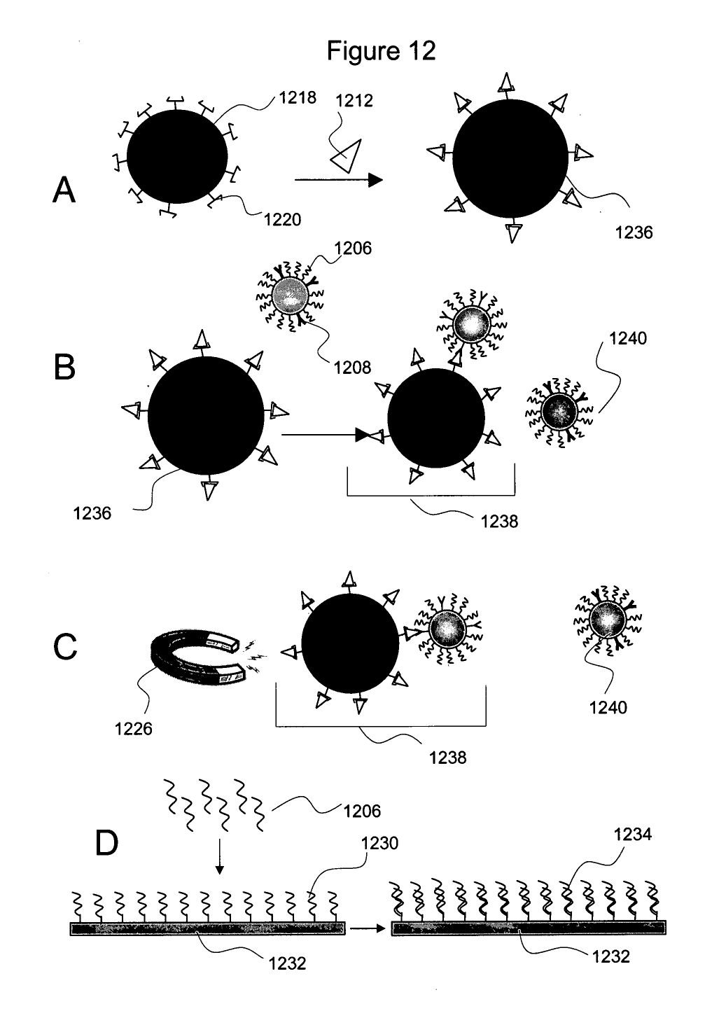

FIG. 12 illustrates a reverse bead/scaffold capture method where an immobilized binding partner is contacted with a target agent to form a first mixture, and this mixture is contacted with a loaded scaffold. In FIG. 12 step A, binding partner (1220) is immobilized on a magnetic bead to form an immobilized binding partner complex (1218) that is then is mixed with or otherwise contacted with a sample suspected of containing target agent (1212) to form reacted immobilized binding partner complex (1236). Reacted immobilized binding partner complex (1236) is comprised of immobilized binding partner complex (1218) with target agent (1212) bound to a binding partner (1220).

In FIG. 12 step B, the product from the reaction in FIG. 12 step A, the reacted immobilized binding partner complex (1236), is mixed or otherwise contacted with loaded scaffold (1208) to form a reacted immobilized binding partner/loaded scaffold complex (1238) and an unbound loaded scaffold (1240). Capture-associated universal oligos (1206) are affixed to the surface of the loaded scaffold (1208) (the manufacture of which is described in FIG. 3). The reacted immobilized binding partner/loaded scaffold complex (1238) comprises a loaded scaffold (1208) which has bound to a different portion of the target agent (1212) than the binding partner (1220) of the reacted immobilized binding partner complex (1236), to form reacted immobilized binding partner/loaded scaffold complex (1238). The unbound loaded scaffold (1240) represents those loaded scaffolds (1208) that did not bind to the target agent (1212) on the reacted immobilized binding partner complex (1236) due to, e.g., the loaded scaffold being in excess of target agent, or because the capture moiety present on the loaded scaffold did not recognize and bind a target agent present in the sample.

In FIG. 12 step C, a magnetic field (1226) is applied across the products of FIG. 12 step B (reacted immobilized binding partner/loaded scaffold complex (1238) and free unbound loaded scaffold (1240)). The magnetic core of the immobilized binding partner complex (1218) of the reacted immobilized binding partner/loaded scaffold complex (1238) is drawn to the magnetic field. The free unbound loaded scaffold (1240) is not bound to an immobilized binding partner complex (1218) and therefore remains in solution. In practice, the reaction represented by FIG. 12 step C may be performed in a reaction container such as a test tube (not shown). Application of the magnetic field (1226) on a side of test tube will draw the magnetized reacted immobilized binding partner/loaded scaffold complexes (1238) to the side of the test tube wall most proximate to the magnetic field (1226), and leave the unmagnetized free unbound loaded scaffold (1240) in solution where it may be separated by methods such as aspiration.

In FIG. 12 step D, capture-associated universal oligos (1206) from the reacted immobilized binding partner/loaded scaffold complexes (1238) that were magnetically separated from the free unbound loaded scaffold (1240) in FIG. 12 step C are released from the reacted immobilized binding partner/loaded scaffold complexes (1238) and applied to an electrochemical detection device (1232). The electrochemical detection device (1232) comprises one or more electrodes on which electrode-associated universal oligos (1230) have been applied. Electrode-associated universal oligos (1230) are complementary to the capture-associated universal oligos (1206). Hybridization of electrode-associated universal oligos (1230) with capture-associated universal oligos (1206) results in a double stranded nucleotide species (1234) which is subsequently detected.

In certain embodiments, a reverse bead/scaffold capture method may be multiplexed. Such an embodiment includes providing (1) an electrochemical detection device comprising electrode-associated oligos, (2) immobilized binding partners corresponding to the target agents, (3) a sample suspected of containing the target agents, and (4) a set of loaded scaffolds where the scaffold comprises a capture-associated oligo, which is complementary to an electrode-associated oligo, and a capture moiety that binds to a different portion of the target agent or to the target agent/binding partner complex. The method comprises mixing/contacting the sample with the immobilized binding partner under reaction conditions that allow the immobilized binding partners to capture a target agent in the sample to form a first mixture. The first mixture is then mixed/contacted with the loaded scaffolds where the loaded scaffolds bind to a different portion of the target agent that has been captured by the immobilized binding partner or to the immobilized binding partner/target agent complex. The loaded scaffold will bind to the reacted immobilized binding partners, leaving the unbound loaded scaffolds in solution. The immobilized phase is separated, and the reacted loaded scaffold complexes are then released into solution. The capture-associated oligos associated with the reacted loaded scaffolds may then undergo optional amplification via linear or logarithmic methods known in the art. The solution is introduced to the oligo chip and to the electrochemical detection device under reaction conditions such that the capture-associated oligos and electrode-associated oligos may hybridize to each other. An electrochemical signal is generated by the hybridization of complementary capture-associated oligos and electrode-associated oligos. In various aspects of this embodiment, the capture-associated oligos that are associated with the reacted loaded scaffolds may be subjected to a cleavage reaction and/or a linear or logarithmic amplification step after being separated from unreacted loaded scaffolds but before being contacted with the electrode-associated oligos.

In a preferred embodiment, the present invention allows for the quantification of one or more target agents. The following example is provided to exemplify the invention without limiting the scope in any manner. In this embodiment, detecting and quantifying the presence of one or more target agents in a sample is accomplished by providing (1) an electrochemical detection device comprising a plurality of electrodes, where each electrode has an immobilized electrode-associated oligo, where each electrode-associated oligo has a known, predetermined sequence, (2) a set of one or more capture-associated oligos, where each of the capture associated oligos is complementary in sequence to one electrode-associated oligo, and where each capture-associated oligo is conjugated to a capture moiety specific for one or more target agents to be detected (or, alternatively, conjugated to a moiety capable of being selectively captured, i.e., a “capturable moiety”) such as, for example, one or more antibodies to drug metabolites, (3) a set of quantifying oligos, where each of the quantifying oligos is complementary to one of the electrode-associated oligos, and where each quantifying oligo is present in a known (e.g., titrated, calibrated verified, validated, etc.) concentration, (4) a sample suspected of containing one or more target agents (in this case, drug metabolites), and (5) immobilized binding partners that specifically associate with the capture moieties that have not bound a target agent.

The method comprises mixing/contacting the sample with the capture-associated oligos under reaction conditions that allow the binding of the capture moiety (antibody to the drug metabolites) to the target agent(s) (drug metabolites) present in the sample, if any, to create a first mixture. The first mixture is then mixed/contacted with the immobilized binding partners, thereby immobilizing any unreacted capture-associated oligos (i.e., conjugated to a capture moiety that has not reacted with a target agent). This results in the formation of an immobilized phase and a solution phase. The immobilized phase comprises the immobilized binding partners and unreacted capture-associated oligos, and the solution phase comprises reacted capture-associated oligos (i.e., conjugated to a capture moiety that has reacted with a target agent). The method further includes contacting/mixing the solution phase with the third quantifying oligos thereby resulting in a second mixture containing the reacted capture-associated oligo complex as well as the quantifying oligos, each of which has a known concentration. The second mixture is contacted with the electrochemical detection device under reaction conditions such that the single capture-associated oligos hybridize to the electrode-associated oligos where electrochemical signals are generated by the hybridization events.

Hybridization of the quantifying oligos, each being of known concentration (and in one embodiment, each is of a different known concentration and in a preferred embodiment, each is present in a known graduated concentration with respect to each other), will generate a signal, the magnitude of which corresponds to its respective known concentration. If the drug metabolites are present in the sample tested, the one or more capture-associated oligos from the reacted capture-associated oligo complexes will hybridize with electrode-associated oligos, thereby resulting in a signal. The magnitude of that electrochemical signal can be used to calculate the concentration of the target agent in the sample by correlation with the magnitude of the electrochemical signal measured for the hybridization of each of the quantifying oligos.

Accordingly, the present method provides an accurate means for determining the concentration of a target agent in a sample with the benefit of the correlative standards being measured in the same reaction mixture, thereby eliminating such variables as sample concentration, mixing errors, temperature variance, or such other factors that are typically encountered when samples are run in separate tests. As with each of the other embodiments of the present invention, the order of the steps may be changed, with additional steps being added and/or eliminated, or such other variations as would be understood by persons having ordinary skill in the art, without deviating from the intent, purpose and/or other benefits of the present invention.

In certain situations, it may be beneficial to use logarithmic or linear amplification methods (e.g., PCR, isothermal amplification, etc.) to increase the number of oligos (e.g., capture-associated universal oligos and/or complements thereof) available for binding to the electrode-associated universal oligos, thus enhancing the signal created through complementary binding. Such methods of amplification are well known in the art and may include polymerase chain reaction (“PCR”) and linear amplification via such polymerases as T7 polymerase. In such embodiments, a capture-associated universal oligo must be designed to incorporate a polymerase (e.g., 5′ to 3′ RNA or DNA polymerase) recognition sequence to allow binding of a polymerase enzyme that can amplify at least the portion of the capture-associated universal oligo that corresponds to (e.g., is complementary or identical to) the electrode-associated universal oligo (e.g., to produce an RNA or DNA amplification product, respectively). If the polymerase binding sequence is in single-stranded form, it is hybridized to its nucleic acid complement to create a double-stranded polymerase binding site prior to addition of an appropriate polymerase, many of which are well known to those of ordinary skill in the art. Alternatively, the capture-associated universal oligo can be engineered to contain a double-stranded portion comprising the polymerase recognition site, thereby eliminating the step of hybridization of an oligonucleotide to create such a double-stranded site.

As noted above, the capture-associated universal oligo may be conjugated to the capture moiety at either the 3′ or 5′ end. If the capture-associated universal oligo is conjugated to the capture moiety at the 3′ end, then the polymerase recognition site is preferably located between the capture moiety and the region corresponding to (e.g., identical or complementary to) a sequence of the electrode-associated universal oligo. If the capture-associated universal oligo is conjugated to the capture moiety at the 5′ end, then the polymerase recognition site is preferably located at the end of the capture-associated universal oligo that is distal to the capture moiety. In certain embodiments, a termination signal is also engineered into the capture-associated universal oligo at the nucleotide position at which the polymerase is to terminate polymerization, e.g., a position after the region of the capture-associated universal oligo that is complementary or identical to an electrode-associated universal oligo.

In some embodiments, the capture-associated universal oligo is used as a template for linear amplification, and the capture-associated universal oligo is therefore designed to encode a) a sequence identical to a sequence of the corresponding electrode-associated universal oligo (as opposed to a sequence complementary to a sequence of the electrode-associated universal oligo, as would be the case if the capture-associated universal oligo were to be hybridized directly to the electrode-associated universal oligo), and b) a sequence corresponding to a polymerase recognition sequence at its 3′ end adjacent to or overlapping with the region identical to a sequence of the electrode-associated universal oligo. Following binding of the target agent to the capture moiety and isolation of the resulting “reacted capture-associated universal oligo complex” from the sample (using, for example, immobilized binding partners as discussed herein), an oligonucleotide encoding the complement to the polymerase recognition sequence encoded by the capture-associated universal oligo is introduced to the reacted capture-associated universal oligo complex, and its binding to the complex creates a double-stranded polymerase recognition site. (Alternatively, as noted above, the capture-associated universal oligo could be engineered to contain a double-stranded portion comprising the polymerase recognition site, thereby eliminating the step of hybridization of an oligonucleotide to create such a double-stranded site.) The reacted capture-associated universal oligo comprising a double-stranded polymerase recognition site (whether by design or hybridization) is exposed to an aqueous solution comprising a polymerase and an excess of NTP or dNTP under conditions that allow the polymerase and reactants to create an intermediate duplex comprising a double-stranded DNA (or RNA-DNA hybrid, depending on, e.g., the polymerase and nucleotides used) having a first end that bears a polymerase recognition site (e.g., a phage-encoded RNA recognition site). As this reaction continues, the polymerase displaces the nascent strand of the double-stranded nucleic acid, resulting in multiple oligos that are complementary to the capture-associated universal oligo and the electrode-associated universal oligo on the universal oligo chip. As noted above, in such an embodiment, the electrode-associated universal oligo will have the same sequence as the capture-associated universal oligo, and both will be complementary to the linear amplification products.

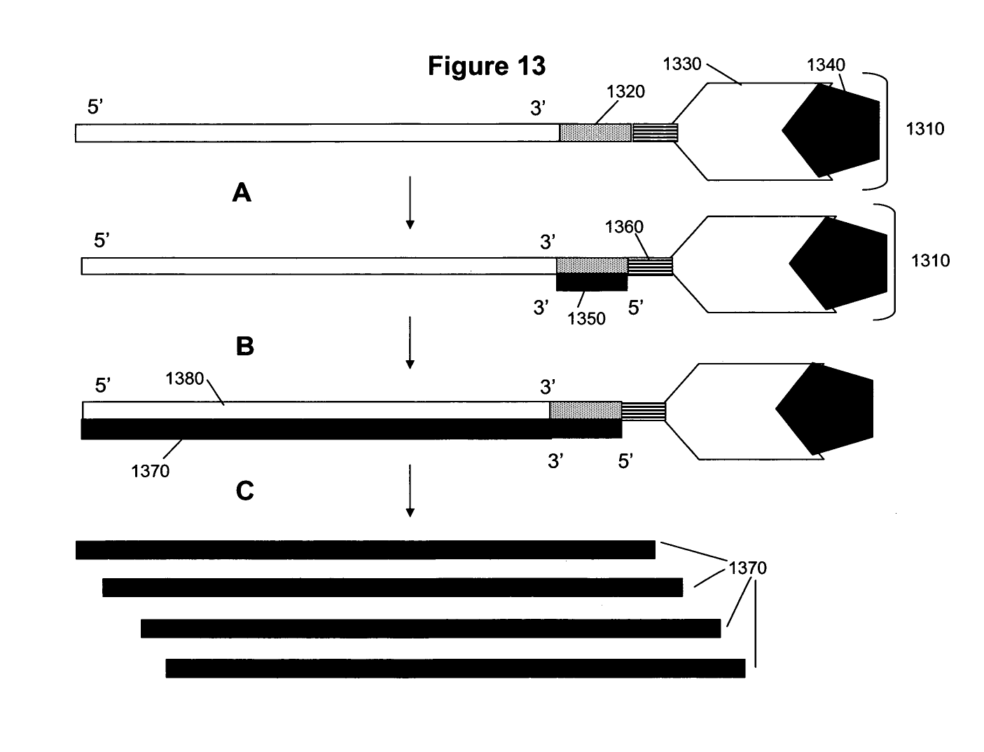

For example, FIG. 13 is a schematic diagram demonstrating the use of an engineered polymerase recognition site to create multiple copies of a nucleic acid for more sensitive detection of a target agent. In step A, a reacted capture-associated oligo complex (1310) comprising polymerase recognition sequence (1320) and capture moiety (1330) bound to target agent (1340) is bound to complementary oligo (1350), which is complementary to and binds to the polymerase recognition sequence (1320) to create a double-stranded polymerase recognition site (1360). In Step B, reacted capture-associated oligo complex (1310) comprising the double-stranded polymerase recognition site (1360) is reacted with the appropriate nucleotides and polymerase to create an oligo (1370) complementary to the capture-associated oligo (1380). In Step C the polymerase reactions are carried out repeatedly to create multiple copies of the complementary oligo (1370) via linear amplification.

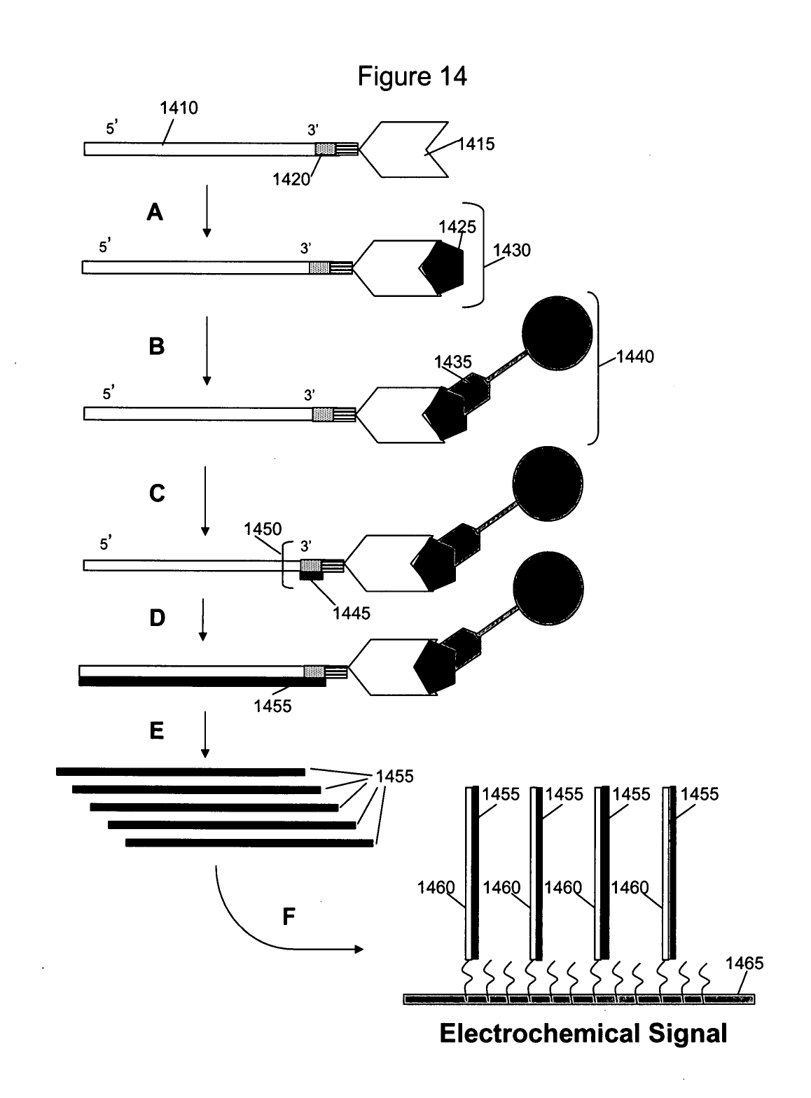

In certain embodiments, the amplification methods disclosed herein are combined with methods to separate reacted capture-associated oligos from unreacted capture-associated oligos. For example, FIG. 14 is a schematic diagram illustrating a further example comprising the combination of isolation using an immobilized binding partner that binds to the target agent and polymerase amplification techniques. In step A, a capture-associated oligo (1410) conjugated to a capture moiety (1415) and further comprising a polymerase recognition sequence (1420) is exposed to a sample comprising target agent (1425) to create reacted capture-associated oligo complex (1430). In step B, reacted capture-associated oligo complex (1430) is exposed to an immobilized binding partner (1435), which specifically binds to the target agent, to create immobilized reacted capture-associated oligo complex (1440). In step C, hybridization of immobilized reacted capture-associated oligo complex (1440) to an oligonucleotide (1445) complementary to the polymerase recognition sequence (1420) provides a double-stranded polymerase recognition site (1450). In step D, the immobilized reacted capture-associated oligo complex (1440) further comprising the double-stranded polymerase recognition site (1450) is reacted with the appropriate nucleotides and polymerase to provide creation of an oligo (1455) complementary to the capture-associated oligo (1410). In step E the reactions are carried out repeatedly to create multiple copies of the complementary oligo (1455) via linear amplification. In step F the complementary oligos (1455) are introduced to electrode-associated oligos (1460) on oligo chip (1465). The binding of the complementary oligos (1455) to the electrode-associated oligos (1460) generates a signal in an electrochemical detection device.

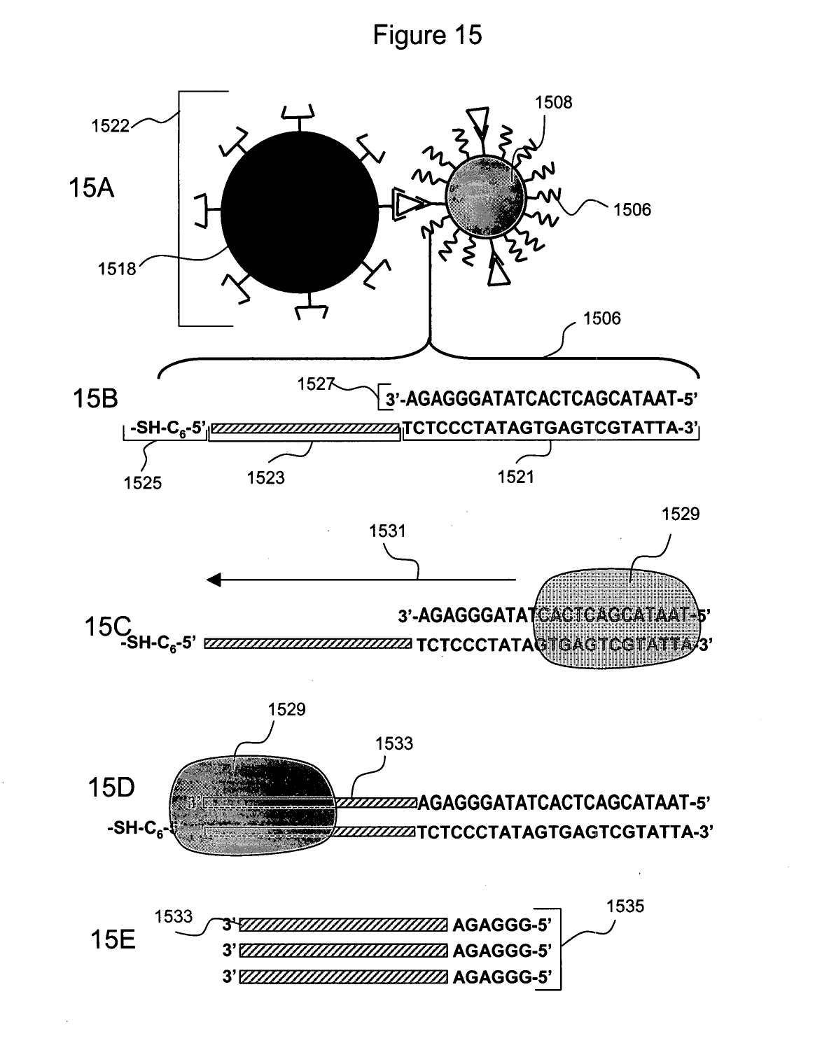

FIG. 15 illustrates a further example in which loaded scaffolds are used in combination with a method of linear amplification of the capture-associated universal oligos on the loaded scaffolds using T7 RNA polymerase. FIG. 15A depicts an immobilized binding partner/reacted loaded scaffold complex (1522) which is comprised of a reacted loaded scaffold (1508) and an immobilized binding partner complex (1518). Reacted loaded scaffold (1508) is shown with an associated capture-associated universal oligos (1506), which is the template nucleic acid to be amplified. Note, in this embodiment, since the RNA transcripts produced will be complementary to the capture-associated universal oligo (1506) attached to the scaffold, the capture-associated universal oligo (1506) has a sequence that is the same or substantially the same as the electrode-associated universal oligo.

In FIG. 15B, a magnified view of capture-associated universal oligo (1506) is shown. Capture-associated universal oligo (1506) is comprised of a functionalized thiol group (1525) (used to link oligonucleotides to scaffold substrates such as gold), universal oligo sequence (1523) and sequence complementary to T7 RNA polymerase promoter sequence (1521). Also shown is a short oligonucleotide sequence (1527) corresponding to the T7 RNA polymerase promoter sequence. Because T7 RNA polymerase requires double-stranded DNA for initiation of transcription, the T7 RNA polymerase promoter sequence (1521) may be either engineered as a double-stranded region on the capture-associated universal oligo (1506) before the oligo is affixed to the scaffold, or oligonucleotide sequence (1527) may be added as a primer to the amplification reaction mix after the capture by the immobilized binding partner complex has been completed (as described in the specification).

FIG. 15C shows T7 RNA polymerase (1529) binding to a double-stranded T7 RNA polymerase promoter sequence (1521) and T7 RNA polymerase promoter sequence. Arrow (1531) depicts the 5′ to 3′ direction of polymerization of T7 RNA polymerase (1529). In FIG. 15D, T7 RNA polymerase (1529) is depicted as having transcribed nascent RNA molecules (1533) using the capture-associated universal oligo as a template for synthesis.

In FIG. 15E, the products of several T7 RNA polymerase amplification reactions are depicted. Amplified capture-associated universal oligo products (1535) are comprised of nascent RNA molecules (1533), and the sequence “AGAGGG” which represents the first bases transcribed T7 RNA polymerase from the T7 RNA promoter sequence (1527) incorporated into RNA during transcription.

In certain embodiments, the polymerase recognition site created by this double-stranded region is a phage-encoded RNA polymerase recognition sequence. Exemplary polymerases useful in such isothermal amplification reactions include RNA phage polymerases, including but not limited to T3 polymerase, SP6 polymerase, Qβ polymerase, and T7 polymerase. In one embodiment of this aspect, T7 RNA polymerase is used to produce RNA transcripts of the capture-associated universal oligos. T7 polymerase requires a double stranded T7 RNA polymerase promoter site for transcription, and such promoter site may be engineered into the capture-associated universal oligo. Alternatively, the T7 promoter may be added as a primer in the amplification reaction mix, and the sequence complementary to the T7 promoter sequence will be engineered into the capture-associated universal oligo. T7 RNA polymerase promoter sites are well known in the art, and one such promoter sequence, provided in the MEGAshortscript™ kit commercially available from Ambion, Inc. (Austin, Tex.), is TAATACGACTCACTATAGGGAGA. The sixth “G” nucleotide from the right is the first base incorporated into the RNA transcript and the next two following G's are used to improve transcription efficiency. In an embodiment where T7 RNA polymerase is used, RNA transcripts may be generated from the capture-associated universal oligo template. The RNA transcripts generated may be hybridized to electrode associated universal oligos, and therefore the capture-associated universal oligos will be the same sequence as the electrode associated universal oligos in order to produce RNA transcripts complementary to the electrode associated universal oligos. In certain embodiments, a mutant phage-encoded polymerase (e.g., the T7 RNA polymerase mutant Y639F or S641A) is used to allow creation of DNA rather than RNA. This will increase the stability of the synthesized nucleic acids for binding to the electrode, and obviate the problem of RNAse activity. Such mutant polymerases include T7 DNA polymerase, as disclosed in U.S. Pat. No. 6,531,300, U.S. Pat. No. 6,107,037, U.S. Pat. No. 5,849,546, and Padilla and Sousa, Nucleic Acids Res 1999 27(6):1561-1563.

A number of different nucleotides can be used in the isothermal linear amplification reaction. These include not only the naturally-occurring nucleoside mono-, di-, and triphosphates: deoxyadenosine mono-, di- and triphosphate; deoxyguanosine mono-, di- and triphosphate; deoxythymidine mono-, di- and triphosphate; and deoxycytidine mono-, di- and triphosphate (referred to herein as dA, dG, dT and dC or A, G, T and C, respectively). Nucleotides also include, but are not limited to, modified nucleotides and nucleotide analogs such as deazapurine nucleotides, e.g., 7-deaza-deoxyguanosine (7-deaza-dG) and 7-deaza-deoxyadenosine (7-deaza-dA) mono-, di- and triphosphates, deutero-deoxythymidine (deutero-dT) mon-, di- and triphosphates, methylated nucleotides e.g., 5-methyldeoxycytidine triphosphate, 13C/15N labeled nucleotides and deoxyinosine mono-, di- and triphosphate. When using dNTPs and a traditional RNA polymerase, dUTP is substituted for dTTP. For those skilled in the art, it will be clear upon reading the present disclosure that modified nucleotides and nucleotide analogs that utilize a variety of combinations of functionality and attachment positions can be used in the present invention.

Asymmetric amplification using a heat stable polymerase such as Thermus aquaticus polymerase can also be used to create multiple copies of a nucleic acid complementary to the electrode-associated universal oligo. Suitable methods of asymmetric amplification are described in U.S. Pat. No. 5,066,584, which is incorporated by reference in its entirety. In such an embodiment, the electrode-associated universal oligo will have the same sequence as the capture-associated universal oligo, and both will be complementary to the asymmetric amplification products.

Amplification using the Phi29 polymerase may also be used to create multiple copies of the nucleic acids complementary to the electrode-associated universal oligo. Such methods are described in U.S. Pat. No. 5,712,124 and U.S. Pat. No. 5,455,166, both of which are incorporated by reference in their entirety. In brief, the Phi29 polymerase method will allow amplification of the capture-associated universal oligo to produce complementary nucleic acids at a single temperature by utilizing the Phi29 polymerase in conjunction with an endonuclease that will nick the polymerized strand, allowing the polymerase to displace the strand without digestion while generating a newly polymerized strand. As with asymmetric amplification, an oligonucleotide complementary to the 3′ end of the capture-moiety capture-associated universal oligo is used under conditions to create a series of single-stranded molecules complementary to the associated nucleic acid. In such an embodiment, the electrode-associated universal oligo will have the same sequence as the capture-associated universal oligo, and both will be complementary to the asymmetric amplification products.

Amplification using the polymerase chain reaction (PCR) may be used to exponentially replicate capture-associated universal oligo templates. Various disclosures involving this technique are found in U.S. Pat. Nos. 4,683,202; 4,683,195; 4,800,159; 4,965,188; and 5,512,462, each of which is incorporated herein by reference. In its most basic form, double stranded nucleic acid is separated, and a nucleic acid polymerase is used to replicate a region of each strand as defined by the polymerase primers by adding nucleotides complementary to the template strand in the 5′ to 3′ direction under varying temperatures to complete one cycle. This cycle is repeated many times over to achieve the necessary amplification of the template nucleic acid. Nucleic acid products of the PCR reaction are used as template nucleic acid for subsequent PCR reactions, and this exponential growth in amplified products can result in upwards of 100 billion nucleic acid molecules being generated from one template nucleic acid molecule.

In embodiments in which the capture-associated universal oligo is used as a template for exponential or logarithmic amplification (e.g., PCR), and the capture-associated universal oligo is therefore designed to encode a sequence complementary to a polymerase recognition sequence at its 3′ end adjacent to or overlapping a region identical or complementary to an electrode-associated universal oligo. Following binding of the target agent to the capture moiety and isolation of the resulting “reacted capture-associated universal oligo complex” from the sample (using, for example, immobilized binding partners as discussed herein), an oligonucleotide encoding the complement to the polymerase recognition sequence encoded by the capture-associated universal oligo is introduced to the reacted capture-associated universal oligo complex, and its binding to the complex creates a double-stranded polymerase recognition site. (Alternatively, as noted above, the capture-associated universal oligo could be engineered to contain a double-stranded portion comprising the polymerase recognition site, thereby eliminating the step of hybridization of an oligonucleotide to create such a double-stranded site.) The capture-associated universal oligo comprising a double-stranded polymerase recognition site (whether by design or hybridization) is exposed to an aqueous solution comprising a polymerase and an excess of NTP or dNTP under conditions that allow the polymerase and reactants to create an intermediate duplex comprising a double-stranded DNA (or RNA-DNA hybrid, depending on, e.g., the polymerase and nucleotides used) having a first end that bears a polymerase recognition site (e.g., Taq polymerase recognition site). As this reaction continues, the polymerase amplifies both the capture-associated oligo and its complement, resulting in double-stranded product. In certain embodiments, the electrode-associated universal oligo can have the same sequence as the capture-associated universal oligo or its complement. In such embodiments, the strand complementary to the electrode-associated oligo is preferably purified away from the strand not complementary to the electrode-associated oligo prior to hybridization. In other embodiments, there are two electrode-associated oligos for each capture-associated oligo, one complementary to the capture-associated universal oligo and one complementary to the complement of the capture-associated universal oligo. These two electrode-associated oligos may be immobilized to the same electrode, or to separate electrodes. The latter scenario may be beneficial, e.g., by providing an internal control for the hybridization reaction. In certain embodiments, the two strands amplified are separated from one another prior to hybridization.

Any logarithmic amplification technique known to those of skill in the art may be used in conjunction with the present invention including, but not limited to, polymerase chain reaction (PCR) techniques. PCR may be carried out using materials and methods well known to those of skill in the art, as are the many modifications to the basic method such as variations in the polymerase, reaction buffer, template nucleic acid, thermal cycling profile, reaction additives, primer design and other modifications. Primers complementary to the beginning and end of the portion of nucleic acid to be amplified are used by the polymerase as binding recognition sites. These primers, along with template nucleic acid, an appropriate nucleic acid polymerase, buffer solution, nucleotides, and water are mixed in a tube. This reaction mix is placed in a thermocycler or similar device capable of raising and lowering the temperature of the reaction mix. The thermocycler will then sequentially change the temperature of the reaction repeatedly according to a thermal cycling profile. An example of such thermal cycling profile is: heat the reaction mix at 96° C. for 5 minutes followed by 20 cycles of 96° C. for 30 seconds, 68° for 30 seconds, and 72° for 30 seconds. Following the reaction in the thermocycler, the reaction mix will contain large numbers of amplified template. See, generally, PCR Technology: Principals and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); and PCR: A Practical Approach (eds. McPherson et al., Oxford University Press, Oxford, UK. Other suitable amplification methods include the ligase chain reaction (LCR) (see Wu and Wallace, Genomics 4: 560 (1989) and Landegren et al., Science 241: 1077 (1988)), transcription amplification (Kwoh et al., Proc. Natl. Acad. Sci. USA 86: 1173 (1989)), self-sustained sequence replication (Guatelli et al., Proc. Nat. Acad. Sci. USA, 87: 1874 (1990)) and nucleic acid-based sequence amplification (NASBA).

In certain embodiments of the invention, a combination of logarithmic and linear amplification is used to increase the amount of oligo to be hybridized to electrode-associated universal oligos. Such reactions may be performed simultaneously or sequentially. For example, logarithmic may used for several cycles to quickly increase the amount of capture-associated universal oligo and its complement, then a linear amplification reaction may be employed to increase the amount of only the strand that is complementary to the electrode-associated universal oligo. In another example, a DNA polymerase is used for logarithmic amplification and an RNA polymerase is used for subsequent linear amplification. Other amplification strategies that may be employed to increase the amount of oligo to be hybridized to electrode-associated universal oligos are known to one of ordinary skill in the art.

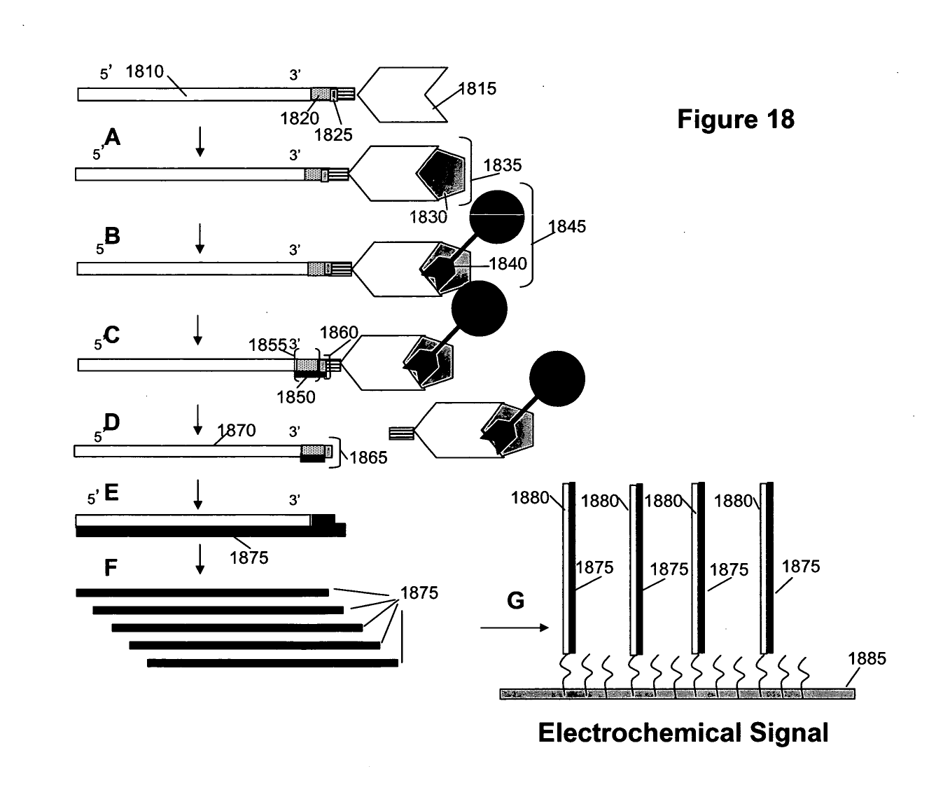

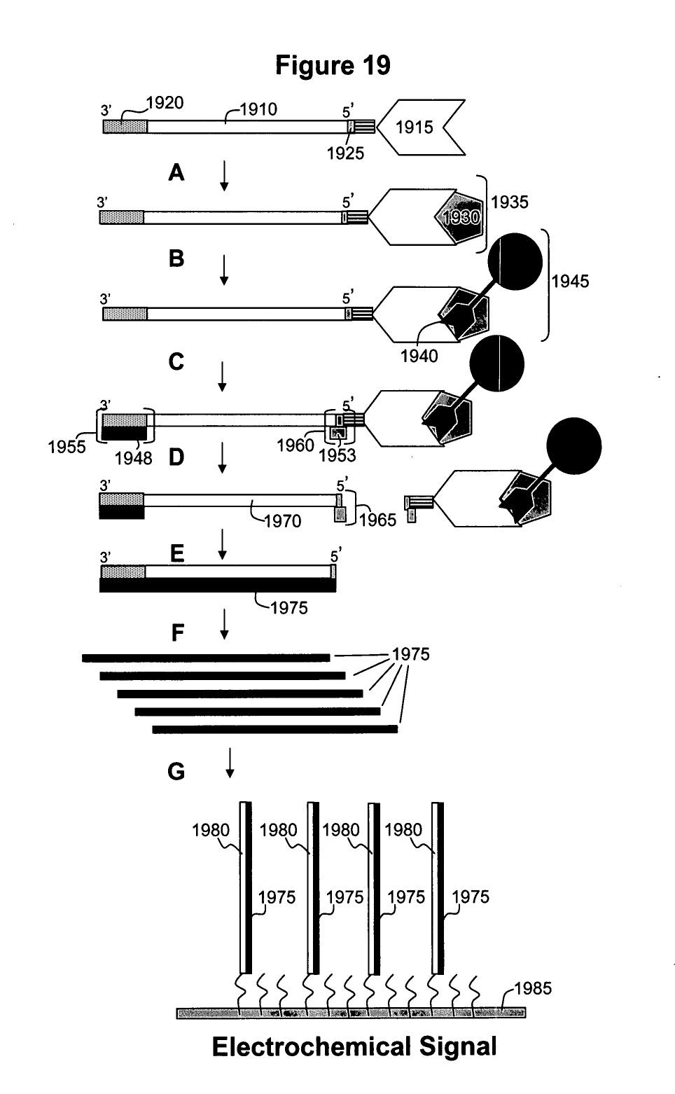

The amplification methods disclosed herein can be combined with any of the described isolation methods of the invention, including those described in FIGS. 4 and 5. For example, FIG. 14 illustrates an embodiment of the invention where a reacted capture-associated oligo complex is isolated using a binding partner that binds to the target agent, and linear amplification using a polymerase recognition site. FIGS. 18 and 19 (described infra) illustrate embodiments of the invention where a reacted capture-associated oligo complex is isolated using an immobilized binding partner that recognizes an epitope specific to the capture moiety/target agent complex, and further comprises cleavage of the reacted capture-associated oligo complex and linear amplification of the released capture-associated oligo.

In an alternate aspect of this embodiment, the amplification products are separated from the solution containing reacted capture-associated oligos before contact with the electrode-associated oligos. The reacted capture-associated oligo complexes, having been bound to an immobilized binding partner, can be removed from solution by such separation mechanisms as magnetic microparticle depletion. In an embodiment where magnetic microparticle depletion is used as a separation mechanism, the immobilized binding partners will have a magnetic substrate. The immobilized reacted capture-associated oligo complexes (i.e., bound to the immobilized binding partners) are separated from the reaction mixture by adding the mixture to a column packed with lattice-type matrix and applying a magnetic field. Such separation devices are known in the art (e.g., MACS® Columns, Miltenyi Biotec). The immobilized reacted capture-associated oligo complexes are retained on the column. The amplification products will pass through the column. Alternatively, depending on the binding partner substrate, the immobilized reacted capture-associated oligo complexes may be removed from liquid phase of the reaction solution by low speed centrifugation procedures well known in the art. Following a low speed centrifugation, the immobilized reacted capture-associated oligo complexes will form a pellet and the amplification products will remain in solution. The resultant solution is then contacted with chip-associated oligos, where a hybridization event between a chip-associated oligo and a capture-associated oligo indicates that a target agent was present in the sample. The hybridization event is detected by, e.g., electrochemical detection. The electrochemical detection can be direct or indirect.

In certain embodiments of the invention, the universal oligo is separated from the capture-associated universal oligo complex prior to linear or logarithmic amplification. In such an embodiment, the capture-associated universal oligo complex is designed to incorporate a sequence complementary to a polymerase recognition sequence at the 3′ end of the universal oligo adjacent to or overlapping with the region identical to the electrode-associated universal oligo, as well as a recognition site for an enzyme to separate the universal oligo from the capture-associated universal oligo complex (e.g., an restriction endonuclease recognition site, a protease cleavage site, etc.) For example, following binding of a target agent to a capture moiety and isolation of the resulting “reacted capture-associated universal oligo complex” from a sample, an oligonucleotide encoding the 5′ to 3′ polymerase recognition sequence and a restriction endonuclease recognition sequence is introduced to the reacted capture-associated universal oligo complex, and the binding of this oligonucleotide to the capture-associated universal oligo creates both a double-stranded polymerase recognition site and a restriction endonuclease recognition site. Following annealing, the reacted capture-associated universal oligo complex is exposed to the appropriate restriction endonuclease under conditions to allow the cleavage of the universal oligo from the capture moiety bound to the target agent. The restriction endonuclease is then optionally inactivated (e.g., through heat inactivation by exposing the solution to a temperature of 65° C. for 10 minutes), and the universal oligo released from the reacted capture-associated universal oligo complex (“released universal oligo”) may then be isolated from the capture moiety bound to the target agent. Following cleavage and optional inactivation or isolation, the released universal oligo is combined with an aqueous solution comprising, buffer, a polymerase and an excess of NTP or dNTP under conditions such that the polymerase and reactants create an intermediate duplex comprising a double-stranded DNA having a first 5′ end which bears a phage-encoded RNA polymerase recognition site. This reaction continues as the polymerase displaces the double-stranded nucleic acid, resulting in multiple copies of oligo complementary to both the released universal oligo and the electrode-associated universal oligo. As such, the electrode-associated universal oligo will have the same sequence as the capture-associated universal oligo, and both will be complementary to the linear amplification products.

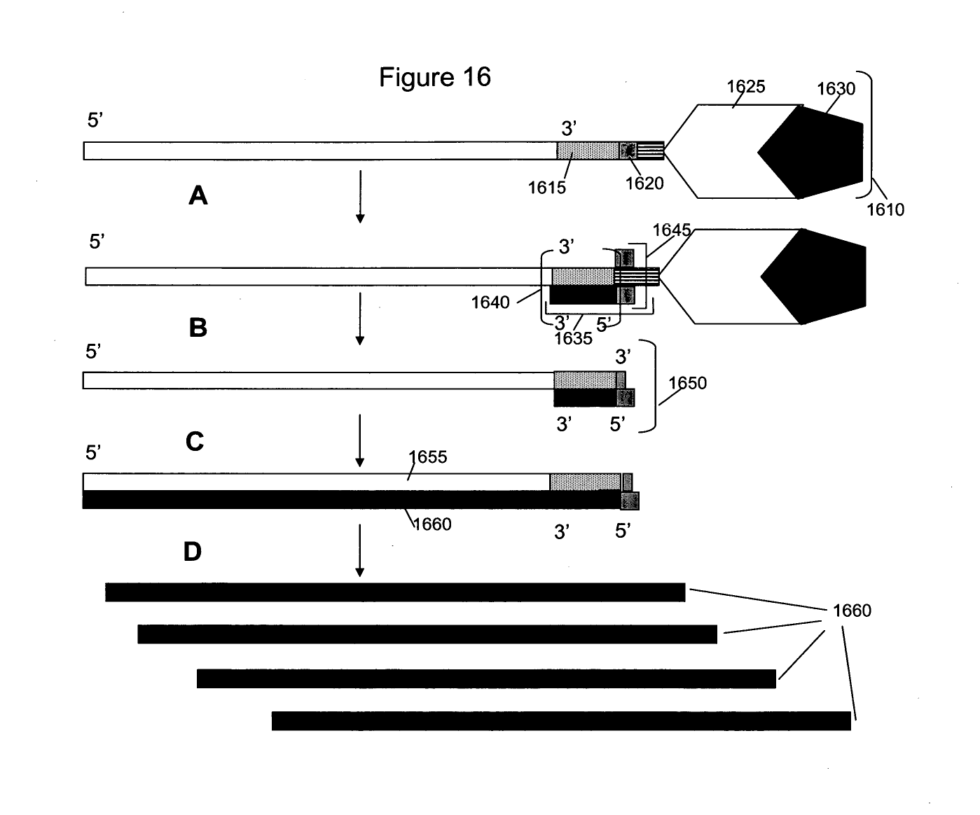

For example, FIG. 16 is a schematic diagram demonstrating the use of a capture-associated oligo comprising a restriction endonuclease recognition sequence and a polymerase recognition sequence. In step A, a reacted capture-associated oligo complex (1610) comprising polymerase recognition sequence (1615), restriction endonuclease recognition sequence (1620), and capture moiety (1625) bound to target agent (1630) is bound to complementary oligo (1635), which is complementary to and binds to the polymerase recognition sequence (1615) and the restriction endonuclease recognition sequence (1620) to create a double-stranded region comprising both a polymerase recognition site (1640) and a restriction endonuclease recognition site (1645). In step B, reacted capture-associated oligo complex (1610) further comprising polymerase recognition site (1640) and restriction endonuclease recognition site (1645) is reacted with the appropriate restriction endonuclease to remove the capture moiety (1625) and target agent (1630) from the complex (1610) to create cleaved capture-associated oligo complex (1650). In Step C, cleaved capture-associated oligo complex (1650) comprising cleaved oligo (1655) is reacted with the appropriate nucleotides and polymerase to create an oligo (1660) complementary to cleaved oligo (1655). In Step D the polymerase reactions are carried out repeatedly to create multiple copies of the complementary oligo (1660) via linear amplification.

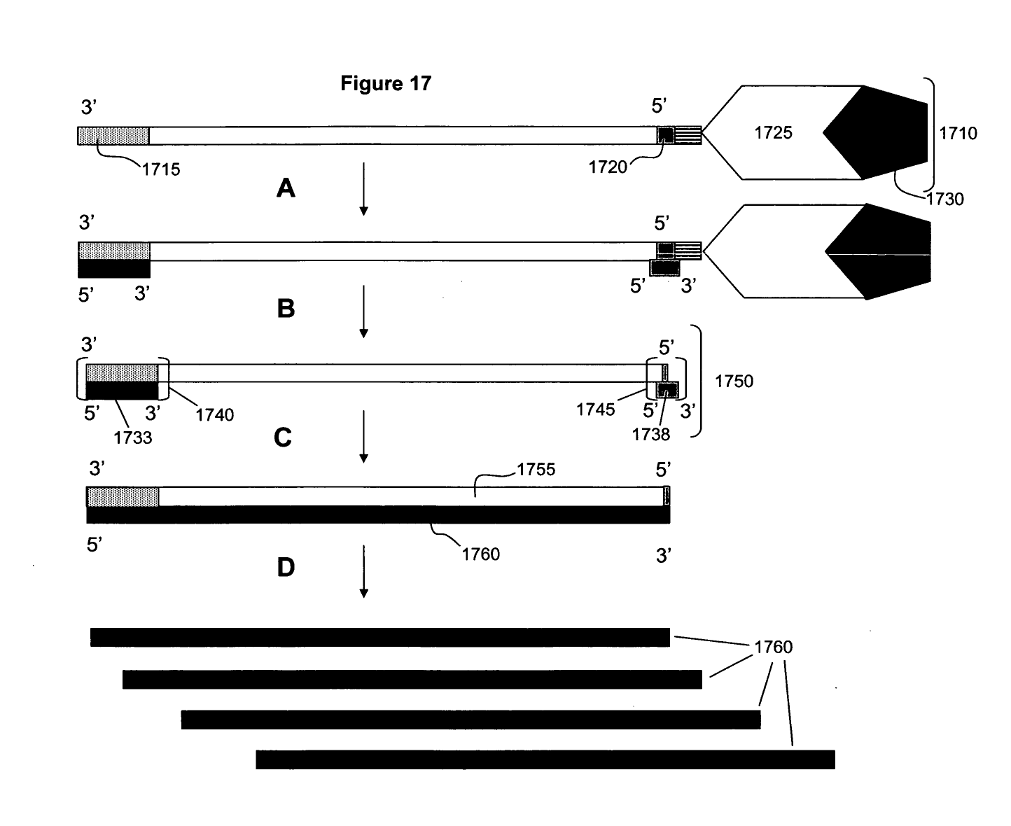

As noted above, conjugation to a capture moiety (whether via a scaffold or not) may be at the 5′ end of a capture-associated oligo. FIG. 17 is a schematic diagram demonstrating the use of such a capture-associated oligo comprising a restriction endonuclease recognition site and a polymerase recognition site. In step A, a reacted capture-associated oligo complex (1710) comprising polymerase recognition sequence (1715), restriction endonuclease recognition sequence (1720), and capture moiety (1725) bound to target agent (1730) is bound to a first complementary oligo (1733), which is complementary to and binds to the polymerase recognition sequence (1715), and a second complementary oligo (1738), which is complementary to and binds to the restriction endonuclease recognition sequence (1720) to create a first double-stranded region comprising a polymerase recognition site (1740) and a second double-stranded region comprising a restriction endonuclease recognition site (1745). In step B, reacted capture-associated oligo complex (1710) further comprising polymerase recognition site (1740) and restriction endonuclease recognition site (1745) is reacted with the appropriate restriction endonuclease to remove the capture moiety (1725) and target agent (30) from the complex (1710) to create cleaved capture-associated oligo complex (1750) comprising cleaved oligo (1755). In Step C, cleaved capture-associated oligo complex (1750) is reacted with the appropriate nucleotides and polymerase to create an oligo (1760) complementary to cleaved oligo (1755). In Step D the polymerase reactions are carried out repeatedly to create multiple copies of the complementary oligo (1760) via linear amplification.

FIG. 18 is a schematic diagram illustrating an example of an embodiment comprising a combination of isolation using an immobilized binding partner that binds to a capture moiety/target agent complex, restriction endonuclease cleavage of the reacted capture-associated oligo complex, and polymerase amplification techniques. In step A, a capture-associated oligo (1810) conjugated to a capture moiety (1815) and further comprising a polymerase recognition sequence (1820) and a restriction endonuclease recognition sequence (1825) is exposed to a sample comprising target agent (1830) to create reacted capture-associated oligo complex (1835). In step B, reacted capture-associated oligo complex (1835) is exposed to an immobilized binding partner (1840), which specifically binds to the capture moiety/target agent complex, to create an immobilized reacted capture-associated oligo complex (1845). In step C, hybridization of the immobilized reacted capture-associated oligo complex (1845) to an oligonucleotide (1850) complementary to the polymerase recognition sequence (1820) and the restriction endonuclease recognition sequence (1825) provides both a double-stranded polymerase recognition site (1855) and a double-stranded restriction endonuclease recognition site (1860). In step D, the immobilized reacted capture-associated oligo complex (1845) hybridized to the complementary oligo (1850) is reacted with an appropriate restriction endonuclease to remove the capture moiety/target agent complex, thereby creating cleaved capture-associated oligo complex (1865) comprising cleaved oligo (1870). In step E, the cleaved capture-associated oligo complex (1865) comprising the double-stranded polymerase recognition site (1855) is reacted with the appropriate nucleotides and polymerase to provide creation of an oligo (1875) complementary to the cleaved oligo (1870). In step F the reactions are carried out repeatedly to create multiple copies of the complementary oligo (1875) via linear amplification. In step G the complementary oligos (1875) are introduced to the electrode-associated oligos (1880) on oligo chip (1885). The binding of the complementary oligos (1875) to the electrode-associated oligos (1880) generates a signal in an electrochemical detection device.

FIG. 19 is a schematic diagram illustrating an example of an embodiment comprising a combination of isolation using an immobilized binding partner that binds to a capture moiety/target agent complex, restriction endonuclease cleavage of the reacted capture-associated oligo, and polymerase amplification techniques. In step A, a capture-associated oligo (1910) conjugated to a capture moiety (1915) and further comprising a polymerase recognition sequence (1920) and a restriction endonuclease recognition sequence (1925) is exposed to a sample comprising target agent (1930) to create reacted capture-associated oligo complex (1935). In step B, reacted capture-associated oligo complex (1935) is exposed to an immobilized binding partner (1940), which specifically binds to the capture moiety/target agent complex, to create an immobilized reacted capture-associated oligo complex (1945). In step C, immobilized reacted capture-associated oligo complex (1945) is hybridized to a first complementary oligo (1948), which is complementary to and binds to the polymerase recognition sequence (1920), and a second complementary oligo (1953), which is complementary to and binds to the restriction endonuclease recognition sequence (1925) to create a first double-stranded region comprising a polymerase recognition site (1955) and a second double-stranded region comprising a restriction endonuclease recognition site (1960). In step D, the immobilized reacted capture-associated oligo complex (1945) hybridized to the complementary oligos (1948 and 1953) is reacted with an appropriate restriction endonuclease to remove the capture moiety/target agent complex, thereby creating cleaved capture-associated oligo complex (1965) comprising cleaved oligo (1970). In step E, the cleaved capture-associated oligo complex (1965) comprising the double-stranded polymerase recognition site (1955) is reacted with the appropriate nucleotides and polymerase to provide creation of an oligo (1975) complementary to the cleaved oligo (1970). In step F the reactions are carried out repeatedly to create multiple copies of the complementary oligo (1975) via linear amplification. In step G the complementary oligos (1975) are introduced to the electrode-associated oligos (1980) on oligo chip (1985). The binding of the complementary oligos (1975) to the electrode-associated oligos (1980) generates a signal in an electrochemical detection device.

As noted above, in certain embodiments of the invention the capture-associated oligo and the chip-associated (e.g., electrode-associated) oligo may be partially or completely noncomplementary. For example, an “intermediary oligo” can be used that has a first region complementary to the capture-associated universal oligo and a second region that is complementary to the chip-associated oligo. After addition of sample to a capture-associated oligo and subsequent formation of a reacted capture-associated oligo complex, the reaction mixture is contacted with immobilized binding partners that specifically immobilize the reacted capture-associated oligo complex (e.g., via binding to the target agent or capture moiety/target agent complex). An intermediary oligo is added that hybridizes to the capture-associated oligo. In certain embodiments, hybridization of the intermediary oligo to the capture-associated oligo creates a double-stranded restriction endonuclease recognition site near the end of the capture-associated oligo that is distal to the capture moiety. Treatment with an appropriate restriction endonuclease releases the portion of the intermediary oligo complementary to the chip-associated oligo into the aqueous phase. Other methods of separation of the second region from the capture-associated oligo/intermediary oligo hybridization complex may also be employed, e.g., as described elsewhere herein. An aqueous phase comprising the second region of the intermediary oligo is transferred to a chip where the oligo complementary to the chip-associated oligo (e.g., electrode-associated oligo) can hybridize to the chip-associated oligo. A signal generated by the detection device is measured. In other embodiments, hybridization of the intermediary oligo to the capture-associated oligo creates a double-stranded polymerase recognition site that may be used to amplify the second region of the intermediary oligo, linearly or logarithmically, by methods disclosed herein or known to one of ordinary skill in the art. In such embodiments, an aqueous phase comprising an oligo complementary to the chip-associated oligo (whether comprising sequence identical or complementary to the second region of the intermediary oligo) is transferred to a chip where the oligo complementary to the chip-associated oligo (e.g., electrode-associated oligo) can hybridize to the chip-associated oligo. In embodiments that involve amplification, whether the second region of the intermediary oligo comprises sequence identical or complementary to the chip-associated oligo is dependent on the type of amplification to be performed, as described herein. In still further embodiments, both separation and amplification steps are performed, and amplification may precede or follow the separation step. Optionally, an intermediary oligo can be labeled with ferrocene or another label that enhances the signal to be measured.

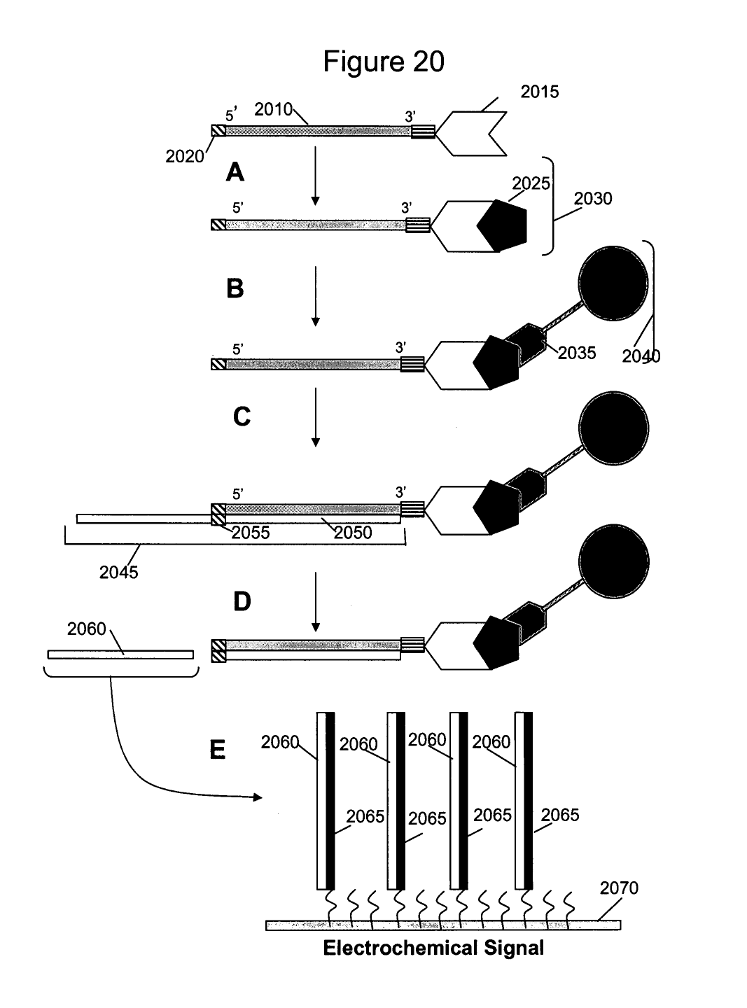

FIG. 20 is a schematic diagram illustrating an example of an embodiment comprising use of an intermediary oligo. In step A, a capture-associated oligo (2010) conjugated to a capture moiety (2015) and further comprising a restriction endonuclease recognition sequence (2020) is exposed to a sample comprising target agent (2025) to create reacted capture-associated oligo complex (2030). In step B, reacted capture-associated oligo complex (2030) is exposed to an immobilized binding partner (2035), which specifically binds to the target agent, to create an immobilized reacted capture-associated oligo complex (2040). In step C, immobilized reacted capture-associated oligo complex (2040) is hybridized to an intermediary oligo (2045), which comprises a first region (2050) complementary to capture-associated oligo (2010), and a second region that is (2055) complementary to and binds to the restriction endonuclease recognition sequence (2020) to create a double-stranded region comprising a restriction endonuclease recognition site. In step D, the immobilized reacted capture-associated oligo complex (2040) hybridized to the intermediary oligo (2045) is reacted with an appropriate restriction endonuclease to remove the capture moiety/target agent complex, thereby creating cleaved oligo (2060). In step E, cleaved oligo (2060) is introduced to electrode-associated oligos (2065) on oligo chip (2070). The binding of the cleaved oligo (2060) to the electrode-associated oligos (2065) generates a signal in an electrochemical detection device.

### Electrochemical Biosensors for Use in the Present Invention

Various biosensors known to those skilled in the art may be used in the present invention to detect the presence and/or abundance of a target agent in a sample. One general type of biosensor for use in the present invention employs an electrode surface in combination with current or impedance measuring elements for detecting a change in current or impedance in response to the presence of a detection moiety brought within an appropriately close distance (“proximity”) of the electrode to enable a distinct and reproducible redox reaction. The distance necessary to achieve a distinct and reproducible redox reaction, and thus electrochemical measurement of binding, will vary depending upon the nature of the detection moiety and the properties of the electrode surface. Determining the necessary proximity of a detection moiety to effect the desired reaction will be well within the skill of one skilled in the art upon reading the present disclosure.

The electrodes of the invention can be produced in a disposable format, intended to be used for a single electrochemical detection experiment or a series of detection experiments and then thrown away. The invention further provides an electrode assembly including both a detection electrode and a reference electrode required for electrochemical detection. Conveniently, the electrode assembly could be provided as a disposable unit comprising a housing or holder manufactured from an inexpensive material equipped with electrical contacts for connection of the detection electrode and reference electrode.

Electrochemical biosensors capable of detecting and quantifying target agents in a sample, such as those described and used in the present invention, offer many advantages over strictly biochemical assay formats. First, electrochemical biosensors may be produced, using conventional microchip technology, in highly reproducible and miniaturized form, with the capability of placing a large number of biosensor elements on a single substrate (e.g., see U.S. Pat. Nos. 5,200,051 and 5,212,050). Secondly, because small electrochemical signals can be readily amplified (and subjected to various types of signal processing if desired), electrochemical biosensors have the potential for measuring minute quantities of a target agent, and proportionately small changes in target agent levels. Importantly, electrochemical biosensors may offer this exquisitely sensitive detection at a lower cost than currently available assay methods.

The preferred biosensor for use in the present invention comprises a conventional electrode with a modified surface allowing oligo attachment, and thus the description herein is focused on the use of such an electrode. Other biosensor systems, however, may be utilized in the assay methods of the invention, as will be apparent to one skilled in the art upon reading this disclosure, and these are intended to be encompassed within the present invention. Examples of other biosensors that may be utilized with the present invention include, but are not limited to, biosensors disclosed, for example, in U.S. Pat. No. 5,567,301; biosensors based on surface plasmon resonance (SPR) (see, e.g., U.S. Pat. No. 5,485,277; and Zezza, F., et al, J Microbiol Methods., 66:529 (2006)); biosensors that utilize changes in optical properties at a biosensor surface (e.g., as described in U.S. Pat. No. 5,268,305); optical detection methods such as fluorescence labeling of oligonucleotides (Pease, et. al., Proc. Natl. Acad. Sci. USA 91:5022 (1994)), chemiluminescence, colorimetric assays, DNA microarrays (Albrecht, V., et al, J Virol Methods., 137:236 (2006)); label free detection methods such as carbon nanotube network field-effect transistors (Star, et al., Proc. Natl. Acad. Sci. 103:921 (2006)); and the like.

The electrode for use in the present invention preferably comprises a mixed monolayer on the conductive surface of the electrode, the monolayer comprising both anchoring groups conjugated to electrode-associated oligos and diluent groups which serve as an insulator on the electrode surface. Depending on the length, sequence, and secondary structure of the oligo, specific spacing of the anchoring groups and the diluent groups can be designed to maximize interaction capabilities. For example, it can be advantageous to have only small sub-monolayer amounts of the electrode-associated oligo present on the surface to enhance the hybridization properties of the electrode-associated oligos with the capture-associated oligos, particularly if the capture-associated oligos are still attached to their capture moieties. Also, several different electrode-associated oligos can be introduced at the same time into the monolayer to create a monolayer with detection capabilities for multiple target agents.

One specific method for enhancing the binding of oligos to a biosensor is thus utilizing a specific ratio of anchoring groups attached to the electrode-associated oligos (together referred to as “anchoring group complexes”) and diluent groups in the monolayer on the electrode. The ratio of bound anchoring group complexes and diluent agents on the electrode can be designed to optimize the access of the electron-associated oligo to any capture-associated oligo present in an assay. The ratio is preferably designed to be a concentration of the electrode-associated oligo that will limit binding interference due to conformational interactions between multiple electrode-associated oligos. Biosensors with specific concentrations of the diluent agents and the anchoring group complexes will enhance the availability of the electrode-associated oligos for binding to the capture-associated oligos while maintaining the insulating monolayer on the electrode. The final ratio of the components of the biosensor is preferably designed to create uniform monolayers with evenly distributed anchoring group complexes and diluent groups. The ratio of anchoring group complexes and diluent groups is preferably designed to maximize access to the electrode-associated oligos, and to provide an enhancement of detection of the hybridization of capture-associated oligos to the biosensor.

In determining the appropriate concentration of the components to be used in depositing the monolayer on the conductive surface, a number of practical issues must be considered. For example, great differences in chain length or size of the electrode-associated oligo on anchoring group complexes can lead to preferential adsorption of the diluent groups. This can also lead to formation of islands of anchoring group complex surrounded by diluent agents (Bain C D, Evall J, Whitesides G M. J Am Chem Soc 1989; 111: 7155-7164; Bain C D, Whitesides G M. J Am Chem Soc 1989; 111: 7164-7175). In addition, as a general rule, the SAM composition will not be deposited on the surface in the same concentration ratio as in the preparation solution. Characterization of the SAM surface with an analytical tool, e.g., infrared spectroscopy, ellipsometry, studies of wetting by different liquids, x-ray photoelectron spectroscopy, electrochemistry, and scanning probe measurements, thus may be necessary to calibrate the mixing ratio and can be used to determine the most appropriate ratio for specific anchoring agent complexes, as will be apparent to one skilled in the art upon reading the present specification. For example, in certain embodiments, the electrostatic repulsion between DNA strands may help suppress island formation; in other embodiments, such as those employing peptide nucleic acids, the electrostatic repulsion will be reduced and may not serve to prevent this phenomenon.

While applicants do not wish to be limited to any particular presumed mechanism for the action of their invention, it is their present understanding that the detection of the capture-associated oligo using an electrode is based on an electrochemical reaction on the conductive detection surface, which requires that electrons tunnel from the electron donor through the insulating monolayer. This would suggest that because the primary mechanism by which electrochemical detection takes place is via “through-bond” electron tunneling rather than interchain electron tunneling, the composition of the linkage of the oligo complex will have a significant effect on the electron transfer rate. To achieve the most accurate and efficient signal, both the anchoring group and the diluent group, which forms the insulator composition, should therefore be selected to maximize the ratio of specific current to non-specific, or “leakage,” current. The efficiency of the tunneling can thus be controlled by manipulation of the molecules which comprise the monolayer.

The insulating properties of the monolayer film will thus depend upon the chemical composition of the molecules forming the monolayer. For example, the properties of an alkane thiol versus an ether thiol can significantly change the rate constant, with the rate constant through the alkane linker shown on an order of four times faster than through the ether linker. The composition of the non-complexed SAM components can impact on the overall electron transfer rate, though not as significantly as with the linkage of the oligo complex. In this case, non-complexed ether thiol molecules (“diluent molecules”) will reduce the overall rate constant slightly versus their alkane counterparts. Ether linkages are more highly insulating than alkane groups, presumably because of an energy effect.

For use in the assays of the invention, the electrodes can be designed so that the anchoring group and the diluent group have the same chemical composition, e.g., both are alkane thiols, or alternatively the anchoring group and the diluent group may have different chemical compositions, e.g., the anchoring group is an alkane thiol and the diluent group is an ether thiol.

In a particular embodiment, the anchoring group comprises a hydrocarbon component (e.g., an alkylthiol) and a polyethylene glycol group, which will impart a greater level of hydrophilicity to the biosensor and provide additional flexibility to the electrode-associated oligo linkage. The hydrocarbon component would be roughly the same length as the alkylthiol diluent molecule, promoting tight packing and perhaps more importantly discouraging so-called “phase separation” into DNA-rich and DNA-poor domains. The PEG component would serve as a hydrophilic “vertical” spacer to create further distance between the oligo and the monolayer surface. For example, synthesis of the biosensor SAM-forming molecules can comprise at least one anchoring group comprising an alkylthiol group linked to a PEG component and an oligo, and at least one substantially hydrophobic alkane diluent group. When provided within suitable (polar) carrier solvents, these molecules are able to self-assemble on the electrode. The characteristics of the hydrophilic domain (e.g., length of the PEG backbone) and the concentration of the anchoring group complex and the diluent group can be independently varied.

Since the diluent agent is likely to be the more reactive component, the solution compositions used to create the monolayer are biased in favor of the DNA-bearing component, and generally range from a 1:1 to a 100:1 ratio of anchoring group complexes to diluent agent. In the methods of the invention related to manufacture of the biosensor, the components of the monolayer may be introduced in a single solution, in two solutions used simultaneously, or introduced sequentially to promote the adherence of the anchoring group complex e.g., the anchoring group complex solution is allowed to bind to the conductive surface for a period before introduction of the solution containing the diluent groups.

The overall concentration of the diluent group and anchoring group complexes, as well as the length of the molecules used in creating the self-assembled monolayer, will also determine the binding angle of the components of the monolayer, which affects both the thickness of the monolayer and the efficiency of the electron tunneling from the detection moiety to the electrode. The optimum binding angle can be designed based on the predicted thickness of the monolayer versus the length of the molecules in the SAM. The desired binding angle can be calculated and the monolayer appropriately designed to maximize the ratio of specific current to leakage current.

In a specific embodiment, the monolayer is composed of diluent groups and anchoring groups of 6-22 carbon atom chains attached at their proximal ends to the detection surface. In certain embodiments, the monolayer may be composed of anchoring group complexes and diluent agents attached at their proximal ends to the detection surface by a thiol linkage at a molecular density of about 3 to 5 chains/nm². In one aspect of this embodiment, the anchoring agent is present on the electrode in approximately a 10:1 to a 50:1 ratio of anchoring group complexes to diluent agent.

In one particular embodiment, the conductive detection surface of the biosensor is gold. Alkanethiol SAMs adsorbed on gold present several advantages. First, gold is a relatively inert metal that resists oxidation and atmospheric contamination fairly well (Chesters M A, Somotjai G A. Surf Sci 1975; 52: 21-28). Second, gold has a strong specific interaction with sulfur, providing a reproducible method for adhering the thiol groups to the surface of a gold detection surface (Nuzzo R G. Fusco F A, Allara D L. J Am Chem Soc 1987; 109: 2358-2368). The predictable binding of sulfur to gold allows the formation of tightly packed monolayers even in the presence of many other functional groups (Bain C D, Troughton E B, Tao Y-T, Evall J, Whitesides G M, Nuzzo R G. J Am Chem Soc 1989; 111: 321-335). Third, long-chain alkanethiols form a densely packed, crystalline or liquid-crystalline monolayer due to strong molecular interactions (van der Waals forces) between the long carbon chains (Strong L, Whitesides G M. Langmuir 1988; 4: 546-558).

In one embodiment, the anchoring group and the diluent group are both terminated with a thiol group that will interact directly with the conductive detection surface, e.g., the electrode. By mixing two or more differently terminated thiols in the preparation solution, a mixed monolayer can be prepared on the conductive surface as a mixed SAM. The relative proportion of the different groups in the assembled SAM will depend upon several parameters, like the mixing ratio in solution, the alkane chain lengths, the solubilities of the thiols in the solvent used, and the properties of the chain-terminating groups.

Preparing a SAM of alkanethiol molecules is a fairly simple process. A gold-coated substrate is immersed in a dilute solution of the alkanethiol in ethanol and a monolayer spontaneously assembles on the surface of the substrate over a period of 1-24 hours. A disordered monolayer is formed within a few minutes, during which time the thickness reaches 80-90% of its final value. Over the next several hours, van der Waals forces on the carbon chains help pack the long alkanethiol chains into a well-ordered, crystalline layer (Dubois L H, Nuzzo R G. Annu Rev Phys Chem 1992; 43: 437-463). In this process contaminants are replaced, solvents are expelled from the monolayer, and defects are reduced while packing is enhanced by lateral diffusion of the alkanethiols (Bain C D, Troughton E B, Tao Y-T, Evall J, Whitesides G M, Nuzzo R G. J Am Chem Soc 1989; 111: 321-335).

The resulting monolayers assemble with the alkanethiolates in a hexagonal-packing arrangement. This chain spacing is larger than the ideal distance needed to maximize van der Waals interactions between the chains. Therefore, a natural tilt develops 30° from the normal surface, maximizing molecular interactions between carbon chains as they pack into their final crystalline monolayer. The importance of van der Waals interactions between the chains is also seen when one considers the chain length. In general, the longer the chain length, the more ordered the monolayer (Bain C D, Evall J, Whitesides G M. J Am Chem Soc 1989; 111: 7155-7164; Holmes-Farley S R, Bain C D, Whitesides G. Langmuir 1988; 4: 921-937).

Contact angle measurements further confirm that alkanethiolate SAMs are very dense and that the contacting liquid only interacts with the topmost chemical groups. Reported advancing contact angles with water range from 111° to 115° for hexadecanethiolate SAMs. At the other end of the wettability scale, there are hydrophilic monolayers, e.g., SAMs of 16-mercaptohexadecanol (HS(CH₂)₁₆OH), that display water contact angles of <10°. These two extremes are only possible to achieve if the SAM surfaces are uniform and expose only the chain-terminating group at the interface. Mixed SAMs of CH₃— and OH-terminated thiols can be tailor-made with any wettability (in terms of contact angle) between these limiting values.

Another SAM preparation method is the formation of two-component molecular gradients, as first described by Liedberg and Tengvall (Langmuir 11 (1995), 3821). By cross-diffusion of two differently terminated thiols through an ethanol-soaked polysaccharide gel (Sephadex LH-20, a chromatography material) that is covering the gold substrate, a continuous gradient of 10-20 mm length may be formed. Ethanol solutions of each of the two thiols are simultaneously injected into two glass filters at opposite ends of the gold substrate. The presence of the polysaccharide gel makes the diffusion and the thiol attachment to the surface slow enough for a gradient of macroscopic dimension (several mm) to form.

The chip-associated oligos are functionalized with the anchoring group to form the anchoring group complex which is attached to the detection surface, e.g., an electrode surface. Such methods are well known in the art. For instance, nucleotides functionalized with thiols at their 3′-termini or 5′-termini readily attach to gold nanoparticles. See Whitesides, Proceedings of the Robert A. Welch Foundation 39th Conference on Chemical Research Nanophase Chemistry, Houston, Tex., pages 109-121 (1995). See also, Mucic et al. Chem. Commun. 555-557 (1996) (describes a method of attaching 3′ thiol DNA to flat gold surfaces). The thiol method can also be used to attach oligos to other metal, semiconductor and magnetic colloids. Other functional anchoring groups for attaching oligos to solid surfaces include phosphorothioate groups (see, e.g., U.S. Pat. No. 5,472,881 for the binding of oligonucleotide-phosphorothioates to gold surfaces), substituted alkylsiloxanes (see, e.g. Burwell, Chemical Technology, 4, 370-377 (1974) and Matteucci and Caruthers, J. Am. Chem. Soc., 103, 3185-3191 (1981) for binding of oligos to silica and glass surfaces, and Grabar et al., Anal. Chem., 67, 735-743 for binding of aminoalkylsiloxanes and for similar binding of mercaptoaklylsiloxanes). Oligos terminated with a 5′ thionucleoside or a 3′ thionucleoside may also be used for attaching oligos to solid surfaces. Oligos may be attached to the electrode using other known binding partners, e.g., using biotin-labeled oligos and streptavidin-gold conjugate colloids; the biotin-streptavidin interaction attaches the colloids to the oligonucleotide. Shaiu et al., Nuc. Acids Res., 21, 99 (1993). The following references describe other anchoring groups which may be employed to attach oligos to electrode surfaces: Nuzzo et al., J. Am. Chem. Soc., 109, 2358 (1987) (disulfides on gold); Allara and Nuzzo, Langmuir, 1, 45 (1985) (carboxylic acids on aluminum); Allara and Tompkins, J. Colloid Interface Sci., 49, 410-421 (1974) (carboxylic acids on copper); Iler, The Chemistry Of Silica, Chapter 6, (Wiley 1979) (carboxylic acids on silica); Timmons and Zisman, J. Phys. Chem., 69, 984-990 (1965) (carboxylic acids on platinum); Soriaga and Hubbard, J. Am. Chem. Soc., 104, 3937 (1982) (aromatic ring compounds on platinum); Hubbard, Acc. Chem. Res., 13, 177 (1980) (sulfolanes, sulfoxides and other functionalized solvents on platinum); Hickman et al., J. Am. Chem. Soc., 111, 7271 (1989) (isonitriles on platinum); Maoz and Sagiv, Langmuir, 3, 1045 (1987) (silanes on silica); Maoz and Sagiv, Langmuir, 3, 1034 (1987) (silanes on silica); Wasserman et al., Langmuir, 5, 1074 (1989) (silanes on silica); Eltekova and Eltekov, Langmuir, 3, 951 (1987) (aromatic carboxylic acids, aldehydes, alcohols and methoxy groups on titanium dioxide and silica); Lec et al., J. Phys. Chem., 92, 2597 (1988) (rigid phosphates on metals).

In one embodiment of the invention, a film of electroconductive polymer is deposited onto the internal surface of an electrically conductive electrode by electrochemical synthesis from a monomer solution introduced onto the structure. Electrodeposition of the electroconductive polymer film can be carried out, e.g., according to the methods disclosed in U.S. Pat. No. 6,770,190 to Milanovski, et al. In such an exemplary method, a solution containing monomers, a polar solvent and a background electrolyte are used for deposition of the polymer.

Electroconductive polymers can be doped at the electrochemical synthesis stage to modify the structure and/or conduction properties of the polymer. A typical dopant anion is sulphate (SO₄²⁻), which is incorporated during the polymerisation process to neutralize any positive charge on the polymer backbone. Sulphate is not readily released by ion exchange and thus helps to maintain the structure of the polymer. Dopant anions having maximum capability for ion exchange with the solution surrounding the polymer can be used to increase the sensitivity of the electrodes. This is accomplished by using a salt with anions having a large ionic radius as the background electrolyte when preparing the electrochemical polymerisation solution, e.g., sodium dodecyl sulphate and dextran sulphate. The concentration of these salts in the electrochemical polymerisation solution is varied according to the type of test within the range 0.005-0.05 M.

In another embodiment, the electroactive polymer is introduced to the surface of the electrode via an introduced functional group, e.g., a sulfide, disulfide, amino, amide, amido, a carboxyl, a hydroxyl, carbonyl, oxide, phosphate, sulfate, aldehyde, keto, thiol, ester or mercapto groups. Other highly reactive functional groups may also be employed using methods readily known to those of ordinary skill in the art. For example, polymers with an associated thiol group can be bound directly to a gold or platinum surface. This embodiment may be preferable for the use of more complex polymers that are difficult to synthesize using monomer deposition.

Adaptor molecules may either be immobilized in the electroconductive polymer film at the electrochemical synthesis stage by adding adaptor molecules to the electrochemical polymerisation solution or may be adsorbed onto the surface of the electroconductive polymer film after electrochemical polymerisation. In the former case, a solution of adaptor molecules may be added to the electrodeposition solution immediately before the deposition process. The deposition process works optimally if the storage time of the finished solution does not exceed 30 minutes. Depending on the particular type of test, the concentration of adaptor molecules in the solution may be varied in the range 5.00-100.00 μ/mL. Procedures for electrodeposition of the electroconductive polymer from the solution containing adaptor molecules are described in the examples included herein. On completion of electrodeposition process, the detection electrode obtained may be rinsed successively with deionised water and 0.01 M phosphate-saline buffer solution and, depending on the type of test, may then be placed in a special storage buffer solution containing microbial growth inhibitors or bactericidal agents (e.g., gentamicin), or dried in dust-free air at room temperature.

Where the adaptor molecules are to be adsorbed after completion of the electrodeposition process the following protocol may be used (although it is hereby stated that the invention is in no way limited to the use of this particular method), the detection electrode is first rinsed with deionised water and placed in freshly prepared 0.02 M carbonate buffer solution, where it is held for 15-60 minutes. The detection electrode is then placed in contact with freshly-prepared 0.02 M carbonate buffer solution containing adaptor molecules at a concentration of 1.00-50.00 μg/mL, by immersing the detection electrode in a vessel filled with solution, or by placing a drop of the solution onto the surface of the detection electrode. The detection electrode is incubated with the solution of adaptor molecules, typically for 1-24 hours at +4° C. After incubation, the detection electrode is rinsed with deionised water and placed for 1-4 hours in a 0.1 M phosphate-saline buffer solution. Depending on the type of test, the detection electrode may then be placed either in a special storage buffer solution containing microbial growth inhibitors or bactericidal agents, or dried in dust-free air at room temperature.

When the adaptor molecules are avidin or streptavidin, the above-described methods of the invention comprise a further step of contacting the coated electrode with a solution comprising specific oligos conjugated with biotin such that said biotinylated oligos bind to molecules of avidin or streptavidin immobilised in or adsorbed to the electroconductive polymer coating of the electrode via a biotin/avidin or biotin/streptavidin binding interaction. Conjugation of biotin with the corresponding oligo, a process known to those skilled in the art as biotinylation, can be carried out using procedures well known in the art.

Biotinylated peptidic spacers, generally from between 0.4 and 2 nm in length, can also be used to couple the adaptor molecule to the oligo. The resulting conjugates can be immobilized on the microdevice electrode surface through specific binding to the adaptor molecule. The electron transfer through multilayers of the conjugates is strongly dependent on the length of the spacer between the oligo (and thus any bound electrochemical detection agent) and the electrode surface. The redox current through the layer is dependent on external parameters such as the applied voltage difference between the two electrode arrays or the temperature.

In one embodiment, the electrostatic interactions between a detection moiety and the SAM can be controlled though the use of immobilized adsorbates on the monolayer and control of the pH in the reaction solution. This will allow enhancement of the electron signaling through better control of the distance between the detection moiety and the electrode monolayer. In one example, the detection moiety is negatively charged, and the monolayer is modified with a deprotonable adsorbate. For example, in the case where the immobilized adsorbate is a carboxylic acid, the deprotonation of the carboxylic acid head leads to repulsion of a negatively charged redox molecule (e.g., Fe(CN)₆^{3-/4-}), leading in turn to a decrease in heterogenous electron transfer. The reaction can thus be enhanced by decreasing the pH of the reaction mixture, allowing the redox reaction to penetrate to the electrode.

In another related embodiment, the detection moiety is positively charged, and the monolayer is modified with an immobilized adsorbate that responds reversibly to pH. For example, the immobilized adsorbates are amine containing adsorbates in combination with a positively charged redox couple (e.g., Ru(NH₃)₆^{2+/3+}). At low pH, when the amines on the dendrimer are protonated, the layer is isolating; at high pH the amines are deprotonated and the redox couple penetrates the dendrimer through which it can reach the electrode.

Other electrochemically active monolayers that combine the reduction of the immobilized adsorbate with protonation include azobenzenes (Caldwell W R et al., J. Am. Chem. Soc. 117:6071 (1995); Wang R et al., J. Electroanal. Chem. 438:213 (1997)), nitrobenzoic acids (Casero E et al., 1999 15:127 (1999)), and mixed acid-ferrocene sulfide molecules (Beulen W J et al., 503 Chem Commun (1999)).

Use of Adaptor Molecules for Conjugation of the Detector Moieties

The assays of the present invention require the conjugation of the detection moiety to the appropriate oligo for the specific embodiment, which may be the electrode-associated oligo, the capture-associated oligo, or a detection-moiety-associated oligo. This is preferentially accomplished through the use of adaptor molecules. The proteins avidin and streptavidin are two preferred adaptor molecules for use in the present invention. Avidin consists of four identical peptide sub-units, each of which has one site capable of bonding with a molecule of the co-factor biotin. Biotin (vitamin H) is an enzyme co-factor present in very minute amounts in every living cell and is found mainly bound to proteins or polypeptides. Biotin molecules have the ability to enter into a binding reaction with molecules of avidin or streptavidin (a form of avidin isolated from certain bacterial cultures, for example Streptomyces avidinii) and to form virtually non-dissociating “biotin-avidin” complexes during this reaction (with a dissociation constant of about 10⁻¹⁵ Mol/L).

Techniques which allow the conjugation of biotin to a wide range of different molecules are well known in the art. Thus detection moieties with immobilized avidin or streptavidin can easily be made specific for a given oligo target merely by binding of the appropriate biotinylated oligo. Other similar members or binding pairs are intended to be within the scope of the present invention, and use of such will be known to one skilled in the art upon reading the present disclosure. For example, biotinylated peptidic spacers, generally from between about 0.14 and 3.02 nm in length, can also be used to couple the detection moiety to the oligo. The electron transfer through multilayers of the conjugates is dependent on the length of the spacer between the electrode-associated oligo (and thus any bound electrochemical detection agent) and the electrode surface. The redox current through the layer is dependent on external parameters such as the applied voltage difference between the two electrode arrays and the temperature.

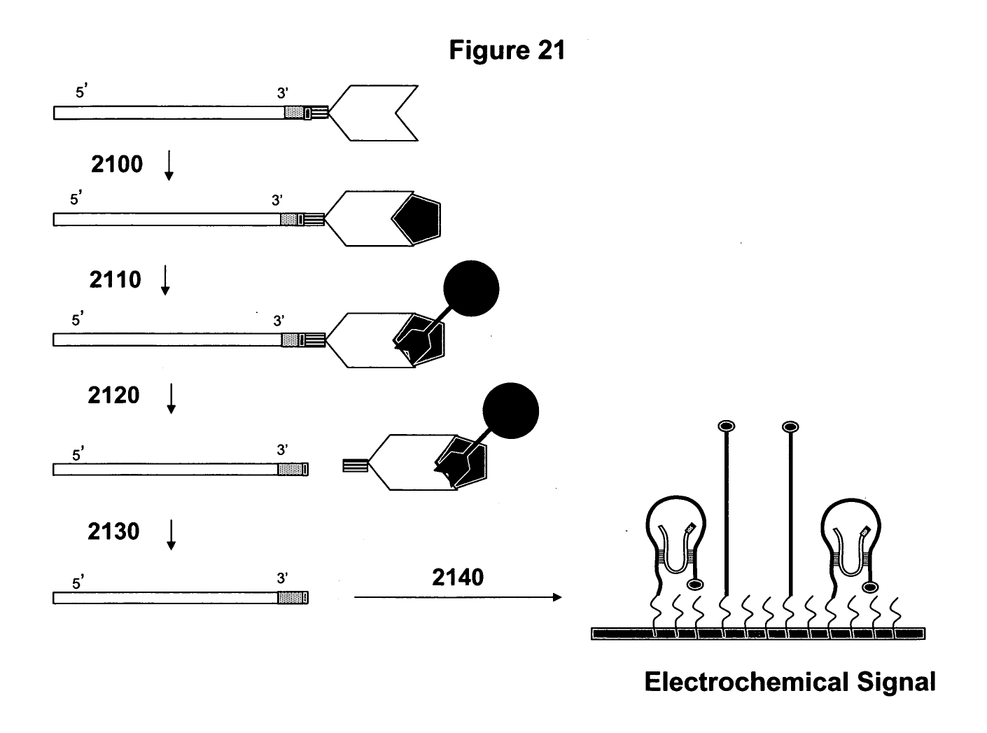

FIG. 21 is a schematic diagram illustrating one embodiment in which the detection assay uses a capture moiety that preferentially binds to the capture moiety/target agent complex and an immobilized binding partner for isolation of the capture moiety/target agent complex. The first step is exposure of the capture moiety to the sample for binding of the target agent in the sample (2100). Isolation of the bound capture moiety/target agent complex is achieved using an immobilized binding partner for isolation (2110). A restriction endonuclease is subsequently used to remove the capture moiety from the capture-associated oligo prior to introduction of the capture-associated oligo to the electrode (2120), and the capture-associated oligonucleotides are isolated from the remainder of the capture moiety (2130). The isolated capture-associated oligonucleotides are then introduced to the electrode-associated oligos, which each comprise a detection moiety at or near the unattached end of the electrode-associated oligos. The binding of the isolated capture-associated oligos to their complementary electrode-associated oligos will induce a circular structure, which will bring the detection moiety in closer proximity to the electrode (2140). This will provide an electrochemical redox reaction that is capable of detection of the target agent.

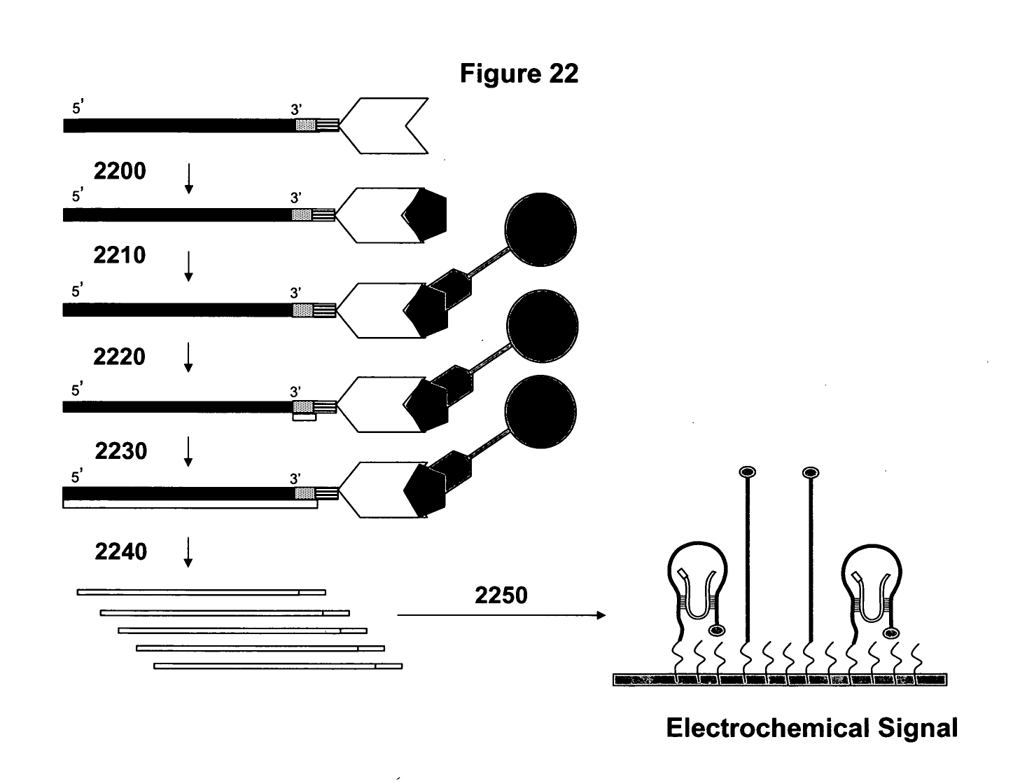

FIG. 22 is a schematic diagram illustrating an embodiment of a detection assay using a capture moiety that preferentially binds to the target agent, an immobilized binding partner for isolation of the target agent/capture moiety complex, and polymerase amplification techniques to enhance the signal. The first step is exposure of the capture moiety to the sample for binding of the target agent in the sample (2200). Once the capture moiety has bound its target agent, the complex is exposed to an immobilized binding partner for isolation (2210). The binding of an oligonucleotide complementary to the encoded single-stranded polymerase recognition sequence provides a double-stranded polymerase recognition site (2220). The complex is reacted with the appropriate nucleotides and polymerase to provide creation of an oligo complementary to the capture-associated oligo (2230). The reactions are carried out to create multiple copies of the complementary oligo via linear amplification (2240). The newly synthesized oligonucleotides are introduced to the electrode-associated oligos, which each comprise a detection moiety at or near the unattached end of the nucleic acid. The binding of the amplified oligonucleotides to the complementary electrode-associated oligos will induce a circular structure, which will bring the detection moiety in closer proximity to the electrode (2250). This will enable an electrochemical redox reaction which is capable of detection of the target agent.

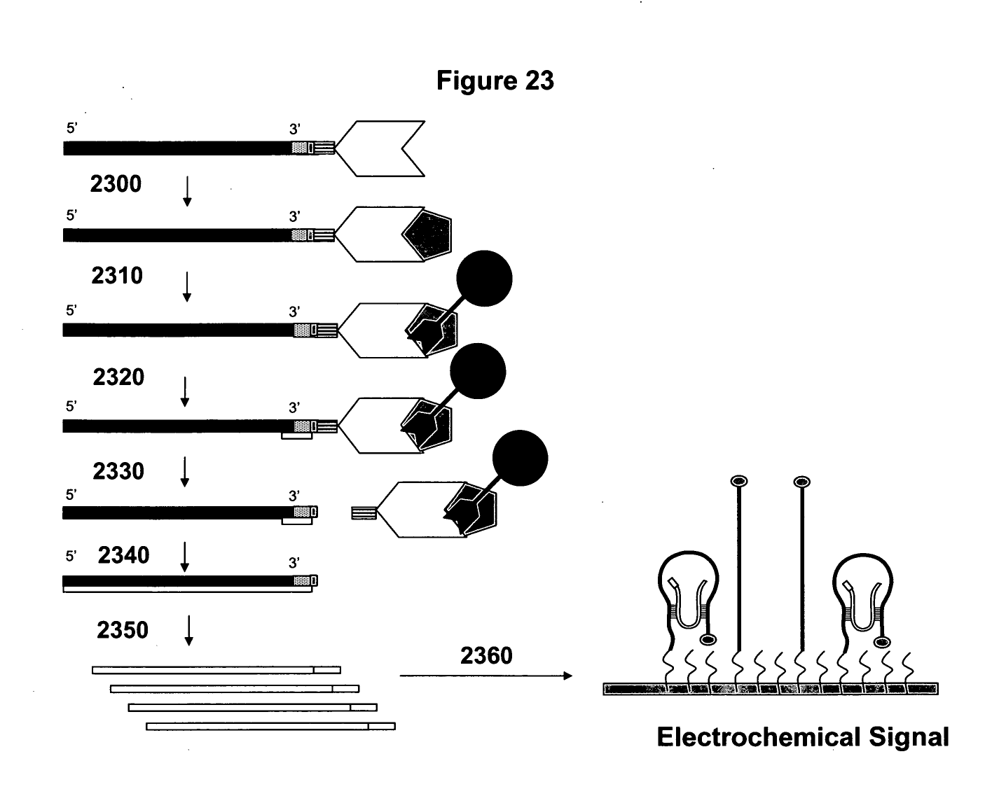

FIG. 23 is a schematic diagram illustrating an embodiment of a detection assay using a capture moiety that preferentially binds to the capture moiety/target agent complex, an immobilized binding partner for isolation of the target agent/capture moiety complex, a restriction endonuclease to remove the capture moiety from the capture-associated oligo, and polymerase amplification techniques to enhance the signal. The first step is exposure of the capture moiety to the sample for binding of the target agent in the sample (2300). Once the capture moiety has bound its target agent, the complex is exposed to an immobilized binding partner for isolation (2310). The binding of a labeled oligonucleotide complementary to the encoded single-stranded polymerase recognition sequence provides a double-stranded polymerase recognition site (2320). The complex is reacted with the appropriate restriction endonuclease to remove the capture moiety from the capture-associated oligo (2330), and the capture-associated oligo is reacted with nucleotides and polymerase to provide creation of an oligonucleotide molecule complementary to the capture-associated oligo (2340). The reactions are carried out to create multiple copies of the complementary oligo via linear amplification (2350). The newly synthesized oligonucleotides are introduced to the electrode-associated oligos, which each comprise a detection moiety at or near the unattached end of the electrode-associated oligo. The binding of the amplified oligonucleotides to the complementary electrode-associated oligos will induce a circular structure, which will bring the detection moiety in closer proximity to the electrode (2360). This will enable an electrochemical redox reaction which is capable of detection of the target agent.

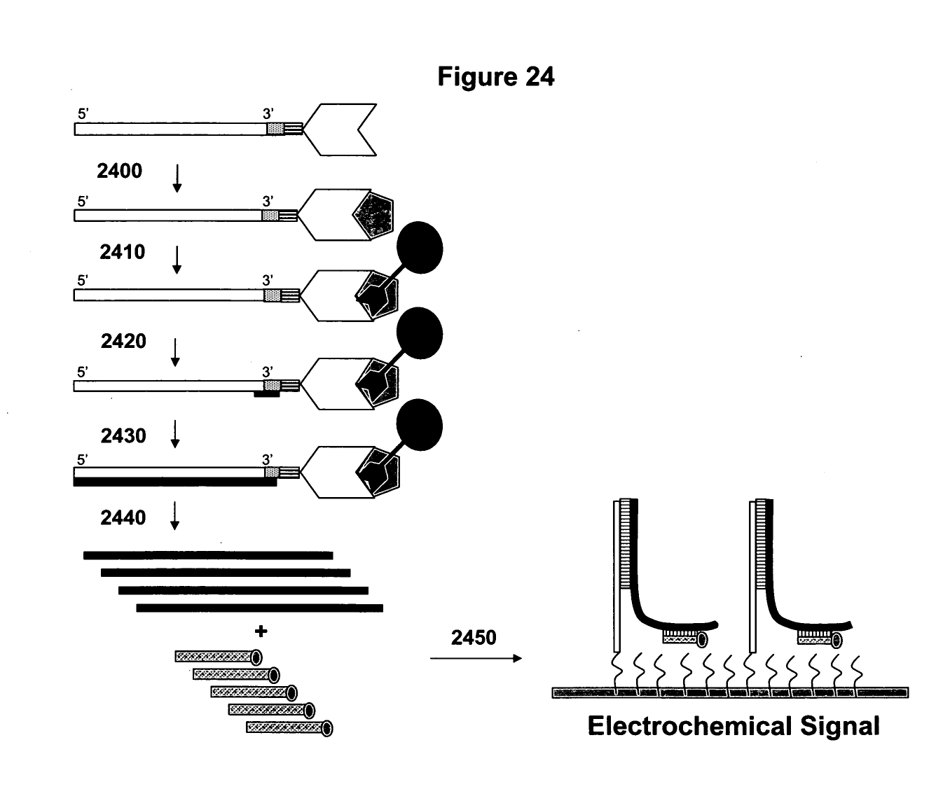

FIG. 24 is a schematic diagram illustrating a detection assay using a capture moiety that preferentially binds to the capture moiety/target agent complex, an immobilized binding partner for isolation of the target agent/capture-moiety complex, and polymerase amplification techniques to enhance the signal. The detection is enabled using a three oligo system: the capture moiety oligo (either the capture-associated oligo or an oligo produced using the capture-associated oligo as a template); the electrode-associated oligo, which is complementary to the capture moiety oligo; and an oligo comprising a detection moiety (the “detection moiety-associated oligo”), which is also complementary to the capture moiety oligo at a different region than is complementary to the electrode-associated oligo. The detection moiety-associated oligo has a detection moiety conjugated to the end of the oligo closest to the electrode following binding to the capture moiety oligo and hybridization of the capture moiety oligo to an electrode-associated oligo.

In a specific embodiment illustrated in FIG. 24, the first step is exposure of the capture moiety-oligo complex to the sample for binding of the target agent in the sample (2400). Once the capture moiety has bound its target agent, the complex is exposed to an immobilized binding partner for isolation (2410). The binding of an oligonucleotide complementary to the encoded single-stranded polymerase recognition sequence provides a double-stranded polymerase recognition site (2420). The complex is reacted with the appropriate nucleotides and polymerase to provide creation of an oligo complementary to the capture-agent associated oligo (2430). Alternatively, the complex may be reacted with the appropriate restriction endonuclease to remove the capture moiety from the capture-associated oligo prior to the polymerase treatment. The polymerase reactions are carried out to create multiple copies of the complementary oligo via linear amplification, each being complementary to both a specific electrode-associated oligo and a detection moiety-associated oligo (2440). The newly-synthesized oligonucleotides and a plurality of detection moiety-associated oligonucleotides complementary to the newly synthesized oligonucleotides are introduced to the electrode-associated oligonucleotides. The binding of the amplified oligonucleotides to both their complementary detection moiety-associated oligos and to the electrode-associated oligos will bring the detection moiety in proximity to the electrode (2450). This will enable an electrochemical redox reaction which is capable of detection of the target agent.

In an alternate embodiment, the diluent groups of the SAM on the electrode are derivatized to comprise an immobilized adsorbate that, when exposed to the appropriate pH, will enhance the attraction of the detection moiety to the electrode. This may further enhance the electrochemical redox reaction which is capable of detection of the target agent. In yet another embodiment, an oligo is derivatized with multiple detector moieties to enhance the electrochemical signal.

In another embodiment, the capture moiety of the assay of FIG. 24 preferentially binds to specific target agents and the detection moiety is tethered between two oligonucleotides. In this embodiment, the detection moiety is associated to the same detection moiety at or near the unattached end of the oligo such that binding of the capture-associated oligos to their complementary electrode-associated oligos will bring the detector moiety in proximity to the electrode.

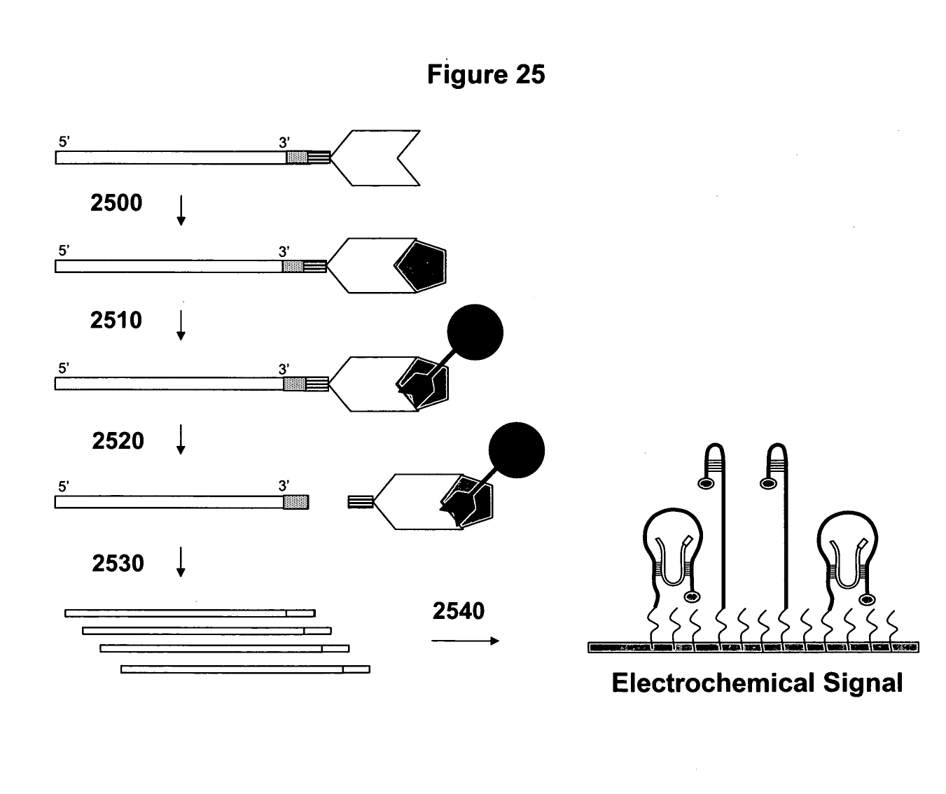

FIG. 25 is a schematic diagram illustrating the detection assay using a capture moiety that preferentially binds to the capture moiety/target agent complex, an immobilized binding partner for isolation of the capture moiety/target agent complex, and a restriction endonuclease to remove the capture-associated oligo from the capture moiety. The first step is exposure of the capture-associated oligo complex to the sample for binding of the target agent in the sample (2500). Once the capture moiety has bound its target agent, the complex is exposed to an immobilized binding partner for isolation (2510). The complex is reacted with the appropriate restriction endonuclease to remove the capture moiety from the capture-associated oligo (2520). The capture-associated oligonucleotides (2530) are introduced to the electrode-associated oligonucleotides which each comprise a detection moiety at or near the unattached end of the electrode-associated oligo, which itself comprises a hairpin loop structure. The binding of the capture-associated oligos to the complementary electrode-associated oligos will induce a circular loop structure in each oligo binding pair and disrupt the hairpin loop of the electrode-associated oligo, which will bring the detection moiety in close proximity to the electrode (2540). This will permit an electrochemical redox reaction which is capable of detection of the target agent.

This embodiment may further comprise linear amplification of the capture-associated oligonucleotide. As such, at step (2520) the complex would not be reacted with the appropriate restriction endonuclease but instead would be bound to an oligo complementary to an encoded single-stranded polymerase recognition sequence present on the capture-associated oligo to provide a double-stranded polymerase recognition site. The complex would then be reacted with nucleotides and polymerase to provide creation of an oligo complementary to the capture-associated oligo. The reactions would be carried out to create multiple copies of the oligo via linear amplification.

### Detection Kits of the Invention

The present invention also contemplates the use of kits to perform the electrochemical detection of target agents in a sample that can be, for example, potentially infectious or disease-causing agents, chemical or biological toxins, proteins (e.g., antibodies), nucleic acids (e.g., genetic loci, RNA expression, RNAi), and the like. The kits can include capture-associated universal oligos (in some embodiments, bound to loaded scaffolds) and immobilized binding partners that specifically associate with the capture moieties and/or target agents. In some embodiments, the immobilized binding partners of the capture moiety are immobilized on a particle, or bead, and in other embodiments, the binding partners are immobilized on a vessel wall.

In certain embodiments, a kit also includes a universal oligo chip comprising a plurality of electrodes and electrode-associated universal oligos. In addition, the kit can include an electrochemical hybridization detector, as discussed above. Optionally, the kit can include an agent for separating a capture-associated oligo from a reacted capture-associated oligo complex. In some embodiments, kits include protocols for carrying out standardized reactions for capture and/or hybridization reactions, and/or instructions for detection by electrochemical, fluorescent, and/or magnetic means. Such protocols and instructions would eliminate or substantially minimize non-specific hybridization and cross-reactivity. In certain preferred embodiments, kits are tailored for specific applications. For example, they may comprise capture moieties directed to target agents associated with a) disease to aid in diagnosis, b) a genetic disorder to aid in prognosis, c) drug response to aid in theranostics, d) microorganisms to aid in identification/strain differentiation (e.g., in an event of food-borne illness, infection, or bioterrorism attack), or e) chemicals to aid in identification of contamination (e.g., environmental monitoring, poisonings).

One embodiment of the present invention as described is specifically directed to kits for use in performing the methods of the invention. The kits of the invention comprise a carrier, such as a box or carton, having one or more vessels, such as vials, tubes, bottles and the like. In the kits of the invention, a first container contains one or more of the capture-associated oligos, capture moieties, and/or detector moieties described herein. The kit may further comprise a biosensor having electrode-associated oligos that can specifically hybridize to the product of the bound capture moiety to enable electrochemical detection of a target agent. The kits of the invention may also comprise, in the same or different containers, at least one component selected from one or more RNA or DNA polymerases (preferably thermostable DNA polymerases), a suitable buffer for nucleic acid synthesis and one or more nucleotides. Specific embodiments utilizing detection moiety-associated oligos may comprise oligos with the conjugated detection moiety, which may be used directly for hybridization or as primers for amplification. Alternatively, the components of the kit may be divided into separate vessels. In one aspect, the kits of the invention comprise a container containing an RNA polymerase in an appropriate buffered solution. In another aspect, the kits of the invention comprise a vessel containing a heat stable polymerase, e.g., Taq polymerase in an appropriate buffered solution. In additional preferred kits of the invention, the enzymes (RNA or DNA polymerases) in the containers are present at optimum working concentrations for the desired amplification reactions.

The following examples serve to illustrate certain embodiments and aspects of the present invention and are not to be construed as limiting the scope thereof.

### EXAMPLE I

### Preparation of Monoclonal Antibodies

A peptide corresponding to amino acid residues in a desired antigen is synthesized with a peptide synthesizer (Applied Biosystems) according to methods known in the art. The peptide emulsified with Freund's complete adjuvant is used as an immunogen and administered to mice by footpad injection for primary immunization (day 0). The booster immunization is performed four times or more in total. The final immunization is carried out by the same procedure two days before the collection of lymph node cells. The lymph node cells collected from each immunized mouse and mouse myeloma cells are mixed at a ratio of 5:1. Hybridomas are prepared by cell fusion using polyethylene glycol 4000 or polyethylene glycol 1500 (GIBCO) as a fusing agent. The lymph node cells of the mouse are fused with mouse myeloma PAI cells (JCR No. B0113; Res. Disclosure Vol. 217, p. 155, 1982), and the resulting hybridomas are selected by culturing the fused cells in an ASF104 medium (Ajinomoto Co. Inc.) containing HAT supplemented with 10% fetal calf serum (FCS) and aminopterin. The reactivity of the culture supernatant of each hybridoma clone is measured by ELISA.

Screening by ELISA is performed by adding the immunogen into each well of a 96-well ELISA microplate (Corning Costar Co.). The plate is incubated at room temperature for 2 hours for the adsorption of the immunogen onto the microplate. The supernatants are discarded and then the blocking reagent (200 μl; phosphate buffer containing 3% BSA) is added into each well. The plate is incubated at room temperature for 2 hours to block free sites on the microplate. Each well is washed three times with 200 μl of phosphate buffer containing 0.1% Tween 20. Supernatant (100 μl) from each hybridoma culture is added into each well of the plate, and the reaction is allowed to proceed for 40 minutes. Each well is then washed three times with 200 μl of phosphate buffer containing 0.1% Tween 20. In the next step, biotin-labeled sheep anti-mouse immunoglobulin antibody (50 μl; Amersham) is added to the wells and the plates are incubated at room temperature for 1 hour.

The microplate is washed with phosphate buffer containing 0.1% Tween 20. A solution of streptavidin-β-galactosidase (50 μl; Gibco-BRL), diluted 1000 times with a solution (pH 7.0) containing 20 mM HEPES, 0.5 M NaCl and bovine serum albumin (BSA, 1 mg/mL), is added into each well. The plate is then incubated at room temperature for 30 minutes. The microplate is then washed with phosphate buffer containing 0.1% Tween 20. A solution of 1% 4-Methyl-umbelliferyl-β-D-galactoside (50 μl; Sigma) in a phosphate buffer (pH 7.0) containing 100 mM NaCl, 1 mM MgCl₂ and 1 mg/mL BSA, is added into each well. The plate is incubated at room temperature for 10 minutes. 1 M Na₂CO₃ (100 μl) is added into each well to stop the reaction. Fluorescence intensity is measured in a Fluoroscan II Microplate Fluorometer (Flow Laboratories Inc.) at a wavelength of 460 nm (excitation wavelength: 355 nm).

### EXAMPLE II

### Preparation of DNA-Antibody Conjugates

Oligonucleotide #109745 (5′ amino-modified, 88 nucleotides in length) was synthesized using standard phosphoramidite chemistry (Biosearch Technologies, Inc., Novato, Calif.) having the following nucleotide sequence:

5′-ATCTGCAGGGAGTCAACCTTGTCCGTCCATTCTAAACCGTTGTGCGT
CCGTCCCGATTAGACCAACCCCCCTATAGTGAGTCGTATTA-3′.

The oligonucleotide was purified using a NAP-5 column (0.1 M/0.15 M buffer of NaHCO₃/NaCl, pH 8.3).

0.2 mL of a 100 μM aqueous solution of oligonucleotide #109745 was loaded onto a column. After 0.3 mL buffer was added, 0.8 mL of eluant was collected and quantified. Based on A₂₆₀ reading, more than 90% of recovery was observed.

The purified oligonucleotide was chemically modified using Succinimidyl 4-formylbenzoate (C6-SFB). 790 μL of purified oligonucleotide and 36 μL of C6-SFB (20 mM in DMF (dimethylformamide)) were mixed (1:40 ratio) and incubated at room temperature for 2 hours. The reaction product was cleaned up using a 5 mL HiTrap desalting column (GE Heathcare) and 1.5 mL eluant was collected. Based on A₂₆₀ reading, more than 80% of oligonucleotide-C6-SFB was recovered.

A Rabbit-anti-Klebsiella antibody (Biodesign, B65891R) was purified using a NAP-5 column (1×PBS buffer, pH 7.2) per manufacturer's instructions. Specifically, 0.25 mL of Rabbit-anti-Klebsiella antibody (4-5 mg/mL) was loaded onto the column, and based on A₂₈₀ reading, 1.27 mg/mL (8.4 μM) antibody was recovered.

Purified Rabbit-anti-Klebsiella antibody was chemically modified using Succinimidyl 4-hydrazinonicotionate acetone hydrazone (C6-SANH). 950 μL of 8.4 μM of Rabbit-anti-Klebsiella antibody and 10.4 μL of C6-SANH (10 mM in DMF) were mixed (1:20 ratio) and incubated at room temperature for 30 minutes. The reaction product was cleaned up using a 5 mL HiTrap desalting column (GE Healthcare) and 1.25 mL eluant was collected. A BCA (“bicinchoninic acid”; Pierce, cat #23225 or #23227) or Bradford (Pierce, cat #23225 or #23236) assay was used to determine the concentration of recovered Rabbit-anti-Klebsiella antibody-C6-SANH (typically ˜1 mg/mL, yield more than 95%). (BSA (bovine serum albumin) was used as the standard for the BCA assay.)

The conjugation of Rabbit-anti-Klebsiella antibody and oligonucleotide was typically achieved by mixing the 1010 μL of Rabbit-anti-Klebsiella antibody-C6-SANH and 750 μL of oligonucleotide-C6-SFB in a molar ratio 1:2 and incubated overnight at room temperature. The resulting conjugates were analyzed on a TBE/UREA gel system, and purified using MiniQ FPLC (fast protein liquid chromatography).

The standard gradient approach was utilized using MiniQ 4.6/50 PE column (GE Healthcare, cat# 17-5177-01; 0.25 mL/min flow rate; detection at 280 nm; Buffer A: 20 mM Tris/HCl, pH 8.1; and Buffer B: 20 mM Tris/HCl, 1 M NaCl, pH 8.1). A BCA or Bradford assay was used to determine the concentration of recovered Rabbit-anti-Klebsiella antibody-oligonucleotide conjugate (˜3 mL of eluant, 0.11 mg/mL).

### EXAMPLE III

### Immobilization of an Electrode-Associated Oligo to a Gold Electrode Surface

The gold electrodes on the chip (Nanostructures, Inc., Santa Clara, Calif.) were cleaned immediately prior to use in UV/ozone cleaner (UVOCS, model T16X16/OES) for 10 minutes. Cleaned chips were stored in container under inert gas (argon).

5′-thiolated oligonucleotides with a C₆ linker were synthesized using standard phosphoramidite chemistry (Biosearch Technologies, Inc. Novato, Calif.).

The spotting solution was prepared by mixing 5′-thiolated C₆ oligonucleotides with mercaptohexanol (MCH) and KHPO₄. Typically, the probe spotting solution consists of a 100 μM thiolated oligo, 1 mM MCH, and 400 mM KHPO₄ (pH 3.8) buffer in aqueous solution.

Chips were printed (30 nl/spot) using BioJet Plus™ series AD3200 non-contact spotter (BioDot, Irvine, Calif.). The relative humidity during the printing was 85%. After incubation of the slides in a humidity chamber for 4 hrs, they were rinsed with an excess of distilled water, dried with argon, and kept in dark under argon at room temperature until use.

### EXAMPLE IV

### Binding of Target Agent and Removal of Excess Capture-Associated Oligo Complexes

### Model System Study

An oligonucleotide AminoR-100003-T7 (5′ amino modified 88 nucleotides long) was synthesized using standard phosphoramidite chemistry (Biosearch Technologies, Inc. Novato, Calif.) and was purified as described in Example II, and had the following sequence:

5′-ATCTGCAGGCCAGGATGACACCTAGATCGTGGTGATCGGGAGTGTGT
CCACGTGACCAACCCCTATAGCCCTATAGTGAGTCGTATTA-3′

The oligonucleotide (AminoR-100003-T7) was conjugated to an anti-Mouse α-Human IL-8 Monoclonal Antibody (ELISA capture, BD Pharmingen, cat# 554716) according to the procedure for conjugation described in Example II. Typically 0.1 mg/mL of the conjugate was obtained. The conjugate therefore contained the AminoR-100003-T7 oligonucleotide (capture-associated oligo) and the anti-Mouse α-Human IL-8 Monoclonal Antibody (capture moiety).

In parallel, NHS(N-hydroxylsuccinimidyl ester) activated agarose beads (GE Healthcare cat. #17090601, medium) were conjugated to an anti-Mouse α-Human IL-8 Monoclonal Antibody (for immunocytochemistry, BD Pharmingen, cat. #550419). First, Mouse α-Human IL-8 Monoclonal Antibody was purified using a NAP-5 column (0.1 M/0.15 M buffer of NaHCO₃/NaCl, pH 8.3), 0.5 mL was loaded, 0.1 mL buffer was added, and 0.7 mL eluate was collected.

0.5 mL of NHS-activated agarose beads (GE, cat #17-0906-01, medium) was sequentially washed with ice-cold 1 mM HCl and ice-cold water. Then, 0.7 mL of Mouse α-Human IL-8 Monoclonal Antibody was added and incubated for 3 hours at room temperature with gentle shaking.

0.5 mL of supernatant from the reaction mixture was passed through a NAP-5 column and a high molecular weight fraction at A₂₈₀ was collected. Subsequently, the agarose beads were blocked with 0.2 M ethanolamine in 0.1 M/0.15 M buffer of NaHCO₃/NaCl (pH 8.3) for 2 hours at room temperature with gentle shaking, and then washed 4 times with 5 mL of 50 mM Tris/HCl, 150 mM NaCl (pH 8.1). After final wash, 0.6 grams of gel was aliquoted, 0.6 mL of 50 mM Tris/HCl, NaCl 150 mM (pH 8.1), 0.1% azide was added, and the mixture was stored at 4° C. Typically, conjugation yields 0.3 mg of antibodies per 1 mL of settled agarose beads. The above-mentioned monoclonal antibodies represent a pair recognizing two different epitopes of recombinant Human IL-8. (The anti-Mouse α-Human IL-8 Monoclonal Antibody on the agarose beads served as an immobilized binding partner.)

To reconstitute a model system, 0.5 μg of target agent, recombinant Human IL-8 (BD Pharmingen, cat#554609, 0.1 mg/mL), was spiked into FBS (Fetal Bovine Serum) along with the AminoR-100003-T7/Mouse α-Human IL-8 Monoclonal AB conjugate (capture-associated oligo complex) (typically 20 μg was used).

15 μg (50 μl) of settled agarose bead-Mouse α-Human IL-8 Monoclonal Antibody conjugate (immobilized binding partner) (100 μL of 50% slurry) was blocked by mixing with Fetal Bovine Serum for 45 minutes at room temperature. The resulting reaction mixture was briefly centrifuged and supernatant was discarded. The above-mentioned reconstituted model system (Human IL-8 and oligo-antibody conjugate) was added to the remaining intact bead bed and the volume of reaction mixture was brought to 500 μL with PBS (phosphate-buffered saline). The reaction mixture, after adding BSA to a final concentration of 1 mg/mL, was incubated at room temperature with continuous mixing.

Unbound oligonucleotide-antibody conjugates (unreacted capture-associated oligo complexes) were removed by washing with PBS (7 times). After the last wash the supernatant was carefully removed and the volume of the bead bed was brought up to 100 μL with PBS. The target-bound conjugates (reacted capture-associated oligo complexes) remained on the agarose beads and were available for detection.

### EXAMPLE V

### Cleavage of a Capture Moiety from a Capture-Associated Oligo

Following the isolation of the target-bound conjugates (reacted capture-associated oligo complexes), it may be desirable in some instances to remove the capture moiety (e.g., antibody) and the target agent from the nucleic acid prior to hybridization. This is accomplished by performing a cleavage reaction to cleave the capture-associated oligo complex between the portion of the capture-associated oligo that will hybridize to the electrode-associated oligo and the capture moiety.

An oligonucleotide is synthesized as described in Example II with a “G-G-C-C” sequence between the capture moiety and the portion of the capture-associated oligo that will hybridize to the electrode-associated oligo. The restriction endonuclease, HaeIII (New England Biolabs), has been shown to cleave single-stranded DNA at this specific sequence (Horiuchi & Zinder, 1975). The cleavage reaction is performed by mixing the HaeII enzyme with the capture-associated oligo complexes in a buffer containing 10 mM Tris-HCl, 50 mM NaCl, 10 mM MgCl₂, and 1 mM dithiothreitol, pH 7.9, and incubating at 37° C. for 30 minutes. The HaeIII enzyme is heat-inactivated at 80° C. for 20 minutes. The cleaved oligos are separated from the remainder of the capture-associated oligo complex by standard techniques such as ethanol precipitation. Briefly, add 2.5-3 volumes of 95% ethanol/0.12 M sodium acetate to the DNA sample contained in a 1.5 mL microcentrifuge tube, invert to mix, and incubate in an ice-water bath for 10 minutes. The resulting mixture is centrifuged at 12,000 r.p.m. in a microcentrifuge for 15 minutes at 4° C., the supernatant is decanted, and the pellet is drained by inversion on a paper towel. Ethanol (80%) (corresponding to about two volume of the original sample) is added and the reaction mixture is incubated at room temperature for 5-10 minutes followed by centrifugation for 5 minutes. The supernatant is then decanted. The sample is air-dried (or alternatively lyophilized) and the pellet of DNA resuspended in 10 mM Tris-HCl, pH 7.6-8.0, 0.1 mM EDTA. For hybridization reactions, the nucleic acid is resuspended in SSC solution.

In an alternative cleavage method, photocleavage is performed. In doing so, an oligonucleotide is synthesized as described in Example II with a photocleavable nucleotide inserted into the sequence. This can be accomplished by using a photocleavable phosphoramidite during the synthesis of the oligonucleotide (Glen Research). The cleavage reaction is essentially performed by exposing the capture-associated oligo complex to a source of ultraviolet (UV) light. The cleaved oligos are separated from the remainder of the capture-associated oligo complex by standard techniques such as ethanol precipitation, membrane filtration, or if the remainder of the capture-associated oligo complex is immobilized, centrifugation, etc.

### EXAMPLE VI

### Hybridization of Nucleic Acid Molecules to the Electrode-Associated Oligos

The hybridization and detection reaction was carried out as follows. The printed DNA chip containing the electrode-associated oligos was assembled into a PAR 2-chamber cartridge (Antara BioSciences Inc., custom design). 500 μL of target hybridization solution and 10 nM single-stranded nucleic acid (60 nucleotides long) in 6×SSPE buffer (0.9 M NaCl, 60 mM NaH₂PO₄, 6 mM EDTA) was injected into the cartridge. The hybridization reaction was carried out in the 55° C. oven for 60 minutes with gentle shaking. Then, the hybridization solution was pipetted off and the chips were rinsed twice with 500 μL of pre-warmed (55° C.) 0.2×SSC (30 mmol/L NaCl, 3 mmol/L trisodium citrate). 500 μL of pre-warmed 0.2×SSC was added into the chip and incubated at 55° C. for 20 minutes with gentle shaking. Stringency wash buffer (0.2×SSC) was removed and the chips were rinsed twice with 500 μL 20 mM NaPO₄/100 mM NaCl, pH 7.0 at room temperature.

Next, 500 μL of 50 μM Hoechst 33258 dye (Invitrogen) in 20 mM NaPO₄/100 mM NaCl, pH 7.0 was added into the chip and incubated at room temperature for 15 minutes. The stain was pipetted off and the chip was rinsed twice with 500 μL of 20 mM NaPO₄/100 mM NaCl, pH 7.0 at room temperature. 500 μL of 20 mM NaPO₄/100 mM NaCl, pH 7.0 was added into the chip and incubated at room temperature for 5 minutes. The buffer was pipetted off, and the chip was rinsed twice with 500 μL of 20 mM NaPO₄/100 mM NaCl, pH 7.0. Then, the hybridization chamber was filled with 1 mL of 500 μL of 20 mM NaPO₄/100 mM NaCl, pH 7.0.

The electrochemical analysis (cyclic voltammetry) was carried out with an electrochemical analyzer (Model VMP3) and software from Princeton Applied Research (PAR). The measurement was performed at 100 mV/sec scan rate at room temperature, and the potential sweep range was from +200 mV to 800 mV and back to 200 mV.

### EXAMPLE VII

### Binding of Target Agent (E. coli O157:H7) and Alternative Method of Removal of Excess Capture-Associated Oligo Complexes

A sample is obtained from a patient suffering from an E. coli O157:H7 infection and is diluted in PBS/Tween20. An oligonucleotide (capture-associated oligo) conjugated to an anti-E. coli O157:H7 antibody (capture moiety) (the procedure for conjugation is described in Example II) is contacted with the diluted sample by adding a one-third volume of bovine serum albumin (12% [wt/vol] in PBS) and 2 μg of antibody-nucleic acid conjugate (capture-associated oligo complex). The resulting reaction is incubated at room temperature for 30 minutes.

Unbound antibody-nucleic acid conjugates (unreacted capture-associated oligo complexes) are removed by magnetic microparticle depletion. Briefly, magnetic microparticles are coated with a second anti-E. coli O157:H7 antibody (immobilized binding partner), specific to another region (epitope) of the same target agent to be detected. These microparticles are prepared, e.g., as described in Example XII. Alternatively, the second antibody (immobilized binding partner) could specifically bind the first antibody/antigen complex (capture moiety/target agent complex). Magnetic beads coated with the second antibody are added to the reaction mixture, in a PBS buffer supplemented with 0.5% BSA and 2 mM EDTA, and incubated at 4° C. for 30 minutes. Only those antibody-nucleic acid conjugates that have bound to E. coli O157:H7 in the sample (reacted capture-associated oligo complexes) are available to bind to the magnetic particle immobilized second anti-E. coli O157:H7 antibody, specific to another region (epitope) of the same target agent to be detected. The magnetically-labeled conjugate is separated from the reaction mixture by adding the mixture to a column packed with lattice-type matrix and applying a magnetic field. Such separation devices are known in the art (e.g., MACS® Columns, Miltenyi Biotec). The magnetically-labeled second antibody-nucleic acid conjugate that is bound to the target agent (immobilized reacted capture-associated oligo complex) is retained on the column. The antibody-nucleic acid conjugate that is not bound to the target agent (unreacted capture-associated oligo complex) will pass through the column.

Subsequently, cleavage of the capture-associated oligo (or a portion thereof) from the magnetically-labeled second antibody-nucleic acid conjugate that is bound to the target agent is performed as described in Example V. This cleavage can be achieved by other approaches, described earlier in this invention. The cleavage products are then subjected to electrochemical detection.

### EXAMPLE VIII

### Binding of Target Agent (Human Anti-Hepatitis Antibodies) without Direct Interaction with the Causative Agent

A sample is obtained from a patient suspected of being infected with hepatitis. The sample is diluted in a diluent such as PBS/tween20. An oligonucleotide conjugated to a hepatitis-specific antigen (or a plurality of different antibodies all specific to different hepatitis-specific antigens) is incubated with the diluted sample by adding a one-third volume of bovine serum albumin (12% [wt/vol] in PBS) and 2 μg of the oligo nucleotide-antigen conjugate (capture-associated oligo complex). Unbound nucleic acid-antigen complex (unreacted capture-associated oligo complex) is removed by magnetic microparticle-antibody affinity depletion. Briefly, magnetic micro-particles are coated with an antibody affinity reagent such as Protein A, Protein G or anti-class antibody which captures antibodies from the sample, a portion of which may be hepatitis antigen specific and bound to the antigen-oligo conjugate. The coated magnetic beads (immobilized binding partner complexes) are added to the reaction mixture, in a PBS buffer supplemented with 0.5% BSA and 2 mM EDTA, and incubated at 40° C. for 30 minutes. Antibodies in the sample will be immobilized on the magnetic beads, but only anti-hepatitis antibodies will contain the oligo-antigen conjugate (i.e., will contain capture-associated oligos). The magnetically-labeled antibody affinity reagent, along with bound oligo-antigen complexes (immobilized reacted capture-associated oligo complexes) are separated from the rest of the sample and extensively washed with PBS/Tween20. Such separation techniques are known in the art (e.g., MACS Columns, Miltenyi Biotec). Subsequent release of the oligo from the antigen is performed as described in Example V and other approaches, described herein.

### EXAMPLE IX

### Creation of Multiple Copies of Capture-Associated Oligos (or Complements Thereof) for More Sensitive Detection of the Target Agent via Linear Amplification

An oligonucleotide AminoR-100003-T7 (5′ amino-modified 88 nucleotides long capture-associated oligo) was synthesized using standard phosphoramidite chemistry (Biosearch Technologies, Inc. Novato, Calif.) and was purified as described in Example II, and had the following nucleotide sequence:

5′-ATCTGCAGGCCAGGATGACACCTAGATCGTGGTGATCGGGAGTGTGT
CCACGTGACCAACCCCTATAGCCCTATAGTGAGTCGTATTA-3′

The oligonucleotide (AminoR-100003-T7, capture moiety) was conjugated to an anti-Mouse α-Human IL-8 Monoclonal Antibody (BD Pharmingen, cat# 554716) according to the procedure for conjugation described in Example II. 0.1 mg/mL of the conjugate was obtained. The conjugate therefore contained the AminoR-100003-T7 oligonucleotide (capture-associated oligo) and the anti-Mouse α-Human IL-8 Monoclonal Antibody (capture moiety). In addition, the 3′ end of the capture-associated oligo contained the specific sequence as follows:

5′-CCCTATAGTGAGTCGTATTA-3′

The methods described in Example IV were performed to immobilize reacted capture-associated oligo complexes (i.e., bound to target agent (Human IL-8)) using a second anti-Mouse α-Human IL-8 Monoclonal Antibody (BD Pharmingen cat# 550419) binding partner, which was specific to another epitope of the same target agent) immobilized on agarose beads. Urea was added to the beads (agarose beads in 100 μL of PBS from a final step of Example IV) to a final concentration of 1 M. The tube containing all Model System components was incubated for 3 minutes at room temperature. In-Vitro Transcription (IVT) reactions were performed according to the manufacturer's user manual (Ambion MEGAshortscript kit, cat. #1354).

2 μL of the supernatant from the beads with urea in the Model System was taken out and mixed with 2 μL of the T7 primer 2 (250 nM), which is complementary to the 3′ end specific sequence of the capture-associated oligo described earlier in this example: 5′-TAATACGACTCACTATAGGG-3′; the reaction mixture was incubated at 65° C. for 5 minutes and then cooled to 37° C., resulting in hybridization of the complementary synthetic 20-mer T7 primer 2 to the 3′ end of the capture-associated oligo, creating double-stranded recognition sites for T7 RNA polymerase.

In parallel, 2 μL of oligonucleotide AminoR-100003-T7 (250 nM) was annealed with 2 μL of the T7 primer 2 (250 nM) at 65° C. for 5 minutes as the IVT control. Additional components of the In-Vitro Transcription were added to the reaction mixtures according to the manufacturer's user manual (Ambion MEGAshortscript kit, cat# 1354) and incubated for 2 hours at 37° C.

After the IVT reactions were completed, a 1:50 dilution of the reaction mixture was made. 3 μL of the diluted transcribed products was mixed with an equal volume of the Gel Loading Buffer, heated at 95° C. for 5 minutes, cooled to room temperature, spun briefly, and loaded onto an 8% TBE/urea denaturing gel system. The gel was stained for 1 minute by SYBR Gold (Invitrogen cat# S11494) by making a 1:10,000 dilution in 1×TBE. The stained gel was rinsed in 1×TBE or Milli-Q water and the image was taken using a UVF BioDoc-It unit. A band, corresponding to the expected size transcript (68 bases long), was observed. Linearly-amplified transcripts were separated from the reacted capture-associated oligo complex by standard techniques such as centrifugation, column purification or ethanol precipitation. Briefly, the resulting mixture was centrifuged at 12,000 r.p.m. in a microcentrifuge for 5 minutes at room temperature. The supernatant, containing the linearly-amplified transcript, was transferred into a separate 1.5 mL microcentrifuge tube. 2.5-3 volumes of 95% ethanol/0.12 M sodium acetate were added to the sample contained in a 1.5 mL microcentrifuge tube, inverted to mix, and incubated in an ice-water bath for 10 minutes. After centrifugation, the supernatant was decanted, and the tube (containing the pelleted material) was drained (by inversion on a paper towel). Ethanol (80%) (corresponding to about two volume of the original sample) was added and the reaction mixture was incubated at room temperature for 5-10 minutes followed by centrifugation for 5 minutes. The supernatant was then decanted. The sample was air-dried and the pellet of nucleic acid (linearly-amplified transcript) was resuspended in 6×SSPE buffer (0.9 M NaCl, 60 mM NaH₂PO₄, and 6 mM EDTA), and was subsequently taken for electrochemical detection.

Alternatively, before the linear amplification reaction, the capture-associated oligo was released from the reacted capture-associated oligo complex by mixing with PstI restriction enzyme. Briefly, 2 μL of the supernatant from the beads with urea in the Model System was taken out and mixed with 2 μL of the Restriction Site Restore Oligo (250 nM), which is complementary to the 5′ end specific sequence of the capture-associated oligo described earlier in this example:

5′-TGTCATCCTGGCCTGCAGAT-3′

The reaction mixture was incubated at 65° C. for 5 minutes and then cooled to 37° C., resulting in hybridization of the complementary synthetic 20-mer Restriction Site Restore Oligo to the 5′ end of the capture-associated oligo, creating double-stranded recognition sites for Pst I restriction enzyme. Restriction digestion with Pst I enzyme (New England Biolabs, cat. #R0140S) was carried out according to the manufacturer's suggested protocol.

After restriction, the Pst I enzyme was heat-inactivated at 80° C. for 20 minutes. Subsequent linear amplification and purification of the linearly-amplified transcript was achieved as described earlier in this example.

### EXAMPLE X

### Quantitation of Interleukin-10

A serum sample is obtained from a patient where the amount of IL-10 is to be determined. The sample is diluted in a diluent such as PBS/tween20. A capture-associated universal oligo conjugated to an IL-10 specific antibody is incubated with the diluted sample by adding a one-third volume of bovine serum albumin (12% [wt/vol] in PBS) and 2 μg of the capture-associated universal oligo conjugated to the IL-10 specific antibody. Unreacted capture-associated universal oligo complex is removed by incubating the sample with immobilized antigen (IL-10) (immobilized binding partner) in PBS/Tween20 buffer at 4° C. for 1 hour. Briefly, a micro-titer plate is used to immobilize IL-10 in a PBS/Tween20 blocking solution. Incubation of the sample in the coated well removes any unreacted capture-associated universal oligo complexes from solution. The solution is removed leaving IL-10 complexed with the reacted capture-associated universal oligo complex in the well. Subsequent release of the capture-associated universal oligo from the reacted capture-associated universal oligo complex is performed as described in example IV and other approaches described herein to create released universal oligo. A unique set of quantifying oligos is added to the sample. The sample with quantifying oligos is contacted with a chip comprising electrode-associated oligos complementary to the quantifying oligos and electrode-associated universal oligos complementary to the released universal oligo. Signal is detected from a) the hybridization of the quantifying oligos with the electrode-associated oligos complementary thereto, and b) the hybridization of the released universal oligo with the electrode-associated universal oligos complementary thereto. The signal generated from hybridization of the released universal oligo is compared to the signal generated from hybridization of the quantifying oligos added to the sample with known concentrations to determine the concentration of the released universal oligo, and this concentration may be used to determine the amount of target agent in the sample by standard statistical methods known to those of ordinary skill in the art.

### EXAMPLE XI

### Preparation and Use of Loaded Scaffolds Using Gold Particles for the Scaffold Substrate and Antibodies as the Capture Moiety

Loaded scaffolds were created by attaching oligonucleotides and capture moieties onto a substrate. In one example, the scaffold substrate was comprised of 20 nm gold particles, and the capture moiety was comprised of a goat anti-rabbit IgG polyclonal antibody. 30 mL (7×10¹¹ particles/mL) of commercially available gold colloid particles (Ted Pellau Inc., Redding, Calif.) was adjusted to pH 9.0 with the addition of 35 □L of 0.2 M borax, pH 9.05. The antibody solution was prepared for conjugation by first diluting the reagent to a final concentration of 0.2 μg/μL solution in 2 mM borax, pH 9.05 and then dialyzing it for at least 4 hours in 1 liter of borax at pH 9.05. 7.5 □g of the dialyzed antibody solution was added to 1 mL of the gold colloid solution, was lightly vortexed for 5 seconds, and was then incubated at room temperature to absorb the protein onto the colloid particles. After 20 minutes, 25 □L of a 96-mer oligonucleotide (0.7 OD) was added to the solution and incubated overnight at 4° C. Oligonucleotides were attached to the antibody-gold particle scaffold through the use of the functionalized chemical group alkylthiol, attached to the 5′ terminus of the oligonucleotide. The 3′ terminus of the oligonucleotide primer contained a promoter sequence for T7 RNA polymerase for downstream analysis of functionality of the loaded scaffolds. The solution of colloid particles, now absorbed with antibody and oligonucleotides onto their surface, were sequentially adjusted with salt to 0.1 M NaCl for five minutes and then to 3% bovine serum albumin at room temperature for 2 hours in order to stabilize the scaffolds. This solution was then purified via centrifugation at 12,000 g for 5 minutes at room temperature, the supernatant was removed, and the pellet was washed twice with a solution comprised of 0.1 M NaCl, 10 mM PO₄, pH7.4. The pellets were resuspended in a minimal volume of phosphate buffered saline solution (PBS) and were stored at 4° C. prior to use.

For experiments, aliquots of the loaded gold scaffold were incubated for 2 hours at room temperature with IgG-containing serum samples derived from either rabbit or mouse, with the latter serving as a negative control for the experiment. Magnetic beads which were conjugated with sheep anti-rabbit IgG polyclonal antibodies were then added into each of the mixtures and incubated for an additional 1 hour at room temperature. The magnetic beads were collected to the side of the tube on a stand which contained a magnet. Typically, the gold colloids possess a distinct dark red color making the solution appear red. However, in samples in which rabbit serum had been added and the magnetic beads ‘captured’ to the side of the tube, the presence of rabbit IgG in the sample allowed for the efficient co-capture of the loaded gold scaffolds to the side of the tube, presumably from the formation of the appropriate antibody sandwich, resulting in a clear and colorless supernatant. In negative control samples in which mouse serum had been added, the supernatant remained a red-colored solution following application of the magnetic field.

In order to further confirm that the loaded gold scaffolds had functioned properly, the captured magnetic beads were washed several times with a phosphate buffered saline solution, the supernatants were discarded, and the beads were resuspended in a minimal volume of dH₂O. The resuspended particles were then added as template material into T7 in vitro RNA transcription reactions using a commercially available kit (Ambion, Austin Tex.). The reaction products of the T7 reactions were loaded onto 15% UREA-TBE gels and the corresponding nucleic acid products were resolved by gel electrophoresis, stained with SyBR-gold (Invitrogen, San Diego, Calif.) and analyzed on a fluorescent scanner (Fujifilm Medical Systems, Stamford, Conn.). Samples that received rabbit serum clearly demonstrated the appearance of a nascent RNA transcript of the appropriate expected length (approximately 76 nucleotides) that corresponded to expected products from the oligonucleotide template that had been absorbed onto the gold particle loaded scaffold. In samples which received the mouse serum, these transcripts were clearly absent.

### EXAMPLE XII

### Preparation of Magnetic Beads with Antibodies Immobilized on the Bead Surface

Magnetic particles (“beads”) may be used as the substrate and antibodies may be attached to form the immobilized binding partner. The use of magnetic beads is well known in the art and these reagents are commercially available from such sources as Ademtech Inc., (New York, N.Y.) and Promega U.S. (Madison, Wis.). “Amino-Adembeads” may be obtained from Ademtech and these beads consist of a magnetic core encapsulated by a hydrophilic polymer shell, along with a surface activated with amine functionality to assist with immobilization of antibodies to the bead surface. The beads are first washed by placing the beads in the included “Amino 1 Activation Buffer,” then this reaction tube is placed in a magnetic device designed for separation. The supernatant is removed, the reaction tube is removed from the magnet, and the beads are resuspended in the included “Amino 1 Activation Buffer.” To assist coupling of the antibody with the magnetic bead, EDC (1-ethyl-3-(3-dimethlaminopropyl) carbodiimide hydrochloride) (4 mg/mL) is dissolved into the included “Amino 1 Activation Buffer”, and an appropriate amount of this solution is added to the beads (80 μL/mg beads), and vortexed gently. 10-50 μg of antibodies is then added per mg of beads, and the solution is vortexed gently. The solution is incubated for 1 to 2 hours at 37° C. under shaking. Bovine serum albumin (BSA) is then dissolved in “Amino I Activation Buffer” to a final concentration of 0.5 mg/mL, and 100 μL of this BSA solution is added to 1 mg of antibody-coated beads, and the solution is vortexed gently and incubated for 30 minutes ant 37° C. under shaking. The beads are then washed in the included “Storage Buffer” twice, and the beads are resuspended.

### EXAMPLE XIII

### Alternative Method of Binding Target Agent (E. coli O157:H7) and Removal of Unreacted Loaded Scaffold

A sample is obtained from a patient suffering from an E. coli O157:H7 infection and is diluted in PBS/Tween20. Antibodies are covalently attached to magnetically labeled microparticles (immobilized binding partner complexes) utilizing techniques standard to those who practice the art. (A procedure for making such magnetic microparticles coated with antibody is described in Example XII.) Densities of antibodies on the magnetic microparticles are fairly standard such that one can expect that 7×108 beads/mL typically results in approximately 10 mg/mL protein concentration. The magnetic microparticles are then washed two times with a solution comprised of 10 mM phosphate buffered saline, pH 7.4 and 100 mM NaCl (PBSNa) and resuspended in a minimal volume of PBSNa (approximately 100 μL) supplemented with BSA to final concentration of 2.75%. The sample suspected of containing the target agent (approximately 10 μL) is added into the mixture with the magnetic microparticles at the proportion of one-tenth the volume of suspension containing the magnetic microparticles. The resultant mixture is incubated at room temperature with gentle shaking for 30-60 minutes. Preferably, one would anticipate that the binding partners immobilized on the surface of the magnetic particles are at concentrations that are in molar excess, preferably at least 10-fold molar excess, of the corresponding target agent present within the added sample mixture. The magnetically-labeled microparticle/target agent complex is separated from the reaction mixture by adding the mixture to a column packed with lattice-type matrix and applying a magnetic field. Such separation devices are known in the art (e.g., MACS® Columns, Miltenyi Biotec). The magnetically-labeled microparticle/target agent complex is retained on the column. The target agent that is not bound to the magnetically-labeled microparticle/target agent complex will pass through the column.

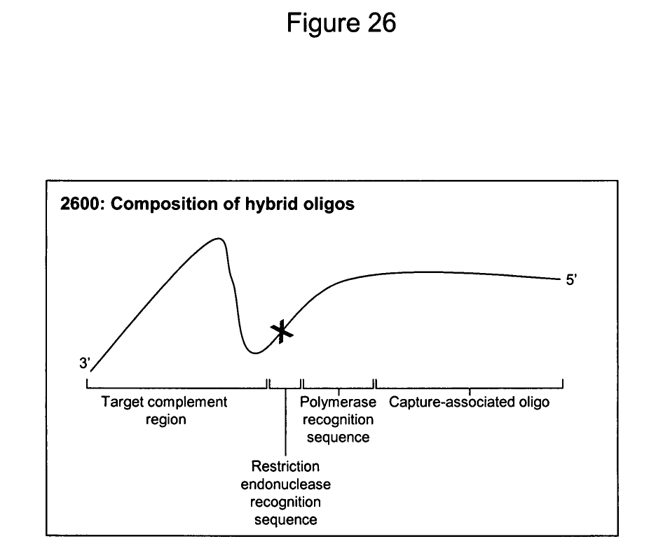

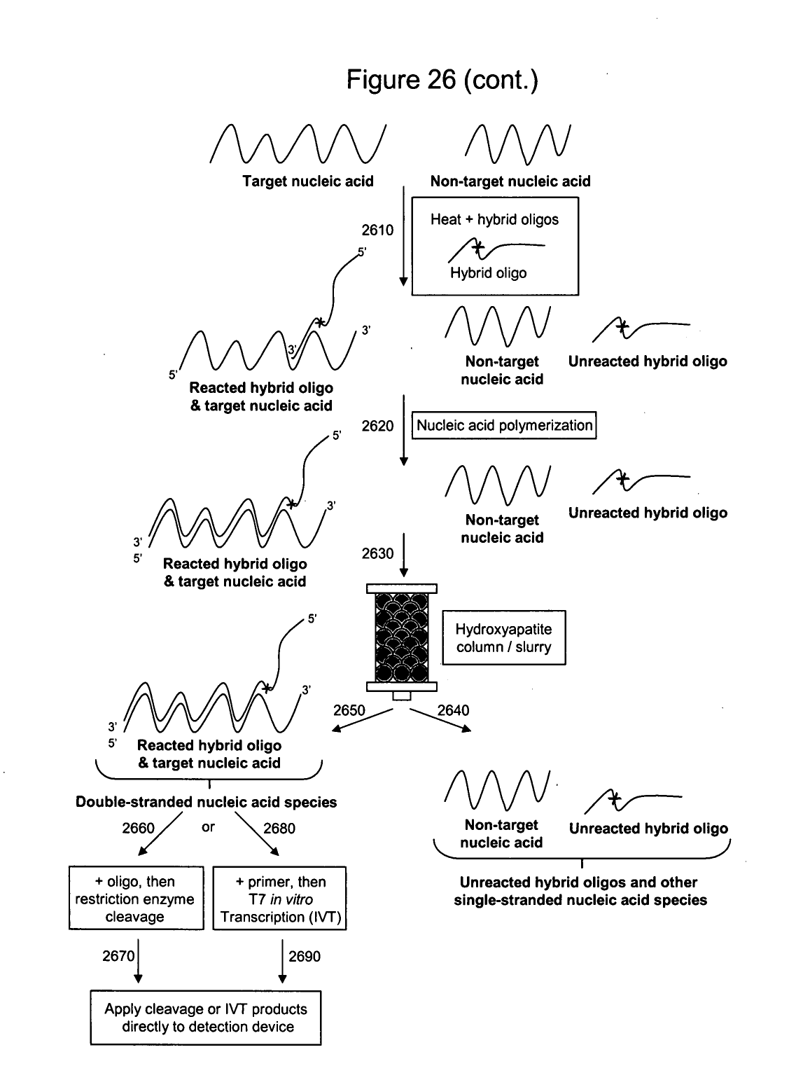

Loaded scaffolds are generated with a second anti-E. coli O157:H7 antibody (capture moiety), (the procedure for making such loaded scaffold is described in Example XI) specific to another region (epitope) of the same target agent to be detected. The second antibody-loaded scaffolds are added to the reaction mixture, in a PBS buffer supplemented with 0.5% BSA and 2 mM EDTA, and incubated at 4° C. for 30 minutes. Following incubation, a magnetic field is applied to separate the magnetically-labeled microparticle/target agent complex away from the remainder of the sample. The magnetic microparticle/target agent complexes are washed twice with PBSNa and resuspended in 20 μL of PBSNa containing 3.75% BSA. 10 μL of loaded scaffolds with a second anti-E. coli O157:H7 antibody are contacted with the microparticle/target agent complex mixture and this mixture is subsequently incubated with gentle shaking for 30-60 minutes at room temperature. Following incubation, a magnetic field is used to separate loaded scaffolds that bound to the microparticle/target agent complexes from those that remain unbound. After two washes, the beads are resuspended in a minimal volume of PBSNa. Only those E. coli O157:H7 target agents that have bound to magnetic particles in the first reaction are available to bind to the second anti-E. coli O157:H7 antibody (on the loaded scaffolds) specific to another region (epitope) of the same target agent to be detected. The magnetically-labeled target agent/loaded scaffold/magnetic particle complex is separated from the reaction mixture by adding the mixture to a column packed with lattice-type matrix and applying a magnetic field. The magnetically-labeled complex is retained on the column. The loaded scaffold that is not bound to the target agent/magnetic particle complex will pass through the column. Capture-associated universal oligos on the loaded scaffolds that are retained on the column are then subjected to electrochemical detection. In certain embodiments of the invention, a target nucleic acid may be detected in a sample without exposing the target nucleic acid to a biosensor or other detection device (e.g., an electrochemical detection device). For example, in some embodiments, a hybrid DNA oligo is synthesized for detection of such a target nucleic acid in a sample. Specifically, such hybrid oligos may comprise multiple regions, each of which serves a different function within the assay. In one such example, the 3′ end of the hybrid oligo comprises a sequence known to be complementary to the target nucleic acid, and this complementary sequence (the “target complement region,” which serves as a capture moiety) comprises a length and sequence of nucleotides sufficient to ensure that binding of the hybrid oligo is specific for detection of the target nucleic acid amongst all other nucleic acids in the sample. Immediately 5′ of the target complement region is a restriction endonuclease recognition sequence. Immediately 5′ of the restriction endonuclease recognition sequence is a polymerase recognition sequence. In certain embodiments, this polymerase recognition sequence is a reverse complement sequence for the promoter that is required for T7 RNA polymerase activity, thereby ensuring that polymerization from this sequence will serve to amplify the capture-associated oligo region of the hybrid oligo, which is located immediately 5′ of the polymerase recognition sequence. The capture-associated oligo comprises a sequence that can be used as a template for a polymerase reaction to produce amplification products that are complementary to chip-associated oligos (e.g., electrode-associated oligos). An example of the composition of a hybrid oligo is shown at 2600 in FIG. 26. These hybrid oligos are useful in certain embodiments of the methods described herein for detecting and/or quantifying the relative levels of a target nucleic acid present within an unknown sample, as described below.

A first mixture is created by adding the hybrid oligos directly into a sample comprising target nucleic acids to be detected (step 2610). The first mixture is heated to high temperatures to simultaneously disassociate any pre-existing double-stranded DNA duplexes and/or minimize the amount of RNA secondary structure (step 2610). Upon cooling of the first mixture, annealing of the hybrid oligo to the target nucleic acids is mediated by the complementary sequences on the 3′ end of the hybrid oligo (step 2610). Once annealed to the target nucleic acid, the hybrid oligo serves as the initiation point of template extension by nucleic acid polymerizing enzymes such as the Klenow fragment of DNA polymerase I or reverse transcriptase (step 2620). The nascent nucleic acid synthesis will proceed in a 5′-3′ direction, thereby generate double-stranded target nucleic acid from the single-stranded target nucleic acid specifically bound to the target complement region of the hybrid oligo (step 2620).

Subsequently, the first mixture can be applied to a column capable of separating double stranded material from single stranded material, e.g., a hydroxyapatite column (step 2630) to purify the double-stranded nucleic acid species (i.e., the hybrid oligo bound to now (at least partially) double-stranded target nucleic acid) away from the single-stranded nucleic acid species (e.g., non-target nucleic acids, unreacted hybrid oligo, i.e., not bound to target nucleic acid) in the first mixture. In this way, the single-stranded nucleic acid species are washed through the column and are discarded (step 2640), while double-stranded nucleic acid species are preferentially retained on the column. Double-stranded target nucleic acids that have re-annealed following the heating/strand disassociation step may also be present, but will not interfere will subsequent analysis. Elution and recovery of the double-stranded nucleic acid species is accomplished by washing the column with a buffer high in phosphate content to produce a second mixture (step 2650).

A primer complementary to the polymerase recognition sequence (e.g., T7 RNA polymerase promoter, as described above) can be added to the second mixture for annealing to the hybrid oligo (step 2680). Once annealed, polymerase reactions (e.g., T7 in vitro transcription reactions) are performed to generate oligos (e.g., RNA transcripts) complementary to chip-associated oligos (step 2680). The oligos generated by the polymerase reactions are then introduced to a chip for subsequent detection (step 2690), which is indicative of presence of a target nucleic acid in the sample.

In alternative embodiments, the hybrid oligo does not comprise a polymerase recognition sequence and the capture-associated oligos are complementary to chip-associated oligos. In such embodiments, the capture-associated oligos are released from the hybrid oligos, separated from the hybrid oligos and target nucleic acids, and are subsequently introduced to the detection device. For example, an oligo complementary to the restriction endonuclease recognition sequence will be added to the second mixture and permitted to hybridize to the hybrid oligo, and an appropriate restriction endonuclease will be used to cleave the capture-associated oligos from the hybrid oligos (step 2660). (Alternately, a restriction endonuclease may be used that is specific for single-stranded DNA, in which case addition of the oligo complementary to the restriction endonuclease recognition sequence is not required.) Once released from the hybrid oligo, the liberated capture-associated oligo (or a portion thereof) can be applied directly to the detection device for quantification (2670).

In yet further embodiments, in order to purify the single-stranded capture-associated oligo sequences away from any double-stranded target nucleic acids retained on the hydroxyapatite column, the hybrid oligo may be subjected to cleavage with a restriction endonuclease prior to elution of the double-stranded nucleic acid species from the column. Thus, the second elution of single-stranded nucleic acid species from the column would contain substantially pure oligo comprising the polymerase recognition sequence and the capture-associated oligo, which can be applied directly to the detection device if complementary to chip-associated oligos, or which can be subjected to linear amplification if the amplification products are complementary to chip-associated oligos.

In a related embodiment, a second elution of single-stranded nucleic acid species from the hydroxyapatite column can be performed after linear amplification of the capture-associated oligo, thereby providing an aqueous solution comprising substantially pure single-stranded amplicons for application to the detection device. In such an embodiment, cleavage of the capture-associated oligo from the hybrid oligo is not required, so the hybrid oligo need not encode a restriction endonuclease recognition sequence.

In alternate embodiments, the separation of the double-stranded nucleic acid species from the single-stranded nucleic acid species may be performed in a hydroxyapatite slurry rather than on a hydroxyapatite chromatography column. In brief, the hydroxyapatite is allowed to bind to the double-stranded nucleic acid species and is spun down, thereby creating an immobilized phase comprising the double-stranded nucleic acid species and an aqueous phase comprising the single-stranded nucleic acid species, which can be subsequently removed (e.g., by aspiration, decanting, etc.) and discarded. Subsequently, the slurry is resuspended and either a) the hybrid oligo may be treated with a restriction endonuclease to remove the portion of the hybrid oligo comprising the polymerase recognition sequence and the capture-associated oligo, or b) the hybrid oligo may be treated with a polymerase (e.g., T7 polymerase) and the appropriate nucleotides to facilitate creation of linear amplification products. The slurry is spun down again and the aqueous phase is recovered. The aqueous phase will contain either a) the portion of the hybrid oligo comprising the polymerase recognition sequence and the capture-associated oligo, or b) linear amplification products, respectively. If the aqueous phase contains the portion of the hybrid oligo comprising the polymerase recognition sequence and the capture-associated oligo, then it may be amplified and the amplification products may be applied to the detection device. If the aqueous phase contains the linear amplification products, they may be applied directly to the detection device.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The abstract and the title are not to be construed as limiting the scope of the present invention, as their purpose is to enable the appropriate authorities, as well as the general public, to quickly determine the general nature of the invention. The scope of the invention will be measured by the appended claims along with the full scope of equivalents to which such claims are entitled. In the claims that follow, unless the term “means” is used, none of the features or elements recited therein should be construed as means-plus-function limitations pursuant to 35 U.S.C. §112, ¶6. All publications mentioned herein are cited for the purpose of describing and disclosing reagents, methodologies and concepts that may be used in connection with the present invention. Nothing herein is to be construed as an admission that these references are prior art in relation to the inventions described herein. Throughout the disclosure various patents, patent applications and publications are referenced. Unless otherwise indicated, each is incorporated by reference in its entirety for all purposes.

TABLE 1

| | |
|---|---|
| SEQ | |
| ID # | SEQUENCE |

| | |
|---|---|
| 1 | AAGGTACGAACGACTAACGGGTCCTAACGGGACGCTACTAGGGCG |
| | ACTAATCAGATCCCT |
| 2 | ACCGAGTAATGAGACGTGTACCCTAGACCGGAGGCACCTTACGAA |
| | CAGTGGTCAGATCCC |
| 3 | ACCTAACGTCGATGCGTCTCGTAACAGTTCGCGTCCTAGTGCAAC |
| | CGCGTGGGTCAGATC |
| 4 | ACGATACTCCCGACTTACAGTTCGCTCCGTGGATTACGACCGACC |
| | CAATTTATCAGATCC |
| 5 | ACGTCGGTCTACTCAACTAACGTAGCGTATGTCGGATTCGCGTTG |
| | TGGAAATTCAGATCC |
| 6 | ACTCACGCGATTAGCGGTTCGCAATACTAGGCGAGCGTTACATAT |
| | CCGAGCGATCTCAGA |
| 7 | AGCCCGATAGTCCGACACTCCTACTTCGCTTAGCCGTATAGTGGC |
| | CTGCTACGGAATCAG |
| 8 | ATCGCTACAGGGTCGCGCTAATACAGGTCGCATATCGGAGTTCAC |
| | CGCAATAGTCAGATC |
| 9 | ATCGGCCCTACGTTACGGACTAACTACGCGGTCCCTACGATTCGC |
| | GGACTAGACAATCAG |
| 10 | TGAGTGCGAGACGAGGGAGGGTATCGACACCGCTCAATACATTCG |
| | TGAGAATCGATCAG |
| 11 | CCGAAACCACAATTCGTCGGTCCGTATTGATCCGGCTCGACACGT |
| | ATGCCTGTGCGACTA |
| 12 | ATCCTCCGAACTAATCCGATATTTCTCGCAACGGTTAGTCGATCC |
| | CACTACGCCGCTTAT |
| 13 | TATGTTGTCGACCATGGCTATCGTACGGGATTGCCTCGGATTATC |
| | GTCTGACGGGTTAAT |
| 14 | CAGACCACATAATCGTACTTGCAACGGAACAAATTTCGACGCCCT |
| | AAGTTGGTTGTCACT |
| 15 | ATTCGAGACGTAAACGGATACGATCCCTGTATTCGTAACGTTGTA |
| | ACGCAACAGACATCA |
| 16 | TGGAACGAACGCTTACGTGTATTGTGGAGCGTATCTTAGGCCACC |
| | GGATTCTCACACTTC |
| 17 | GGAGTCAACCTTGTCCGTCCATTCTAAACCGTTGTGCGTCCGTCC |
| | CGATTAGACCAACCC |
| 18 | CACGACGACTTGACGGCAGTGCGTACCGGCGACTGTTACGGTGGG |
| | TCGAACAAGTCGCTA |
| 19 | CACTCCGCACCCTTACGCACCGGACTAAAGGTATTCGCGGGAAAC |
| | TGCATACCGCCCGTT |
| 20 | GCGAATGCGTCTAATACCGCTACCTTCGAGCGCAGGTTGACCGTC |
| | CGTACTATCAGTCAA |
| 21 | TCTTGGCGTTAACCGTTAGATCGACTTCCCGTATTGAGAACGGAA |
| | CGGCATGAACGGAGG |
| 22 | TAACCTCAAGGTCTACGATAGCGGATAAGTCGTGAAGAGCGTGAA |
| | CGATATTGCAATCAC |
| 23 | GAGCGTAAATTCTACGCGATAGTTCGTATCTATGGCTTCGAAACC |
| | GTCTGCATCGATGAC |
| 24 | CTGGGAATTGTAGCGCCAGTGGATATAGCCCGTCAATGCCGCGTA |
| | AGTCTACGTACAGCC |
| 25 | TCACTACGTAACCTCGACAAACCGTATTCCCGTGGCGCAAGTCTG |
| | GATCGGCGACCTCGG |
| 26 | TCCCGAGGTTATACCGACGTTTAGTTACACCGATTTGGCGGATAG |
| | TGATCGTAACGAAAC |
| 27 | TTCGAAGCACAACCCGTCTTCAACCGCCTATCGAACTCAAACGTT |
| | GGTAGTAGGTGCACG |
| 28 | AGCGACCAATGAACGAGGCGGCACTCTACAGCGTGAAGATCGTCT |
| | GCTTACGATACAATA |
| 29 | AACACCTAGCCGGACGTATCACGGGTAATAAGTCGCGTACTAATC |
| | ACGGTAGGTCCGACA |
| 30 | ACGCATCTTCAGTCGGGCTACCTATTCAGGTACCTATCACGCCGA |
| | GCTAGTAATGGTATG |
| 31 | GTATCTCGCGGTAATACGAGGCCACAACTCGTTCATACGTCCTCG |
| | AATTAGGGCACTTGT |
| 32 | CTCACGCATGGGGTCGCCTAACTAGTTCCGTCAAGATGTGGGTGG |
| | TCCGACGTATCGAAT |
| 33 | GAAAGGGTCTCAACGTATCGTTAGGGGAGCTATTCGATCTCGTGT |
| | ACTATCATTTGTGAG |
| 34 | GCTGGAGGGACTTCGTAACTAGACGTTCGGAAGTTTACTACGGTT |
| | GCAGGCAGATCACCG |
| 35 | TTGCCCACGTTATACACCCCTAGTGTCACCGACGAGTGTGCATGA |
| | TCCGAATTTCTAATA |
| 36 | ATACGTGTAACCACCGCGTTACGACCTACCACGTAAGATCTGTCG |
| | AGTGTCTGTCCTTCG |
| 37 | TTAGGTACGCTAGGAGCCACGCTGACCGACTTAGGGGACTACCGG |
| | CTACCGCTTGAAGGT |
| 38 | CATCCACCGAGGATACCCCTTCATAACGAGGTGTTAATCCGAGAA |
| | ACGTAGCCAAGCGAT |
| 39 | GGATAGGTTATTTCTACGAGCCTAAGCCGAGACCGCTACTTACTC |
| | CCAAACGTAACGTAT |
| 40 | TTACTGAGTGCTACGTAGTTGTATCGTCCGACCTAGACGTGTATC |
| | CAAGTGAATCGTGGC |
| 41 | CTATGAGACCTCTGACCCGGACAATAGTTCGGTCGATATGGAGCC |
| | TAATCTCCGCGATGT |
| 42 | GCAGAACAATACGCGAGTATAGCTTCGTTTCCCCGAGTAATATCC |
| | CGTTCGTCCAGAGGA |
| 43 | ACGCCTTTCCGGAGAGACACGCCCAATAGCACAGGACCGGTTCTA |
| | CTCAACGGCGCACGG |
| 44 | TTGAGGCCTGTACGAGTGACGCAGTACGATGAAGGGCGTTTAAGC |
| | CTAAGGACGGTATCT |
| 45 | ACGAAGTTGTTAGGACGCAACCAGTAGGGTACCGAAGTTACACTC |
| | GATGCCCCTTTGAAA |
| 46 | TCACTGGATGGAGCGATAATTCGGCCTGATAAATCCGTTCGACGG |
| | TCTTATATGAGGGTG |
| 47 | GCCAAGCAGTTACGCTAGCTACATTCGAACGGTTCTCGTATTATC |
| | CGCTTACTGCTGCGT |
| 48 | GAACTAGGCATCGGCTCAAACGACACCAAGCGACTTAGCATACCG |
| | GGAGTAGCATACTGA |
| 49 | GCCGGACAATTTACGTCGTCAAATGGGGACTACTACGTATTAAGG |
| | CTCTGCACGCTAGTA |
| 50 | GTTACGCCCTGTTCACGTTGAGGCTAATCGGCATACACCCCGCGT |
| | TCAAAGAGCAGGTAT |
| 51 | AGATGAGCATTCTTTGTGTTTTGTAGAACGATGCCTGTCCAATGG |
| | AAGTACGCTACAAGC |
| 52 | TGCCAGTTACCGCTAGCAGGTTCGTAATCCCACGGCCTACAATAG |
| | ATACTCCGACGAGCA |
| 53 | GAGCAGTTGGGAGGGCGATGTTCCTCGAAGACTCGCTTTCCGTTA |
| | GTTATATGCGCGTAA |
| 54 | CTAGACGGGGATTCGCATAGGTCTCGGTTCTACGAAATGTACGCG |
| | AGGGTAGGGGTTAGC |
| 55 | TCACATGGAGTCCTATACATGCGGGACGTTCTTATCTAGTCGGCG |
| | TCGGATTGCTTTGTT |
| 56 | GAAGTCACAATTTACGGTGACGCTGACTTAACCGAACTTACAGTA |
| | CCACTCGGAGTAGAT |
| 57 | TAACCTGAGGCATGTCCAACGGTACGACTACGAAGGGTAGAGTCG |
| | CTAAGGACATCGGAG |
| 58 | TTCCCATGCAGCGCAATGAGTAGACGCGAATTAAAATCCGCATAG |
| | GGTTGACGGGGCACC |
| 59 | GTCCGCATAAACCCGGTCTTAATCTCGGCCACGGAAGTCCGATGT |
| | ACGTTATTGGAGAAA |
| 60 | ATCAGCCTAGGGACCTACTTGTGATCAGTCGTAGGTAGTATTGAC |
| | CCGTAATCAAGTACA |
| 61 | AGTACCTAAGGTCTCGATATATGAATCGTACGTACACGCATTTGC |
| | TAGGAGTGCTATGCG |
| 62 | GTTGTGAGAGTACCCATACGTGTGATGTAGGTCCGCGTGTTTAGT |
| | AACCGTGGATAGTAC |
| 63 | TTTTCCTGCTCGTGGACTCTTATAAGTCGCTCCTGACCTTATATC |
| | ACGATCCGATGCTTG |
| 64 | TACCAAGTGTAGCTCCCGAACCTGGACAGTACGGATGAACTACCG |
| | ACGTCTGATGTATAC |
| 65 | GACACGGGAGACTAACCGAAGGGCTCGTCCCACAAAATCGCTAGT |
| | ACGCTGGTCGGTTGG |
| 66 | TGCATCGGTAGAACAGCGGCCTACGTTCTTAGGTAGTATGTCGGG |
| | GTTAGTCCTCACACC |
| 67 | CGGTGTGACGTATTGCATAAGCCTTCGTCGAATGTCGCATACCCC |
| | TCTAGTAATCGTTGT |
| 68 | TCGCACATGGGGCACGTAATTACTCCGTCAGATTACTTTAGGCGC |
| | TTCGGTGTTATGAAC |
| 69 | TCGCACATGGGGCACGTAATTACTCCGTCAGATTACTTTAGGCGC |
| | TTCGGTGTTATGAAC |
| 70 | ATTTGACACGTTCCGGTAGGATTATCTGCGAGTTCACTATGCGAC |
| | GTCAACCTACAACTA |
| 71 | AGCGGAGTCAAAGATAGCAAGCTATCAGCGCCCACGCAGGTACGT |
| | TTGTATTAAGACAGT |
| 72 | AGTTCTAAACCGACACGTATTGTAGTGATATGTGCGAGAGTCGTG |
| | GACAATATTGGTTGC |
| 73 | AACTGATACGCTAAGGGTGTTAGTACGTAATTCGCCTGACGAGAT |
| | AGACCCCTAAAGACG |
| 74 | CGCTCCTTCCCGATATAGGACCACTAGTGAACGCTCCTAAGATGC |
| | ACGTTACGACATTTC |
| 75 | GGTCGAGCACGATTTAGGACACTATCCCGTACTCATCCGTAGTAT |
| | TATGCGATTCCCAAC |
| 76 | CTGATCTATGCAGGGTAATCGTAGAGTACGCTTAGCTCGATAGTA |
| | GCACGTTGGTTCCTG |
| 77 | ATGCATCCTCAATCGACCCGTGTATGATGTACCCGGATGTGAATC |
| | GACACCCTAGTCAAC |
| 78 | GTAAGCTCCAAACTGAACAGGTACAGCGTTGCCCCATCCCAAAAC |
| | CACTCATCCGAAGGA |
| 79 | ATTTAGTCTGCACCTTGGTCAGAGCTGTTCTCGATTTATTACCTG |
| | GAATAGTGATTGGTC |
| 80 | CAACCCTGAGAACCAAGATCATCAAGACAGTCCAAGCTCTTATAC |
| | GGATCATACATACTT |
| 81 | TCGCCAGCACTATCGGTTTGTGCAAATGGGAGTATGAGAATAAGA |
| | CCAGCCCACACCTGC |
| 82 | AGGTTCGGTGAGATAGGGATTTAAGACGAGGAATAGCCGTACTTT |
| | AGCCCTGTGTAATCG |
| 83 | CGTATTCACCTGGTTGGATGCAAACAACACAAACGTCACGGCTTC |
| | CTACCCTTTAACGCA |
| 84 | GGACCATGATCGTTGCACACAAACATTCAAATTATCCCGGAAAGA |
| | AAATCAGCACCTTCG |
| 85 | AGGCCACTCCCCCATATTTGTAGATAGGAGGTCCGTGGTGACTAA |
| | GCAATGGCTTCAGTT |
| 86 | GGTATAGTTTTAGCAGGGAGCGTTGTCAATATCGAGTCAGAACGT |
| | CAGAGACCTGTCAGA |
| 87 | CTATACTTCCACTAACAGACACGTTTAATACGAAACCCAAACAGA |
| | CTCGTGAGTACCCCC |
| 88 | CTTTAAGCTACTTGTTGTAATCCAGCGGAGGACTTATTTGTTGCG |
| | ATAGACTTGGACAGT |
| 89 | TAGCCCTGCGTAGAAACAAAAAGAGGAACTGGCGAGTGCTGCTCA |
| | TTTTAGGTAAAGCAA |
| 90 | CGTGCTATTATTCGTTATTCTTCTTAAATCTAGTGGGCTAGGAGG |
| | TCGCTTTGCACCCGG |
| 91 | TATCCCCCAACGTCTGTAGTTGAAGCCTTGAGGATCGAGCGAAAA |
| | CCCCTGCCTATGGGA |
| 92 | TCTTGTGCTCCCCTGTATCGGTTGGTTCATTAGGGTCATTCTGAT |
| | GTTTCTGAGAGGCTA |
| 93 | CTGTCTAGCGTAGAAGCGCGTTCCTCAACTTTCCAGGTAGGCGAA |
| | ATGAATTTGCATTCG |
| 94 | AGGACACGAGACAGCTTTGCTAATGTGGAGCCAGTACGAACACCC |
| | CAGGAGACAGCCGTA |
| 95 | TTCGTGCGACAGTAAGTGCATTATTGTCTCTCACACCAACACCAT |
| | CTTTGGGCTGCTGTG |
| 96 | ATTAAAGGAGCCTACCACATATTTTCTTGTTCGCTAACTAAATGC |
| | GACTGTCTTTTCTCT |
| 97 | GATGCAGAATGTGGCCGGGGTTCTCATGTTTACCGAAACTTAGGC |
| | ATAGCTTAGAAGTAC |
| 98 | GAATAAACCTGTTCTTTTGTGTGGGGCGGAATCCTACATAACTGC |
| | CACCCTTGTGGTCGT |
| 99 | CTGCTTGTCATTGGTGTTGTTGCTGCTAATTCTAGTTTTGCCCAA |
| | GGCCCAGTCAGTTTC |
| 100 | CGGCGCTCATCCCTCGGCAAACGTGGAGACACTCTCAAGCTGGCA |
| | TTAAATGGATCTTGG |
| 101 | GGGAAAAAGCAGGAATATGGCAAAGGTAAAGCAACTCGTGGCTTA |
| | TGAACTGAACTAACC |
| 102 | TACAGGCTTGAAGATAACGGAGGAGCCCACTTGTTGACGTGCCGA |
| | AGATCATCTAGTTTA |
| 103 | TCTTGCCGTTTGTCAGATTTTGCTGGTTTTTCCATGATCTCTTTT |
| | GCCAGTAATGCTTTA |
| 104 | TCAAACTCTCGTTGACGATGTCCCTACACCTGTATGCGTGCGTGC |
| | TGTCCGTATGTCCAC |
| 105 | GCTGGTTGGAATGCAAGACCGATTGTCGCTGACGGATAATGAGAT |
| | GTAAAAAGTTAAAAC |
| 106 | CTTCGGCAACCCCTTCCACCACCCATTGATATTGAACTTTTCTTT |
| | TTGTAGTTATTACAG |
| 107 | ACACTGGACTTCGGATCTTTGAAATGGCGGTTCTAAATCGCATAA |
| | AGTAGACCACTGGTT |
| 108 | TCTATAGATATGTGACCCCCTTAATTGGATGTGGAAGGAAGAGCT |
| | ATGAGCAACGAAAAG |
| 109 | ACGATAGATAAACCTGAGTTGAACCGTTATTCTGTGGTACAATGG |
| | CAAAGTTTGGACGTA |
| 110 | CATACACAGAAGGGCAAACGCCAGGCAGTGACTCCTTTGGATAAG |
| | CCACATGAGGGCATA |
| 111 | GCAATACGACCCCTCAGAGCCTACAAACATGGGAAAAGTTCATCA |
| | TTATATTTCGCCACC |
| 112 | TAATATAAGTGGACCGAAAACTTTGGAGCTAACCTGACTCAATGA |
| | CGCAAATGGTCAACT |
| 113 | CATCAACAGAACAGCCTACGCATAGTAGAGCAATTAGTAAATCAT |
| | CGCTTAGGTTCACTG |
| 114 | GTTATCTTAATAGCAAGTTCTGCCCTTAGACCACAGAGTAGATCC |
| | GAAAACAGGAACATT |
| 115 | CATGGGGGCTTTGCTGAAGGACCATTCCAAGTTATAGTGTTACTG |
| | ACATCCAGACAAGAT |
| 116 | AACCTAGAACATAACACAAGTTGTTTGTTTCCGCACATACCGTTT |
| | TTCCAAAGTACCACC |
| 117 | CGAGTATTGTATAGGGACACGGCATCGAACACAAGTAAGATAACC |
| | CAGTGATGATAGACA |
| 118 | TGGAGATCTAGGTTGGAATTTCAACAGGTAGTTAGCCGTTATCTG |
| | CTCGCTGTATCTAGG |
| 119 | GCTTGGACATCAACTGCTGTATTCACATAGACTATACGTCATATC |
| | AACAACCCAAAAGCA |
| 120 | GAAAGTTCAAGGGAACCTGAAAACGGCTACAACAACCTATAATGA |
| | TGAGAGTAGAGATAA |
| 121 | TCATTAGGACTCGGAATTTGGAGAAGGGTGAACCGAACCACTTAG |
| | CTGGAGTTTCTATTT |
| 122 | GGGGTACTCACATTTGCTCTGTATTATATTTTTATACGGCAGAAA |
| | TCCTAAGGGCACGGG |
| 123 | TCTGTTGCTTAAATGACGCTCTTGGTGAACCTGTGGTGAAAACCC |
| | GAGTCTCTAAAACGA |
| 124 | TCCTATAGTGTGGTATTACTTCTGCTAGAATCTTGTAGACTTCTT |
| | TTTGGACGGAAGCTC |
| 125 | GGTTCTTGCGATGGGGGCCAGAAATACATTGCTCTTCTCTCGTCG |
| | TTGTGGTAAAACGGA |
| 126 | CTGGTAAACTGACTAATAGTTGGTGGCTAAGGTGCTACTTATTTG |
| | TCCGCTGTATGGTCC |
| 127 | TTGGAATTTCCTTGTGAACCCAGCTTAGATAACAATGATAGGGAT |
| | GTCAGCGGCTAGATA |
| 128 | GTTTTGGCCAGTTGGAACATTATCATCCTATGCTGAAGATTGTAT |
| | GCTGTATCACTATAC |
| 129 | GCATGAACTTTTCTCCCCTTTCTTCTCAGCCTTCTTCTAGGTAGA |
| | CATCCCTGTTATCAC |
| 130 | GCGGAAAATGTAGTTGTCATGCGCTTTAACCAGGTGGGTGTTCTA |
| | GTGCGGTTGTAGTCA |
| 131 | GAGGTCGATTAGTCCATATTTAATACTGTCAGCTTTAGCTTGTCC |
| | CGTGAGTACACCCAC |
| 132 | CAAGTCATTTTCCTGTGCATTCGGGTATTCTCATAATGTGTGGTT |
| | AAAGATCGTTATCTG |
| 133 | AATTGATTCTGAGAACTACTGCCCGGAATTGGTTTTACTCCTAGT |
| | CTGGTATCGCCGTAT |
| 134 | GAAACCGTTCATAGAAAACAGCTACCAAGTTGTGACGATTTGAAT |
| | ACCATCAGTTAAGAA |
| 135 | GTCCCGAAGGAGACATTTGTCGAAGGATCAGGTTTGTGGTATTAG |
| | GCTAACTATATGATG |
| 136 | GAGAGTACTGTCTTGGCTATTGTTATGTGTCGTATATGACCTAGA |
| | GCTAAAGGCAAGCCT |
| 137 | CAACTTCACCTTGAACAGCCTAGAACATAATGTGAGTTTCTTTCC |
| | GATTGGTGGGATTCC |
| 138 | CCATACTATGTCCCCCTCGAACTGATAATCTAAAGGAGGAGTGGG |
| | AGACTGAGTGAGTGA |
| 139 | CGATTTGAGACTCCAGGACATGCAGGCTACCCTTTTATGCCAACC |
| | GGAAGGAAGATACTG |
| 140 | TGGAGTGGAACACACAAAAATAGGTGAATGCTCTGAGCCTTTTAA |
| | CTGGATGTTTTATCA |
| 141 | TCTGCCCTCAGAACCCAACAAGTTAAAAATGGATATGCACTCAAT |
| | AGGATAAATTAGGGG |
| 142 | ACTGATTTACGTGAATGCACATCCGAGTCTGGTTCGTGAGTTAGA |
| | GGTTTGTAGAGGGTG |
| 143 | CCAATCTGTGTAGGTAAGTTCTGATGGGGGTTTTTGGGTGGGATA |
| | CTTTCGTCTCACATT |
| 144 | GGCCTGAATTTGGACATCCTGAAGATCACCCTGATTTCTTTGGGT |
| | ATCAAGCAGCAAAAC |
| 145 | CGATAATGCAGCACCTAATTGCGCGATCAGTCCCATATAAGGGCA |
| | CATAGAAAGTGTACT |
| 146 | TCCAGAACTGAGTGTAATAATGAGGGGCGCAACTGAATTTGTAAC |
| | TGGGGAAGGATTTCA |
| 147 | TTAACTATTGGACTGATGTAGAGACGGTGAGCCCTATGTGTCCTA |
| | ACCTTGGTGATTGTC |
| 148 | ACGGTGTTTCTATCTTCGCTACATAACTTTATACCCACAGACTAA |
| | CAAGCCAGCTTACGC |
| 149 | TCACCGTCACAGACTGGAGCACGTACACACCTAATGATGTCACTG |
| | GGACGACCTTTTGTC |
| 150 | ATACAAGATCCTAAACCATTGATTCGGGTGTACCACACTGGAAAG |
| | AAAAATACTGTGATT |
| 151 | GGCGACGATAAAGGATGATACGAAAAACGGTCTTGGACGGGAGGC |
| | TGTTAGAATTGCGGT |
| 152 | CGGGTAGTGCATTATGTCTTATCACTCTTTGGGTCCTCATGCCAA |
| | TCCTGGAATGGTTTC |
| 153 | CTCAGTGGAAAGAAGATGCCAACCAAAGTTATTAAGTCTAATCAA |
| | TTCGAGCCTATGGGG |
| 154 | TTCTACACGTCACCCACCCCAAGGTTAAGACTCGGTCGGTAAGAT |
| | ACCATGTGGTCACCT |
| 155 | ACAGATGAGTTTGGAGGTCATTAAGAGTAGAAGGTCCTTGTTTTA |
| | CAGTATTCAGCGAGG |
| 156 | CAACAGTAAGCTATCTTAAACTCTTGTACCAGCTACTCTGTACCT |
| | CATCGCAGGTCGATA |
| 157 | TTTCGGTGATAGCAACCACAACGTACTTCTTACACTAATACTCTA |
| | ACAGTGAAGGCTAGG |
| 158 | AGAAGTAATACTGAGCCTGCCCGATTTATTTCCTGAATGAACAAA |
| | AACTGACACCGAGGG |
| 159 | ATGCCTCACGATAAAAAGTTGGGACGGTGGTAATGATCCTAAGGC |
| | TGCATCTACCCATGC |
| 160 | GCCAAGCAATCGGGGATTACAGGTCCACTCGTTCCCGGAATTTGG |
| | GTCACACATAGCATC |
| 161 | CTGTCGCTGTATAAGGAGACCATCGCCAGAAGAAATTATGCAAAT |
| | TGACGAGTAGTGTGA |
| 162 | CAGTCTCTCGATAAGCCGATAAATTCTCCACACAACCAAAAGAGG |
| | TGATTATTCCGGTTT |
| 163 | CCAAGAAATGTTGGGTTGGCCCGGCTTAAAAACGATGTGATATAG |
| | CTCAATAGATCCATT |
| 164 | TGTCCCACCTATGTCCTCAGCAGAAGAGATATGTCCACCCCCTAA |
| | AACAGAGGCATCCAT |
| 165 | TAATTCTTTCGGCCTTAATTCCAAGGTACGTCTCAGCTCCCTTCC |
| | ATACAGCTATACCCT |
| 166 | ATCCTTAAACAAAGACCCAAAACTTAATGGAATAGCAGAGGGATC |
| | ACACTACAAACTTCA |
| 167 | GTTCGGCCAAGAAAAGACGACGGGTACTCAGAACGACGCGAAAAA |
| | CCTTGAATAAAATGC |
| 168 | TTAGCGATGTGTACCATTCAACGTGGGTGAAGGGTTGTTGGAATC |
| | TAGTGGACAGGGGAA |
| 169 | TTGATCATTTGATACCCTGCCGGATGAGAGGATCGAATGCAGCGT |
| | TCTTGCTATGGTCTC |
| 170 | CGCAATACAACCTACCCGAATTTATGAACCCTCCTCCAGACGCCA |
| | ATCCATCGCCCGCAT |
| 171 | CTTTGCAAGAAAATACTTCTGATTAAACAATCCCTTGGCTAACCT |
| | ACCGATTAAGAAACG |
| 172 | AATTCTGGTTAGCTGCTTCATCTGGCACATAAGACTTCACCTCCA |
| | CCACCACGAAGACAA |
| 173 | TTAAATTGTTGGAAAGAGGCTCACCTATACTGGGCAGTTACTCAG |
| | TTCACCATTTTCTTA |
| 174 | ACGGTACCTCAAGCATTTACTTTTCTTTTTAACCAAAATTCACTG |
| | ACGATAACTCACAGG |
| 175 | ACCATCCTGTACTTGTCCTTGTTCCCTTTATCAAACTCATGTTCT |
| | GATGAACGTCTTACT |
| 176 | GCCCCTTGAAACTGTCTTTAAGGCGTCTCACCAAGATTCTGATCC |
| | TTGAGCATCTGAACT |
| 177 | CACGCTTGTAAACCCCAATACGACCTGGACTGATACCAGAGATTG |
| | CGGAATAATATAATC |
| 178 | GAAACTAATGTTGTTTTAATAACCAATAGGGCTTCGGCGGAGGAG |
| | TATGTACTTAGAGTT |
| 179 | TGTACGCAATCGTTTTCTCCGGCAGAAGACTTCGTGGTTGCATTG |
| | AAAGCGGCTATTTAC |
| 180 | GTATACGTGACTCATTAGCTCAATAAATATAAAGCGGTCGTCAGT |
| | GTCATCTTAGTCTAT |
| 181 | CAAATGTGTGAGTTCTGCAACGCCCAGGACAGAGGTTGGGTATGA |
| | TGTGGCCTTTTTGAT |
| 182 | CCATTCTTGATTAGGATGACGAAAGGAATAGAAACAACACCAGAG |
| | GTAATTGCCGAAGAG |
| 183 | GAGGTAAATAAGACATGACCCAGTGGCAGACCGTTTGTTCGGGGT |
| | TCATTAGTGGCTGTG |
| 184 | GAGAAACAGTCGTATCTGACTTACCGAGAAGACTTGTCATAACTG |
| | CCCCTCTCCTCACAA |
| 185 | TTCTTAACCATACCTACTAATCTAAACAGAAACATCGAGATATAC |
| | ATCCGGTGAACCAGA |
| 186 | CATCTGGGGAGGTGTGCTGCGATAGAGAGAGATAATTTAACGAAC |
| | TCAGAAAACACTGGT |
| 187 | TACCCTCGTAGGATGCGTTCTGGCAGCTTTATGGGTTCCAAATGC |
| | TCTTAACAAGCAGAG |
| 188 | AGGTCATGAGGCTACAACTTTCTTGATAGTCCCCCGTACTACAGA |
| | TTTGTCTCTTGCCGG |
| 189 | TATCGGAATAAGACGTTCACTTTCGTACTCAGGCCCCGGTTGAGC |
| | ACACCACTTCTATTT |
| 190 | ACTGACTCTATCGGCTGAGGTCACTGATCTTACTCCACGACCCAA |
| | CAACAGCACGAACAC |
| 191 | GTATTGCCCATTAAGCCTTGATACTGGGACCCTGGGGAACAACAT |
| | TCCATAAAGTTGCAC |
| 192 | TTGCTTCATCGAGTAGTTCGTTGGTCTGCTTGGTTAGGGTTTCTA |
| | GTAGGGAGACTGGAA |
| 193 | CACGCTTCGTCATAGACACTACCATATCGCCATGAATCGGAAATA |
| | AGAATTATCTCCGCC |
| 194 | GGTTGACACTCTTGCTCGTTCACACATTGTCTATTATGTTTTCCA |
| | TATTTTCCTTCACCG |
| 195 | TTCCTGAAGGTGGAGAAAAGAAAGACCTACAGCTCCTAGTCCTAA |
| | TTGTGCCATCGAGAC |
| 196 | CAACTATTGCCGGAAACCTTTTTATAGGAATGGTGGTTGTGACTC |
| | TGATGTCATTATGAT |
| 197 | CGGGAGATCGTGCATGAGCTAATCGTCCTTGGCCTAGCAATACTT |
| | CAAAAGGGCTGAATC |
| 198 | TGGGCAGTTATATCAAACTACTCTCATACAATTCATACCCCAAAC |
| | TCCTGCGTCGGGACG |
| 199 | GTAGGAGTCTCATACCAACTAATCTGCGGATATGGGCAATAGCAT |
| | CAAAACGGGGGTCAA |
| 200 | AGCTCTCGGGGTTGATTAACTAATAAACCTTCCTTTGTGTCCGAT |
| | ACTATAAAGACAGCC |

1. A method of determining a presence of a target agent in a sample comprising:
(a) mixing said sample with capture-associated oligos conjugated to capture moieties specific for said target agent, thereby producing a first mixture comprising reacted capture-associated oligo complexes that are associated with said target agent and unreacted capture-associated oligo complexes that are not associated with said target agent;
(b) contacting said first mixture with immobilized binding partners, wherein said immobilized binding partners facilitate separation of said unreacted capture-associated oligo complexes from said reacted capture-associated oligo complexes to produce a second mixture comprising said unreacted capture-associated oligo complexes and a third mixture comprising said reacted capture-associated oligo complexes;
(c) providing a detection device comprising oligos complementary to said capture-associated oligos, wherein said detection device produces a signal if there is a hybridization event between said capture-associated oligos and said oligos complementary to said capture-associated oligos;
(d) introducing said third mixture to said detection device; and
(e) detecting said signal, wherein said signal is indicative of said presence of said target agent in said sample.

2. The method of claim 1, wherein said capture-associated oligo is a capture-associated universal oligo.

3. The method of claim 1, wherein said capture-associated oligo is conjugated to said capture moiety through a scaffold.

4. The method of claim 3, wherein said scaffold is attached to a plurality of capture moieties.

5. The method of claim 3, wherein said scaffold is attached to a plurality of capture-associated oligos.

6. The method of claim 3, wherein said reacted capture-associated oligo complexes are reacted loaded scaffolds and said unreacted capture-associated oligo complexes are unreacted loaded scaffolds.

7. The method of claim 3, wherein said scaffolds are composed of material selected from the group consisting of: gold, aluminum, copper, platinum, silica, titanium dioxide, carbon nanotubes, polystyrene particles, polyvinyl particles, acrylate and methacrylate particles, glass particles, latex particles, Sepharose beads and other like particles, polymer coated magnetic beads, semiconducting materials, and radio frequency identification substrates.

8. The method of claim 1, wherein neither said capture-associated oligos nor said oligos complementary to said capture-associated oligos hybridize to nucleic acid sequences present in said sample.

9. The method of claim 1, wherein said capture moiety is selected from the group consisting of antibodies, antigens, proteins, ligands, receptors, nucleic acids, toxins, immunoglobulins, metabolites, and hormones.

10. The method of claim 1, wherein said detection device is an electrochemical detection device comprising electrodes and a circuit, and further wherein said oligo complementary to said capture-associated oligo is an electrode-associated universal oligo.

11. The method of claim 10, wherein an electrochemical hybridization detector is used to enhance signal production by said electrochemical detection device.

12. The method of claim 11, wherein said electrochemical hybridization detector is an agent that binds more strongly to double-stranded nucleic acid than single-stranded nucleic acid.

13. The method of claim 12, wherein said electrochemical hybridization detector is an agent that binds differently to double-stranded nucleic acid than it does to single-stranded nucleic acid in such a way that an electrochemical signal produced from a double-stranded nucleic acid bound to said agent is enhanced relative to a single-stranded nucleic acid bound to said agent.

14. The method of claim 11, wherein said electrochemical hybridization detector is selected from the group comprising a minor groove binder, a major groove binder, an intercalator, and a transition metal complex.

15. The method of claim 14, wherein said electrochemical hybridization detector is an intercalating agent, and said intercalating agent is selected from the group consisting of: ethidium, ethidium bromide, acridine, aminoacridine, acridine orange, proflavin, ellipticine, actinomycin D, daunomycin, mitomycin C, Hoechst 33342, Hoechst 33258, aclarubicin, DAPI, Adriamycin, pirarubicin, actinomycin, tris (phenanthroline) zinc salt, tris(phenanthroline) ruthenium salt, tris(phenanthroline) cobalt salt, di(phenanthroline) zinc salt, di(phenanthroline) ruthenium salt, di (phenanthroline) cobalt salt, bipyridine platinum salt, terpyridine platinum salt, phenanthroline platinum salt, tris(bipyridyl) zinc salt, tris(bipyridyl) ruthenium salt, tris (bipyridyl) cobalt salt, di(bipyridyl) zinc salt, di(bipyridyl) ruthenium salt, di(bipyridyl) cobalt salt, and intercalators containing metal ions.

16. The method of claim 11, wherein said electrochemical hybridization detector is conjugated onto said capture-associated oligos.

17. The method of claim 16, wherein said electrochemical hybridization detector is an electroactive marker, and said electroactive marker is selected from the group consisting of: ferrocene derivatives, ferritin derivatives, anthraquinone, silver, silver derivatives, gold, gold derivatives, osmium, osmium derivatives, ruthenium, ruthenium derivatives, cobalt, and cobalt derivatives.

18. The method of claim 16, wherein said electrochemical hybridization detector is a detection moiety, and whereby said method further comprises creating a circular structure by molecular interactions between said capture-associated oligos and said electrode-associated oligos.

19. The method of claim 11, wherein a combination of electrochemical hybridization detectors are used to enhance signal production by said electrochemical detection device.

20. The method of claim 10, wherein said electrochemical detection device comprises a surface on which said electrode-associated oligos are immobilized, wherein said surface is a substrate selected from the group consisting of: fiberglass, Teflon™, ceramics, glass, silicon, mica, plastic, acrylics, polystyrene and copolymers of styrene, polypropylene, polyethylene, polybutylene, polycarbonate, polyurethanes, GETEK, polypropylene oxide, and mixtures thereof.

21. The method of claim 10, wherein said electrochemical detection device comprises a surface that is an oligo chip comprising a plurality of electrodes, where at least one electrode is independently addressable, and where said electrode comprises materials selected from the group consisting of: gold, aluminum, platinum, palladium, rhodium, ruthenium, silicon, titanium, platinum oxide, titanium oxide, tin oxide, indium tin oxide, palladium oxide, silicon oxide, aluminum oxide, molybdenum oxide (Mo₂, O₆), tungsten oxide (WO₃) and ruthenium oxides; carbon, graphite, pyrolytic graphite, carbon fiber, carbon paste, Si, Ge, ZnO, CdS, TiO₂ and GaAs.

22. The method of claim 10, wherein said electrochemical detection device comprises a semi-flexible polymer substrate and a conductive metal layer.

23. The method of claim 22, wherein said conductive metal layer comprises gold.

24. The method of claim 22, wherein said conductive metal layer comprises platinum.

25. The method of claim 22, wherein said semi-flexible polymer substrate is polyethylene terephthalate.

26. The method of claim 10, wherein said electrochemical detection device further comprises a functional element.

27. The method of claim 26, wherein said functional element is a microsensor.

28. The method of claim 26, wherein said functional element is a microheater.

29. The method of claim 10, wherein said electrochemical detection device comprises ninety-six electrodes.

30. The method of claim 10, wherein at least one of said electrodes is a flat planar electrode with addressable locations for synthesis and/or detection.

31. The method of claim 10, wherein at least one of said electrodes is independently addressable, whereby a voltage is applied to each electrode, wherein said voltage is the same voltage, and said electrochemical detection device comprises at least one switch circuit, a decoder circuit, or timing circuit to apply the voltage to the individual electrodes and to receive the output signal from the electrodes.

32. The method of claim 10, wherein at least one of said electrodes is coated with a biocompatible substance selected from the group consisting of: dextran, carboxylmethyldextran, hydrogels, polypeptides, polynucleotides, biocompatible matrices, bio-inert matrices, and mixtures thereof.

33. The method of claim 1, wherein said immobilized binding partners comprise an epitope of said target agent, wherein said epitope specifically reacts with said capture moieties.

34. The method of claim 1, wherein said immobilized binding partners specifically interact with said capture moiety when said capture moiety has not bound said target agent, thereby immobilizing unreacted capture-associated oligo complexes in an immobilized phase and leaving said reacted capture-associated oligo complexes in a solution phase, wherein said solution phase comprises said third mixture.

35. The method of claim 1, further comprising
(a) specific binding of said immobilized binding partners with said capture moieties when said capture moieties have bound said target agent, thereby immobilizing said reacted capture-associated oligo complexes in an immobilized phase and leaving said unreacted capture-associated oligo complexes in a solution phase, wherein said second mixture comprises said solution phase; and
(b) separating said second mixture from said immobilized phase, wherein said third mixture comprises said reacted capture-associated oligo complexes in said immobilized phase.

36. The method claim 35, further comprising liberating said capture-associated oligos from said immobilized phase prior to introducing said third mixture to said detection device in step (d).

37. The method of claim 1, further comprising
(a) specific binding of said immobilized binding partners with said target agent or capture moiety/target agent complex, thereby immobilizing said reacted capture-associated oligo complexes in an immobilized phase and leaving said unreacted capture-associated oligo complexes in a solution phase, wherein said second mixture comprises said solution phase; and
(b) separating said second mixture from said immobilized phase, wherein said third mixture comprises said reacted capture-associated oligo complexes in said immobilized phase.

38. The method claim 37, further comprising liberating said capture-associated oligos from said immobilized phase prior to introducing said third mixture to said detection device in step (d).

39. The method claim 1 wherein said immobilized binding partners are immobilized on a particle.

40. The method of claim 39, wherein said particle is a bead.

41. The method of claim 1, wherein said immobilized binding partners are selected from the group consisting of ligands of said capture moieties, substrates for enzymes, receptors for signaling molecules, antigens specific for said capture moieties, antibodies specific for said capture moieties, and nucleic acids complementary to said capture moieties.

42. The method of claim 1, further comprising adding to the mixture an agent to reduce background signal.

43. The method of claim 42, wherein said agent to reduce background signal is selected from the group consisting of: a single-stranded nuclease, mung bean nuclease, nuclease P1, exonuclease I, exonuclease VII, S1 nuclease and single-stranded DNA binding proteins.

44. The method of claim 1, further comprising adding to the mixture an enzyme to increase strength of said signal.

45. The method of claim 1, further comprising separating said capture-associated oligos from said reacted capture-associated oligo complexes to produce released capture-associated complexes, wherein said third mixture comprises said released capture-associated complexes.

46. The method of claim 45, wherein said separating involves use of a digestive enzyme.

47. The method of claim 46, wherein said digestive enzyme is an endonuclease.

48. The method of claim 45, wherein said separating involves photocleavage.

49. The method of claim 1 wherein said target agent is an antibody and said immobilized binding partners are selected from the group consisting of protein A, protein G, a thiophilic resin, and an anti-class-specific antibody specific for a class of antibodies comprising said target agent.

50. The method of claim 1, further comprising
(i) adding quantifying oligos to said third mixture, wherein each of said quantifying oligos is present in a known concentration, and further wherein said detection device further comprises oligos complementary to said quantifying oligos;
(ii) detecting quantifying signals for each of said quantifying oligos upon hybridization to said oligos complementary to said quantifying oligos; and
(iii) comparing said quantifying signals to the signal detected in (e) to determine an amount of said target agent in said sample.

51. The method of claim 50, wherein at least two of said quantifying oligos are present in different concentrations.

52. The method of claim 50, wherein at least three of said quantifying oligos are present in graduated concentrations.

53. The method of claim 1, wherein there are multiple capture-associated oligos and multiple capture moieties in said first mixture.

54. The method of claim 53, wherein
(a) said sample comprises multiple target agents;
(b) each of said multiple capture moieties is specific for a different one of said multiple target agents in said sample;
(c) each of said multiple capture moieties is conjugated to a different one of said multiple capture-associated oligos;
(d) said third mixture comprises multiple reacted capture-associated oligo complexes; and
(e) said detection device comprises oligonucleotides complementary to each of said multiple capture-associated oligos, thereby allowing simultaneous detection of said multiple target agents in said detection device.

55. The method of claim 54, wherein said multiple target agents include members selected from at least two of the classes consisting of proteins, ligands, receptors, nucleic acids, toxins, immunoglobulins, metabolites, and hormones.

56. The method of claim 1, wherein said detection device does not produce a signal indicating that said target agent is absent from said sample.

57. A method of determining a presence of a target agent in a sample comprising:
(a) mixing said sample with capture-associated oligos conjugated to capture moieties specific for said target agent, thereby producing a first mixture comprising reacted capture-associated oligo complexes that are associated with said target agent and unreacted capture-associated oligo complexes that are not associated with said target agent;
(b) contacting said first mixture with immobilized binding partners, wherein said immobilized binding partners specifically associate with said target agent or capture moiety/target agent complex, thereby immobilizing said reacted capture-associated oligo complexes in an immobilized phase and leaving said unreacted capture-associated oligo complexes in a solution phase;
(c) separating said solution phase from said immobilized phase;
(d) hybridizing an intermediary oligo to said capture-associated oligo, wherein said intermediary oligo comprises a first region complementary to said capture-associated oligo and a second region, and further wherein hybridization of said intermediary oligo to said capture-associated oligo creates a restriction endonuclease recognition site;
(e) adding a restriction endonuclease that cleaves at said restriction endonuclease recognition site, thereby releasing a portion of said intermediary oligo comprising said second region;
(f) providing a detection device comprising oligos complementary to said second region of said intermediary oligo, wherein said detection device produces a signal if there is a hybridization event between said second region and said oligos complementary to said second region;
(d) introducing said portion of said intermediary oligo released in (e) to said detection device; and
(e) detecting said signal, wherein said signal is indicative of said presence of said target agent in said sample.

58. The method of claim 57, wherein said capture moiety is selected from the group consisting of antibodies, proteins, ligands, receptors, nucleic acids, toxins, immunoglobulins, metabolites, and hormones.

59. The method of claim 57, wherein said detection device is an electrochemical detection device comprising electrodes and a circuit.

60. The method of claim 59, wherein an electrochemical hybridization detector is used to enhance signal production by said electrochemical detection device.

61. The method of claim 60, wherein said electrochemical hybridization detector is selected from the group comprising a minor groove binder, a major groove binder, an intercalator, and a transition metal complex.

62. The method of claim 60, wherein said electrochemical hybridization detector is conjugated onto said second region of said intermediary oligo.

63. The method of claim 62, wherein said electrochemical hybridization detector is ferrocene or a derivative thereof.

64. The method of claim 60, wherein a combination of electrochemical hybridization detectors are used to enhance signal production by said electrochemical detection device.

65. The method claim 1, wherein said immobilized binding partners are immobilized on a particle.

66. The method of claim 65, wherein said particle is a bead.

67. The method of claim 57, wherein said immobilized binding partners are selected from the group consisting of ligands of said capture moieties, antibodies specific for said capture moieties, and nucleic acids complementary to said capture moieties.

68. The method of claim 57, wherein said target agent is an antibody and said immobilized binding partners are selected from the group consisting of protein A, protein G, a thiophilic resin, and an anti-class-specific antibody specific for a class of antibodies comprising said target agent.

69. The method of claim 57, wherein said detection device does not produce a signal indicating that said target agent is absent from said sample.

70. A method of determining a presence of a target agent in a sample comprising:
(a) mixing said sample with capture-associated oligos conjugated to capture moieties specific for said target agent, thereby producing a first mixture comprising reacted capture-associated oligo complexes that are associated with said target agent and unreacted capture-associated oligo complexes that are not associated with said target agent;
(b) contacting said first mixture with immobilized binding partners, wherein said immobilized binding partners facilitate separation of said unreacted capture-associated oligo complexes from said reacted capture-associated oligo complexes to produce a second mixture comprising said unreacted capture-associated oligo complexes and a third mixture comprising said reacted capture-associated oligo complexes;
(c) adding an oligonucleotide comprising a polymerase recognition sequence to said third mixture, wherein said reacted capture-associated oligo complexes in said third mixture comprise a complement to said polymerase recognition sequence, thereby producing a double-stranded polymerase recognition site;
(d) adding a polymerase and nucleotides to said third mixture under conditions to allow amplification of said capture-associated oligo to produce amplified oligos;
(e) providing a detection device comprising oligos complementary to said amplified oligos, wherein said detection device produces a signal if there is a hybridization event between said amplified oligos and said oligos complementary to said amplified oligos;
(d) introducing said third mixture to said detection device; and
(e) detecting said signal, wherein said signal is indicative of said presence of said target agent in said sample.

71. The method of claim 70, wherein said polymerase is selected from the group consisting of T3 polymerase, 17 polymerase, SP6 polymerase, T7 polymerase Y639F and T7 polymerase S641A.

72. The method of claim 70, wherein said polymerase is an RNA polymerase that has been modified to allow linear amplification using deoxyribonucleotides.

73. The method of claim 70, wherein said capture-associated oligo is a capture-associated universal oligo.

74. The method of claim 70, wherein said capture moiety is selected from the group consisting of antibodies, proteins, ligands, receptors, nucleic acids, toxins, immunoglobulins, metabolites, and hormones.

75. The method of claim 70, wherein said detection device is an electrochemical detection device comprising electrodes and a circuit, and further wherein said oligo complementary to said capture-associated oligo is an electrode-associated universal oligo.

76. The method of claim 75, wherein an electrochemical hybridization detector is used to enhance signal production by said electrochemical detection device.

77. The method of claim 76, wherein said electrochemical hybridization detector is selected from the group comprising a minor groove binder, a major groove binder, an intercalator, and a transition metal complex.

78. The method of claim 76, wherein said electrochemical hybridization detector is conjugated onto said capture-associated oligos.

79. The method of claim 76, wherein a combination of electrochemical hybridization detectors are used to enhance signal production by said electrochemical detection device.

80. The method of claim 70, wherein said immobilized binding partners comprise an epitope of said target agent, wherein said epitope specifically reacts with said capture moieties.

81. The method of claim 70, wherein said immobilized binding partners specifically interact with said capture moiety when said capture moiety has not bound said target agent, thereby immobilizing unreacted capture-associated oligo complexes in an immobilized phase and leaving said reacted capture-associated oligo complexes in a solution phase, wherein said solution phase comprises said third mixture.

82. The method of claim 70, further comprising
(a) specific binding of said immobilized binding partners with said capture moieties when said capture moieties have bound said target agent, thereby immobilizing said reacted capture-associated oligo complexes in an immobilized phase and leaving said unreacted capture-associated oligo complexes in a solution phase, wherein said second mixture comprises said solution phase; and
(b) separating said second mixture from said immobilized phase, wherein said third mixture comprises said reacted capture-associated oligo complexes in said immobilized phase.

83. The method claim 82, further comprising liberating said capture-associated oligos from said immobilized phase prior to introducing said third mixture to said detection device in step (d).

84. The method of claim 70, further comprising
(a) specific binding of said immobilized binding partners with said target agent or capture moiety/target agent complex, thereby immobilizing said reacted capture-associated oligo complexes in an immobilized phase and leaving said unreacted capture-associated oligo complexes in a solution phase, wherein said second mixture comprises said solution phase; and
(b) separating said second mixture from said immobilized phase, wherein said third mixture comprises said reacted capture-associated oligo complexes in said immobilized phase.

85. The method claim 84, further comprising liberating said capture-associated oligos from said immobilized phase prior to introducing said third mixture to said detection device in step (d).

86. The method claim 70, wherein said immobilized binding partners are immobilized on a particle.

87. The method of claim 86, wherein said particle is a bead.

88. The method of claim 70, wherein said immobilized binding partners are selected from the group consisting of ligands of said capture moieties, antibodies specific for said capture moieties, and nucleic acids complementary to said capture moieties.

89. The method of claim 70, further comprising separating said capture-associated oligos from said reacted capture-associated oligo complexes to produce released capture-associated complexes, wherein said third mixture comprises said released capture-associated complexes.

90. The method of claim 89, wherein said separating involves use of a digestive enzyme.

91. The method of claim 90, wherein said digestive enzyme is an endonuclease.

92. The method of claim 89, wherein said separating involves photocleavage.

93. The method of claim 70, wherein said target agent is an antibody and said immobilized binding partners are selected from the group consisting of protein A, protein G, a thiophilic resin, and an anti-class-specific antibody specific for a class of antibodies comprising said target agent.

94. The method of claim 70, wherein said detection device does not produce a signal indicating that said target agent is absent from said sample.

95. A method for selecting universal oligo pairs comprising:
(a) generating a candidate oligo of length X;
(b) screening said candidate oligo against one or more reference sequences to determine sequence similarity;
(c) discarding said candidate oligo if said sequence similarity is at or above a first threshold;
(d) extending the length of said candidate oligo if said sequence similarity is below said first threshold to produce an extended candidate oligo;
(e) screening said extended candidate oligo against one or more reference sequences to determine sequence similarity;
(e) discarding said extended candidate oligo if said sequence similarity is at or above a second threshold;
(g) extending the length of said extended candidate oligo if said sequence similarity is below said second threshold;
(h) repeating steps (e) through (g) until said extended candidate oligo has a length Y, where Y>25, thereby producing a candidate oligo of length Y;
(i) placing said candidate oligo of length Y in a first group;
(j) repeating steps (a) through (i) until a desired number of candidate oligos of length Y populate said first group, wherein any oligos in or added to said first group are first group oligos;
(k) generating complementary oligos to said first group oligos;
(l) adding said complementary oligos to said first group, thereby creating first group oligo pairs, each of which comprises one of said candidate oligos of length Y and a complementary oligo thereto generated in step (k);
(m) screening each of said first group oligo pairs for sequence similarity against all other of said first group oligo pairs;
(n) discarding each of said first group oligo pairs that has sequence similarity to another of said first group oligo pairs at or above a third threshold; and
(o) adding each of said first group oligo pairs that is not discarded in step (n) to a second group, wherein each of said first group oligo pairs added to said second group is a universal oligo pair, and wherein said second group is a universal oligo set.

96. The method of claim 95, further comprising screening said universal oligo set for additional parameters selected from melting temperature (T/m), existence of duplexes, existence of a GC clamp, existence of hairpins, existence of sequence repeats, dissociation minimum for a 3′ dimer, dissociation minimum for a 3′ terminal stability range, dissociation minimum for a minimum acceptable loop, maximum number of acceptable sequence repeats, and frequency threshold.

97. The method of claim 95, wherein length X is between 8 and 25.

98. The method of claim 97, wherein length X is between 10 and 18.

99. The method of claim 95, wherein one nucleotide is added in at least one of steps (d) and (g).

100. The method of claim 99, wherein each of A, T, G, and C are added in parallel in said extending step.

101. The method of claim 95, wherein said reference sequences include those at ncbi.nlm.nih.gov/BLAST and said first threshold is any value greater than zero.

102. A method for selecting a universal oligo pairs comprising:
(a) generating a candidate oligo of length X;
(b) calculating a GC content of said candidate oligo, wherein if said GC content is outside of a first threshold said candidate oligo is discarded and a new candidate oligo is generated in (a), and wherein if said GC content is inside of said first threshold said candidate oligo is a GC-approved oligo;
(c) screening said GC-approved oligo for a mononucleotide repeat whose length exceeds a second threshold, wherein if said mononucleotide repeat whose length exceeds said second threshold occurs said GC-approved oligo is discarded and a new candidate oligo is generated in (a), and wherein if said mononucleotide repeat whose length exceeds said second threshold does not occur said GC-approved oligo is a repeat-approved oligo;
(d) performing a further screening of said repeat-approved oligo, wherein if said repeat-approved oligo does not pass said further screening such repeat-approved oligo is discarded and a new candidate oligo is generated in (a), and wherein if said repeat-approved oligo passes said further screening said repeat-approved oligo is a further screening-approved oligo;
(e) screening said further screening-approved oligo against one or more reference sequences to determine sequence similarity,
(f) discarding said screening-approved oligo if said sequence similarity is at or above a third threshold;
(g) placing said screening-approved oligo in a first group if said sequence similarity is below said third threshold;
(h) repeating steps (a) through (g) until a desired number of screening-approved oligos populate said first group, wherein any oligos in or added to said first group are first group oligos;
(i) generating complementary oligos to said first group oligos;
(j) adding said complementary oligos to said first group, thereby creating first group oligo pairs, each of which comprises one of said screening-approved oligos and a complementary oligo thereto generated in step (i);
(k) screening each of said first group oligo pairs for sequence similarity against all other of said first group oligo pairs;
(l) discarding each of said first group oligo pairs that has sequence similarity to another of said first group oligo pairs at or above a fourth threshold; and
(m) adding each of said first group oligo pairs that is not discarded in step (l) to a second group, wherein each of said first group oligo pairs added to said second group is a universal oligo pair, and wherein said second group is a universal oligo set.

103. The method of claim 102, wherein said further screening is selected from the group consisting of melting temperature (Tₘ), existence of duplexes, existence of a GC clamp, existence of hairpins, existence of sequence repeats, dissociation minimum for a 3′ dimer, dissociation minimum for a 3′ terminal stability range, dissociation minimum for a minimum acceptable loop, maximum number of acceptable sequence repeats, and frequency threshold.

104. The method of claim 102, wherein length X is between 40 and 100.

105. The method of claim 103, wherein length X is between 50 and 80.

106. The method of claim 103, wherein said reference sequences include those at ncbi.nlmih.gov/BLAST and said third threshold is any value greater than zero

107. A universal oligo comprising a sequence selected from SEQ ID NO 1 through SEQ ID NO 200.

108. A universal oligo set comprising two or more sequences selected from SEQ ID NO 1 through SEQ ID NO 200.

109. A method for using a universal oligo chip to determine a presence of a target agent in a sample by electrochemical detection, said method comprising:
(a) mixing said sample with capture-associated universal oligos conjugated to capture moieties specific for said target agent, thereby producing a first mixture comprising reacted capture-associated universal oligo complexes that are associated with said target agent and unreacted capture-associated universal oligo complexes that are not associated with said target agent;
(b) contacting said first mixture with immobilized binding partners, wherein said immobilized binding partners specifically interact with said unreacted capture-associated universal oligo complexes, thereby immobilizing said unreacted capture-associated universal oligo complexes in an immobilized phase and leaving said reacted capture-associated oligo complexes in a solution phase;
(c) providing a detection device comprising universal oligos complementary to said capture-associated universal oligos, wherein said detection device produces a signal if there is a hybridization event between said capture-associated universal oligos and said universal oligos complementary to said capture-associated universal oligos;
(d) introducing said solution phase to said detection device; and
(e) detecting said signal, wherein said signal is indicative of said presence of said target agent in said sample.

110. A composition comprising
(a) an electrode;
(b) an electrode-associated oligo hybridized to a capture-associated oligo;
(c) a capture moiety conjugated to said capture-associated oligo; and
(d) a target agent bound to said capture moiety.

111. The composition of claim 110, further comprising a binding partner bound to said capture moiety, said target agent, or a complex thereof.

112. The composition of claim 110, wherein said capture moiety is an antibody.

113. The composition of claim 110, further comprising an electrochemical hybridization detector.

114. A biosensor comprising at least one electrode and current or impedance measuring elements, where said measuring elements are enabled to detect changes in current or impedance in response to the presence of a reaction produced when a detection moiety is brought within proximity to said electrode.

115. The biosensor of claim 114, whereby said at least one electrode is in a disposable format, whereby said electrode can be used for a single electrochemical detection experiment of one or more samples and discarded.

116. The biosensor of claim 114, whereby said at least one electrode has a conductive detection surface, and further whereby said at least one electrode comprises a mixed monolayer comprising anchoring groups conjugated to electrode-associated oligos and diluent groups, which serve as insulators on a surface of said electrode.

117. The biosensor of claim 116, whereby said anchoring groups and said diluent groups are chosen to provide approximately uniform distance between enforcing groups to maximize interaction capabilities.

118. The biosensor of claim 116, whereby a plurality of electrode-associated oligos can be located on said biosensor to enable detection of multiple target agents.

119. The biosensor of claim 116, whereby a specific ratio of said anchoring groups and said diluent groups is used for said monolayer on said electrode to enable a uniform monolayer with evenly distributed anchoring group complexes and diluent groups, thereby optimizing the access of the electrode-associated oligo to any capture-associated oligo present in an assay.

120. The biosensor of claim 116, whereby the concentration and type of said anchoring groups and said diluent groups is selected to maximize the ratio of specific current to non-specific current.

121. The biosensor of claim 116, whereby said conductive detection surface is gold.

122. The biosensor of claim 116, whereby said monolayers comprise hexadecanethiolate and have a contact angle with water from 110° to 115°.

123. The biosensor of claim 116, whereby said monolayers are hydrophilic and have a contact angel with water of <10°.

124. A system for determining a presence of a target agent in a sample comprising:
(a) a sample containing said target agent;
(b) capture-associated oligos conjugated to capture moieties specific for said target agent;
(c) electrode-associated oligos that are complementary to said capture-associated oligos or complements thereto;
(d) immobilized binding partners; and
(e) a surface comprising at least one electrode, whereon said electrode associated oligos are attached.

125. The system of claim 124, further comprising one or more electrochemical hybridization detectors.

126. The system of claim 124, further comprising an electrochemical detection device.

127. A method of doing business wherein the system of claim 124 is queried remotely to collect information on results.

128. A diagnostic tool for detecting a target agent in a sample, comprising:
(a) a capture moiety which binds preferentially to a target agent;
(b) a first nucleic acid associated with said capture moiety, and
(c) a recognition sequence in said first nucleic acid for linear amplification of said first nucleic acid, wherein said first nucleic acid comprises a sequence substantially the same as a sequence of a second nucleic acid associated with an electrode.

129. The diagnostic tool of claim 128, wherein said capture moiety is an antibody.

130. The diagnostic tool of claim 128, wherein said capture moiety is a ligand.

131. The diagnostic tool of claim 128, wherein said recognition sequence is for an RNA phage polymerase.

132. The diagnostic tool of claim 128, wherein said recognition sequence allows amplification using asymmetric polymerase chain reaction.

133. The diagnostic tool of claim 128, wherein said first nucleic acid further comprises a restriction endonuclease recognition sequence.

134. The diagnostic tool of claim 128, wherein said first nucleic acid further comprises a polymerase recognition sequence.

135. A kit for use in detecting the presence of a target agent in a sample, said kit comprising:
a biosensor comprising at least one disposable electrode and current or impedance measuring elements, wherein said at least one disposable electrode comprises a conductive detection surface, and a mixed monolayer comprising anchoring groups conjugated to electrode-associated oligos and diluent groups;
a first single-stranded nucleic acid molecule that is complementary to a second single-stranded nucleic acid molecule, where said first single-stranded nucleic acid molecule is immobilized on said biosensor, and further where said second single-stranded nucleic acid molecule is conjugated to a capture moiety specific for said target agent;
at least one immobilized binding partner; and
at least one container.

136. A kit for use in detecting the presence of a target agent in a sample, said kit comprising:
a biosensor comprising at least one disposable electrode and current or impedance measuring elements, wherein said at least one disposable electrode comprises a conductive detection surface, and a mixed monolayer comprising anchoring groups conjugated to electrode-associated oligos and diluent groups;
a first single-stranded nucleic acid molecule that is complementary to a second single-stranded nucleic acid molecule, where said first single-stranded nucleic acid molecule is immobilized on said biosensor, and further where said second single-stranded nucleic acid molecule is conjugated to a capture moiety specific for said target agent;
at least one immobilized binding partner; and
at least one container.

137. A method of electrochemically detecting and quantifying a presence of a target agent of interest in a sample comprising:
(a) mixing:
(i) said sample with at least one loaded scaffold comprising a capture-associated universal oligo, a capture moiety specific for said target agent of interest and a scaffold; and
(ii) a sample suspected of containing said target agent of interest, thereby producing a mixture comprising reacted loaded scaffolds and unreacted loaded scaffolds;
(b) contacting the mixture of step (a)(ii) with immobilized binding partners to said capture moieties of said loaded scaffolds so as to allow any of said unreacted loaded scaffolds to bind with said immobilized binding partners resulting in an immobilized phase and a solution phase;
(c) separating the immobilized phase and solution phase;
(d) providing an electrochemical detection device comprising electrodes, electrode-associated universal oligos, and a circuit, wherein said detection device produces a signal if there is a hybridization event between said electrode-associated universal oligos and other nucleic acid molecules;
(e) introducing said solution phase from step (c) to the electrochemical detection device from step (d); and
(h) detecting an electrochemical signal generated by capture-associated universal oligos from said reacted loaded scaffolds and electrode-associated universal oligos.

138. A method of electrochemically detecting and quantify a presence of a target agent of interest in a sample comprising:
(a) mixing:
(i) said sample with a loaded scaffold comprising a capture-associated universal oligo, a capture moiety specific for said target agent of interest and a scaffold; and
(ii) a sample suspected of containing said target agent of interest, thereby producing a mixture comprising reacted loaded scaffolds and unreacted loaded scaffolds;
(b) contacting the mixture of step (a) with immobilized binding partners to said target agents or to capture moiety/target agent complexes so as to allow any of said reacted loaded scaffolds to bind with said immobilized binding partners resulting in an immobilized phase and a solution phase;
(c) separating the immobilized phase and solution phase;
(d) providing an electrochemical detection device comprising electrodes, electrode-associated universal oligos, and a circuit, wherein said detection device produces a signal if there is a hybridization event between said electrode-associated universal oligos and other nucleic acid molecules;
(e) liberating said capture-associated universal oligos into a second solution phase from said immobilized phase;
(f) introducing said solution phase from step (e) to the electrochemical detection device from step (d); and
(h) detecting an electrochemical signal generated by capture-associated universal oligos and electrode-associated universal oligos.

139. A method of doing business, said method comprising use of a electrical signal to determine appropriate medical intervention for a patient, said method comprising:
(a) obtaining a sample from a patient whereby a target agent may be present in said sample:
(b) mixing said sample with capture-associated oligos conjugated moieties specific for said target agent, thereby producing a first mixture comprising reacted capture-associated oligo complexes that are associated with said target agent and unreacted capture-associated oligo complexes that are not associated with said target agent;
(c) contacting said first mixture with immobilized binding partners, wherein said immobilized binding partners facilitate separation of said unreacted capture-associated oligo complexes from said reacted capture-associated oligo complexes to produce a second mixture comprising said reacted capture-associated oligo complexes;
(d) providing a detection device comprising oligos complementary to said capture-associated oligos, wherein said detection device produces a signal if there is a hybridization event between said capture-associated universal oligos and said oligos;
(e) introducing said third mixture to said detection device;
(f) detecting said signal, wherein said signal is indicative of said presence of said target agent in said sample; and
(g) determining the appropriate medical intervention for the patient based upon the presence or absence of said target agent in said sample.

140. An electrical signal used to determine appropriate medical intervention for a patient, whereby said electrical signal is indicative of the concentration of a target agent in a sample taken from said patient; where the concentration of said target agent is calculated by a software algorithm that correlates the magnitude of said electrical signal of the magnitude of a second electrical signal from a pre-determined set of quantifying target agent; and where said electrical signal is dependent upon the presence of an electrode, an electrode-associated oligo hybridized to a capture-associated oligo, a capture moiety conjugated to said capture-associated oligo and a target agent bound to said capture moiety.

141. A detection device comprising:
one or more electrochemical chips,
electrode-associated oligos complementary to capture-associated oligos,
a signaling generator for producing a signal upon occurrence of a hybridization event between said capture-associated oligos and said electrode-associated oligos, a receiver for receiving said signal a processor for processing said signal, and a display for displaying the results of said processing.

142. A method of determining a presence of a target nucleic acid in a sample wherein said target nucleic acid is not contacted with a detection device, comprising:
(a) providing hybrid oligos, each of which comprises 1) a region complementary to a target nucleic acid and ii) a capture-associated oligo;
(b) mixing said sample with said hybrid oligos, thereby producing a first mixture comprising reacted hybrid oligo complexes that are associated with said target nucleic acid and unreacted hybrid oligo complexes that are not associated with said target nucleic acid;
(c) contacting said first mixture with a polymerase and nucleotides under conditions to facilitate creation of double-stranded target nucleic acid on said reacted hybrid oligo complexes to create a second mixture;
(d) exposing said second mixture to a hydroxyapatite matrix, wherein said hydroxyapatite matrix facilitates separation of said double-stranded target nucleic acid on said reacted hybrid oligo complexes from single-stranded nucleic acid species in said second mixture;
(e) removing said single-stranded nucleic acid species from said hydroxyapatite matrix and discarding;
(f) removing said double-stranded target nucleic acid on said reacted hybrid oligo complexes from said hydroxyapatite matrix;
(g) separating said capture-associated oligos from said reacted hybrid oligo complexes removed from said hydroxyapatite matrix in step (f);
(h) providing said detection device comprising oligos complementary to said capture-associated oligos, wherein said detection device produces a signal if there is a hybridization event between said capture-associated oligos and said oligos complementary to said capture-associated oligos;
(i) introducing said capture-associated oligos to said detection device; and
(j) detecting said signal, wherein said signal is indicative of said presence of said target nucleic acid in said sample.


SEQUENCE LISTING (200 sequences)

SEQ ID NO: 1 - DNA, 60 bp, Artificial
  Universal oligo
aaggtacgaacgactaacgggtcctaacgggacgctactagggcgactaatcagatccct

SEQ ID NO: 2 - DNA, 60 bp, Artificial
  Universal oligo
accgagtaatgagacgtgtaccctagaccggaggcaccttacgaacagtggtcagatccc

SEQ ID NO: 3 - DNA, 60 bp, Artificial
  Universal oligo
acctaacgtcgatgcgtctcgtaacagttcgcgtcctagtgcaaccgcgtgggtcagatc

SEQ ID NO: 4 - DNA, 60 bp, Artificial
  Universal oligo
acgatactcccgacttacagttcgctccgtggattacgaccgacccaatttatcagatcc

SEQ ID NO: 5 - DNA, 60 bp, Artificial
  Universal oligo
acgtcggtctactcaactaacgtagcgtatgtcggattcgcgttgtggaaattcagatcc

SEQ ID NO: 6 - DNA, 60 bp, Artificial
  Universal oligo
actcacgcgattagcggttcgcaatactaggcgagcgttacatatccgagcgatctcaga

SEQ ID NO: 7 - DNA, 60 bp, Artificial
  Universal oligo
agcccgatagtccgacactcctacttcgcttagccgtatagtggcctgctacggaatcag

SEQ ID NO: 8 - DNA, 60 bp, Artificial
  Universal oligo
atcgctacagggtcgcgctaatacaggtcgcatatcggagttcaccgcaatagtcagatc

SEQ ID NO: 9 - DNA, 60 bp, Artificial
  Universal oligo
atcggccctacgttacggactaactacgcggtccctacgattcgcggactagacaatcag

SEQ ID NO: 10 - DNA, 59 bp, Artificial
  Universal oligo
tgagtgcgagacgagggagggtatcgacaccgctcaatacattcgtgagaatcgatcag

SEQ ID NO: 11 - DNA, 60 bp, Artificial
  Universal oligo
ccgaaaccacaattcgtcggtccgtattgatccggctcgacacgtatgcctgtgcgacta

SEQ ID NO: 12 - DNA, 60 bp, Artificial
  Universal oligo
atcctccgaactaatccgatatttctcgcaacggttagtcgatcccactacgccgcttat

SEQ ID NO: 13 - DNA, 60 bp, Artificial
  Universal oligo
tatgttgtcgaccatggctatcgtacgggattgcctcggattatcgtctgacgggttaat

SEQ ID NO: 14 - DNA, 60 bp, Artificial
  Universal oligo
cagaccacataatcgtacttgcaacggaacaaatttcgacgccctaagttggttgtcact

SEQ ID NO: 15 - DNA, 60 bp, Artificial
  Universal oligo
attcgagacgtaaacggatacgatccctgtattcgtaacgttgtaacgcaacagacatca

SEQ ID NO: 16 - DNA, 60 bp, Artificial
  Universal oligo
tggaacgaacgcttacgtgtattgtggagcgtatcttaggccaccggattctcacacttc

SEQ ID NO: 17 - DNA, 60 bp, Artificial
  Universal oligo
ggagtcaaccttgtccgtccattctaaaccgttgtgcgtccgtcccgattagaccaaccc

SEQ ID NO: 18 - DNA, 60 bp, Artificial
  Universal oligo
cacgacgacttgacggcagtgcgtaccggcgactgttacggtgggtcgaacaagtcgcta

SEQ ID NO: 19 - DNA, 60 bp, Artificial
  Universal oligo
cactccgcacccttacgcaccggactaaaggtattcgcgggaaactgcataccgcccgtt

SEQ ID NO: 20 - DNA, 60 bp, Artificial
  Universal oligo
gcgaatgcgtctaataccgctaccttcgagcgcaggttgaccgtccgtactatcagtcaa

SEQ ID NO: 21 - DNA, 60 bp, Artificial
  Universal oligo
tcttggcgttaaccgttagatcgacttcccgtattgagaacggaacggcatgaacggagg

SEQ ID NO: 22 - DNA, 60 bp, Artificial
  Universal oligo
taacctcaaggtctacgatagcggataagtcgtgaagagcgtgaacgatattgcaatcac

SEQ ID NO: 23 - DNA, 60 bp, Artificial
  Universal oligo
gagcgtaaattctacgcgatagttcgtatctatggcttcgaaaccgtctgcatcgatgac

SEQ ID NO: 24 - DNA, 60 bp, Artificial
  Universal oligo
ctgggaattgtagcgccagtggatatagcccgtcaatgccgcgtaagtctacgtacagcc

SEQ ID NO: 25 - DNA, 60 bp, Artificial
  Universal oligo
tcactacgtaacctcgacaaaccgtattcccgtggcgcaagtctggatcggcgacctcgg

SEQ ID NO: 26 - DNA, 60 bp, Artificial sequence
  Universal oligo
tcccgaggttataccgacgtttagttacaccgatttggcggatagtgatcgttacgaaac

SEQ ID NO: 27 - DNA, 60 bp, Artificial
  Universal oligo
ttcgaagcacaacccgtcttcaaccgcctatcgaactcaaacgttggtagtaggtgcacg

SEQ ID NO: 28 - DNA, 60 bp, Artificial
  Universal oligo
agcgaccaatgaacgaggcggcactctacagcgtgaagatcgtctgcttacgatacaata

SEQ ID NO: 29 - DNA, 60 bp, Artificial
  Universal oligo
aacacctagccggacgtatcacgggtaataagtcgcgtactaatcacggtaggtccgaca

SEQ ID NO: 30 - DNA, 60 bp, Artificial
  Universal oligo
acgcatcttcagtcgggctacctattcaggtacctatcacgccgagctagtaatggtatg

SEQ ID NO: 31 - DNA, 60 bp, Artificial
  Universal oligo
gtatctcgcggtaatacgaggccacaactcgttcatacgtcctcgaattagggcacttgt

SEQ ID NO: 32 - DNA, 60 bp, Artificial
  Universal oligo
ctcacgcatggggtcgcctaactagttccgtcaagatgtgggtggtccgacgtatcgaat

SEQ ID NO: 33 - DNA, 60 bp, Artificial
  Universal oligo
gaaagggtctcaacgtatcgttaggggagctattcgatctcgtgtactatcatttgtgag

SEQ ID NO: 34 - DNA, 60 bp, Artificial
  Universal oligo
gctggagggacttcgtaactagacgttcggaagtttactacggttgcaggcagatcaccg

SEQ ID NO: 35 - DNA, 60 bp, Artificial
  Universal oligo
ttgcccacgttatacacccctagtgtcaccgacgagtgtgcatgatccgaatttctaata

SEQ ID NO: 36 - DNA, 60 bp, Artificial
  Universal oligo
atacgtgtaaccaccgcgttacgacctaccacgtaagatctgtcgagtgtctgtccttcg

SEQ ID NO: 37 - DNA, 60 bp, Artificial
  Universal oligo
ttaggtacgctaggagccacgctgaccgacttaggggactaccggctaccgcttgaaggt

SEQ ID NO: 38 - DNA, 60 bp, Artificial
  Universal oligo
catccaccgaggataccccttcataacgaggtgttaatccgagaaacgtagccaagcgat

SEQ ID NO: 39 - DNA, 60 bp, Artificial
  Universal oligo
ggataggttatttctacgagcctaagccgagaccgctacttactcccaaacgtaacgtat

SEQ ID NO: 40 - DNA, 60 bp, Artificial
  Universal oligo
ttactgagtgctacgtagttgtatcgtccgacctagacgtgtatccaagtgaatcgtggc

SEQ ID NO: 41 - DNA, 60 bp, Artificial
  Universal oligo
ctatgagacctctgacccggacaatagttcggtcgatatggagcctaatctccgcgatgt

SEQ ID NO: 42 - DNA, 60 bp, Artificial
  Universal oligo
gcagaacaatacgcgagtatagcttcgtttccccgagtaatatcccgttcgtccagagga

SEQ ID NO: 43 - DNA, 60 bp, Artificial
  Universal oligo
acgcctttccggagagacacgcccaatagcacaggaccggttctactcaacggcgcacgg

SEQ ID NO: 44 - DNA, 60 bp, Artificial
  Universal oligo
ttgaggcctgtacgagtgacgcagtacgatgaagggcgtttaagcctaaggacggtatct

SEQ ID NO: 45 - DNA, 60 bp, Artificial
  Universal oligo
acgaagttgttaggacgcaaccagtagggtaccgaagttacactcgatgcccctttgaaa

SEQ ID NO: 46 - DNA, 60 bp, Artificial
  Universal oligo
tcactggatggagcgataattcggcctgataaatccgttcgacggtcttatatgagggtg

SEQ ID NO: 47 - DNA, 60 bp, Artificial
  Universal oligo
gccaagcagttacgctagctacattcgaacggttctcgtattatccgcttactgctgcgt

SEQ ID NO: 48 - DNA, 60 bp, Artificial
  Universal oligo
gaactaggcatcggctcaaacgacaccaagcgacttagcataccgggagtagcatactga

SEQ ID NO: 49 - DNA, 60 bp, Artificial
  Universal oligo
gccggacaatttacgtcgtcaaatggggactactacgtattaaggctctgcacgctagta

SEQ ID NO: 50 - DNA, 60 bp, Artificial
  Universal oligo
gttacgccctgttcacgttgaggctaatcggcatacaccccgcgttcaaagagcaggtat

SEQ ID NO: 51 - DNA, 60 bp, Artificial
  Universal oligo
agatgagcattctttgtgttttgtagaacgatgcctgtccaatggaagtacgctacaagc

SEQ ID NO: 52 - DNA, 60 bp, Artificial
  Universal oligo
tgccagttaccgctagcaggttcgtaatcccacggcctacaatagatactccgacgagca

SEQ ID NO: 53 - DNA, 60 bp, Artificial
  Universal oligo
gagcagttgggagggcgatgttcctcgaagactcgctttccgttagttatatgcgcgtaa

SEQ ID NO: 54 - DNA, 60 bp, Artificial
  Universal oligo
ctagacggggattcgcataggtctcggttctacgaaatgtacgcgagggtaggggttagc

SEQ ID NO: 55 - DNA, 60 bp, Artificial
  Universal oligo
tcacatggagtcctatacatgcgggacgttcttatctagtcggcgtcggattgctttgtt

SEQ ID NO: 56 - DNA, 60 bp, Artificial
  Universal oligo
gaagtcacaatttacggtgacgctgacttaaccgaacttacagtaccactcggagtagat

SEQ ID NO: 57 - DNA, 60 bp, Artificial
  Universal oligo
taacctgaggcatgtccaacggtacgactacgaagggtagagtcgctaaggacatcggag

SEQ ID NO: 58 - DNA, 60 bp, Artificial
  Universal oligo
ttcccatgcagcgcaatgagtagacgcgaattaaaatccgcatagggttgacggggcacc

SEQ ID NO: 59 - DNA, 60 bp, Artificial
  Universal oligo
gtccgcataaacccggtcttaatctcggccacggaagtccgatgtacgttattggagaaa

SEQ ID NO: 60 - DNA, 60 bp, Artificial
  Universal oligo
atcagcctagggacctacttgtgatcagtcgtaggtagtattgacccgtaatcaagtaca

SEQ ID NO: 61 - DNA, 60 bp, Artificial
  Universal oligo
agtacctaaggtctcgatatatgaatcgtacgtacacgcatttgctaggagtgctatgcg

SEQ ID NO: 62 - DNA, 60 bp, Artificial
  Universal oligo
gttgtgagagtacccatacgtgtgatgtaggtccgcgtgtttagtaaccgtggatagtac

SEQ ID NO: 63 - DNA, 60 bp, Artificial
  Universal oligo
ttttcctgctcgtggactcttataagtcgctcctgaccttatatcacgatccgatgcttg

SEQ ID NO: 64 - DNA, 60 bp, Artificial
  Universal oligo
taccaagtgtagctcccgaacctggacagtacggatgaactaccgacgtctgatgtatac

SEQ ID NO: 65 - DNA, 60 bp, Artificial
  Universal oligo
gacacgggagactaaccgaagggctcgtcccacaaaatcgctagtacgctggtcggttgg

SEQ ID NO: 66 - DNA, 60 bp, Artificial
  Universal oligo
tgcatcggtagaacagcggcctacgttcttaggtagtatgtcggggttagtcctcacacc

SEQ ID NO: 67 - DNA, 60 bp, Artificial
  Universal oligo
cggtgtgacgtattgcataagccttcgtcgaatgtcgcatacccctctagtaatcgttgt

SEQ ID NO: 68 - DNA, 60 bp, Artificial
  Universal oligo
ccagacttttggggccgtaccatcggtaagcgcacaatagacatcgatacggaatgctat

SEQ ID NO: 69 - DNA, 60 bp, Artificial
  Universal oligo
tcgcacatggggcacgtaattactccgtcagattactttaggcgcttcggtgttatgaac

SEQ ID NO: 70 - DNA, 60 bp, Artificial
  Universal oligo
atttgacacgttccggtaggattatctgcgagttcactatgcgacgtcaacctacaacta

SEQ ID NO: 71 - DNA, 60 bp, Artificial
  Universal oligo
agcggagtcaaagatagcaagctatcagcgcccacgcaggtacgtttgtattaagacagt

SEQ ID NO: 72 - DNA, 60 bp, Artificial
  Universal oligo
agttctaaaccgacacgtattgtagtgatatgtgcgagagtcgtggacaatattggttgc

SEQ ID NO: 73 - DNA, 60 bp, Artificial
  Universal oligo
aactgatacgctaagggtgttagtacgtaattcgcctgacgagatagacccctaaagacg

SEQ ID NO: 74 - DNA, 60 bp, Artificial
  Universal oligo
cgctccttcccgatataggaccactagtgaacgctcctaagatgcacgttacgacatttc

SEQ ID NO: 75 - DNA, 60 bp, Artificial
  Universal oligo
ggtcgagcacgatttaggacactatcccgtactcatccgtagtattatgcgattcccaac

SEQ ID NO: 76 - DNA, 60 bp, Artificial
  Universal oligo
ctgatctatgcagggtaatcgtagagtacgcttagctcgatagtagcacgttggttcctg

SEQ ID NO: 77 - DNA, 60 bp, Artificial
  Universal oligo
atgcatcctcaatcgacccgtgtatgatgtaccccgatgtgaatcgacaccctagtcaac

SEQ ID NO: 78 - DNA, 60 bp, Artificial
  Universal oligo
gtaagctccaaactgaacaggtacagcgttgccccatcccaaaaccactcatccgaagga

SEQ ID NO: 79 - DNA, 60 bp, Artificial
  Universal oligo
atttagtctgcaccttggtcagagctgttctcgatttattacctggaatagtgattggtc

SEQ ID NO: 80 - DNA, 60 bp, Artificial
  Universal oligo
caaccctgagaaccaagatcatcaagacagtccaagctcttatacggatcatacatactt

SEQ ID NO: 81 - DNA, 60 bp, Artificial
  Universal oligo
tcgccagcactatcggtttgtgcaaatgggagtatgagaataagaccagcccacacctgc

SEQ ID NO: 82 - DNA, 60 bp, Artificial
  Universal oligo
aggttcggtgagatagggatttaagacgaggaatagccgtactttagccctgtgtaatcg

SEQ ID NO: 83 - DNA, 60 bp, Artificial
  Universal oligo
cgtattcacctggttggatgcaaacaacacaaacgtcacggcttcctaccctttaacgca

SEQ ID NO: 84 - DNA, 60 bp, Artificial
  Universal oligo
ggaccatgatcgttgcacacaaacattcaaattatcccggaaagaaaatcagcaccttcg

SEQ ID NO: 85 - DNA, 60 bp, Artificial
  Universal oligo
aggccactcccccatatttgtagataggaggtccgtggtgactaagcaatggcttcagtt

SEQ ID NO: 86 - DNA, 60 bp, Artificial
  Universal oligo
ggtatagttttagcagggagcgttgtcaatatcgagtcagaacgtcagagacctgtcaga

SEQ ID NO: 87 - DNA, 60 bp, Artificial
  Universal oligo
ctatacttccactaacagacacgtttaatacgaaacccaaacagactcctgagtaccccc

SEQ ID NO: 88 - DNA, 60 bp, Artificial
  Universal oligo
ctttaagctacttgttgtaatccagcggaggacttatttgttgcgatagacttggacagt

SEQ ID NO: 89 - DNA, 60 bp, Artificial
  Universal oligo
tagccctgcgtagaaacaaaaagaggaactggcgagtgctgctcattttaggtaaagcaa

SEQ ID NO: 90 - DNA, 60 bp, Artificial
  Universal oligo
cgtgctattattcgttattcttcttaaatctagtgggctaggaggtcgctttgcacccgg

SEQ ID NO: 91 - DNA, 60 bp, Artificial
  Universal oligo
tatcccccaacgtctgtagttgaagccttgaggatcgagcgaaaacccctgcctatggga

SEQ ID NO: 92 - DNA, 60 bp, Artificial
  Universal oligo
tcttgtgctcccctgtatcggttggttcattagggtcattctgatgtttctgagaggcta

SEQ ID NO: 93 - DNA, 60 bp, Artificial
  Universal oligo
ctgtctagcgtagaagcgcgttcctcaactttccaggtaggcgaaatgaatttgcattcg

SEQ ID NO: 94 - DNA, 60 bp, Artificial
  Universal oligo
aggacacgagacagctttgctaatgtggagccagtacgaacaccccaggagacagccgta

SEQ ID NO: 95 - DNA, 60 bp, Artificial
  Universal oligo
ttcgtgcgacagtaagtgcattattgtctctcacaccaacaccatctttgggctgctgtg

SEQ ID NO: 96 - DNA, 60 bp, Artificial
  Universal oligo
attaaaggagcctaccacatattttcttgttcgctaactaaatgcgactgtcttttctct

SEQ ID NO: 97 - DNA, 60 bp, Artificial
  Universal oligo
gatgcagaatgtggccggggttctcatgtttaccgaaacttaggcatagcttagaagtac

SEQ ID NO: 98 - DNA, 60 bp, Artificial
  Universal oligo
gaataaacctgttcttttgtgtggggcggaatcctacataactgccacccttgtggtcgt

SEQ ID NO: 99 - DNA, 60 bp, Artificial
  Universal oligo
ctgcttgtcattggtgttgttgctgctaattctagttttgcccaaggcccagtcagtttc

SEQ ID NO: 100 - DNA, 60 bp, Artificial
  Universal oligo
cggcgctcatccctcggcaaacgtggagacactctcaagctggcattaaatggatcttgg

SEQ ID NO: 101 - DNA, 60 bp, Artificial
  Universal oligo
gggaaaaagcaggaatatgccaaaggtaaagcaactcgtgccttatgaactgaactaacc

SEQ ID NO: 102 - DNA, 60 bp, Artificial
  Universal oligo
tacaggcttgaagataacggaggagcccacttgttgacgtgccgaagatcatctagttta

SEQ ID NO: 103 - DNA, 60 bp, Artificial
  Universal oligo
tcttgccgtttgtcagattttgctggtttttccatgatctcttttgccagtaatgcttta

SEQ ID NO: 104 - DNA, 60 bp, Artificial
  Universal oligo
tcaaactctcgttgacgatgtccctacacctgtatgcgtgcgtgctgtccgtatgtccac

SEQ ID NO: 105 - DNA, 60 bp, Artificial
  Universal oligo
gctggttggaatgcaagaccgattgtcgctgacggataatgagatgtaaaaagttaaaac

SEQ ID NO: 106 - DNA, 60 bp, Artificial
  Universal oligo
cttcggcaaccccttccaccacccattgatattgaacttttctttttgtagttattacag

SEQ ID NO: 107 - DNA, 60 bp, Artificial
  Universal oligo
acactggacttcggatctttgaaatggcggttctaaatcgcataaagtagaccactggtt

SEQ ID NO: 108 - DNA, 60 bp, Artificial
  Universal oligo
tctatagatatgtgacccccttaattggatgtggaaggaagagctatgagcaacgaaaag

SEQ ID NO: 109 - DNA, 60 bp, Artificial
  Universal oligo
acgatagataaacctgagttgaaccgttattctgtggtacaatggcaaagtttggacgta

SEQ ID NO: 110 - DNA, 60 bp, Artificial
  Universal oligo
catacacagaagggcaaacgccaggcagtgactcctttggataagccacatgagggcata

SEQ ID NO: 111 - DNA, 60 bp, Artificial
  Universal oligo
gcaatacgacccctcagagcctacaaacatgggaaaagttcatcattatatttcgccacc

SEQ ID NO: 112 - DNA, 60 bp, Artificial
  Universal oligo
taatataagtggaccgaaaactttggagctaacctgactcaatgacgcaaatggtcaact

SEQ ID NO: 113 - DNA, 60 bp, Artificial
  Universal oligo
catcaacagaacagcctacgcatagtagagcaattagtaaatcatcgcttaggttcactg

SEQ ID NO: 114 - DNA, 60 bp, Artificial
  Universal oligo
gttatcttaatagcaagttctgcccttagaccacagagtagatccgaaaacaggaacatt

SEQ ID NO: 115 - DNA, 60 bp, Artificial
  Universal oligo
catgggggctttgctgaaggaccattccaagttatagtgttactgacatccagacaagat

SEQ ID NO: 116 - DNA, 60 bp, Artificial
  Universal oligo
aacctagaacataacacaagttgtttgtttccgcacataccgtttttccaaagtaccacc

SEQ ID NO: 117 - DNA, 60 bp, Artificial
  Universal oligo
cgagtattgtatagggacacggcatcgaacacaagtaagataacccagtgatgatagaca

SEQ ID NO: 118 - DNA, 60 bp, Artificial
  Universal oligo
tggagatctaggttggaatttcaacaggtagttagccgttatctgctcgctgtatctagg

SEQ ID NO: 119 - DNA, 60 bp, Artificial
  Universal oligo
gcttggacatcaactgctgtattcacatagactatacgtcatatcaacaacccaaaagca

SEQ ID NO: 120 - DNA, 60 bp, Artificial
  Universal oligo
gaaagttcaagggaacctgaaaacggctacaacaacctataatgatgagagtagagataa

SEQ ID NO: 121 - DNA, 60 bp, Artificial
  Universal oligo
tcattaggactcggaatttggagaagggtgaaccgaaccacttagctggagtttctattt

SEQ ID NO: 122 - DNA, 60 bp, Artificial
  Universal oligo
ggggtactcacatttgctctgtattatatttttatacggcagaaatcctaagggcacggg

SEQ ID NO: 123 - DNA, 60 bp, Artificial
  Universal oligo
tctgttgcttaaatgacgctcttggtgaacctgtggtgaaaacccgagtctctaaaacga

SEQ ID NO: 124 - DNA, 60 bp, Artificial
  Universal oligo
tcctatagtgtggtattacttctgctagaatcttgtagacttctttttggacggaagctc

SEQ ID NO: 125 - DNA, 60 bp, Artificial
  Universal oligo
ggttcttgcgatgggggccagaaatacattgctcttctctcgtcgttgtggtaaaacgga

SEQ ID NO: 126 - DNA, 60 bp, Artificial
  Universal oligo
ctggtaaactgactaatagttggtggctaaggtgctacttatttgtccgctgtatggtcc

SEQ ID NO: 127 - DNA, 60 bp, Artificial
  Universal oligo
ttggaatttccttgtgaacccagcttagataacaatgatagggatgtcagcggctagata

SEQ ID NO: 128 - DNA, 60 bp, Artificial
  Universal oligo
gttttggccagttggaacattatcatcctatgctgaagattgtatgctgtatcactatac

SEQ ID NO: 129 - DNA, 60 bp, Artificial
  Universal oligo
gcatgaacttttctcccctttcttctcagccttcttctaggtagacatccctgttatcac

SEQ ID NO: 130 - DNA, 60 bp, Artificial
  Universal oligo
gcggaaaatgtagttgtcatgcgctttaaccaggtgggtgttctagtgcggttgtagtca

SEQ ID NO: 131 - DNA, 60 bp, Artificial
  Universal oligo
gaggtcgattagtccatatttaatactgtcagctttagcttgtcccgtgagtacacccac

SEQ ID NO: 132 - DNA, 60 bp, Artificial
  Universal oligo
caagtcattttcctgtgcattcgggtattctcataatgtgtggttaaagatcgttatctg

SEQ ID NO: 133 - DNA, 60 bp, Artificial
  Universal oligo
aattgattctgagaactactgcccggaattggttttactcctagtctggtatcgccgtat

SEQ ID NO: 134 - DNA, 60 bp, Artificial
  Universal oligo
gaaaccgttcatagaaaacagctaccaagttgtgacgatttgaataccatcagttaagaa

SEQ ID NO: 135 - DNA, 60 bp, Artificial
  Universal oligo
gtcccgaaggagacatttgtcgaaggatcaggtttgtggtattaggctaactatatgatg

SEQ ID NO: 136 - DNA, 60 bp, Artificial
  Universal oligo
gagagtactgtcttggctattgttatgtgtcgtatatgacctagagctaaaggcaagcct

SEQ ID NO: 137 - DNA, 60 bp, Artificial
  Universal oligo
caacttcaccttgaacagcctagaacataatgtgagtttctttccgattggtgggattcc

SEQ ID NO: 138 - DNA, 60 bp, Artificial
  Universal oligo
ccatactatgtccccctcgaactgataatctaaaggaggagtgggagactgagtgagtga

SEQ ID NO: 139 - DNA, 60 bp, Artificial
  Universal oligo
cgatttgagactccaggacatgcaggctacccttttatgccaaccggaaggaagatactg

SEQ ID NO: 140 - DNA, 60 bp, Artificial
  Universal oligo
tggagtggaacacacaaaaataggtgaatgctctgagccttttaactggatgttttatca

SEQ ID NO: 141 - DNA, 60 bp, Artificial
  Universal oligo
tctgccctcagaacccaacaagttaaaaatggatatgcactcaataggataaattagggg

SEQ ID NO: 142 - DNA, 60 bp, Artificial
  Universal oligo
actgatttacgtgaatgcacatccgagtctggttcgtgagttagaggtttgtagagggtg

SEQ ID NO: 143 - DNA, 60 bp, Artificial
  Universal oligo
ccaatctgtgtaggtaagttctgatgggggtttttgggtgggatactttcgtctcacatt

SEQ ID NO: 144 - DNA, 60 bp, Artificial
  Universal oligo
ggcctgaatttggacatcctgaagatcaccctgatttctttgggtatcaagcagcaaaac

SEQ ID NO: 145 - DNA, 60 bp, Artificial
  Universal oligo
cgataatgcagcacctaattgcgcgatcagtcccatataagggcacatagaaagtgtact

SEQ ID NO: 146 - DNA, 60 bp, Artificial
  Universal oligo
tccagaactgagtgtaataatgaggggcgcaactgaatttgtaactggggaaggatttca

SEQ ID NO: 147 - DNA, 60 bp, Artificial
  Universal oligo
ttaactattggactgatgtagagacggtgagccctatgtgtcctaaccttggtgattgtc

SEQ ID NO: 148 - DNA, 60 bp, Artificial
  Universal oligo
acggtgtttctatcttcgctacataactttatacccacagactaacaagccagcttacgc

SEQ ID NO: 149 - DNA, 60 bp, Artificial
  Universal oligo
tcaccgtcacagactggagcacgtacacacctaatgatgtcactgggacgaccttttgtc

SEQ ID NO: 150 - DNA, 60 bp, Artificial
  Universal oligo
atacaagatcctaaaccattgattcgggtgtaccacactggaaagaaaaatactgtgatt

SEQ ID NO: 151 - DNA, 60 bp, Artificial
  Universal oligo
ggcgacgataaaggatgatacgaaaaacggtcttggacgggaggctgttagaattgcggt

SEQ ID NO: 152 - DNA, 60 bp, Artificial
  Universal oligo
cgggtagtgcattatgtcttatcactctttgggtcctcatgccaatcctggaatggtttc

SEQ ID NO: 153 - DNA, 60 bp, Artificial
  Universal oligo
ctcagtggaaagaagatgccaaccaaagttattaagtctaatcaattcgagcctatgggg

SEQ ID NO: 154 - DNA, 60 bp, Artificial
  Universal oligo
ttctacacgtcacccaccccaaggttaagactcggtcggtaagataccatgtggtcacct

SEQ ID NO: 155 - DNA, 60 bp, Artificial
  Universal oligo
acagatgagtttggaggtcattaagagtagaaggtccttgttttacagtattcagcgagg

SEQ ID NO: 156 - DNA, 60 bp, Artificial
  Universal oligo
caacagtaagctatcttaaactcttgtaccagctactctgtacctcatcgcaggtcgata

SEQ ID NO: 157 - DNA, 60 bp, Artificial
  Universal oligo
tttcggtgatagcaaccacaacgtacttcttacactaatactctaacagtgaaggctagg

SEQ ID NO: 158 - DNA, 60 bp, Artificial
  Universal oligo
agaagtaatactgagcctgcccgatttatttcctgaatgaacaaaaactgacaccgaggg

SEQ ID NO: 159 - DNA, 60 bp, Artificial
  Universal oligo
atgcctcacgataaaaagttgggacggtggtaatgatcctaaggctgcatctacccatgc

SEQ ID NO: 160 - DNA, 60 bp, Artificial
  Universal oligo
gccaagcaatcggggattacaggtccactcgttcccggaatttgggtcacacatagcatc

SEQ ID NO: 161 - DNA, 60 bp, Artificial
  Universal oligo
ctgtcgctgtataaggagaccatcgccagaagaaattatgcaaattgacgagtagtgtga

SEQ ID NO: 162 - DNA, 60 bp, Artificial
  Universal oligo
cagtctctcgataagccgataaattctccacacaaccaaaagaggtgattattccggttt

SEQ ID NO: 163 - DNA, 60 bp, Artificial
  Universal oligo
ccaagaaatgttgggttggcccggcttaaaaacgatgtgatatagctcaatagatccatt

SEQ ID NO: 164 - DNA, 60 bp, Artificial
  Universal oligo
tgtcccacctatgtcctcagcagaagagatatgtccaccccctaaaacagaggcatccat

SEQ ID NO: 165 - DNA, 60 bp, Artificial
  Universal oligo
taattctttcggccttaattccaaggtacgtctcagctcccttccatacagctataccct

SEQ ID NO: 166 - DNA, 60 bp, Artificial
  Universal oligo
atccttaaacaaagacccaaaacttaatggaatagcagagggatcacactacaaacttca

SEQ ID NO: 167 - DNA, 60 bp, Artificial
  Universal oligo
gttcggccaagaaaagacgacgggtactcagaacgacgcgaaaaaccttgaataaaatgc

SEQ ID NO: 168 - DNA, 60 bp, Artificial
  Universal oligo
ttagcgatgtgtaccattcaacgtgggtgaagggttgttggaatctagtggacaggggaa

SEQ ID NO: 169 - DNA, 60 bp, Artificial
  Universal oligo
ttgatcatttgataccctgccggatgagaggatcgaatgcagcgttcttgctatggtctc

SEQ ID NO: 170 - DNA, 60 bp, Artificial
  Universal oligo
cgcaatacaacctacccgaatttatgaaccctcctccagacgccaatccatcgcccgcat

SEQ ID NO: 171 - DNA, 60 bp, Artificial
  Universal oligo
ctttgcaagaaaatacttctgattaaacaatcccttggctaacctaccgattaagaaacg

SEQ ID NO: 172 - DNA, 60 bp, Artificial
  Universal oligo
aattctggttagctgcttcatctggcacataagacttcacctccaccaccacgaagacaa

SEQ ID NO: 173 - DNA, 60 bp, Artificial
  Universal oligo
ttaaattgttggaaagaggctcacctatactgggcagttactcagttcaccattttctta

SEQ ID NO: 174 - DNA, 60 bp, Artificial
  Universal oligo
acggtacctcaagcatttacttttctttttaaccaaaattcactgacgataactcacagg

SEQ ID NO: 175 - DNA, 60 bp, Artificial
  Universal oligo
accatcctgtacttgtccttgttccctttatcaaactcatgttctgatgaacgtcttact

SEQ ID NO: 176 - DNA, 60 bp, Artificial
  Universal oligo
gccccttgaaactgtctttaaggcgtctcaccaagattctgatccttgagcatctgaact

SEQ ID NO: 177 - DNA, 60 bp, Artificial
  Universal oligo
cacgcttgtaaaccccaatacgacctggactgataccagagattgcggaataatataatc

SEQ ID NO: 178 - DNA, 60 bp, Artificial
  Universal oligo
gaaactaatgttgttttaataaccaatagggcttcggcggaggagtatgtacttagagtt

SEQ ID NO: 179 - DNA, 60 bp, Artificial
  Universal oligo
tgtacgcaatcgttttctccggcagaagacttcgtggttgcattgaaagcggctatttac

SEQ ID NO: 180 - DNA, 60 bp, Artificial
  Universal oligo
gtatacgtgactcattagctcaataaatataaagcggtcgtcagtgtcatcttagtctat

SEQ ID NO: 181 - DNA, 60 bp, Artificial
  Universal oligo
caaatgtgtgagttctgcaacgcccaggacagaggttgggtatgatgtggcctttttgat

SEQ ID NO: 182 - DNA, 60 bp, Artificial
  Universal oligo
ccattcttgattaggatgacgaaaggaatagaaacaacaccagaggtaattgccgaagag

SEQ ID NO: 183 - DNA, 60 bp, Artificial
  Universal oligo
gaggtaaataagacatgacccagtggcagaccgtttgttcggggttcattagtggctgtg

SEQ ID NO: 184 - DNA, 60 bp, Artificial
  Universal oligo
gagaaacagtcgtatctgacttaccgagaagacttgtcataactgcccctctcctcacaa

SEQ ID NO: 185 - DNA, 60 bp, Artificial
  Universal oligo
ttcttaaccatacctactaatctaaacagaaacatcgagatatacatccggtgaaccaga

SEQ ID NO: 186 - DNA, 60 bp, Artificial
  Universal oligo
catctggggaggtgtgctgcgatagagagagataatttaacgaactcagaaaacactggt

SEQ ID NO: 187 - DNA, 60 bp, Artificial
  Universal oligo
taccctcgtaggatgcgttctggcagctttatgggttccaaatgctcttaacaagcagag

SEQ ID NO: 188 - DNA, 60 bp, Artificial
  Universal oligo
aggtcatgaggctacaactttcttgatagtcccccgtactacagatttgtctcttgccgg

SEQ ID NO: 189 - DNA, 60 bp, Artificial
  Universal oligo
tatcggaataagacgttcactttcgtactcaggccccggttgagcacaccacttctattt

SEQ ID NO: 190 - DNA, 60 bp, Artificial
  Universal oligo
actgactctatcggctgaggtcactgatcttactccacgacccaacaacagcacgaacac

SEQ ID NO: 191 - DNA, 60 bp, Artificial
  Universal oligo
gtattgcccattaagccttgatactgggaccctggggaacaacattccataaagttgcac

SEQ ID NO: 192 - DNA, 60 bp, Artificial
  Universal oligo
ttgcttcatcgagtagttcgttggtctgcttggttagggtttctagtagggagactggaa

SEQ ID NO: 193 - DNA, 60 bp, Artificial
  Universal oligo
cacgcttcgtcatagacactaccatatcgccatgaatcggaaataagaattatctccgcc

SEQ ID NO: 194 - DNA, 60 bp, Artificial
  Universal oligo
ggttgacactcttgctcgttcacacattgtctattatgttttccatattttccttcaccg

SEQ ID NO: 195 - DNA, 60 bp, Artificial
  Universal oligo
ttcctgaaggtggagaaaagaaagacctacagctcctagtcctaattgtgccatcgagac

SEQ ID NO: 196 - DNA, 60 bp, Artificial
  Universal oligo
caactattgccggaaacctttttataggaatggtggttgtgactctgatgtcattatgat

SEQ ID NO: 197 - DNA, 60 bp, Artificial
  Universal oligo
cgggagatcgtgcatgagctaatcgtccttggcctagcaatacttcaaaagggctgaatc

SEQ ID NO: 198 - DNA, 60 bp, Artificial
  Universal oligo
tgggcagttatatcaaactactctcatacaattcataccccaaactcctgcgtcgggacg

SEQ ID NO: 199 - DNA, 60 bp, Artificial
  Universal oligo
gtaggagtctcataccaactaatctgcggatatgggcaatagcatcaaaacgggggtcaa

SEQ ID NO: 200 - DNA, 60 bp, Artificial
  Universal oligo
agctctcggggttgattaactaataaaccttcctttgtgtccgatactataaagacagcc